(19) United States
(12) Patent Application Publication
(10) Pub. No.: US 2012/0022116 A1
(43) Pub. Date: Jan. 26, 2012

(54) COMPOSITIONS AND METHODS FOR THE TREATMENT OF PATHOLOGICAL CONDITION(S) RELATED TO GPR35 AND/OR GPR35-HERG COMPLEX

(76) Inventors: Huayun Deng, Corning, NY (US); Ye Fang, Painted Post, NY (US); Mingqian He, Horseheads, NY (US); Haibei Hu, Corning, NY (US); Weijun Niu, Painted Post, NY (US); Haiyan Sun, Baltimore, MD (US)

(21) Appl. No.: 13/182,019

(22) Filed: Jul. 13, 2011

Related U.S. Application Data
(60) Provisional application No. 61/365,861, filed on Jul. 20, 2010.

(51) Int. Cl. A61K 31/44; C12Q 1/02; A61K 31/341; C07D 495/04; A61K 31/381; C07D 333/40; C07D 409/04; C07C 229/56; A61K 31/196; C07C 229/58; C07D 213/74; C07D 307/66; C07D 333/36; C07D 207/34; A61K 31/40; C07C 255/42; A61K 31/277; C40B 30/06; A61P 29/00; A61P 3/10; A61P 9/06; A61P 3/00; A61P 9/00; A61P 35/00; A61P 3/06; A61P 3/04; A61P 9/10; A61P 9/12; G01N 21/64; C07D 307/68

(52) U.S. Cl. 514/352; 549/474; 435/29; 514/461; 549/50; 514/443; 549/71; 514/448; 549/59; 562/457; 514/444; 514/567; 514/563; 546/312; 549/480; 514/472; 549/68; 514/447; 548/557; 514/426; 558/409; 514/523; 506/10; 435/7.21

(57) ABSTRACT

Disclosed are compositions and methods for the prevention and/or treatment of diseases which are pathophysiologically related to GPR35, and/or GPR35-hERG signaling complex. For example, disclosed are compounds for preventing and/or treating diseases which are pathophysiologically related to GPR35 in a subject. The compounds having a formula (I), (II) or (III):

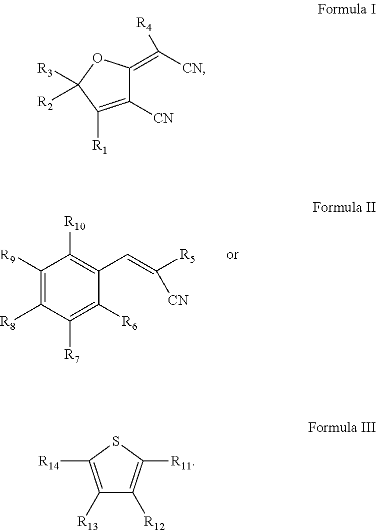

### CROSS-REFERENCE TO RELATED APPLICATIONS

This application claims the benefit of priority under 35 U.S.C. §119 of U.S. Provisional Application No. 61/365,861 filed on Jul. 20, 2010 the content of which is relied upon and incorporated herein by reference in its entirety.

### SEQUENCE LISTING

This application contains a Sequence Listing electronically submitted via EFS-Web to the United States Patent and Trademark Office as text filed named “20110712_SP10_{—}204_ST25.txt” having a size of 37,594 bytes and created on Jul. 13, 2011. Due to the electronic filing of the Sequence Listing, the electronically submitted Sequence Listing serves as both the paper copy required by 37 CFR §1.821(c) and the CRF required by §1.821(e). The information contained in the Sequence Listing is hereby incorporated herein by reference and does not go beyond the disclosure in the International Application as filed.

### BACKGROUND

G protein-coupled receptors (GPCRs) have been, and continue to be, one of the richest families of drug targets. There are at least two key drivers for this. The first driver is the increasing numbers of orphan receptors being deorphanized, some of which have implications for human diseases. Examples are GPR3 for Alzheimer's disease and GPR40 for diabetes. The second driver is associated with the recent realization that GPCRs are competent to elicit a rich array of cell signaling pathways (i.e., pleiotropic signaling), and ligands may give operational biases to activate the receptor. These pathway biased ligands may open new revenues for drug discovery.

GPR35 is a rhodopsin-like GPCR first identified in 1998 [B. F. O'Dowd, T. Nguyen, A. Marchese, R. Cheng, K. R. Lynch, H. H. Heng, L. F. Kolakowski Jr, S. R. George (1998) Discovery of three novel G-protein-coupled receptor genes, Genomics 47: 310-313]. The human GPR35 gene encodes a protein of 309 amino acids. GPR35 is expressed in various mammalian tissues, such as the gastrointestinal tissues, lymphoid tissues and the central and peripheral nervous tissues.

Several investigators have reported GPR35 to be involved in the development of gastric cancer [S. Okumura, H. Baba, T. Kumada, K. Nanmoku, H. Nakajima, Y. Nakane, K. Hioki, K. Ikenaka (2004) Cloning of a G-protein-coupled receptor that shows an activity to transform NIH3T3 cells and is expressed in gastric cancer cells, Cancer Sci. 95: 131-135], the regulation of neuronal excitability and synaptic release [J. Guo, D. J. Williams, H. L. Puhl III, S. R. Ikeda (2008) Inhibition of N-type calcium channels by activation of GPR35, an orphan receptor, heterologously expressed in rat sympathetic neurons, J. Pharmacol. Exp. Ther. 324: 342-351], nociception [H. Ohshiro, H. Tonai-Kachi, K. Ichikawa (2008) GPR35 is a functional receptor in rat dorsal root ganglion neurons, Biochem. Biophys. Res. Commun. 365: 344-348.], the pathogenesis of brachydactyl)-mental retardation syndrome [A. E. Shrimpton, B. R. Braddock, L. L. Thomson, C. K. Stein, J. J. Hoo (2004) Molecular delineation of deletions on 2q37.3 in three cases with an Albright hereditary osteodystrophy-like phenotype, Clin. Genet. 66: 537-544], and the regulation of blood pressure [K. D. Min, M. Asakura, Y. Liao, K. Nakamaru, H. Okazaki, T. Takahashi, K. Fujimoto, S. Ito, A. Takahashi, H. Asanuma, S. Yamazaki, T. Minamino, S. Sanada, O. Seguchi, A. Nakano, Y. Ando, T. Otsuka, H. Furukawa, T. Isomura, S. Takashima, N. Mochizuki, M. Kitakaze (2010) Identification of genes related to heart failure using global gene expression profiling of human failing myocardium, Biochem. Biophys. Res. Commun. 393: 55-60.].

To date, there are four agonists for GPR35 reported so far, including kynurenic acid, NPPB, zaprinast, and lysophosphatidic acid (LPA). Both kynurenic acid and LPA were speculated to be an endogenous ligand for GPR35 [J. Wang, N. Simonavicius, X. Wu, G. Swaminath, J. Reagan, H. Tian, L. Ling, (2006) Kynurenic acid as a ligand for orphan G protein-coupled receptor GPR35, J. Biol. Chem. 281: 22021-22028; S. Oka, R. Ota, M. Shima, A. Yamashita, T. Sugiura (2010) GPR35 is a novel lysophosphatidic acid receptor. Biochem. Biophys. Res. Comm. 395: 232-237]. Both kynurenic acid and LPA elicited several cellular responses in HEK293 cells and/or CHO cells expressing GPR35. For example, in HEK-293 cells expressing GPR35, 2-acyl LPA markedly enhanced the Ca¹⁺ response, the activation of RhoA and the phosphorylation of ERK in GPR35-expressing cells. 2-Acyl LPA also induced the internalization of the receptor molecule. Nevertheless, it remains unclear whether kynurenic acid or LPA is the natural agonist for GPR35.

The hERG gene encodes the pore-forming α subunit of a voltage gated potassium channel (Kv11.1). HERG channels are expressed in various tissues including cardiac myocytes, neurons, pancreatic β cells, smooth muscles and some cancer cells. Currently, hERG is best known as the major component of the delayed rectifier current Iₖᵣ in the heart which is important for the action potential repolarization. Genetic mutations in hERG channel have been known to cause the inherited long QT syndrome (LQT), a disease which may result in patient sudden death. Drugs that can block hERG current, or inhibit hERG channel protein trafficking, may cause the acquired LQTs. Conversely, mutations of hERG channel protein were reported to cause short QT syndrome.

Besides playing a critical role in cardiac myocytes, increasing evidence has shown that hERG channel expression level was elevated in several types of cancer cells including leukemia, colon cancer, gastric cancer, breast cancer and lung cancer cells. It is not clear why the hERG channel is overexpressed in cancer cells, but it is suggested that hERG channel may play a role in cancer cell proliferation.

HERG channel has a unique pore region that can accommodate structurally diverse channel blockers. A comparatively large inner cavity and the presence of particular aromatic amino acid residues (Y652 and F656) on the inner (S6) helices of the channel are important features that allow hERG to accommodate and bind disparate drugs. In addition to the various hERG channel blockers, seven hERG channel activators have been identified, including RPR260243, NS1643, NS3623, PD-118057, PD-307243, mallotoxin and A-935142 (see Su, Z., et al. Biochem Pharm 77:1383, 2009). These hERG activators have diverse chemical structures and enhance the hERG channel activity by different mechanisms. Among them, mallotoxin (MTX) and A-935142 can shift the voltage dependent channel activation to less depolarized voltages. Electrophysiology studies showed that 10 μM MTX could shift the half maximal activation voltage (V_{1/2}) to the hyperpolarizing direction for more than 25 mV.

hERG channels have been shown to form signaling complexes with a few other receptors, including beta1 integrin receptor and VEGFR-1 (FLT-1), in certain types of cells (e.g., Pillozzi, S. et al., (2007) Blood. 110: 1238-1250). However, there is no report in the literature suggesting that hERG and GPCRs including GPR35 can physically interact with each other to form signaling complexes.

There is a strong need for new drug therapies for the treatment of subjects suffering from or susceptible to pathological conditions or diseases associated with GPR35, hERG or a GPR35-hERG complex. In particular, a need still exists for new drugs having one or more improved properties (such as safety profile, efficacy, or physical properties) relative to those currently available.

### SUMMARY

Disclosed are compositions and methods for the prevention and/or treatment of diseases which are pathophysiologically related to GPR35 and/or GPR35-hERG complex. For example, disclosed are a class of compounds, including the pharmaceutically acceptable salts of the compounds, having a formula (I), (II) or (III):

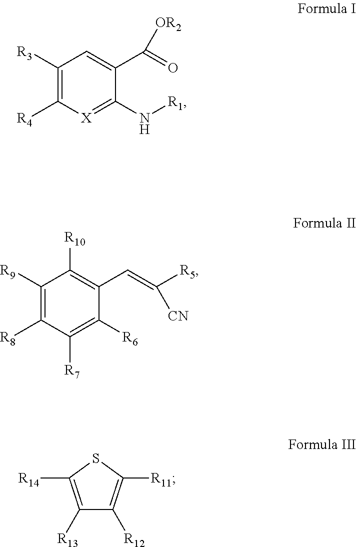

wherein:
X is C or N
R₁, R₂, R₃ and R₄ are each independently selected from a group consisting of hydrogen, halogen, cyano, —NO₂, —OR¹⁰¹, alkyl, alkenyl, alkynyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰²; or R₃ and R₄, together with the adjacent carbon atoms of the ring, form an fused or non-fused mono, bicyclic or tricyclic heterocyclic or carbocyclic ring which is optionally independently substituted with one or more substituents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰².

R₅ is CN, —C(O)NR¹⁰¹R¹⁰², —C(O)R¹⁰¹, —C(O)OR¹⁰¹, —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹, R¹⁰² or

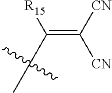

wherein R₁₅ is amino, alkylamino, dialkylamino, alkyl, hydroxy, cyano, or nitro;

R¹⁰¹ and R¹⁰² are each independently selected from the group consisting of hydrogen, alkyl, alkenyl, alkynyl, cycloalkyl, aryl, heterocycloalkyl and heteroaryl; wherein each R¹⁰¹ and R¹⁰² alkyl, alkenyl, alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;

R₆, R₇, R₈, R₉ and R₁₀ are each independently selected from a group consisting of hydrogen, halogen, cyano, —NO₂, —OR¹⁰¹, alkyl, alkenyl, alkynyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰²; or any two of R₆, R₇, R₈, R₉ and R₁₀, together with the adjacent carbon atoms of the phenyl ring, form an fused or non-fused mono, bicyclic or tricyclic heterocyclic or carbocyclic ring which is optionally independently substituted with one or more substituents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², NR¹⁰¹R¹⁰², —NR¹⁰¹S(O)₂R¹⁰², —NR¹⁰¹C(O)R¹⁰², —S(O)₂R¹⁰², —SR¹⁰¹, S(O)₂NR¹⁰¹R¹⁰², R¹⁰¹ and R¹⁰².

R₁₁, R₁₂, R₁₃ and R₁₄ are each independently selected from a group consisting of hydrogen, halogen, cyano, —NO₂, —OR¹⁰¹, alkyl, alkenyl, alkynyl, cycloalkyl, cycloalkenyl, aryl, alkylaryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰²;

wherein each of R₁, R₂, R₃, R₄, R₅, R₆, R₇, R₈, R₉, R₁₀, R₁₁, R₁₂, R₁₃, R₁₄, R₁₅, R¹⁰¹ and R¹⁰² is optionally independently substituted with one or more substituents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰².

Also disclosed are methods of preventing and/or treating diseases which are pathophysiologically related to GPR35 and/or GPR35-hERG complex in a subject, comprising administering to said subject a therapeutically effective amount of a compound of formula (I), (II) or (III), or a pharmaceutically acceptable salt thereof.

Also disclosed are pharmaceutical compositions for preventing and/or treating diseases which are pathophysiologically related to GPR35 and/or GPR35-hERG complex in a subject, comprising a therapeutically effective amount of a compound of formula (I), (II) or (III), or a pharmaceutically acceptable salt thereof.

Also disclosed are methods of screening for a modulator of GPR35 and/or GPR35-hERG complex.

Also disclosed are methods to classify GPR35-hERG signaling complex modulators, and the uses of the GPR35-hERG modulators for therapeutic prevention or treatment of diseases to which the activity of GPR35-hERG signaling complexes is pathophysiologically related.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the expression and location of GPR35 in human colon cancer cell line HT-29. (a) Western blotting showing the expression of GPR35a and GPR35b, two splicing variants of GPR35 in HT29 whole cell lysates. (b) Confocal imaging showing the staining pattern of GPR35 by anti-GPR35 antibody, which GPR35 is primarily located at the cell surface.

FIG. 2 shows pharmacological characterization of GPR35 in HT29 cells using the known GPR35 agonist zaprinast. (A) Confocal imaging showing that GPR35, once activated by its agonist zaprinast, underwent internalization in HT-29 cells. The staining was achieved using anti-GPR35 antibody. (B) The dose dependent DMR response of HT29 cells upon stimulation with zaprinast. (C) The amplitudes of the positive-DMR event of the zaprinast DMR signals in HT29 as a function of zaprinast doses, which showed that zaprinast triggered a saturable DMR signal in HT29, leading to an EC₅₀ of 137±15 nM.

FIG. 3 shows pharmacological characterization of GPR35 in HT29 cells using the agonist 6-bromo-3-methylthieno[3,2-b]thiophene-2-carboxylic acid (“YE210”). (A) Confocal imaging showing that in un-permeabilized HT29 cells, a small portion of GPR35, after activated by the agonist 6-bromo-3-methylthieno[3,2-b]thiophene-2-carboxylic acid, remained at the cell plasma surface. (B) Confocal imaging showing that in the permeabilized HT29 cells, some GPR35, after activated by the agonist 6-bromo-3-methylthieno[3,2-b]thiophene-2-carboxylic acid, underwent internalization (i.e., localized inside the cells). The staining in (A) and (B) were achieved using anti-GPR35 antibody. (C) The dose dependent DMR response of HT29 cells upon stimulation with 6-bromo-3-methylthieno[3,2-b]thiophene-2-carboxylic acid. (D) The amplitudes of the positive-DMR event of the 6-bromo-3-methylthieno[3,2-b]thiophene-2-carboxylic acid DMR signals in HT29 as a function of 6-bromo-3-methylthieno[3,2-b]thiophene-2-carboxylic acid doses, which showed that 6-bromo-3-methylthieno[3,2-b]thiophene-2-carboxylic acid triggered a saturable DMR signal in HT29, leading to an EC₅₀ of 165±45 nM.

FIG. 4 shows that signaling mediated through the activation of GPR35 by its known agonist zaprinast in HT29 cell was linked to the G_{12/13}-ROCK pathway. (A) Zaprinast of 10 micromolar did not result in any detectable Ca²⁺ mobilization signal in HT29, as measured by the Fluo-4 assay. (B) The DMR signal of zaprinast of 400 nM in HT29 was insensitive to the phospholipase C inhbitior U73122 (10 micromolar). Phopsholipase C is a downstream cascade protein in G_{q}-mediated signaling. (C) The DMR signal of zaprinast of 400 nM in HT29 was also insensitive to the pretreatment of cholera toxin (CTX; 1 microgram/ml). The cells were pretreated with cholera toxin overnight before the zaprinast stimulation. (D) The DMR signal of zaprinast of 400 nM in HT29 was also insensitive to the pretreatment of Pertussis toxin (PTX; 100 nanogram/ml). The cells were pretreated with PTX overnight before the zaprinast stimulation. (E) The DMR signal of zaprinast of 400 nM in HT29 was completely attenuated by the actin filament disruption agent cytochalasin D (10 micromolar). And (F) the DMR signal of zaprinast of 400 nM in HT29 was partially attenuated by the ROCK inhibitor Y27632 (10 micromolar).

FIG. 5 shows GPR35 signaling in engineered HEK293 cells, as measured by the Fluo-4 Ca²⁺ mobilization assays. (A) The real time kinetics of engineered cells upon stimulation with the endogenous muscrunic M₃ receptor agonist carbachol (10 μM). (B) The real time kinetics of engineered cells upon stimulation with the known GPR35 agonsit zaprinast (10 μM). And (C) The real time kinetics of engineered cells upon stimulation with 6-bromo-3-methylthieno[3,2-b]thiophene-2-carboxylic acid, the GPR35 agonist identified according to the present disclosure (10 μM). The HEK293 cells were trainstly transfected, thus expressing G_{qo5} (Gₒ), GPR35 (GPR35), or both G_{qo5} and GPR35 (GPR35Gₒ), respectively. There were duplicates for each measurement, as indicated as A and B in the graph.

FIG. 6 shows a two-step Epic® cellular assay to screen GPR35 modulators. (A) An example of agonism mode (HT29 was stimulated with 6-bromo-3-methylthieno[3,2-b]thiophene-2-carboxylic acid at 10 micromolar only), wherein 6-bromo-3-methylthieno[3,2-b]thiophene-2-carboxylic acid resulted in a robust DMR signal that is similar to zaprinast, and the buffer vehicle (i.e., the negative control) did not cause any obvious DMR. (B) An example of antagonist mode, wherein the pretreatment of cells with 6-bromo-3-methylthieno[3,2-b]thiophene-2-carboxylic acid caused desensitization of the cells to sequential stimulation with zaprinast, but the buffer vehicle had little impact on the zaprinast DMR. (C) The amplitudes of the DMR signal, at 5 min post simulation, as a function of compound in the agonism mode. (D) The percentages of the zaprinast DMR signals inhibited by the pretreatment with compounds, each at 10 micromolar. Zaprinast was examined at 400 nanomolar in all experiments.

FIG. 7 shows the characterization of hERG-GPR35 signaling complexes in HT29 cells. The hERG-GPR35 oligomers can be immunoprecipitated from HT29 cells using either anti-GPR35 antibody (IP-GPR35) or anti-hERG antibody (IP-hERG). (A) Western blotting for GPR35; (B) Western blotting for hERG. Cell lysate samples obtained using immunoprecipitation with anti-IgG was used as a negative control.

FIG. 8 shows assays characterizing the GPR35-hERG signaling complexes in HT29 cells. The dose-dependent desensitization of the NPPB or zaprinast treated cells to subsequent stimulation with either zaprinast (400 nM) or NPPB (1 μM). The amplitudes of the positive-DMR event of the zaprinast DMR signals in HT29 as a function of NPPB or zaprinast doses. NPPB or Zaprinast at different doses were used to pretreat the cells.

FIG. 9 shows assays characterizing GPR35-hERG signaling complexes in engineered CHO cells (CHO-hERG) using automated patch clamping. (A) The protocol for automated patch clamping. (B) Electrophysiological recording of hERG channels. (C) The impact of zaprinast on the tail current of the hERG channel.

FIG. 10 shows assays characterizing GPR35-hERG signaling complexes in native and engineered HEK cells (HEK-hERG) using label-free biosensor cellular assays. (A) The DMR signal of HEK293 induced by zaprinast (10 μM). (B) The DMR signal of HEK-hERG cells induced by zaprinast (10 μM). (C) The impact of zaprinast (10 μM) on the hERG activator mallotoxin induced DMR signal in HEK-hERG cells. The cells treated with buffer only (buffer) were used as a positive control.

FIG. 11 shows assays characterizing GPR35-hERG signaling complexes in HT29 cells. (A) The impact of zaprinast (10 μM) on the mallotoxin (16 μM) DMR signal in HT29 cells. (B) The impact of mallotoxin (10 μM) on the zaprinast (400 nM) DMR signal in HT29 cells. The cells treated with buffer only (buffer) were used as a positive control.

FIG. 12 is a schematic drawing of a hERG-GPR35 signaling complex. The receptor signaling complex is formed and located at the plasma membrane of cells. The activation of hERG channel by a hERG ligand leads to signaling A, whereas the activation of GPR35 by a GPR35 agonist leads to signaling B. The activation of the hERG-GPR35 signaling complex by a hERG-GPR35 agonist leads to signaling C.

FIG. 13 is a diagram representing the classification of hERG-GPR35 signaling complex modulators. From left to right: (1) A hERG-GPR35 complex activator that is a GPR35 agonist and also a hERG activator (hERG-GPR35 complex activator). (2) A functionally selective GPR35 agonist that is able to transactivate hERG channel (hERG transactivating GPR35 agonist). (3) A functionally selective hERG activator that is able to transactivate GPR35 (GPR35 transactivating hERG activator). (4) A GPR35-specific agonist that is not able to transactivate hERG (hERG non-transactivating GPR35 agonsit). (5) A hERG specific activator that is not able to transactivate GPR35 (GPR35 non-transactivating hERG activator).

FIG. 14 shows GPR35 signaling in engineered HEK293 cells, as measured by the Fluo-4 Ca²⁺ mobilization assays. The representative real time kinetics of engineered cells upon stimulation with the known GPR35 agonist zaprinast (10 μM). The HEK293 cells were transiently transfected, thus expressing G_{qo5}, GPR35, or both G_{qo5} and GPR35, respectively.

FIG. 15 shows the dose-dependent DMR signals of HT29 cells in response to the GPR35 agonist zaprinast (A) and the hERG activator mallotoxin (B).

FIG. 16 shows that the GPR35 agonist zaprinast dose-dependently, partially attenuated the hERG activator mallotoxin-induced DMR signal in HT29 cells. Mallotoxin of 16 micromolar was used. Zaprinast of different doses was used to pretreat the cells.

FIG. 17 shows the hERG activator mallotoxin dose-dependently, partially inhibited the GPR35 agonist zaprinast-induced DMR signal in HT29 cells. Zaprinast of 400 nanomolar was used. Mallotoxin of different doses was used to pretreat the cells.

### DETAILED DESCRIPTION

Before the present compounds, compositions, articles, devices, and/or methods are disclosed and described, it is to be understood that they are not limited to specific synthetic methods or specific treatment methods unless otherwise specified, or to particular reagents unless otherwise specified, as such may, of course, vary. It is also to be understood that the terminology used herein is for the purpose of describing particular embodiments only and is not intended to be limiting.

### BACKGROUND

### A. Voltage-Dependent Ion Channels

Voltage-dependant ion channels are proteins that span cell surface membranes in excitable tissue such as heart and nerves. Ions passing through channels form the basis of the cardiac action potential Influx of Na⁺ and Ca²⁺ ions, respectively, control the depolarizing upstroke and plateau phases of the action potential. K⁺ ion efflux repolarizes the cell membrane, terminates the action potential, and allows relaxation of the muscle. A rapid component of the repolarizing current flows through the K⁺ channel encoded by the human ether-à-go-go-related gene (hERG). Impaired repolarization can prolong the duration of the action potential, delay relaxation and promote disturbances of the heartbeat. Action potential prolongation is detected clinically as a lengthening of the QT interval measured on the electrocardiogram (ECG). Drug-induced QT prolongation is a serious complication of drugs due to impaired repolarization, which is associated with an increased risk of lethal ventricular arrhythmias. Drug-induced QT prolongation is almost always associated with block of the hERG K⁺ channel. A plethora of drugs, such as methanesulfonanilides, dofetilide, MK-499, and E-4031 are known to block K⁺ ion channels such as hERG on the heart causing a life threatening ventricular arrhythmia and heart attack in susceptible individuals. Unfortunately, incidence of drug-induced ventricular arrhythmia is often too low to be detected in clinical trials.

1. hERG

The KCNH2 or human-ether-'à-go-go Related Gene (hERG) encodes Kv11.1 α-subunits that combine to form Kv11.1 potassium channels. The hERG gene is translated as a core-glycosylated immature 135 kDa protein (Kv11.1) in the endoplasmic reticulum and is converted to a complexly-glycosylated mature 155 kDa protein in the Golgi apparatus. Warmke et al. (Proc. Natl. Acad. Sci. 1994. 91(8), 3438-3442; incorporated by reference) discloses the sequence and structure of the hERG gene and its wild type translation product, Kv11.1. The sequence of hERG protein is disclosed in (NP_{—}00229). The sequence of hERG gene is disclosed in (NM_{—}00238).

A sudden death due to the blocking of hERG channels by noncardiovascular drugs such as terfenadine (antihistamine), astemizole (antihistamine), and cisapride (gastrokinetic) led to their withdrawal from the market. Recently, drugs like Vioxx, Celebrex and Bextra were also pulled out of the market for concerns relating to dangerous cardiac side effects. Consequently, cardiac safety relating to K⁺ channels has become a major concern of regulatory agencies. In order to prevent costly attrition, it has therefore become a high priority in drug discovery to screen out inhibitory activity on hERG channels in lead compounds as early as possible.

Current methods for testing potential drug molecules for hERG blocking activity have several limitations. Technologies based on cell-based patch clamp electrophysiology or animal tests are technically difficult and do not meet the demand for throughput and precision for preclinical cardiac safety tests. Other assays use radio-labeled, fluorescent, dye-conjugated, or biotinylated markers for detection and quantification of binding. However, many of these markers have reduced activity after labeling. In addition, the use of radio-labeled analogs poses practical limitations such as requirements for complex infrastructure and licenses for operating radioactive compounds. The promiscuous nature of this channel, referred to herein as the hERG K⁺ channel, or hERG, or hERG ion channel, or hERG channel, leads to it binding a diverse set of chemical structures (Cavalli, A et al., J Med Chem 2002, 45(18), 3844-53), coupled with the potential fatal outcome that may emerge from that interaction. These realities have resulted in the recommendation from the International Congress of Harmonization and the U.S. Food and Drug Administration that all new drug candidates undergo testing in a functional patch-clamp assay using the human hERG protein, either in native form or expressed in recombinant form (Bode, G., et al., Fundam Clin Pharmacol 2002, 16(2), 105-18). Although automated, high-throughput patch-clamp methods have been recently developed, such systems require specialized operators, live cells, and a substantial capital investment (Bridgland-Taylor, M. et al., J. Pharmacol. Toxicol. Methods 2006, 54(2), 189-99; Dubin, A. et al., J. Biomol. Screen. 2005, 10, (2), 168-81). More significantly, these electrophysiology approaches are limited to measure ion flux and membrane potential, but not the possible functional consequences of hERG modulations by small molecules. Accordingly, there is a need to develop new compositions and methods for characterizing and quantifying the binding of molecules, such as drug candidates, to hERG channels.

### B. G Protein-Coupled Receptors (GPCRs)

G protein coupled receptors are intrinsic membrane proteins which comprise a large superfamily of receptors. The family of G protein-coupled receptors (GPCRs) has at least 250 members (Strader et al. FASEB J., 9:745-754, 1995; Strader et al. Annu. Rev. Biochem., 63:101-32, 1994). It has been estimated that one percent of human genes may encode GPCRs. Many GPCRs share a common molecular architecture and common signaling mechanism. Historically, GPCRs have been classified into six families, originally thought to be unrelated, three of which are found in vertebrates. Recent work has identified several new GCPR families and suggested the possibility of a common evolutionary origin for all of them.

One characteristic feature of most GPCRs is that seven clusters of hydrophobic amino acid residues, or transmembrane regions (TMs, the 7 transmembrane (7_{TM}) regions are designated as TM1, TM2, TM3, TM4, TMS, TM6, and TM7) are located in the primary structure and pass through (span) the cell membrane at each region thereof. The domains are believed to represent transmembrane alpha-helices connected by three intracellular loops (i1, i2, and i3), three extracellular loops (e1, e2, and e3), and amino (N)- and carboxyl (C)-terminal domains (Palczewski et al., Science 289, 739-45 (2000)). Most GPCRs have single conserved cysteine residues in each of the first two extracellular loops which form disulfide bonds that are believed to stabilize functional protein structure. It is well known that these structures detailed above are common among G protein coupled receptor proteins and that the amino acid sequences corresponding to the area where the protein passes through the membrane (membrane-spanning region or transmembrane region) and the amino acid sequences near the membrane-spanning region are often highly conserved among the receptors. Thus, due to the high degree of homology in GPCRs, the identification of novel GPCRs, as well identification of both the intracellular and the extracellular portions of such novel members, is readily accomplished by those of skill in the art.

1. GPR35

GPR35 is a rhodopsin-like GPCR first identified in 1998 [B. F. O'Dowd, T. Nguyen, A. Marchese, R. Cheng, K. R. Lynch, H. H. Heng, L. F. Kolakowski Jr, S. R. George (1998) Discovery of three novel G-protein-coupled receptor genes, Genomics 47: 310-313]. The human GPR35 gene encodes a protein of 309 amino acids. GPR35 is expressed in various mammalian tissues, such as the gastrointestinal tissues, lymphoid tissues and the central and peripheral nervous tissues.

Several investigators have reported GPR35 to be involved in the development of gastric cancer (Okumura, S., et al., (2004) Cancer Sci, 95: 131-135), the regulation of neuronal excitability and synaptic release (J. Guo, et al., (2008) J. Pharmacol. Exp. Ther. 324: 342-351), nociception (H. Ohshiro et al., (2008) Biochem. Biophys. Res. Commun. 365: 344-348), the pathogenesis of brachydactyl-mental retardation syndrome (A. E. Shrimpton, et al., (2004) Clin. Genet. 66: 537-544), and the regulation of blood pressure (K. D. Min, et al., (2010) Biochem. Biophys. Res. Commun. 393: 55-60).

GPR35 ligands that activate GPR35 have long remained to be identified, particularly the endogenous ligands. To date, there are four agonists for GPR35 reported so far, including kynurenic acid, NPPB, zaprinast, and lysophosphatidic acid (LPA). Both kynurenic acid and LPA were speculated to be an endogenous ligand for GPR35 [J. Wang, N. Simonavicius, X. Wu, G. Swaminath, J. Reagan, H. Tian, L. Ling, (2006) Kynurenic acid as a ligand for orphan G protein-coupled receptor GPR35, J. Biol. Chem. 281: 22021-22028; S. Oka, R. Ota, M. Shima, A. Yamashita, T. Sugiura (2010) GPR35 is a novel lysophosphatidic acid receptor. Biochem. Biophys. Res. Comm. 395: 232-237]. Both kynurenic acid and LPA elicited several cellular responses in HEK293 cells and/or CHO cells expressing GPR35. For example, in HEK-293 cells expressing GPR35, 2-acyl LPA markedly enhanced the Ca²⁺ response, the activation of RhoA and the phosphorylation of ERK in GPR35-expressing cells. 2-Acyl LPA also induced the internalization of the receptor molecule. Nevertheless, it remains unclear whether kynurenic acid or LPA is the natural agonist for GPR35.

There is a strong need for new drug therapies for the treatment of subjects suffering from or susceptible to pathological conditions or diseases associated with GPR35. In particular, a need still exists for new drugs having one or more improved properties (such as safety profile, efficacy, or physical properties) relative to those currently available.

C. GPR35-hERG Complex

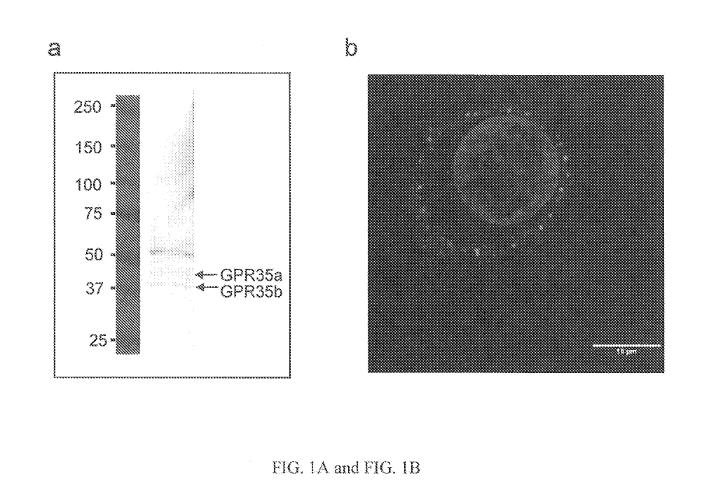

The present invention relates to hERG-GPR35 signaling complexes. The hERG-GPR35 signaling complex is a signaling complex formed between hERG and GPR35 via a physical receptor-receptor interaction, and locates at the cell plasma membrane (see FIGS. 1, 7, and 12). The signaling complex is formed in either engineered cells (e.g., HEK-hERG-GPR35 cell that recombinantly co-expresses hERG channel and GPR35), or native cells (e.g., colon cancer cell HT29, leukemia cell HL-60, or gastric cancer cell MKN45, or primary heart cells such as cardiomyctes derived from heart failure species). The cells can be animal cells or human cells. The HL-60 expresses primarily GPR35, with relatively lower expression of hERG.

### Materials

### A. Compounds

Disclosed are compounds, or pharmaceutically acceptable salts thereof, having a formula (I), (II) or (III):

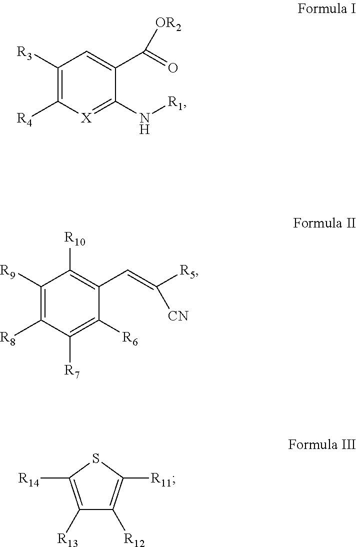

wherein:

X is C or N

R₁, R₂, R₃ and R₄ are each independently selected from a group consisting of hydrogen, halogen, cyano, —NO₂, —OR¹⁰¹, alkyl, alkenyl, alkynyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —S(O)₂NR¹⁰¹R¹⁰², R¹⁰¹ and R¹⁰²; or R₃ and R₄, together with the adjacent carbon atoms of the ring, form an fused or non-fused mono, bicyclic or tricyclic heterocyclic or carbocyclic ring which is optionally independently substituted with one or more substituents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², SR¹⁰¹, —S(O)₂NR¹⁰¹R¹⁰², —R¹⁰¹ and R¹⁰².

R₅ is CN, —C(O)NR¹⁰¹R¹⁰², —C(O)R¹⁰¹, —C(O)OR¹⁰¹, —NR¹⁰¹R¹⁰², NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹, R¹⁰² or

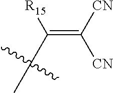

wherein R₁₅ is amino, alkylamino, dialkylamino, alkyl, hydroxy, cyano, or nitro;

R¹⁰¹ and R¹⁰² are each independently selected from the group consisting of hydrogen, alkyl, alkenyl, alkynyl, cycloalkyl, aryl, heterocycloalkyl and heteroaryl; wherein each R¹⁰¹ and R¹⁰² alkyl, alkenyl, alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;

R₆, R₇, R₈, R₉ and R₁₀ are each independently selected from a group consisting of hydrogen, halogen, cyano, —NO₂, —OR¹⁰¹, alkyl, alkenyl, alkynyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰²; or any two of R₆, R₇, R₈, R₉ and R₁₀, together with the adjacent carbon atoms of the phenyl ring, form an fused or non-fused mono, bicyclic or tricyclic heterocyclic or carbocyclic ring which is optionally independently substituted with one or more substituents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰².

R₁₁, R₁₂, R₁₃ and R₁₄ are each independently selected from a group consisting of hydrogen, halogen, cyano, —NO₂, —OR¹⁰¹, alkyl, alkenyl, alkynyl, cycloalkyl, cycloalkenyl, aryl, alkylaryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰²;

wherein each of R₁, R₂, R₃, R₄, R₅, R₆, R₇, R₈, R₉, R₁₀, R₁₁, R₁₂, R₁₃, R₁₄, R₁₅, R¹⁰¹ and R¹⁰² is optionally independently substituted with one or more substituents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰².

In some forms, the compounds as presently disclosed are compounds of formula (I), or pharmaceutically acceptable salts thereof. In some other forms, the compounds as presently disclosed are compounds of formula (II), or pharmaceutically acceptable salts thereof. In some other forms, the compounds as presently disclosed are compounds of formula (III), or pharmaceutically acceptable salts thereof.

In some forms, the compounds as presently disclosed are compounds of formula (I), or pharmaceutically acceptable salts thereof, wherein the compound of formula (I) is a compound selected from the group consisting of:

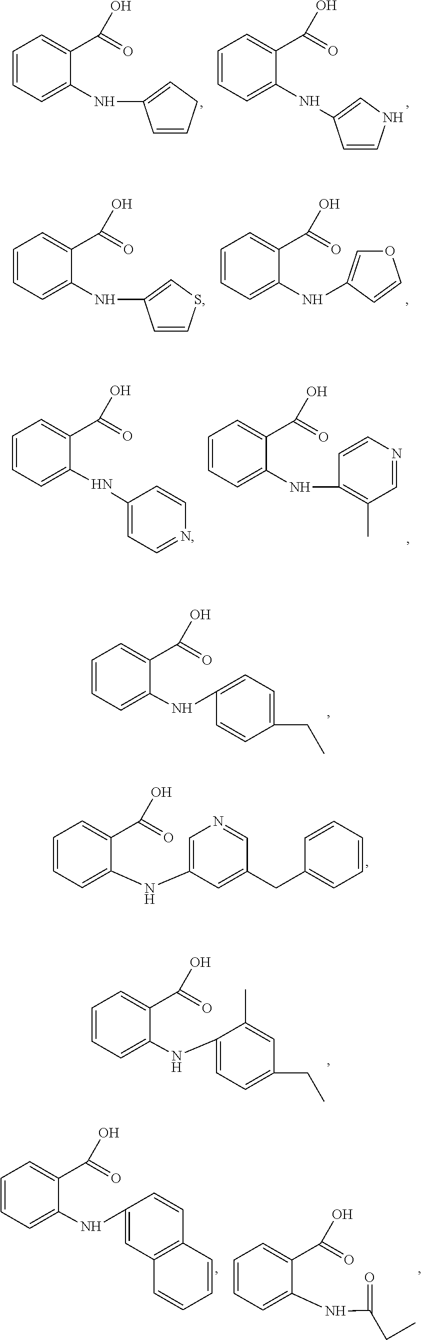

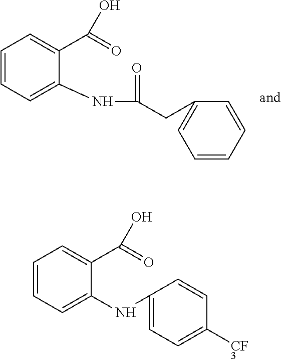

In some forms, the compounds as presently disclosed are compounds of formula (II), or pharmaceutically acceptable salts thereof, wherein the compound of formula (II) is a compound selected from the group consisting of:

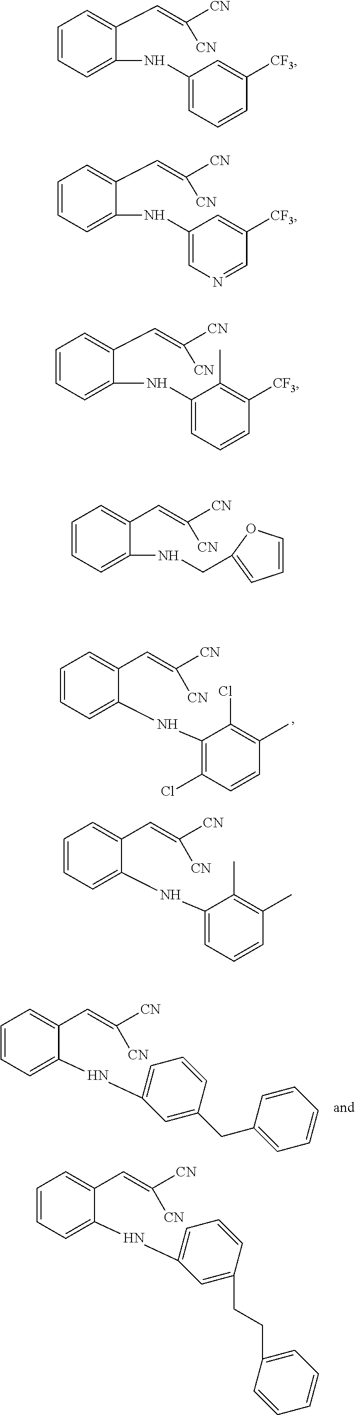

In some forms, the compounds as presently disclosed are compounds of formula (III), or pharmaceutically acceptable salts thereof, wherein the compound of formula (III) is a compound selected from the group consisting of:

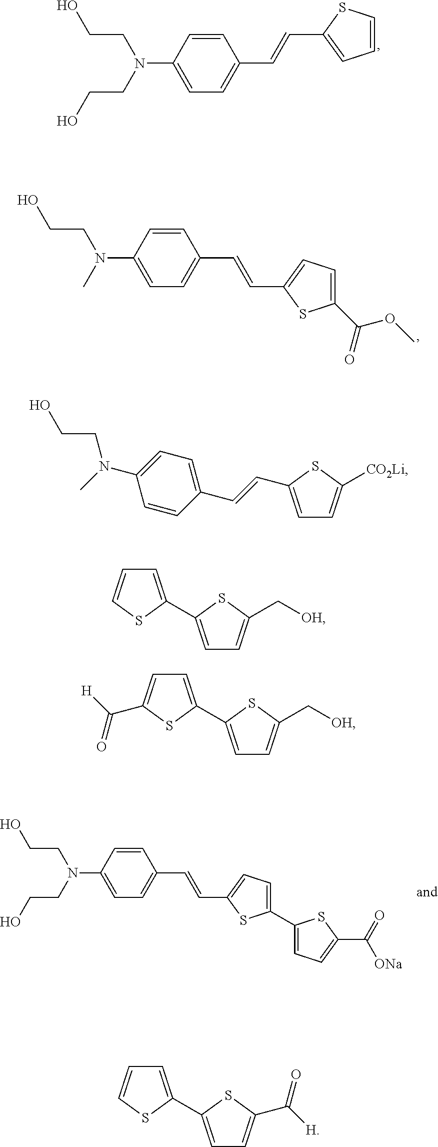

### 1. Isomers

When an asymmetric center is present in a compound of formula (I), (II) or (III), hereinafter referred to as the disclosed compounds, the compound may exist in the form of optical isomers (enantiomers). In some forms, the disclosed compounds and compositions can comprise enantiomers and mixtures, including racemic mixtures of the compounds of formula (I), (II) or (III). In some forms, for compounds of formula (I), (II) or (III) that contain more than one asymmetric center, the disclosed compounds and compositions can comprise diastereomeric forms (individual diastereomers and mixtures thereof) of compounds. When a compound of formula (I), (II) or (III) contains an alkenyl group or moiety, geometric isomers may arise.

### 2. Tautomeric Forms

The disclosed compositions and compounds comprise the tautomeric forms of compounds of formula (I), (II) or (III). Where structural isomers are interconvertible via a low energy barrier, tautomeric isomerism (‘tautomerism’) can occur. This can take the form of proton tautomerism in compounds of formula (I), (II) or (III) containing, for example, an imino, keto, or oxime group, or so-called valence tautomerism in compounds which contain an aromatic moiety. It follows that a single compound may exhibit more than one type of isomerism. The various ratios of the tautomers in solid and liquid form are dependent on the various substituents on the molecule as well as the particular crystallization technique used to isolate a compound.

### 3. Salts

The disclosed compositions and compounds can be used in the form of salts derived from inorganic or organic acids. Depending on the particular compound, a salt of the compound can be advantageous due to one or more of the salt's physical properties, such as enhanced pharmaceutical stability in differing temperatures and humidities, or a desirable solubility in water or oil. In some instances, a salt of a compound also can be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound, such as the disclosed compounds, with an acid whose anion, or a base whose cation, is generally considered suitable for human consumption. Pharmaceutically acceptable salts are particularly useful as products of the disclosed methods because of their greater aqueous solubility relative to the parent compound. For use in medicine, the salts of the disclosed compounds are non-toxic “pharmaceutically acceptable salts.” Salts encompassed within the term “pharmaceutically acceptable salts” refer to non-toxic salts of the disclosed compounds which are generally prepared by reacting the free base with a suitable organic or inorganic acid.

Suitable pharmaceutically acceptable acid addition salts of the disclosed compounds, when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclylic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Furthermore, where the disclosed compounds carry an acidic moiety, suitable pharmaceutically acceptable salts thereof can include alkali metal salts, e.g., sodium or potassium salts; alkaline earth metal salts, e.g., copper, calcium or magnesium salts; and salts formed with suitable organic ligands, e.g., quaternary ammonium salts. In some forms, base salts are formed from bases which form non-toxic salts, including aluminum, arginine, benzathine, choline, diethylamine, diolamine, glycine, lysine, meglumine, olamine, tromethamine and zinc salts.

Organic salts can be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as lower alkyl(C1-C6) halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), arylalkyl halides (e.g., benzyl and phenethyl bromides), and others. In some forms, hemisalts of acids and bases can also be formed, for example, hemisulphate and hemicalcium salts. The disclosed compounds can exist in both unsolvated and solvated forms. A “solvate” as used herein is a nonaqueous solution or dispersoid in which there is a noncovalent or easily dispersible combination between solvent and solute, or dispersion means and disperse phase.

In some forms, the pharmaceutically acceptable salts, particularly for —COOH containing compounds as presently disclosed, are in copper (II) or Zn (II) chelating forms.

### 4. Prodrugs

Also disclosed are so-called “prodrugs” of the disclosed compounds. Thus, certain derivatives of the disclosed compounds which have little or no pharmacological activity themselves can, when administered into or onto the body, be converted into the disclosed compounds having the desired activity, for example, by hydrolytic cleavage. Such derivatives are referred to as “prodrugs.” Further information on the use of prodrugs can be found in “Pro-drugs as Novel Delivery Systems, Vol. 14, ACS Symposium Series (T Higuchi and W Stella) and “Bioreversible Carriers in Drug Design,” Pergamon Press, 1987 (ed. E B Roche, American Pharmaceutical Association). Prodrugs as disclosed herein can, for example, be produced by replacing appropriate functionalities present in the compounds of formula I with certain moieties known to those skilled in the art as “pro-moieties” as described, for example, in “Design of Prodrugs” by H Bundgaard (Elsevier, 1985).

### 5. Isotopes

Also disclosed are isotopically labeled compounds, which are identical to those compounds recited in formula (I), (II), (III), (IV), (V) or (VI), but for the fact that one or more atoms are replaced by an atom having an atomic mass or mass number different from the atomic mass or mass number usually found in nature. Examples of isotopes that can be incorporated into disclosed compounds include isotopes of hydrogen, carbon, nitrogen, oxygen, phosphorous, sulfur, fluorine and chlorine, such as ²H, ³H, ¹³C, ¹⁴C, ¹⁴C, ¹⁵N, ¹⁸O, ¹⁷O, ³¹P, ³²P, ³⁵S, ¹⁸F, and ³⁶Cl, respectively. Disclosed compounds, prodrugs thereof, and pharmaceutically acceptable salts of said compounds or of said prodrugs which contain the aforementioned isotopes and/or other isotopes of other atoms are contemplated. Certain isotopically labeled disclosed compounds, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula (I), (II), (III), (IV), (V) or (VI) (and other disclosed compounds) and prodrugs thereof can generally be prepared by carrying out the procedures disclosed in the Schemes and/or in the Examples and Preparations below, by substituting a readily available isotopically labeled reagent for a non-isotopically labeled reagent.

### 6. General Synthetic Schemes

The compounds of the formula (I), (II) or (III) (and other disclosed compounds), or their pharmaceutically acceptable salts, can be prepared by the methods as illustrated by examples described in the “Examples” section, together with synthetic methods known in the art of organic chemistry, or modifications and derivatisations that are familiar to those of ordinary skill in the art. The starting materials used herein are commercially available or can be prepared by routine methods known in the art (such as those methods disclosed in standard reference books such as the COMPENDIUM OF ORGANIC SYNTHETIC METHODS, Vol. I-VI (published by Wiley-Interscience)). Preferred methods include, but are not limited to, those described below. During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference. Isolation and purification of the products is accomplished by standard procedures, which are known to a chemist of ordinary skill

### 7. Definition of Terms

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms; in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert-butyl), pentyl, iso-amyl, hexyl and the like.

The term “alkenyl” refers to a linear or branched-chain hydrocarbyl substituent containing one or more double bonds and from two to twenty carbon atoms; in another embodiment, from two to twelve carbon atoms; in another embodiment, from two to six carbon atoms; and in another embodiment, from two to four carbon atoms. Examples of alkenyl include ethenyl (also known as vinyl), allyl, propenyl (including 1-propenyl and 2-propenyl) and butenyl (including 1-butenyl, 2-butenyl and 3-butenyl). The term “alkenyl” embraces substituents having “cis” and “trans” orientations, or alternatively, “E” and “Z” orientations.

The term “benzyl” refers to methyl radical substituted with phenyl, i.e., the following structure:

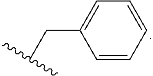

The term “carbocyclic ring” refers to a saturated cyclic, partially saturated cyclic, or aromatic ring containing from 3 to 14 carbon ring atoms (“ring atoms” are the atoms bound together to form the ring). A carbocyclic ring typically contains from 3 to 10 carbon ring atoms. Examples include cyclopropyl, cyclobutyl, cyclopentyl, cyclopentenyl, cyclopentadienyl, cyclohexyl, cyclohexenyl, cyclohexadienyl, and phenyl. A “carbocyclic ring system” alternatively may be 2 or 3 rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluorenyl, and decalinyl.

The term “heterocyclic ring” refers to a saturated cyclic, partially saturated cyclic, or aromatic ring containing from 3 to 14 ring atoms (“ring atoms” are the atoms bound together to form the ring), in which at least one of the ring atoms is a heteroatom that is oxygen, nitrogen, or sulfur, with the remaining ring atoms being independently selected from the group consisting of carbon, oxygen, nitrogen, and sulfur.

The term “cycloalkyl” refers to a saturated carbocyclic substituent having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “cycloalkyl” also includes substituents that are fused to a C₆-C₁₀ aromatic ring or to a 5-10-membered heteroaromatic ring, wherein a group having such a fused cycloalkyl group as a substituent is bound to a carbon atom of the cycloalkyl group. When such a fused cycloalkyl group is substituted with one or more substituents, the one or more substituents, unless otherwise specified, are each bound to a carbon atom of the cycloalkyl group. The fused C₆-C₁₀ aromatic ring or to a 5-10-membered heteroaromatic ring may be optionally substituted with halogen, C₁-C₆ alkyl, C₃-C₁₀ cycloalkyl, or ═O.

The term “cycloalkenyl” refers to a partially unsaturated carbocyclic substituent having three to fourteen carbon atoms, typically three to ten carbon atoms. Examples of cycloalkenyl include cyclobutenyl, cyclopentenyl, and cyclohexenyl.

A cycloalkyl or cycloalkenyl may be a single ring, which typically contains from 3 to 6 ring atoms. Examples include cyclopropyl, cyclobutyl, cyclopentyl, cyclopentenyl, cyclopentadienyl, cyclohexyl, cyclohexenyl, cyclohexadienyl, and phenyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄-C₁₀ carbocyclic ring, such as a C₅ or a C₆ carbocyclic ring, or to a 4-10-membered heterocyclic ring, wherein a group having such a fused aryl group as a substituent is bound to an aromatic carbon of the aryl group. When such a fused aryl group is substituted with one more substituents, the one or more substituents, unless otherwise specified, are each bound to an aromatic carbon of the fused aryl group. The fused C₄-C₁₀ carbocyclic or 4-10-membered heterocyclic ring may be optionally substituted with halogen, C₁-C₆ alkyl, C₃-C₁₀ cycloalkyl, or ═O. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl.

In some instances, the number of carbon atoms in a hydrocarbyl substituent (e.g., alkyl, alkenyl, cycloalkyl, cycloalkenyl, aryl, etc.) is indicated by the prefix “Cₓ—C_{y}-,” wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl refers to saturated cycloalkyl containing from 3 to 6 carbon ring atoms.

In some instances, the number of atoms in a cyclic substituent containing one or more heteroatoms (e.g., heteroaryl or heterocycloalkyl) is indicated by the prefix “X-Y-membered”, wherein x is the minimum and y is the maximum number of atoms forming the cyclic moiety of the substituent. Thus, for example, 5-8-membered heterocycloalkyl refers to a heterocycloalkyl containing from 5 to 8 atoms, including one or more heteroatoms, in the cyclic moiety of the heterocycloalkyl.

The term “hydrogen” refers to hydrogen substituent, and may be depicted as —H.

The term “hydroxy” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol.

The term “hydroxyalkyl” refers to an alkyl that is substituted with at least one hydroxy substituent. Examples of hydroxyalkyl include hydroxymethyl, hydroxyethyl, hydroxypropyl and hydroxybutyl.

The term “nitro” means —NO₂.

The term “cyano” (also referred to as “nitrile”)—CN, which also may be depicted:

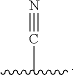

The term “carbonyl” means —C(O)—, which also may be depicted as:

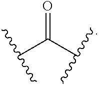

The term “amino” refers to —NH₂.

The term “alkylamino” refers to an amino group, wherein at least one alkyl chain is bonded to the amino nitrogen in place of a hydrogen atom. Examples of alkylamino substituents include monoalkylamino such as methylamino (exemplified by the formula —NH(CH₃)), which may also be depicted:

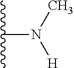

and dialkylamino such as dimethylamino, (exemplified by the formula —N(CH₃)₂), which may also be depicted:

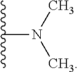

The term “aminocarbonyl” means —C(O)—NH₂, which also may be depicted as:

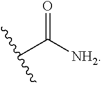

The term “halogen” refers to fluorine (which may be depicted as —F), chlorine (which may be depicted as —Cl), bromine (which may be depicted as —Br), or iodine (which may be depicted as —I). In one embodiment, the halogen is chlorine. In another embodiment, the halogen is a fluorine.

The prefix “halo” indicates that the substituent to which the prefix is attached is substituted with one or more independently selected halogen substituents. For example, haloalkyl refers to an alkyl that is substituted with at least one halogen substituent. Where more than one hydrogen is replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 2,2,2-trifluoroethyl, difluoroethyl, pentafluoroethyl, difluoropropyl, dichloropropyl, and heptafluoropropyl. Illustrating further, “haloalkoxy” refers to an alkoxy that is substituted with at least one halogen substituent. Examples of haloalkoxy substituents include chloromethoxy, 1-bromoethoxy, fluoromethoxy, difluoromethoxy, trifluoromethoxy (also known as “perfluoromethyloxy”), and 2,2,2-trifluoroethoxy. It should be recognized that if a substituent is substituted by more than one halogen substituent, those halogen substituents may be identical or different (unless otherwise stated).

The prefix “perhalo” indicates that each hydrogen substituent on the substituent to which the prefix is attached is replaced with an independently selected halogen substituent. If all the halogen substituents are identical, the prefix may identify the halogen substituent. Thus, for example, the term “perfluoro” means that every hydrogen substituent on the substituent to which the prefix is attached is replaced with a fluorine substituent. To illustrate, the term “perfluoroalkyl” refers to an alkyl substituent wherein a fluorine substituent is in the place of each hydrogen substituent. Examples of perfluoroalkyl substituents include trifluoromethyl (—CF₃), perfluorobutyl, perfluoroisopropyl, perfluorododecyl, and perfluorodecyl. To illustrate further, the term “perfluoroalkoxy” refers to an alkoxy substituent wherein each hydrogen substituent is replaced with a fluorine substituent. Examples of perfluoroalkoxy substituents include trifluoromethoxy (—O—CF₃), perfluorobutoxy, perfluoroisopropoxy, perfluorododecoxy, and perfluorodecoxy.

The term “oxo” refers to ═O.

The term “oxy” refers to an ether substituent, and may be depicted as —O—.

The term “alkoxy” refers to an alkyl linked to an oxygen, which may also be represented as —O—R, wherein the R represents the alkyl group. Examples of alkoxy include methoxy, ethoxy, propoxy and butoxy.

The term “alkylthio” means —S-alkyl. For example, “methylthio” is —S—CH₃. Other examples of alkylthio include ethylthio, propylthio, butylthio, and hexylthio.

The term “alkylcarbonyl” means —C(O)-alkyl. For example, “ethylcarbonyl” may be depicted as:

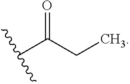

Examples of other alkylcarbonyl include methylcarbonyl, propylcarbonyl, butylcarbonyl, pentylcabonyl, and hexylcarbonyl.

The term “aminoalkylcarbonyl” means —C(O)-alkyl-NH₂. For example, “aminomethylcarbonyl” may be depicted as:

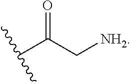

The term “alkoxycarbonyl” means —C(O)—O-alkyl. For example, “ethoxycarbonyl” may be depicted as:

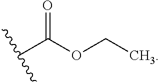

Examples of other alkoxycarbonyl include methoxycarbonyl, ethoxycarbonyl, propoxycarbonyl, butoxycarbonyl, pentoxycarbonyl, and hexyloxycarbonyl. In another embodiment, where the carbon atom of the carbonyl is attached to a carbon atom of a second alkyl, the resulting functional group is an ester.

The terms “thio” and “thia” mean a divalent sulfur atom and such a substituent may be depicted as —S—. For example, a thioether is represented as “alkyl-thio-alkyl” or, alternatively, alkyl-5-alkyl.

The term “thiol” refers to a sulfhydryl substituent, and may be depicted as —SH.

The term “thione” refers to ═S.

The term “sulfonyl” refers to —S(O)₂—, which also may be depicted as:

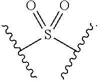

Thus, for example, “alkyl-sulfonyl-alkyl” refers to alkyl-S(O)₂-alkyl. Examples of alkylsulfonyl include methylsulfonyl, ethylsulfonyl, and propylsulfonyl.

The term “aminosulfonyl” means —S(O)₂—NH₂, which also may be depicted as:

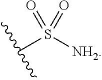

The term “sulfinyl” or “sulfoxido” means —S(O)—, which also may be depicted as:

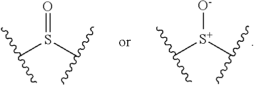

Thus, for example, “alkylsulfinylalkyl” or “alkylsulfoxidoalkyl” refers to alkyl-S(O)-alkyl. Exemplary alkylsulfinyl groups include methylsulfinyl, ethylsulfinyl, butylsulfinyl, and hexylsulfinyl.

The term “heterocycloalkyl” refers to a saturated or partially saturated ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur. A heterocycloalkyl alternatively may comprise 2 or 3 rings fused together, wherein at least one such ring contains a heteroatom as a ring atom (e.g., nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom.

The term “heterocycloalkyl” also includes substituents that are fused to a C₆-C₁₀ aromatic ring or to a 5-10-membered heteroaromatic ring, wherein a group having such a fused heterocycloalkyl group as a substituent is bound to a heteroatom of the heterocyclocalkyl group or to a carbon atom of the heterocycloalkyl group. When such a fused heterocycloalkyl group is substituted with one more substituents, the one or more substituents, unless otherwise specified, are each bound to a heteroatom of the heterocyclocalkyl group or to a carbon atom of the heterocycloalkyl group. The fused C₆-C₁₀ aromatic ring or to a 5-10-membered heteroaromatic ring may be optionally substituted with halogen, C₁-C₆ alkyl, C₃-C₁₀ cycloalkyl, or ═O.

The term “heteroaryl” refers to an aromatic ring structure containing from 5 to 14 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N-oxide ring.

Examples of single-ring heteroaryls include furanyl, dihydrofuranyl, tetradydrofuranyl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiaediazolyl, oxathiazolyl, oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

Examples of 2-fused-ring heteroaryls include, indolizinyl, pyrindinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include 5,6-dihydro-4H-imidazo[4,5,1-ij]quinoline, 4,5-dihydroimidazo[4,5,1-hi]indole, 4,5,6,7-tetrahydroimidazo[4,5,1-jk][1]benzazepine, and dibenzofuranyl.

Other examples of fused-ring heteroaryls include benzo-fused heteroaryls such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “heteroaryl” also includes substituents such as pyridyl and quinolinyl that are fused to a C₄-C₁₀ carbocyclic ring, such as a C₅ or a C₆ carbocyclic ring, or to a 4-10-membered heterocyclic ring, wherein a group having such a fused aryl group as a substituent is bound to an aromatic carbon of the heteroaryl group or to a heteroatom of the heteroaryl group. When such a fused heteroaryl group is substituted with one more substituents, the one or more substituents, unless otherwise specified, are each bound to an aromatic carbon of the heteroaryl group or to a heteroatom of the heteroaryl group. The fused C₄-C₁₀ carbocyclic or 4-10-membered heterocyclic ring may be optionally substituted with halogen, C₁-C₆ alkyl, C₃-C₁₀ cycloalkyl, or ═O.

The term “ethylene” refers to the group —CH₂—CH₂—. The term “ethynelene” refers to the group —CH═CH—. The term “propylene” refers to the group —CH₂—CH₂—CH₂—. The term “butylene” refers to the group —CH₂—CH₂—CH₂—CH₂—. The term “methylenoxy” refers to the group —CH₂—O—. The term “methylenethioxy” refers to the group —CH₂—S—. The term “methylenamino” refers to the group —CH₂—N(H)—. The term “ethylenoxy” refers to the group —CH₂—CH₂—O—. The term “ethylenethioxy” refers to the group —CH₂—CH₂—S—. The term “ethylenamino” refers to the group —CH₂—CH₂—N(H)—.

A substituent is “substitutable” if it comprises at least one carbon, sulfur, oxygen or nitrogen atom that is bonded to one or more hydrogen atoms. Thus, for example, hydrogen, halogen, and cyano do not fall within this definition. If a substituent is described as being “substituted,” a non-hydrogen substituent is in the place of a hydrogen substituent on a carbon, oxygen, sulfur or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R,″ wherein R′ and R″ together with the nitrogen atom to which they are attached, may form a heterocyclic ring. The heterocyclic ring formed from R′ and R″ together with the nitrogen atom to which they are attached may be partially or fully saturated. In one embodiment, the heterocyclic ring consists of 3 to 7 atoms. In another embodiment, the heterocyclic ring is selected from the group consisting of pyrrolyl, imidazolyl, pyrazolyl, triazolyl, tetrazolyl, isoxazolyl, pyridyl and thiazolyl.

This specification uses the terms “substituent,” “radical,” and “group” interchangeably. If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents. If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

A prefix attached to a multi-moiety substituent only applies to the first moiety. To illustrate, the term “alkylcycloalkyl” contains two moieties: alkyl and cycloalkyl. Thus, a C₁-C₆— prefix on C₁-C₆-alkylcycloalkyl means that the alkyl moiety of the alkylcycloalkyl contains from 1 to 6 carbon atoms; the C₁-C₆— prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If the halogen substitution may only occur on the alkyl moiety, the substituent would be described as “alkoxyhaloalkyl.” If the halogen substitution may occur on both the alkyl moiety and the alkoxy moeity, the substituent would be described as “haloalkoxyhaloalkyl.”

When a substituent is comprised of multiple moieties, unless otherwise indicated, it is the intention for the final moiety to serve as the point of attachment to the remainder of the molecule. For example, in a substituent A-B-C, moiety C is attached to the remainder of the molecule. In a substituent A-B-C-D, moiety D is attached to the remainder of the molecule. Similarly, in a substituent aminocarbonylmethyl, the methyl moiety is attached to the remainder of the molecule, where the substituent may also be be depicted as

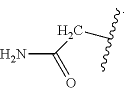

In a substituent trifluoromethylaminocarbonyl, the carbonyl moiety is attached to the remainder of the molecule, where the substituent may also be depicted as

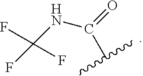

If substituents are described as being “independently selected” from a group, each substituent is selected independent of the other. Each substituent therefore may be identical to or different from the other substituent(s).

Pharmaceutical Compositions

Disclosed are pharmaceutical compositions for preventing and/or treating a subject comprising a therapeutically effective amount of a compound of formula (I), (II) or (III), or pharmaceutically acceptable salts thereof. In some forms, the disclosed pharmaceutical compositions are compositions wherein the compound or a pharmaceutically acceptable salt thereof, is effective in the prevention and/or treatment of diseases which are pathophysiologically related to GPR35, wherein said compound is a compound of formula (I), (II) or (III). In some other forms, the disclosed pharmaceutical compositions are compositions wherein the compound or a pharmaceutically acceptable salt thereof, is effective in the prevention and/or treatment of diseases which are pathophysiologically related to GPR35-hERG complex, wherein said compound is a compound of formula (I), (II) or (III). In some other forms, the disclosed pharmaceutical compositions are compositions which further comprise one or more therapeutic agents.

In some forms, the disclosed pharmaceutical compositions are compositions wherein the compound or a pharmaceutically acceptable salt thereof, and one or more therapeutic agents produces synergistic effect in preventing and/or treating diseases which are pathophysiologically related to GPR35 and/or GPR35-hERG complex in a subject. In some other forms, the disclosed pharmaceutical compositions are compositions wherein the weight ratio of the compound or a pharmaceutically acceptable salt thereof, to said one or more therapeutic agents ranges from about 1:100 to about 100:1, or from about 1:50 to about 50:1, or from about 1:10 to about 10:1, or from about 1:5 to about 5:1.

In some other forms, the disclosed pharmaceutical compositions are compositions wherein said one or more therapeutic agents are selected from the group consisting of anti-inflammation agent, anti-metabolic-disorder agent, anti-congestive-heart-failure agent, anti-cancer agent, kynurenic acid, NPPB, zaprinast and lysophosphatidic acid (LPA). In some other forms, the disclosed pharmaceutical compositions are compositions wherein the subject is a mammal.

Also disclosed are pharmaceutical compositions for treating a subject comprising a therapeutically effective amount of a molecule identified in the disclosed methods of identifying GPR35-hERG complex interfering molecules.

Also disclosed are therapeutic agents for GPR35-hERG complex-associated disorders wherein a GPR35-hERG interaction can be prevented. Also disclosed are therapeutic agents for GPR35-hERG complex-associated disorders wherein a GPR35-hERG interaction can be disrupted.

In some forms, the pharmaceutical compositions as described above can further comprise a pharmaceutically acceptable carrier or excipient. By “pharmaceutically acceptable”, it is meant a material that is not biologically or otherwise undesirable, i.e., the material can be administered to a subject without causing any undesirable biological effects or interacting in a deleterious manner with any of the other components of the pharmaceutical composition in which it is contained. The carrier can be selected to minimize any degradation of the active ingredient and to minimize any adverse side effects in the subject, as would be well known to one of skill in the art. The carrier can be a solid, a liquid, or both, and can be formulated with the compound as a unit-dose composition, for example, a tablet, which can contain, for example, from 0.05% to 100%, from 0.05% to 99%, from 0.05% to 98%, from 0.05% to 97%, from 0.05% to 96%, from 0.05% to 95%, from 0.05% to 94%, from 0.05% to 93%, from 0.05% to 92%, from 0.05% to 91%, from 0.05% to 90%, from 0.05% to 85%, from 0.05% to 80%, from 0.05% to 75%, from 0.05% to 70%, from 0.05% to 65%, from 0.05% to 60%, from 0.05% to 55%, from 0.05% to 50%, from 0.05% to 45%, from 0.05% to 40%, from 0.05% to 35%, from 0.05% to 30%, from 0.05% to 25%, from 0.05% to 20%, from 0.05% to 15%, from 0.05% to 10%, from 0.05% to 5%, from 0.05% to 4%, from 0.05% to 3%, from 0.05% to 2%, from 0.05% to 1%, from 0.05% to 0.8%, from 0.05% to 0.6%, from 0.05% to 0.5%, from 0.05% to 0.4%, from 0.05% to 0.3%, from 0.05% to 0.2%, from 0.05% to 0.1%, from 0.1% to 100%, from 0.2% to 100%, from 0.3% to 100%, from 0.4% to 100%, from 0.5% to 100%, from 0.6% to 100%, from 0.8% to 100%, from 1% to 100%, from 2% to 100%, from 3% to 100%, from 4% to 100%, from 5% to 100%, from 10% to 100%, from 15% to 100%, from 20% to 100%, from 25% to 100%, from 30% to 100%, from 35% to 100%, from 40% to 100%, from 45% to 100%, from 50% to 100%, from 55% to 100%, from 60% to 100%, from 65% to 100%, from 70% to 100%, from 75% to 100%, from 80% to 100%, from 85% to 100%, from 90% to 100%, 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.8%, 0.6%, 0.5%, 0.4%, 0.3%, 0.2%, 0.1%, 0.05% by weight of the active compounds. A disclosed compound can be coupled with suitable polymers as targetable drug carriers. Other pharmacologically active substances can also be present.

Any suitable route of administration can be used for the disclosed compositions. Suitable routes of administration can, for example, include topical, enteral, local, systemic, or parenteral. For example, administration can be epicutaneous, inhalational, enema, conjunctival, eye drops, ear drops, alveolar, nasal, intranasal, vaginal, intravaginal, transvaginal, ocular, intraocular, transocular, enteral, oral, intraoral, transoral, intestinal, rectal, intrarectal, transrectal, injection, infusion, intravenous, intraarterial, intramuscular, intracerebral, intraventricular, intracerebroventricular, intracardiac, subcutaneous, intraosseous, intradermal, intrathecal, intraperitoneal, intravesical, intracavernosal, intramedullar, intraocular, intracranial, transdermal, transmucosal, transnasal, inhalational, intracisternal, epidural, peridural, intravitreal, etc.

Suitable carriers and their formulations are described in Remington: The Science and Practice of Pharmacy (19th ed.) ed. A. R. Gennaro, Mack Publishing Company, Easton, Pa., 1995. Typically, an appropriate amount of a pharmaceutically acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing, for example, the antiviral agent, which matrices can be in the form of shaped articles, e.g., films, liposomes or microparticles. It will be apparent to those persons skilled in the art that certain carriers may be more preferable depending upon, for instance, the route of administration and concentration of composition being administered. Pharmaceutical carriers are known to those skilled in the art. These most typically would be standard carriers for administration of drugs to humans, including solutions such as sterile water, saline, and buffered solutions at physiological pH. Examples of the pharmaceutically acceptable excipient include, but are not limited to, thickeners, diluents, buffers, preservatives, surface active agents and the like.

The disclosed compounds can be administered by any suitable route, preferably in the form of a pharmaceutical composition adapted to such a route, and in a dose effective for the treatment or prevention intended. The active compounds and compositions, for example, can be administered orally, rectally, parenterally, ocularly, inhalationaly, or topically.

Oral administration of a solid dose form can be, for example, presented in discrete units, such as hard or soft capsules, pills, cachets, lozenges, or tablets, each containing a predetermined amount of at least one of the disclosed compound or compositions. In some forms, the oral administration can be in a powder or granule form. In some forms, the oral dose form is sub-lingual, such as, for example, a lozenge. In such solid dosage forms, the compounds of formula I are ordinarily combined with one or more adjuvants. Such capsules or tablets can contain a controlled-release formulation. In the case of capsules, tablets, and pills, the dosage forms also can comprise buffering agents or can be prepared with enteric coatings.

In some forms, oral administration can be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also can comprise adjuvants, such as wetting, emulsifying, suspending, flavoring (e.g., sweetening), and/or perfuming agents.

In some forms, the disclosed compositions can comprise a parenteral dose form. “Parenteral administration” includes, for example, subcutaneous injections, intravenous injections, intraperitoneally, intramuscular injections, intrasternal injections, and infusion. Injectable preparations (e.g., sterile injectable aqueous or oleaginous suspensions) can be formulated according to the known art using suitable dispersing, wetting agents, and/or suspending agents.

In some forms, the disclosed compositions can comprise a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation can include a compound which enhances absorption or penetration of the active ingredient through the skin or other affected areas. When the compounds and compositions are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes can also be used. Typical carriers include alcohol, water, mineral oil, liquid petrolatum, white petrolatum, glycerin, polyethylene glycol and propylene glycol. Penetration enhancers can be incorporated—see, for example, J Pharm Sci, 88 (10), 955-958, by Finnin and Morgan (October 1999).

Formulations suitable for topical administration to the eye include, for example, eye drops wherein the disclosed compound or composition is dissolved or suspended in suitable carrier. A typical formulation suitable for ocular or aural administration can be in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, can be incorporated together with a preservative, such as benzalkonium chloride. Such formulations can also be delivered by iontophoresis.

For intranasal administration or administration by inhalation, the active disclosed compounds are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder can comprise a bioadhesive agent, for example, chitosan or cyclodextrin.

In some forms, the disclosed compositions can comprise a rectal dose form. Such rectal dose form can be in the form of, for example, a suppository. Cocoa butter is a traditional suppository base, but various alternatives can be used as appropriate.

Other carrier materials and modes of administration known in the pharmaceutical art can also be used. The disclosed pharmaceutical compositions can be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

The disclosed compounds of formula (I), (II) or (III) can be used, alone or in combination with other therapeutic agents, in the treatment or prevention of various conditions or disease states. The disclosed compound(s) and composition(s) and other therapeutic agent(s) can be administered simultaneously (either in the same dosage form or in separate dosage forms) or sequentially. An exemplary therapeutic agent can be, for example, one selected from the group consisting of anti-inflammation agent, anti-metabolic-disorder agent, anti-congestive-heart-failure agent, anti-cancer agent, kynurenic acid, NPPB, zaprinast, lysophosphatidic acid (LPA) and a compound of formula (I), (II) or (III) as presently disclosed.

The administration of two or more compounds “in combination” means that the two compounds are administered closely enough in time that the presence of one alters the biological effects of the other. The two or more compounds can be administered simultaneously, concurrently or sequentially. Additionally, simultaneous administration can be carried out by mixing the compounds prior to administration or by administering the compounds at the same point in time but at different anatomic sites or using different routes of administration. The phrases “concurrent administration,” “co-administration,” “simultaneous administration,” and “administered simultaneously” mean that the compounds are administered in combination.

The dosage regimen for the compounds and/or compositions containing the compounds can be based on a variety of factors, including the type, age, weight, sex and medical condition of the patient; the severity of the condition; the route of administration; and the activity of the particular compound employed. Thus the dosage regimen can vary widely. Dosage levels of the order from about 0.001 mg to about 100 mg per kilogram of body weight per day are useful in the treatment or prevention of the above-indicated conditions. Other effective dosages regimens of a disclosed compounds (administered in single or divided doses) include but are not limited to: from about 0.01 to about 100 mg/kg/day, from about 0.1 to about 50 mg/kg/day, from about 0.5 to about 30 mg/kg/day, from about 0.01 to about 10 mg/kg/day, and from about 0.1 to about 1.0 mg/kg/day. Dosage unit compositions can contain such amounts or submultiples thereof to make up the daily dose. In many instances, the administration of the compound will be repeated a plurality of times in a day. Multiple doses per day typically can be used to increase the total daily dose, if desired.

For oral administration, the compositions can be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or from about 1 mg to about 100 mg of active ingredient. Intravenously, doses can range from about 0.1 to about 10 mg/kg/minute during a constant rate infusion.

Disclosed are pharmaceutical compositions comprising an effective amount of a compound of the invention or a pharmaceutically accepted salt, solvate, clathrate, or prodrug thereof; and a pharmaceutically acceptable carrier or vehicle. These compositions may further comprise additional agents. These compositions are useful for modulating the activity of hERG-GPR35 complex, thus to improve the prevention and treatment of hERG-GPR35 associated human diseases such as metabolic disorders.

Cells

Disclosed are engineered cells comprising an exogenous GPR35 gene and an exogenous hERG gene. In some forms, the exogenous GPR35 gene and the exogenous hERG gene are present in the cell on a separate nucleic acid from the host cell. In some forms, the exogenous nucleic acid has recombined with host cells' nucleic acid.

Also disclosed are engineered cells comprising an endogenous GPR35 gene and an exogenous hERG gene.

Also disclosed are engineered cells comprising an exogenous GPR35 gene and endogenous hERG gene.

In some forms of the disclosed cells, the GPR35 gene, the hERG gene or both genes can be mutant genes. The mutant genes can either prevent GPR35-hERG interaction or allow complex formation but prevent or inhibit the downstream signaling pathway.

In some forms of the disclosed cells, the GPR35 gene expresses a GPR35 protein and the hERG gene expresses a hERG protein. The GPR35 protein can be a GPR35 fusion protein. In some forms, the GPR35 fusion protein can be myc-GPR35. The GPR35 fusion partner can be, but is not limited to, GST, HA, GFP, HRP, His. The fusion partner can be, but is not limited to, affinity tags or visual tags.

In some forms of the disclosed cells, the GPR35 and hERG proteins interact to form a GPR35-hERG signaling complex when both proteins are co-expressed in the same cell. A GPR35-hERG signaling complex is formed via physical interaction between hERG channel protein and GPR35 protein at the cell surface in a hERG and GPR35 co-expressing cell.

In some forms, the disclosed cells further comprise a regulatable promoter operatively linked to the coding region of GPR35. The regulatable promoter can be, but is not limited to a tetracyline inducible promoter, a T-RExTM promoter, a heat shock inducible promoter, a heavy metal ion promoter or a nuclear hormone receptor inducible promoter, or other promoter element whose activity is conditionally regulated. In some forms, the regulatable promoter comprises a tet operator. In some form, the regulatable promoter comprises a CMV promoter element.

In some forms, the disclosed cells further comprise a regulatable promoter operatively linked to the coding region of hERG.

In some forms, the disclosed cells further comprise a selectable marker. The selectable marker can be, but is not limited to, tetracycline, ampicillin, neomycin, G418 or gentamicin. In some embodiments, the selectable marker and GPR35 coding region are on the same nucleic acid. In some aspects of the invention, the GPR35 and selectable marker coding regions are operatively linked with an IRES or 2A-like sequence. In some embodiments, a GPR35 and selectable marker coding regions are operatively linked to different promoters. In some aspects of the invention, the selectable marker and GPR35 coding region are on different nucleic acids. In some aspects of the invention, the GPR35 coding region is from a cDNA.

In some forms of the disclosed cells, the cell can be, but is not limited to, an animal cell, or a mammalian cell. In some forms, the cell can be selected from the group consisting of a 293 cell, a HEK cell, a CHO cell, a Hela cell, a COS cell, a A431 cell, a A549 cell, a Jurkat cell, a PC12 cells, a human T-lymphocyte cell, a Cos7 cell and a murine cell or derivatives of any of these cells.

In some forms of the disclosed cells, the GPR35 gene and the hERG gene are on the same nucleic acid. In some forms, the GPR35 gene and the hERG gene are on different nucleic acids.

The invention also provides a cell line that can be used to screen for compounds (e.g., small molecules) that modulate either GPR35 alone, or hERG alone, or both. Methods and cells of the invention provide a method of assaying the signaling complex formed between GPR35 and hERG, and classifying modulators acting on the signaling complex.

The invention further provides related cells, nucleic acids and methods for constructing the cells of the invention.

One embodiment of the invention provides a cell comprising a nucleic acid comprising GPR35 or a GPR35 mutant, a nucleic acid comprising a hERG or a hERG mutant, or any combinations.

In one embodiment, the nucleic acid is a DNA or RNA. In one embodiment, the nucleic acid is a viral vector. Viral vectors include, but are not limited to, those derived from a baculovirus, an adenovirus, an Adeno-associated virus, a lentivirus, a retrovirus, or other virus for delivery of genes into cells. In one embodiment, the nucleic acid is a plasmid. In some embodiments, the nucleic acid comprises a transposon. In some embodiments, the nucleic acid is a synthetic microchromosome.

In some embodiments, a cell further comprises a nucleic acid comprising a second promoter operatively linked to a coding region for a hERG. In one embodiment, the regulatable promoter operatively linked to a GPR35 coding region and the second promoter operatively linked to a coding region for a hERG are on the same nucleic acid. In one embodiment, the regulatable promoter operatively linked to a GPR35 coding region and the second promoter operatively linked to a coding region for a hERG are on different nucleic acids. In some embodiments, the regulatable promoter is operatively linked to a GPR35 coding region pre-existing in the genome of the cell.

In some embodiments, a cell of the invention does not contain a coding region. Many GPCRs cause detectable changes in cellular levels of certain signaling molecules, e.g., calcium and/or cAMP levels. In addition, label-free biosensor cellular assays offer a pathway unbiased but pathway sensitive measure of cellular responses upon stimulation. One skilled in the art can readily detect these changes without a coding region. In some aspects of the invention, the cell is stable. In other embodiments of the invention, the cell is not stable at least for one signaling component (e.g., transiently transfected GPR35 or hERG).

In some embodiments, the cell further comprises and/or is contacted with a compound known to bind to either GPR35 or hERG channel.

Complexes

Disclosed are isolated G-protein coupled receptor (GPCR)-hERG complexes comprising one or more GPCRs and hERG. The disclosed complexes and compositions can be used to screen for molecules that bind the complex. One of skill in the art would be aware of a variety of applications of an isolated complex or composition comprising this complex.

Also disclosed are compositions comprising the GPCR-hERG complex disclosed herein.

In some forms of the disclosed complexes and compositions, the GPCR-hERG complex can be GPR35-hERG and the GPCR is GPR35. In some forms, the GPR35-hERG complex comprises a label. The label can be, but is not limited to, a fluorescent label, a radioactive label, an enzyme label, or an affinity label.

Kits

Also disclosed are kits that are suitable for use in performing the methods of treatment or prevention described below. In some forms, the kit contains a first dosage form comprising one or more of the disclosed compounds and a container for the dosage, in quantities sufficient to carry out the disclosed methods. In some forms, a kit can comprise one or more disclosed compounds, and one or more other therapeutic agents. An exemplary therapeutic agent can be, for example, an anti-cancer agent.

Also disclosed are kits comprising a GPR35-hERG expressing engineered cell line and instructions for handling the cell line. In some forms, the kits further comprise instructions for screening of compounds that modulate a GPR35-hERG complex.

Also disclosed are kits comprising a GPR35-hERG expressing engineered cell line and instructions for handling the cell line, further comprising a composition comprising a molecule identified in molecule identified in the disclosed methods of identifying GPR35-hERG complex interfering molecules.

### Methods

All of the methods of the invention may be practice with a compound of the invention alone, or in combination with other agents, or other anticancer drugs.

### A. Screening

1. hERG Modulators

Also disclosed are methods of screening a hERG-specific modulator, comprising the steps of: (a) incubating a compound individually with two different types of cells consisting of a cell expressing hERG and a cell without expressing hERG; (b) monitoring the compound induced cellular response on each cell type with a label-free biosensor cellular assay; (c) incubating a label-free biosensor hERG activator with the hERG expressing cell in the presence of the compound; (d) monitoring the label-free biosensor hERG activator induced cellular response on the hERG expressing cell in the presence of the compound; and (e) generating a biosensor index of the compound which indicates whether the compound is a hERG modulator or not.

In some forms, the methods of screening a hERG-specific modulator further comprises step (f): confirming the compound to be a hERG modulator using an electrophysiology method. In some other forms, the methods of screening a hERG-specific modulator are methods wherein the label-free biosensor hERG activator is a hERG activator, hERG ion channel activator, or hERG pathway activator. In some other forms, the methods of screening a hERG-specific modulator are methods wherein the hERG activator is selected from the group consisting of mallotoxin, RPR260243, NS1643, NS3623, PD-118057, PD-307243, A-935142, flufenamic acid, niflumic acid, and diflunisal.

In some forms, the methods of screening a hERG-specific modulator are methods wherein the hERG expressing cell line is selected from the group consisting of a leukemia cell line, a gastric cancer cell line, a neuroblastoma cell line, a mammary carcinoma cell line, and a human colon carcinoma cell line, a cardiovascular cell line, and a neuronal cell line. In some other forms, the methods of screening a hERG-specific modulator are methods wherein the hERG expressing cell line is selected from the group consisting of cell line HL60, cell line SGC7901, cell line MGC803, cell line SH-SY5Y, cell line MCF-7, cell line HT-29, cell line HCT8, and cell line HCT116.

In some other forms, the methods of screening a hERG-specific modulator are methods wherein the hERG non-expressing cell line is selected from the group consisting of a human embryonic kidney cell line and Chinese Ovary hamster cell line. In some forms, the methods of screening a hERG-specific modulator are methods wherein the hERG non-expressing cell line is selected from the group consisting of cell line HEK-293 and cell line CHO-K1. In some other forms, the methods of screening a hERG-specific modulator are methods wherein step (e) involves comparing the biosensor index of the compound to the biosensor index of a known hERG modulator.

2. GPR35 Modulators

Also disclosed are methods of screening for a GPR35-specific modulator, comprising the steps of: (a) providing a cell that express GPR35; (b) contacting said cell with a compound; and (3) profiling said compound using label-free biosensor cellular assay. In some forms, the methods of screening for a GPR35-specific modulator are methods wherein said profiling comprising generating a biosensor index of the compound and determining whether the compound is a GPR35 modulator or not.

In some other forms, the methods of screening for a GPR35-specific modulator are methods wherein said determination involves comparing the biosensor index of said compound with the biosensor index of a known GPR35 modulator. In some other forms, the methods of screening for a GPR35-specific modulator are methods wherein said compound is a GPR35 agonist. In some other forms, the methods of screening for a GPR35-specific modulator are methods wherein said compound is a GPR35 modulator when the biosensor index of said compound is similar to the biosensor index of said known GPR35 modulator.

In some forms, the methods of screening for a GPR35-specific modulator are methods wherein said known GPR35 agonist is selected grom the group consisting of kynurenic acid, NPPB, zaprinast and lysophosphatidic acid (LPA). In some other forms, the methods of screening for a GPR35-specific modulator are methods wherein said step (b) involves contacting said cell with a compound and a known GPR35 agonist. In some forms, the methods of screening for a GPR35-specific modulator are methods wherein said compound is a GPR35 antagonist when the biosensor index of said compound is similar to the biosensor index of a known GPR35 antagonist.

In some forms, the methods of screening for a GPR35-specific modulator are methods wherein said compound is a GPR35 modulator having a formula (I), (II) or (III). In some forms, the methods of screening for a GPR35-specific modulator are methods wherein said compound is a GPR35 modulator having a chemical structure selected from the group consisting of:

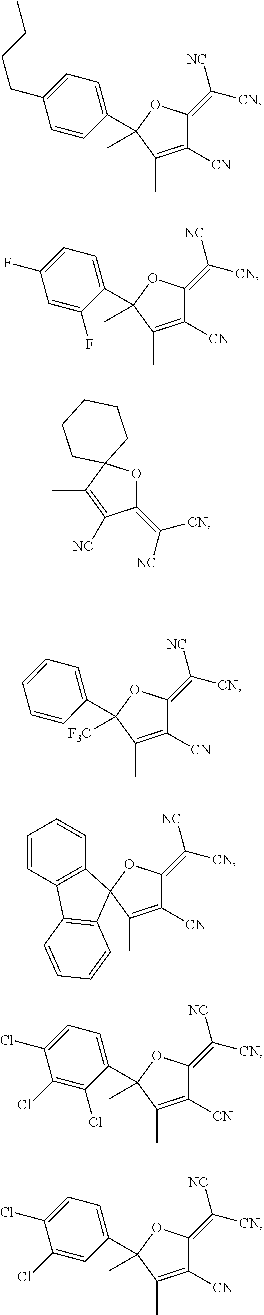

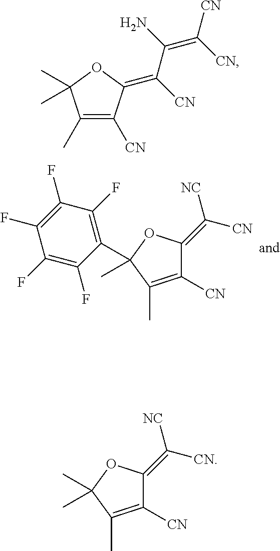

In some forms, the methods of screening for a GPR35-specific modulator are methods wherein said compound is a GPR35 modulator having a chemical structure selected from the group consisting of:

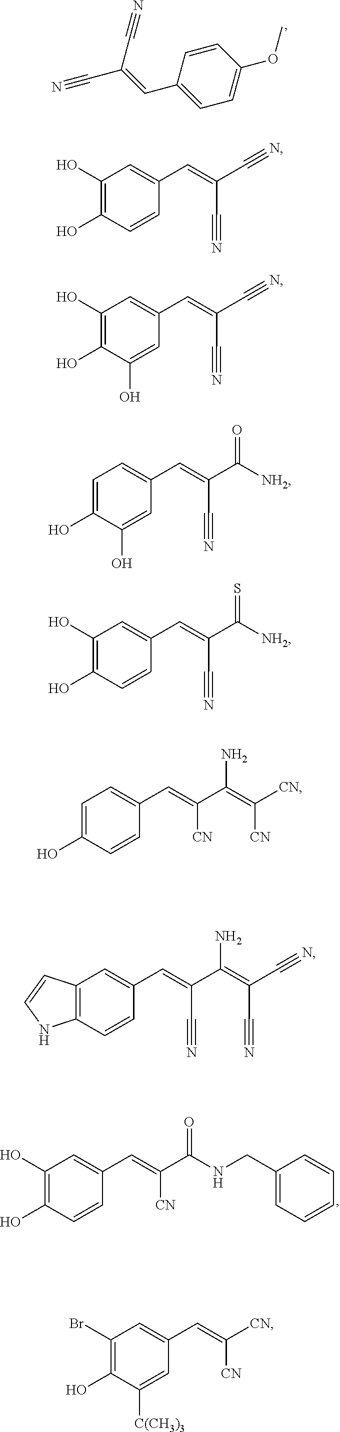

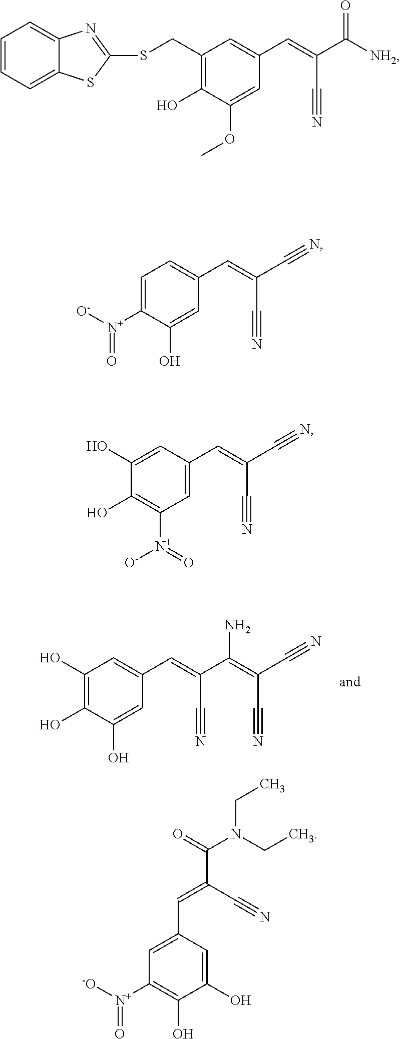

In some forms, the methods of screening for a GPR35-specific modulator are methods wherein said compound is a GPR35 modulator having a chemical structure selected from the group consisting of:

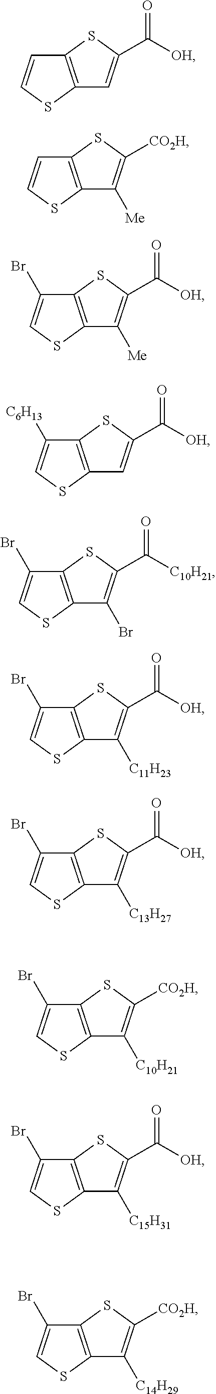

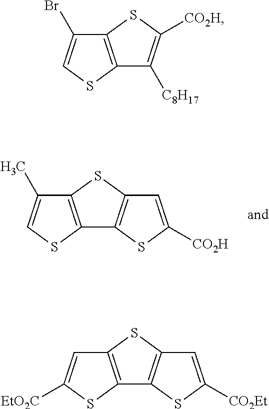

In some forms, the methods of screening for a GPR35-specific modulator are methods wherein said compound is a GPR35 modulator having a chemical structure selected from the group consisting of:

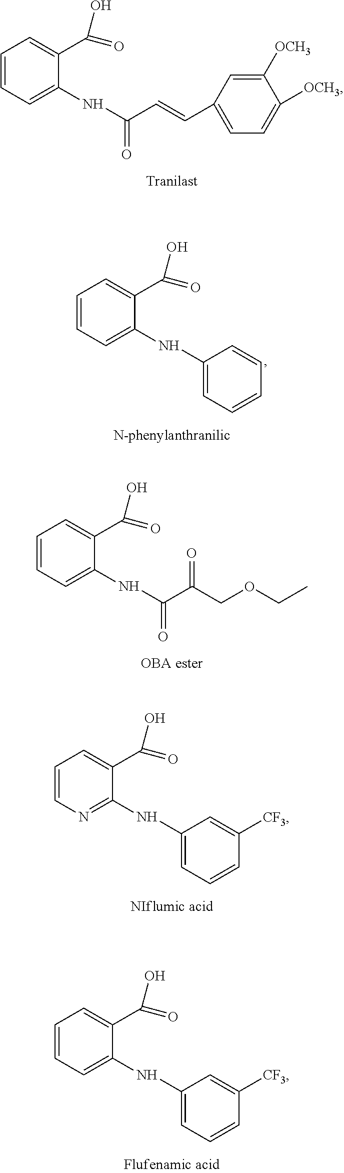

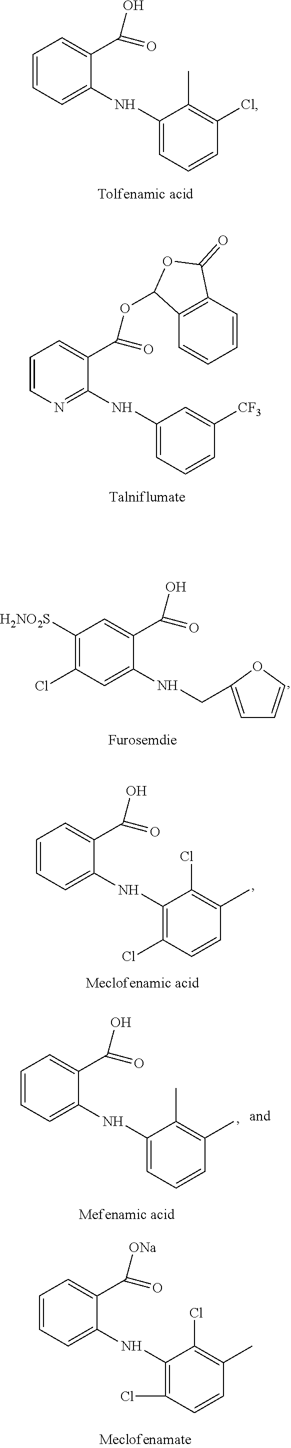

In some forms, the methods of screening for a GPR35-specific modulator are methods wherein said GPR35 is human.

3. GPR35-hERG Complex Modulators

Also disclosed are methods of screening for a GPR35-hERG signaling complex modulator, comprising the steps of: (a) determining if a compound is a GPR35-specific modulator or a hERG-specific modulator or neither; and (b) determining if said compound is a GPR35-hERG signaling complex modulator. In some forms, the methods of screening for a GPR35-hERG signaling complex modulator are methods wherein step (a) is according to the method of screening a hERG-specific modulator, and/or the method of screening a GPR35-specific modulator as disclosed above.

In some forms, the methods of screening for a GPR35-hERG signaling complex modulator are methods wherein step (b) comprises: (i) providing a cell comprising GPR35-hERG complex; (ii) contacting said cell with said compound; and (iii) profiling said compound by using one or more suitable assays.

In some forms, the methods of screening for a GPR35-hERG signaling complex modulator are methods wherein said profiling of step (iii) comprises analyzing the signal with said assay and determining if the compound is a GPR35-hERG signaling complex modulator. In some forms, the methods of screening for a GPR35-hERG signaling complex modulator are methods wherein said assay is a label-free biosensor cellular assay. In some forms, the methods of screening for a GPR35-hERG signaling complex modulator are methods wherein said assay is a cross-desensitization DMR assay.

In some forms, the methods of screening for a GPR35-hERG signaling complex modulator are methods wherein said assay is conducted to determine agonism action of said modulator acting via said GPR35-hERG complex. In some forms, the methods of screening for a GPR35-hERG signaling complex modulator are methods wherein said assay is conducted to determine the antagonism action of said modulator acting via said GPR35-hERG complex against a GPR35-specific agonist marker, or against a hERG-specific activator marker.

In some forms, the methods of screening for a GPR35-hERG signaling complex modulator are methods wherein said GPR35-hERG signaling complex modulator is a GPR35 agonist and also a hERG activator. In some forms, the methods of screening for a GPR35-hERG signaling complex modulator are methods wherein said GPR35-hERG signaling complex modulator is a GPR35-specific agonist which transactivates hERG. In some forms, the methods of screening for a GPR35-hERG signaling complex modulator are methods wherein said GPR35-hERG signaling complex modulator is a GPR35-specific agonist which does not transactivate hERG. In some forms, the methods of screening for a GPR35-hERG signaling complex modulator are methods wherein said GPR35-hERG signaling complex modulator is a hERG-specific activator which transactivates GPR35. In some forms, the methods of screening for a GPR35-hERG signaling complex modulator are methods wherein said GPR35-hERG signaling complex modulator is a hERG-specific activator which does not transactivate GPR35. In some forms, the methods of screening for a GPR35-hERG signaling complex modulator are methods wherein said GPR35-hERG signaling complex modulator is neither a GPR35 agonist nor a hERG activator. In some forms, the methods of screening for a GPR35-hERG signaling complex modulator are methods wherein said GPR35-hERG signaling complex modulator is a secondary modulator which modulates the modulators as discussed above.

In some forms, the methods of screening for a GPR35-hERG signaling complex modulator are methods wherein a signaling pathway is modulated when said GPR35-hEGR signaling complex modulator acts on said complex. In some forms, the methods of screening for a GPR35-hERG signaling complex modulator are methods wherein said signaling pathway is substantially similar to the signaling pathway formed when a GPR35-specific modulator acts on GPR35 alone. In some forms, the methods of screening for a GPR35-hERG signaling complex modulator are methods wherein said signaling pathway is substantially similar to the signaling pathway formed when a hERG-specific modulator acts on hERG alone.

In some forms, the methods of screening for a GPR35-hERG signaling complex modulator are methods wherein said signaling pathway is different from the signaling pathway formed when a GPR35-specific modulator acts on GPR35 alone, or when a hERG-specific modulator acts on hERG alone. In some forms, the methods of screening for a GPR35-hERG signaling complex modulator are methods wherein said signaling pathway is a combination of the signaling pathway formed when a GPR35-specific modulator acts on GPR35 alone and the signaling pathway modulated when a hERG-specific modulator acts on hERG alone.

In some forms, the methods of screening for a GPR35-hERG signaling complex modulator are methods wherein said compound is a GPR35-hEGR signaling complex modulator having a formula (I), (II) or (III).

In some forms, the methods of screening for a GPR35-hERG signaling complex modulator are methods wherein said compound is a GPR35-hEGR signaling complex modulator having a chemical structure selected from the group consisting of:

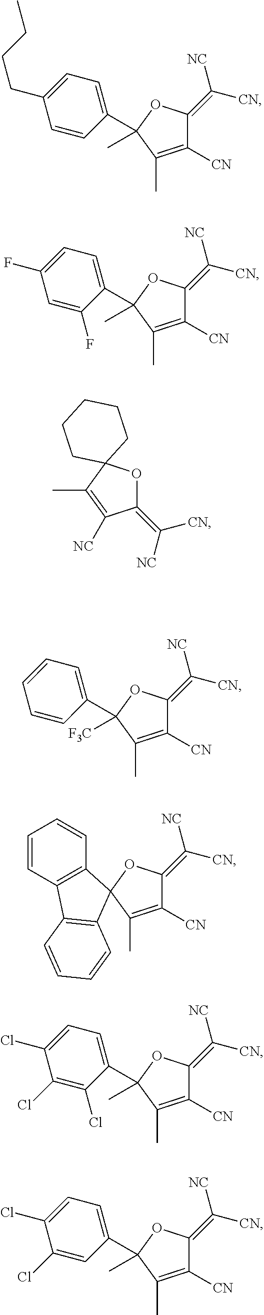

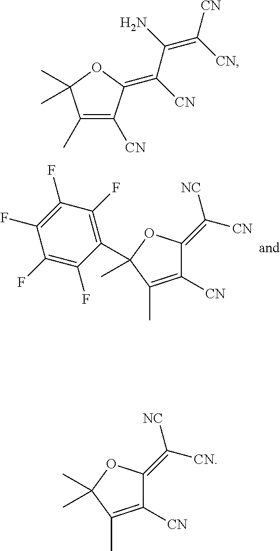

In some forms, the methods of screening for a GPR35-hERG signaling complex modulator are methods wherein said compound is a GPR35-hEGR signaling complex modulator having a chemical structure selected from the group consisting of:

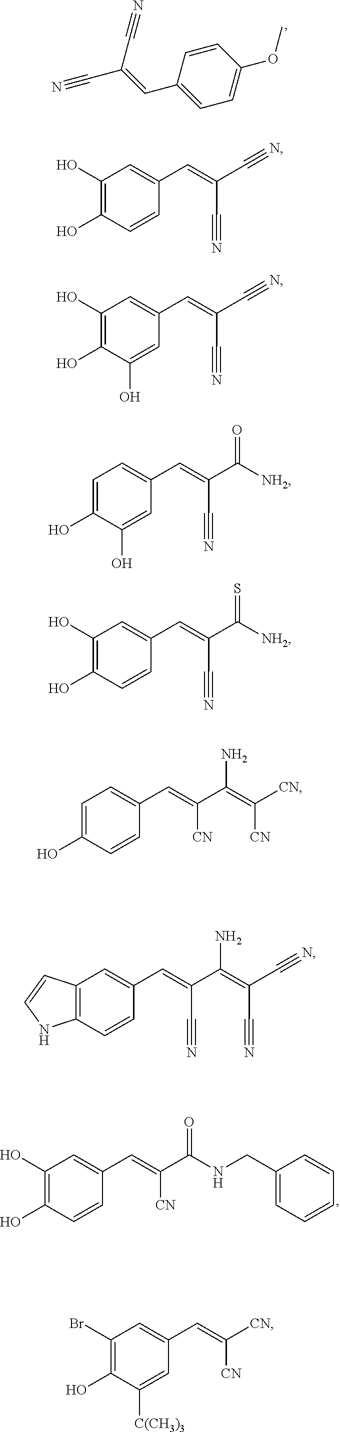

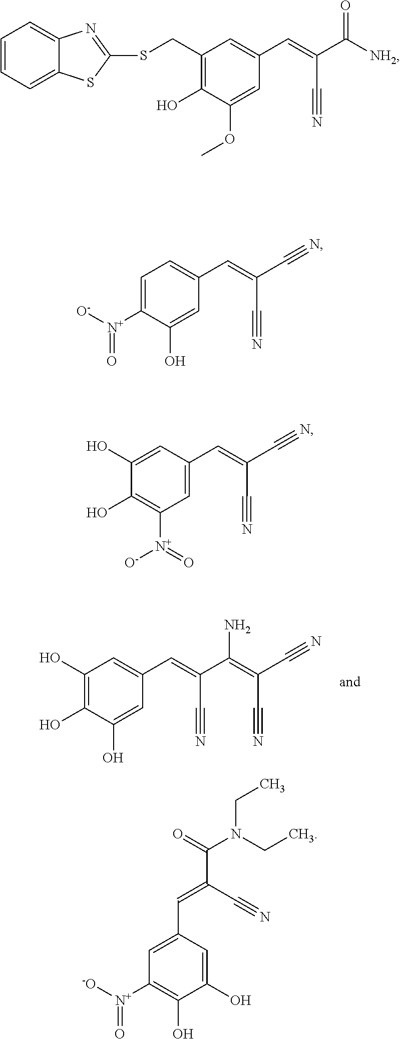

In some forms, the methods of screening for a GPR35-hERG signaling complex modulator are methods wherein said compound is a GPR35-hEGR signaling complex modulator having a chemical structure selected from the group consisting of:

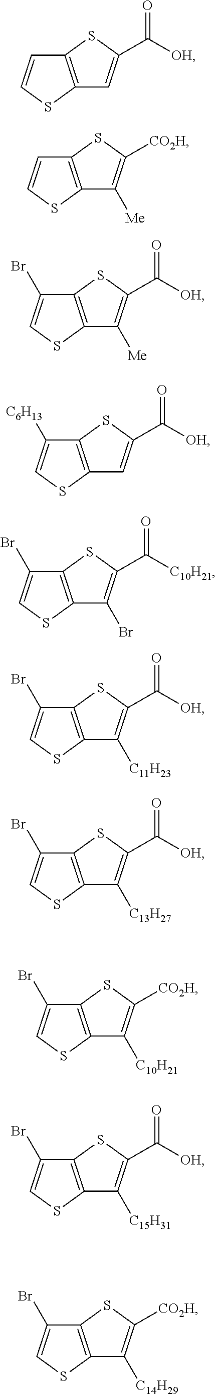

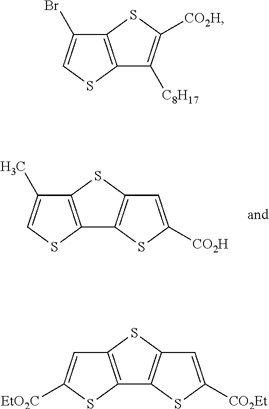

In some forms, the methods of screening for a GPR35-hERG signaling complex modulator are methods wherein said compound is a GPR35-hEGR signaling complex modulator having a chemical structure selected from the group consisting of:

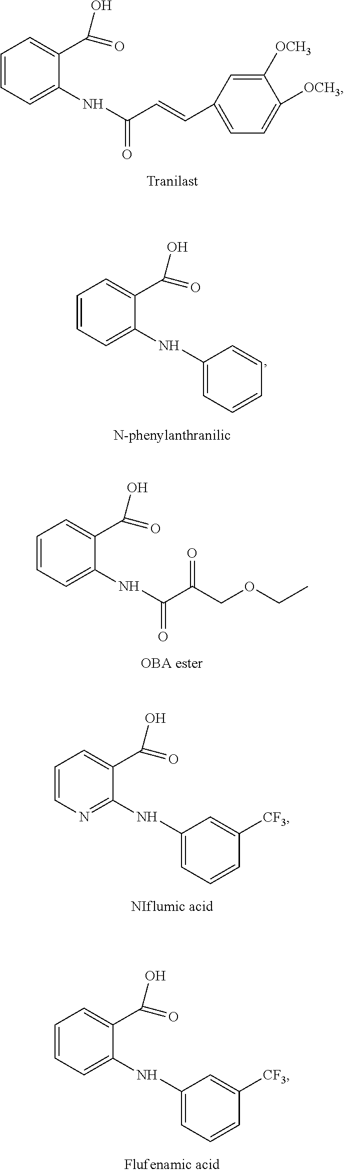

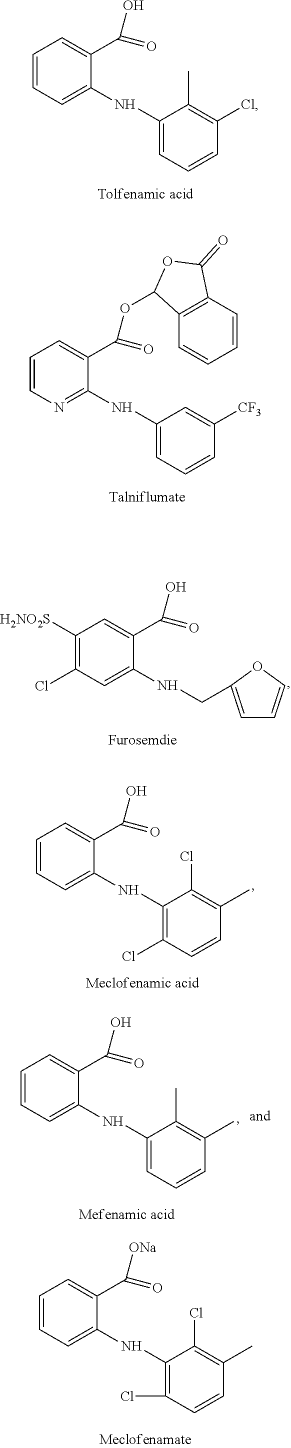

The disclosed compositions and methods relate, in part, to assays for identifying modulators (e.g., activators, blockers, agonists, inverse agonists, or antagonists) of signaling pathways, as well as compositions used in such assays. Specifically, disclosed is relate to identifying and classifying modulators acting on GPR35-hERG signaling complexes, as well as compositions used in such assays.

In some aspects, disclosed involve the detection of an expression product. The detection includes, but is not limited to, detecting the expression of corresponding mRNA, the expression of proteins, the location of the proteins, and the interactions between GPR35 and hERG, and the signaling consequence of each component and the whole signaling complex being activated.

In some embodiments, disclosed include assays which function by contacting a cell with a potential modulator of a component of the signaling hERG-GPR35 complex followed by measuring a downstream activity of the signaling pathway. Examples of effects which can be measured include, but are not limited to, transcription of a particular cellular nucleic acid, translation of a particular gene and changes in concentrations of a compound(s) (e.g., calcium or cAMP), translocation of a protein, and integrated cellular responses as measured by label-free biosensor cellular assay.

Some embodiments of the disclosure provide functional cell-based assays e.g., for high throughput screening or detection of small molecules that act as modulators of the GPR35/hERG signaling complexes. Some embodiments of the invention provide coupled reactions wherein a signal from a receptor (e.g., GPR35) modulates the activity of another receptor (e.g., hERG), and/or modulates a cellular response wherein the change can be measured (e.g., calcium and/or cAMP levels, or DMR signal). The disclosed provide various methods as described herein. For clarity, the disclosed can be used to screen for modulators of any component in the signaling complex and its associated pathway.

According to the present disclosure, any mutations of GPR35, including constitutive isoforms, can be expressed. Alternatively, any mutations of hERG channels can be expressed so that many different combinations can be achieved.

i. Classifying

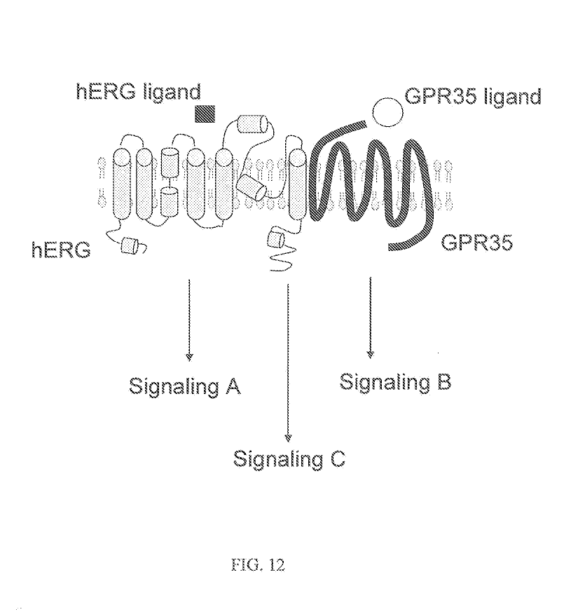

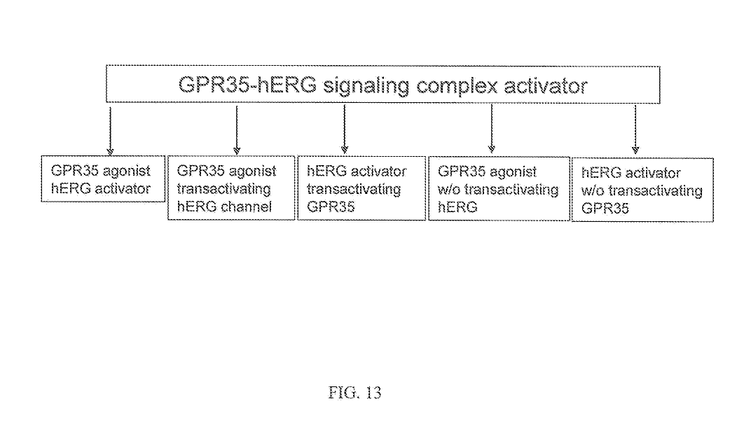

Disclosed relate to classes of hERG-GPR35 signaling complex activators. These activators can be (1) a hERG-GPR35 complex activator that is a GPR35 agonist and also a hERG activator, or (2) a hERG transactivating GPR35 agonist (i.e., a functionally selective GPR35 agonist that is able to transactivate hERG channel); or (3) a GPR35 transactivating hERG activator (i.e., a functionally selective hERG activator that is able to transactivate GPR35); or (4) a hERG non-transactivating GPR35 agonist (i.e., a GPR35-specific agonist that is not able to transactivate hERG); or (5) A GPR35 non-transactivating hERG activator (i.e., a hERG specific activator that is not able to transactivate GPR35) (see FIG. 12 and FIG. 13). The activation of hERG channel by a hERG ligand may or may not transactivate GPR35 in the same complex, whereas the activation of GPR35 by a GPR35 agonist may or may not transactivate hERG channel in the same complex. The activation of the hERG-GPR35 signaling complex by a hERG-GPR35 agonist can lead to the activation of both hERG and GPR35 within the same complex (see FIG. 12).

Disclosed relates to methods to classify hERG-GPR35 signaling complex activators. Disclosed methods are combinations of a panel of cellular assays to define the classes of hERG-GPR35 complex modulators (see examples shown in FIGS. 1 to 11, and FIGS. 14 and 17): (1) a cellular assay including point-of-contact assays (e.g., receptor translocation, Ca²⁺ mobilization, cAMP changes, protein phosphorylation) or integrative assays (e.g., label-free biosensor cellular assays) to determine the action of a modulator acting via GPR35 in a GPR35 presenting cell; (2) a patch clamping recording to determine the action of a modulator acting via hERG channel in a hERG presenting cell; (3) an integrative assay (e.g., label-free biosensor cellular assays) to determine the agonism action of a modulator acting via the hERG-GPR35 complex in a hERG and GPR35 co-expressing cell; and (4) an integrative assay to determine the antagonism action of a modulator acting via the hERG-GPR35 complex against the hERG activator marker, and/or the GPR35 agonist marker, in the hERG and GPR35 co-expressing cell. The hERG activator marker is preferably mallotoxin, while the GPR35 agonist marker is preferably NPPB, kynurenic acid, zaprinast, or YE210.

4. GPR35-hERG Complex Interfering Molecules

Disclosed are methods of identifying GPR35-hERG complex interfering molecules comprising contacting a composition comprising a GPR35-hERG complex with a test agent and assaying for GPR35-hERG interaction.

In some forms of the disclosed methods, the absence of a GPR35-hERG interaction indicates the test agent is a GPR35-hERG complex interfering molecule.

In some forms of the disclosed methods, assaying for GPR35-hERG interaction comprises isolating GPR35 and detecting the presence of hERG, wherein the presence of hERG indicates a GPR35-hERG interaction. In some forms, assaying for GPR35-hERG interaction comprises isolating hERG and detecting the presence of GPR35, wherein the presence of GPR35 indicates a GPR35-hERG interaction.

In some forms of the disclosed methods, assaying for GPR35-hERG interaction comprises comparing the GPR35-hERG interaction in the presence versus the absence of test agent. The presence of a GPR35-hERG interaction in the absence of test agent and in the presence of the test agent indicates the test agent is not a GPR35-hERG complex interfering molecule. The presence of GPR35-hERG interaction in the absence of test agent and the absence of GPR35-hERG interaction in the presence of test agent indicates the test agent is a GPR35-hERG interfering molecule.

In some forms of the disclosed methods, the composition comprising a GPR35-hERG complex is a cell. In some forms, the cell can be an animal cell. For example, the cell can be human or mouse. In some forms the cell is a recombinantly engineered cell.

In some forms of the disclosed methods, the GPR35-hERG complex interfering molecules prevent the GPR35-hERG interaction. In some forms of the disclosed methods, the GPR35-hERG complex interfering molecules disrupt the GPR35-hERG interaction. The prevention or disruption of the GPR35-hERG complex can occur in a variety of ways. The interfering molecule can comprise an identical sequence to the GPR35 binding region of hERG and thus compete for binding. For example, a peptide comprising the amino acids of the hERG binding region on GPR35 can prevent or disrupt the interaction of GPR35 and hERG. Though not identical, the interfering molecule can mimic the GPR35 binding region of hERG or the hERG binding region of GPR35 in order to compete for binding. Alternatively, the interfering molecule can simply block the interaction by blocking one of the interaction sites. For example, an antibody to the hERG binding region on GPR35 can block the ability of hERG to interact with GPR35. One of skill in the art would know the common mechanisms of interfering molecules and how to assay for these.

In some forms of the disclosed methods, the GPR35-hERG complex interfering molecule can be an antibody or fragment thereof. In some forms, the GPR35-hERG complex interfering molecule can be a protein or a peptide. In some forms, the GPR35-hERG complex interfering molecule can be a compound, or a pharmaceutically acceptable salt thereof. In some forms, the GPR35-hERG complex interfering molecule can be a nucleic acid.

### B. Treating

The above-described compounds and compositions are useful for the inhibition, reduction, prevention, and/or treatment of diseases which are pathophysiologically related to GPR35, hERG and/or GPR35-hERG complex. Accordingly, in some forms, disclosed are methods of preventing and/or treating diseases which are pathophysiologically related to GPR35, comprising administering to a subject a therapeutically effective amount of a compound as disclosed above, or a pharmaceutically acceptable salt thereof.

Suitable subjects can include mammalian subjects. Mammals include, but are not limited to, canine, feline, bovine, caprine, equine, ovine, porcine, rodents, lagomorphs, primates, and the like, and encompass mammals in utero. In some forms, humans are the subjects. Human subjects can be of either gender and at any stage of development.

1. hERG Modulators

A hERG modulator is a molecule that can modulate the activity of hERG ion channel directly or indirectly. A hERG modulator that modulates the activity of hERG channel directly is a molecule that binds to hERG channels, thus causing the alteration in hERG activity, such as hERG current, ion flux via hERG, and/or cell signaling via hERG (This type modulator is referred to the hERG-specific modulator). A hERG modulator that modulates the activity of hERG channel indirectly is a molecule that binds to a hERG-associated signaling complex in cells, thus causing the alteration in hERG activity, such as hERG current, ion flux via hERG, and/or cell signaling via hERG channel or hERG-associated signaling complex (this type modulator is referred to the hERG pathway modulator). The alteration in hERG activity is referenced to the basal activity of hERG channel or hERG-associated signaling complex in cells in the absence of a modulator.

2. GPR35 Modulators

In some forms, disclosed are methods of preventing and/or treating a subject, comprising administering to said subject a therapeutically effective amount of a compound of formula (II) or (III), or a pharmaceutically acceptable salt thereof, wherein the subject has a disease which is pathophysiologically related to GPR35.

In some other forms, disclosed are methods of preventing and/or treating a subject, comprising administering to said subject a therapeutically effective amount of a compound, or a pharmaceutically acceptable salt thereof, wherein the subject has a disease which is pathophysiologically related to GPR35, and wherein the compound having a chemical structure selected from the group consisting of:

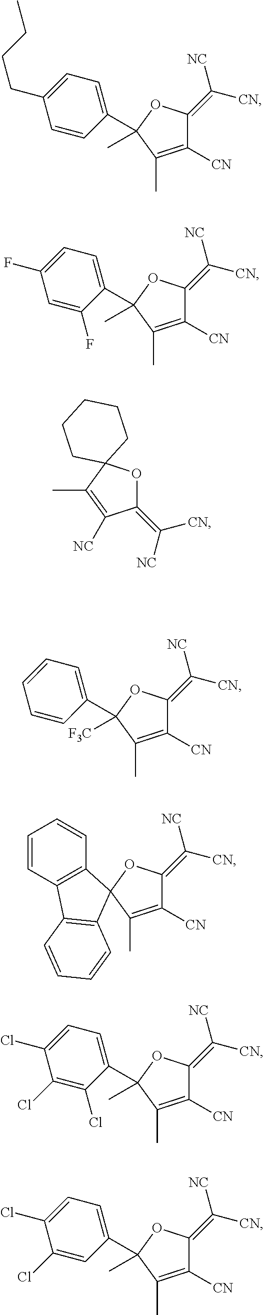

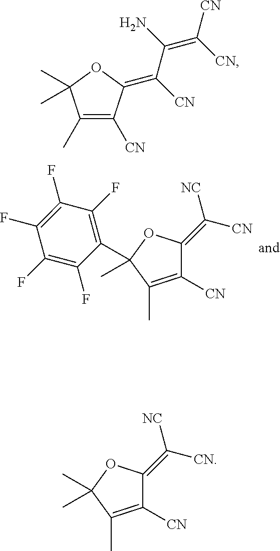

In some other forms, disclosed are methods of preventing and/or treating a subject, comprising administering to said subject a therapeutically effective amount of a compound, or a pharmaceutically acceptable salt thereof, wherein the subject has a disease which is pathophysiologically related to GPR35, and wherein the compound having a chemical structure selected from the group consisting of:

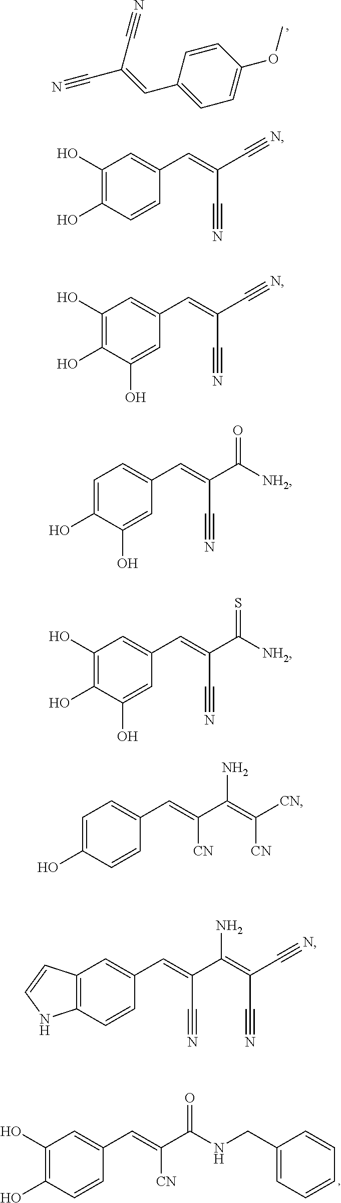

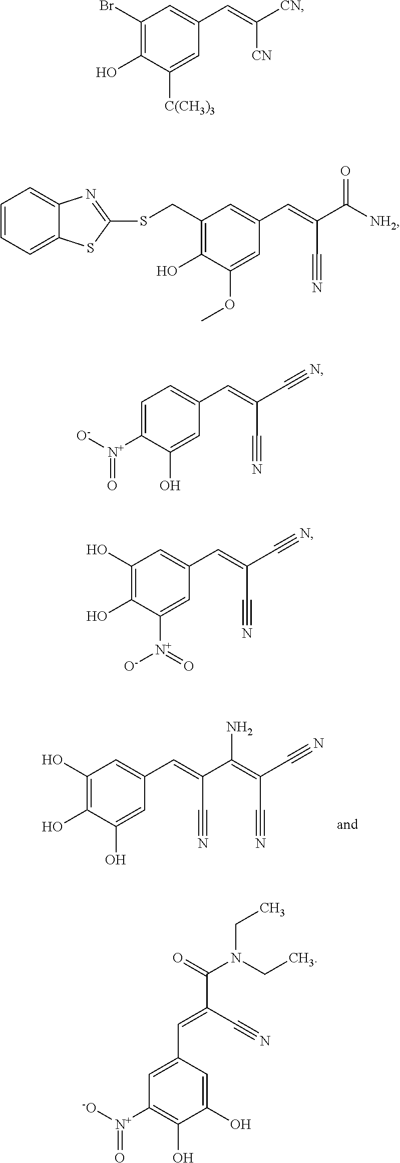

In some other forms, disclosed are methods of preventing and/or treating a subject, comprising administering to said subject a therapeutically effective amount of a compound, or a pharmaceutically acceptable salt thereof, wherein the subject has a disease which is pathophysiologically related to GPR35, and wherein the compound having a chemical structure selected from the group consisting of:

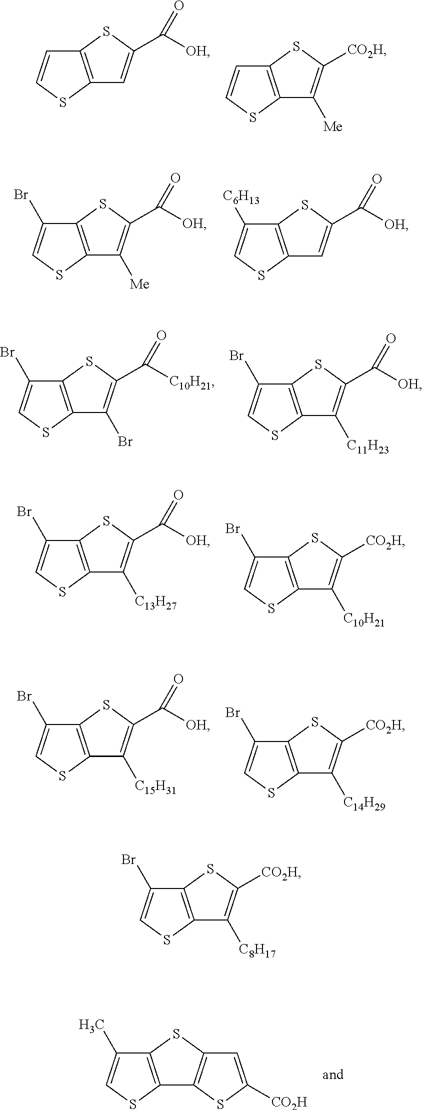

In some

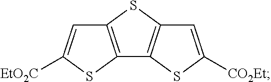

closed are methods of preventing and/or treating a subject, comprising administering to said subject a therapeutically effective amount of a compound, or a pharmaceutically acceptable salt thereof, wherein the subject has a disease which is pathophysiologically related to GPR35, and wherein the compound having a chemical structure selected from the group consisting of:

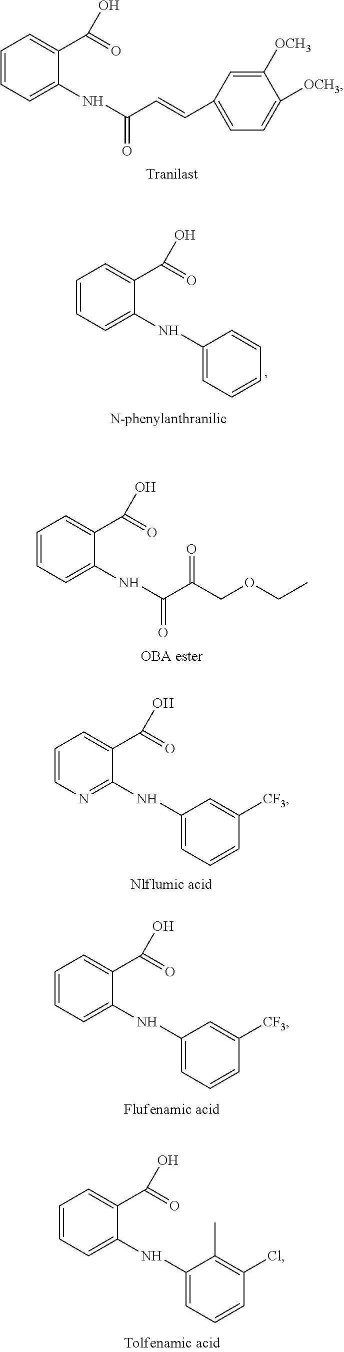

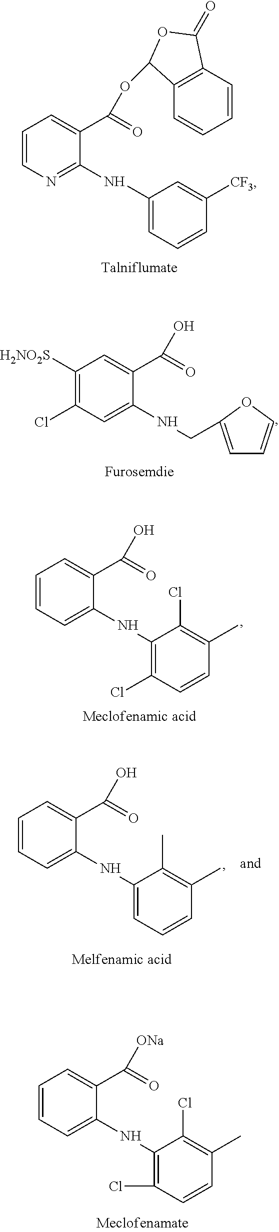

In some other forms, the methods of preventing and/or treating a subject as disclosed above are methods wherein said compound or a pharmaceutically acceptable salt thereof, is a GPR35 modulator. In some other forms, the methods of preventing and/or treating a subject as disclosed above are methods wherein said compound or a pharmaceutically acceptable salt thereof, is a GPR35 agonist.

In some other forms, the methods of preventing and/or treating a subject as disclosed above are methods wherein said GPR35 mediates cell signaling via G_{12/13}-ROCK (RhoA and Rho kinase) pathway. In some other forms, the methods of preventing and/or treating a subject as disclosed above are methods wherein said disease is selected from the group consisting of inflammation, asthma, metabolic disorder, congestive heart failure, and cancer. In some other forms, the methods of preventing and/or treating a subject as disclosed above are methods wherein said metabolic disorder is selected from the group consisting of diabetes, Type I diabetes, Type II diabetes, inadequate glucose tolerance, insulin resistance, hyperglycemia, hyperinsulinemia, hyperlipidemia, hypertriglyceridemia, hypercholesterolemia, dyslipidemia, obesity, aging, Syndrome X, atherosclerosis, heart disease, stroke, hypertension and peripheral vascular disease.

In some other forms, the methods of preventing and/or treating a subject as disclosed above are methods wherein said cancer is selected from the group consisting of prostate cancer, leukemia, hormone dependent cancers, breast cancer, colon cancer, lung cancer, epidermal cancer, liver cancer, esophageal cancer, stomach cancer, cancer of the brain, and cancer of the kidney.

In some other forms, the methods of preventing and/or treating a subject as disclosed above are methods wherein said compound is a GPR35 antagonist.

In some other forms, the methods of preventing and/or treating a subject as disclosed above are methods wherein the compound or a pharmaceutically acceptable salt thereof, is administered by one or more routes selected from a group consisting of rectal, buccal, sublingual, intravenous, subcutaneous, intradermal, transdermal, intraperitoneal, oral, eye drops, parenteral and topical administration.

In some forms, the methods of preventing and/or treating a subject as disclosed above are methods wherein the administration is accomplished by administering an oral form of said compound or a pharmaceutically acceptable salt thereof. In some other forms, the methods of preventing and/or treating a subject as disclosed above are methods wherein the administration is administration of an injectable form of said compound or a pharmaceutically acceptable salt thereof. In some other forms, the methods of preventing and/or treating a subject as disclosed above are methods wherein the administration is administration of a suppository form of said compound or a pharmaceutically acceptable salt thereof. In some other forms, the methods of preventing and/or treating a subject as disclosed above are methods wherein the administration is administration of an intra-operative instillation of a gel, cream, powder, foam, crystals, liposomes, spray or liquid suspension form of said compound, or a pharmaceutically acceptable salt thereof. In some other forms, the methods of preventing and/or treating a subject as disclosed above are methods wherein the administration is administration of said compound, or a pharmaceutically acceptable salt thereof, in a form of a transdermal patch or a transdermal pad.

In some forms, the methods of preventing and/or treating a subject as disclosed above are methods wherein the compound or a pharmaceutically acceptable salt thereof, is administered in an amount of about 0.001 to about 100 mg/kg body weight, or about 0.01 to about 100 mg/kg body weight on days of administration, or about 0.1 to about 100 mg/kg body weight on days of administration, or about 1 to about 100 mg/kg body weight on days of administration, or about 1 to about 50 mg/kg body weight on days of administration, or about 10 to about 50 mg/kg body weight on days of administration.

In some other forms, the methods of preventing and/or treating a subject as disclosed above are methods which further comprise one or more therapeutic agents. In some forms, the methods of preventing and/or treating a subject as disclosed above are methods wherein the therapeutic agent is an anti-inflammation, anti-metabolic-disorder, anti-congestive-heart-failure or anti-cancer agent. In some other forms, the methods of preventing and/or treating a subject as disclosed above are methods wherein the therapeutic agent is a compound of formula (II), or (III), or a pharmaceutically acceptable salt thereof. In some other forms, the methods of preventing and/or treating a subject as disclosed above are methods wherein the therapeutic agent is kynurenic acid, NPPB, zaprinast or lysophosphatidic acid (LPA).

In some forms, the methods of preventing and/or treating a subject as disclosed above are methods wherein the compound or a pharmaceutically acceptable salt thereof, and said one or more therapeutic agents are administered in separate formulation. In some other forms, the methods of preventing and/or treating a subject as disclosed above are methods wherein the compound or a pharmaceutically acceptable salt thereof, and said one or more therapeutic agents are administered in the same formulation. In some forms, the methods of preventing and/or treating a subject as disclosed above are methods wherein the compound or a pharmaceutically acceptable salt thereof, and said one or more therapeutic agents are administered concurrently or sequentially.

In some other forms, the methods of preventing and/or treating a subject as disclosed above are methods wherein the compound or a pharmaceutically acceptable salt thereof, and said one or more therapeutic agents are administered by the same or different routes. In some other forms, the methods of preventing and/or treating a subject as disclosed above are methods wherein the compound or a pharmaceutically acceptable salt thereof, and said one or more therapeutic agents, produce synergistic effect in preventing and/or treating disease which is pathophysiologically related to GPR35.

In some forms, the methods of preventing and/or treating a subject as disclosed above are methods wherein the disease is insensitive, resistant or refractory to treatment with said compound or a pharmaceutically acceptable salt thereof, or said one or more therapeutic agents administered as a single agent. In some other forms, the methods of preventing and/or treating a subject as disclosed above are methods wherein the compound or a pharmaceutically acceptable salt thereof, and said one or more therapeutic agents are each administered in an amount of from 1/100 to less than ½ of their normal individual therapeutic doses, or from 1/10 to less than ¼ of their normal individual therapeutic doses.

In some forms, the methods of preventing and/or treating a subject as disclosed above are methods wherein the subject is a mammal. In some other forms, the methods of preventing and/or treating a subject as disclosed above are methods wherein the subject has been identified as needing treatment for the disease or the administration. In some other forms, the methods of preventing and/or treating a subject as disclosed above are methods which further comprise the step of monitoring the subject for efficacy of the treatment. In some other forms, the methods of preventing and/or treating a subject as disclosed above are methods wherein monitoring the subject comprise analyzing a tissue sample obtained from the subject.

3. GPR35-hERG Complex Modulators

In some forms, disclosed are methods of preventing and/or treating a subject, comprising administering to said subject a therapeutically effective amount of a compound of formula (I), (II) or (III) or a pharmaceutically acceptable salt thereof, wherein the subject has a disease which is pathophysiologically related to GPR35-hEGR signaling complex.

In some forms, disclosed are methods of preventing and/or treating a subject, comprising administering to said subject a therapeutically effective amount of a compound, or a pharmaceutically acceptable salt thereof, wherein the subject has a disease which is pathophysiologically related to GPR35-hEGR signaling complex, wherein the compound having a chemical structure selected from the group consisting of:

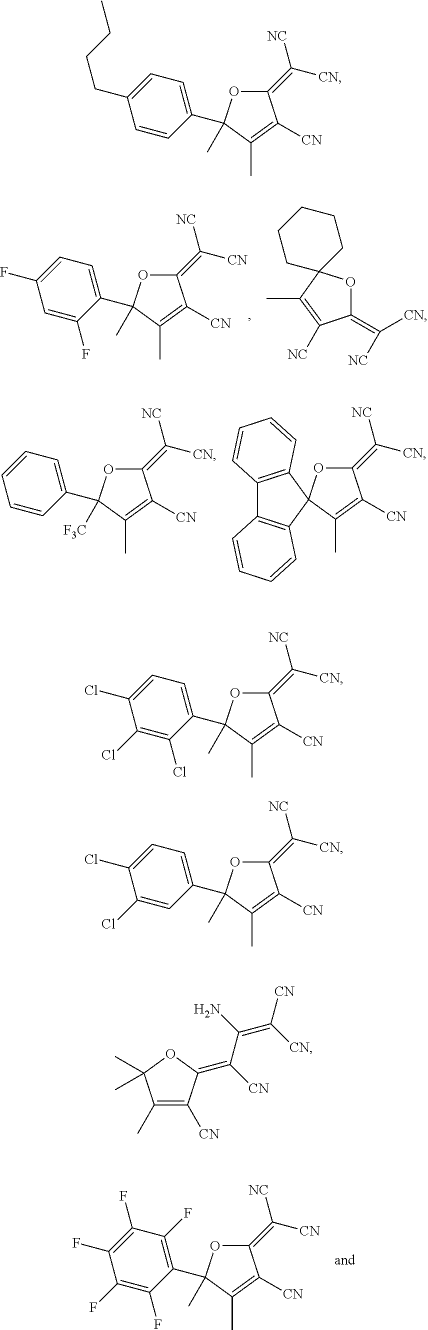

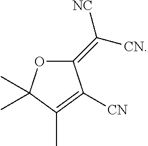

In some other forms, disclosed are methods of preventing and/or treating a subject, comprising administering to said subject a therapeutically effective amount of a compound, or a pharmaceutically acceptable salt thereof, wherein the subject has a disease which is pathophysiologically related to GPR35-hEGR signaling complex, and wherein the compound having a chemical structure selected from the group consisting of:

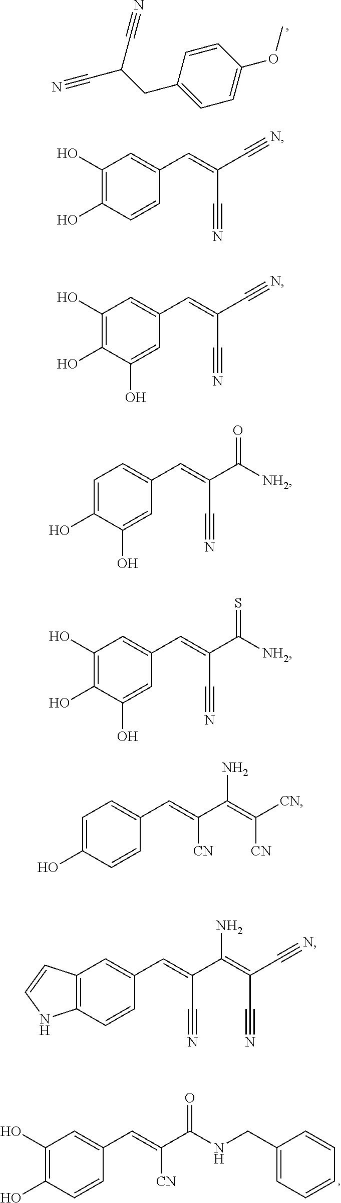

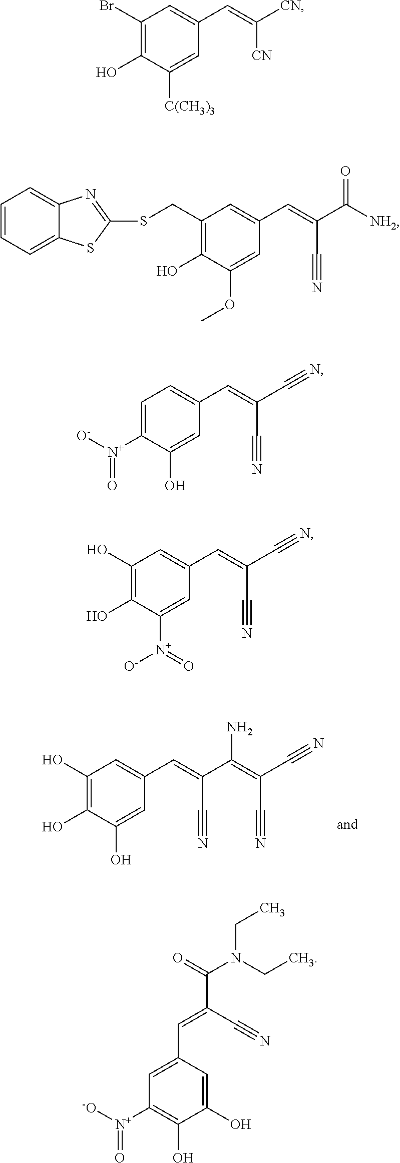

In some other forms, disclosed are methods of preventing and/or treating a subject, comprising administering to said subject a therapeutically effective amount of a compound, or a pharmaceutically acceptable salt thereof, wherein the subject has a disease which is pathophysiologically related to GPR35-hEGR signaling complex, and wherein the compound having a chemical structure selected from the group consisting of:

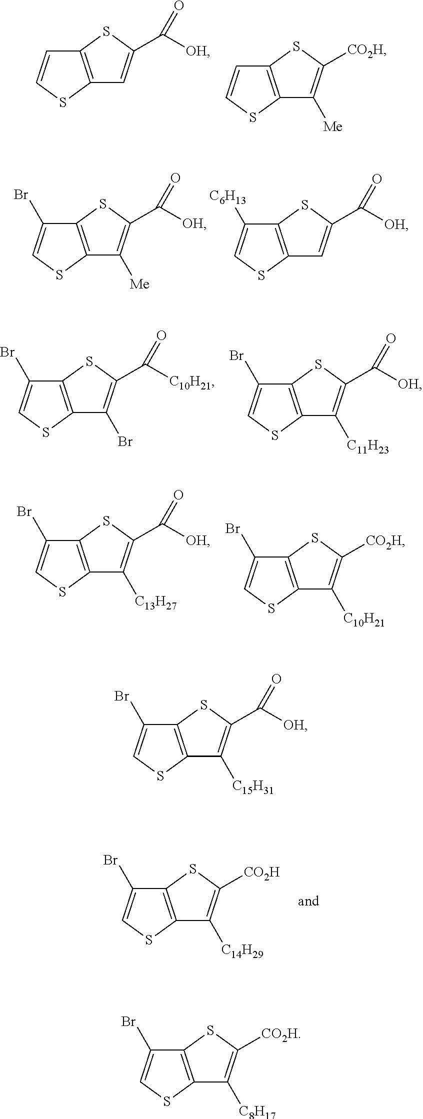

In some other forms, disclosed are methods of preventing and/or treating a subject, comprising administering to said subject a therapeutically effective amount of a compound, or a pharmaceutically acceptable salt thereof, wherein the subject has a disease which is pathophysiologically related to to GPR35-hEGR signaling complex, and wherein the compound having a chemical structure selected from the group consisting of:

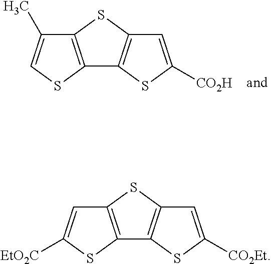

In some other forms, disclosed are methods of preventing and/or treating a subject, comprising administering to said subject a therapeutically effective amount of a compound, or a pharmaceutically acceptable salt thereof, wherein the subject has a disease which is pathophysiologically related to to GPR35-hEGR signaling complex, and wherein the compound having a chemical structure selected from the group consisting of:

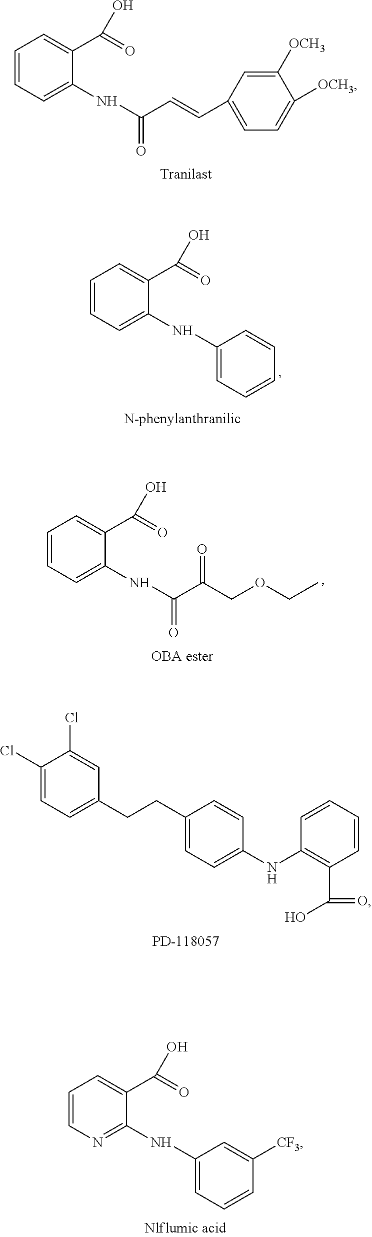

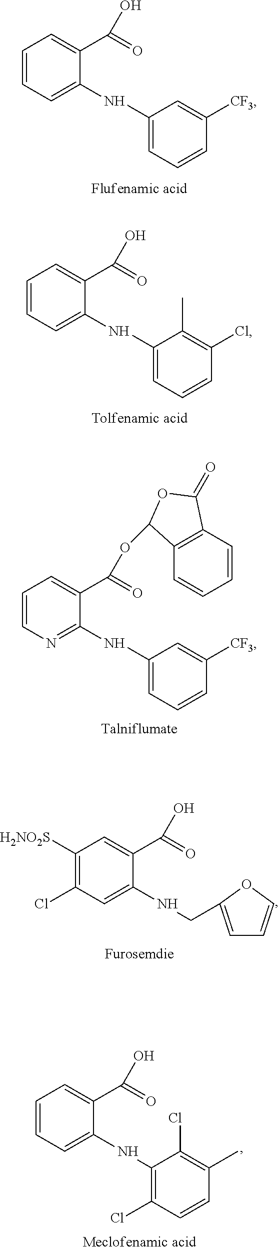

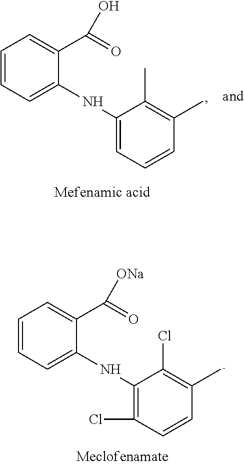

In some other forms, the methods of preventing and/or treating a subject as disclosed above are methods wherein said compound or a pharmaceutically acceptable salt thereof, is a GPR35-hEGR signaling complex modulator.

In some other forms, the methods of preventing and/or treating a subject as disclosed above are methods wherein said disease is selected from the group consisting of inflammation, asthma, metabolic disorder, congestive heart failure, and cancer. In some other forms, the methods of preventing and/or treating a subject as disclosed above are methods wherein said metabolic disorder is selected from the group consisting of diabetes, Type I diabetes, Type II diabetes, inadequate glucose tolerance, insulin resistance, hyperglycemia, hyperinsulinemia, hyperlipidemia, hypertriglyceridemia, hypercholesterolemia, dyslipidemia, obesity, aging, Syndrome X, atherosclerosis, heart disease, stroke, hypertension and peripheral vascular disease.

In some other forms, the methods of preventing and/or treating a subject as disclosed above are methods wherein said cancer is selected from the group consisting of prostate cancer, leukemia, hormone dependent cancers, breast cancer, colon cancer, lung cancer, epidermal cancer, liver cancer, esophageal cancer, stomach cancer, cancer of the brain, and cancer of the kidney.

In some other forms, the methods of preventing and/or treating a subject as disclosed above are methods wherein the compound or a pharmaceutically acceptable salt thereof, is administered by one or more routes selected from a group consisting of rectal, buccal, sublingual, intravenous, subcutaneous, intradermal, transdermal, intraperitoneal, oral, eye drops, parenteral and topical administration.

In some forms, the methods of preventing and/or treating a subject as disclosed above are methods wherein the administration is accomplished by administering an oral form of said compound or a pharmaceutically acceptable salt thereof. In some other forms, the methods of preventing and/or treating a subject as disclosed above are methods wherein the administration is administration of an injectable form of said compound or a pharmaceutically acceptable salt thereof. In some other forms, the methods of preventing and/or treating a subject as disclosed above are methods wherein the administration is administration of a suppository form of said compound or a pharmaceutically acceptable salt thereof. In some other forms, the methods of preventing and/or treating a subject as disclosed above are methods wherein the administration is administration of an intra-operative instillation of a gel, cream, powder, foam, crystals, liposomes, spray or liquid suspension form of said compound, or a pharmaceutically acceptable salt thereof. In some other forms, the methods of preventing and/or treating a subject as disclosed above are methods wherein the administration is administration of said compound, or a pharmaceutically acceptable salt thereof, in a form of a transdermal patch or a transdermal pad.

In some forms, the methods of preventing and/or treating a subject as disclosed above are methods wherein the compound or a pharmaceutically acceptable salt thereof, is administered in an amount of about 0.001 to about 100 mg/kg body weight, or about 0.01 to about 100 mg/kg body weight on days of administration, or about 0.1 to about 100 mg/kg body weight on days of administration, or about 1 to about 100 mg/kg body weight on days of administration, or about 1 to about 50 mg/kg body weight on days of administration, or about 10 to about 50 mg/kg body weight on days of administration.

In some other forms, the methods of preventing and/or treating a subject as disclosed above are methods which further comprise one or more therapeutic agents. In some forms, the methods of preventing and/or treating a subject as disclosed above are methods wherein the therapeutic agent is an anti-inflammation, anti-metabolic-disorder, anti-congestive-heart-failure or anti-cancer agent. In some other forms, the methods of preventing and/or treating a subject as disclosed above are methods wherein the therapeutic agent is a compound of formula (I), (II), or (III), or a pharmaceutically acceptable salt thereof. In some other forms, the methods of preventing and/or treating a subject as disclosed above are methods wherein the therapeutic agent is kynurenic acid, NPPB, zaprinast or lysophosphatidic acid (LPA).

In some forms, the methods of preventing and/or treating a subject as disclosed above are methods wherein the compound or a pharmaceutically acceptable salt thereof, and said one or more therapeutic agents are administered in separate formulation. In some other forms, the methods of preventing and/or treating a subject as disclosed above are methods wherein the compound or a pharmaceutically acceptable salt thereof, and said one or more therapeutic agents are administered in the same formulation. In some forms, the methods of preventing and/or treating a subject as disclosed above are methods wherein the compound or a pharmaceutically acceptable salt thereof, and said one or more therapeutic agents are administered concurrently or sequentially.

In some other forms, the methods of preventing and/or treating a subject as disclosed above are methods wherein the compound or a pharmaceutically acceptable salt thereof, and said one or more therapeutic agents are administered by the same or different routes. In some other forms, the methods of preventing and/or treating a subject as disclosed above are methods wherein the compound or a pharmaceutically acceptable salt thereof, and said one or more therapeutic agents, produce synergistic effect in preventing and/or treating disease which is pathophysiologically related to GPR35-hERG singling complex.

In some forms, the methods of preventing and/or treating a subject as disclosed above are methods wherein the disease is insensitive, resistant or refractory to treatment with said compound or a pharmaceutically acceptable salt thereof, or said one or more therapeutic agents administered as a single agent. In some other forms, the methods of preventing and/or treating a subject as disclosed above are methods wherein the compound or a pharmaceutically acceptable salt thereof, and said one or more therapeutic agents are each administered in an amount of from 1/100 to less than ½ of their normal individual therapeutic doses, or from 1/10 to less than ¼ of their normal individual therapeutic doses.

In some forms, the methods of preventing and/or treating a subject as disclosed above are methods wherein the subject is a mammal. In some other forms, the methods of preventing and/or treating a subject as disclosed above are methods wherein the subject has been identified as needing treatment for the disease or the administration. In some other forms, the methods of preventing and/or treating a subject as disclosed above are methods which further comprise the step of monitoring the subject for efficacy of the treatment. In some other forms, the methods of preventing and/or treating a subject as disclosed above are methods wherein monitoring the subject comprise analyzing a tissue sample obtained from the subject.

Disclosed relates to the use of the hERG-GPR35 complex modulators for improve prevention and treatment of hERG-GPR35 complex associated diseases such as inflammation, metabolic disorders, diabetes, congestive heart failure, or cancers. The hERG-GPR35 complex modulators include, but not limited to, molecules shown in Formulas I to III.

Disclosed further encompasses methods for treating or preventing hERG-GPR35 associated human diseases such as metabolic disorders and cancers, comprising administering to a subject in need thereof a compound of the invention or a pharmaceutically acceptable salt, solvate, clathrate, or prodrug thereof, or a pharmaceutical composition comprising a compound of the invention or a pharmaceutically acceptable salt, solvate, clathrate, or prodrug thereof. These methods may also comprise administering to the subject an additional agent separately or in a combination composition with the compound of the invention or a pharmaceutically acceptable salt, solvate, clathrate, or prodrug thereof.

Disclosed further encompasses methods for treating hERG-GPR35 associated human diseases such as metabolic disorders and cancers in vivo or in vitro using an effective amount of a compound of the invention, or a pharmaceutically acceptable salt, solvate, clathrate or prodrug thereof, or a pharmaceutical composition comprising an effective amount of a compound of the invention or a pharmaceutically acceptable salt, solvate, clathrate or prodrug thereof.

4. GPR35-hERG Complex Interfering Molecules

Also disclosed are methods of treating a subject comprising administering to said subject a therapeutically effective amount of a molecule identified in the disclosed methods, wherein the subject has a disease which is pathophysiologically related to the GPR35-hERG complex. For example, treating a subject with a GPR35-hERG complex interfering molecule can prevent or disrupt GPR35-hERG interaction which can affect the GPR35-hERG complex signaling pathway. Diseases which require this pathway can be treated by interupting the signaling pathway.

In some forms of the disclosed methods, the subject has been identified as needing treatment for the disease. In some forms of the disclosed methods, the methods further comprise the step of monitoring the subject for efficacy of the treatment.

C. Interfering with GPR35-hERG Complex Formation

Also disclosed are methods of preventing GPR35-hERG complex formation comprising contacting a GPR35 and hERG expressing cell with a GPR35-hERG complex interfering molecule.

Also disclosed are methods of disrupting GPR35-hERG complex formation comprising contacting a composition comprising a GPR35-hERG complex with a GPR35-hERG complex interfering molecule.

Also disclosed are methods of identifying GPR35-hERG complex binding molecules comprising contacting an isolated GPR35-hERG complex or composition comprising the GPR35-hERG complex with a test molecule; and determining if the test molecule binds to the GPR35-hERG complex. Some molecules can affect or regulate the GPR35-hERG signaling complex without direct binding to the complex and some molecules require direct binding. The disclosed complexes and compositions can be used for binding studies. After determining if a molecule binds the GPR35-hERG complex, one can then determine if the molecule modulates the complex or vice versa. The use of an isolated complex or composition can allow for more convenient and inexpensive experiments for binding studies than using a cell line that comprises the complex.

The disclosed methods can be used for treating subjects as well as for studying the GPR35-hERG complex signaling pathway.

### D. Making and Using Engineered Cells

The present invention additionally provides various related methods. The cells of the invention can be utilized for various methods, e.g., related assays. One aspect of the invention provides, methods of expressing a GPCR from a cell comprising introducing into the cell a nucleic acid comprising a promoter operatively linked to a receptor coding region. In some embodiments, the method comprises introducing the nucleic acid by transfection, electroporation, microinjection, or infection with a viral vector. In one embodiment, the promoter operatively linked to the receptor coding region is a regulatable promoter.

Some aspects of the invention provide methods of detecting or monitoring activity of GPR35/hERG signaling complex: (a) culturing a cell of the invention under conditions wherein the GPR35/hERG signaling complex is present; and (b) detecting the cellular response.

Some aspects of the invention provide methods for measuring the ability of a compound(s) to affect or modulate activation of a GPR35/hERG signaling complex comprising: (a) culturing a cell of the invention under conditions wherein the GPR35/hERG signaling complex is present; (b) contacting the cell with the compound(s); and (c) measuring the cellular response.

According to the present invention, any mutations of GPR35, including constitutive isoforms, can be expressed. Alternatively, any mutations of hERG channels can be expressed so that many different combinations can be achieved.

For example, engineered cells expressing a mutant GPR35 which prevents an active GPR35 agonist stimulated GPR35 pathway can be used to screen for hERG transactivating GPR35 agonists which do not require activation of the GPR35 pathway. This would indicate that the hERG transactivating ability of the GPR35 agonist uses something other than the GPR35 pathway to transactivate.

### E. More Definitions of Terms

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### 1. A, AN, THE

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

### 2. ABBREVIATIONS

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

### 3. ABOUT

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

### 4. ANALYTICAL METHODS

An analytical method is for example, a method which measures a molecule or substance. For example, gas chromatography, gel permeation chromatography, high resolution gas chromoatography, high resolution mass spectrometry, or mass spectrometry is analytical methods.

### 5. ASSAYING

Assaying, assay, or like terms refers to an analysis to determine a characteristic of a substance, such as a molecule or a cell, such as for example, the presence, absence, quantity, extent, kinetics, dynamics, or type of an a cell's optical or bioimpedance response upon stimulation with one or more exogenous stimuli, such as a ligand or marker. Producing a biosensor signal of a cell's response to a stimulus can be an assay.

### 6. ASSAYING THE RESPONSE

“Assaying the response” or like terms means using a means to characterize the response. For example, if a molecule is brought into contact with a cell, a biosensor can be used to assay the response of the cell upon exposure to the molecule.

### 7. AGONISM AND ANTAGONISM MODE

The agonism mode or like terms is the assay wherein the cells are exposed to a molecule to determine the ability of the molecule to trigger biosensor signals such as DMR signals, while the antagonism mode is the assay wherein the cells are exposed to a maker in the presence of a molecule to determine the ability of the molecule to modulate the biosensor signal of cells responding to the marker.

### 8. BIOSENSOR

Biosensor or like terms refer to a device for the detection of an analyte that combines a biological component with a physicochemical detector component. The biosensor typically consists of three parts: a biological component or element (such as tissue, microorganism, pathogen, cells, or combinations thereof), a detector element (works in a physicochemical way such as optical, piezoelectric, electrochemical, thermometric, or magnetic), and a transducer associated with both components. The biological component or element can be, for example, a living cell, a pathogen, or combinations thereof. In embodiments, an optical biosensor can comprise an optical transducer for converting a molecular recognition or molecular stimulation event in a living cell, a pathogen, or combinations thereof into a quantifiable signal. Typical biosensors used for label-free cellular assays are surface plasmon resonance, plasmon resonance imaging, resonant waveguide grating biosensor, photonic crystal biosensor, and electric impedance biosensors.

### 9. BIOSENSOR RESPONSE

A “biosensor response”, “biosensor output signal”, “biosensor signal” or like terms is any reaction of a sensor system having a cell to a cellular response. A biosensor converts a cellular response to a quantifiable sensor response. A biosensor response is an optical response upon stimulation as measured by an optical biosensor such as RWG or SPR or it is a bioimpedence response of the cells upon stimulation as measured by an electric biosensor. Since a biosensor response is directly associated with the cellular response upon stimulation, the biosensor response and the cellular response can be used interchangeably, in embodiments of disclosure.

### 10. BIOSENSOR SIGNAL

A “biosensor signal” or like terms refers to the signal of cells measured with a biosensor that is produced by the response of a cell upon stimulation.

### 11. BIOSENSOR INDEX

A “biosensor index” or like terms is an index made up of a collection of biosensor data. A biosensor index can be a collection of biosensor profiles, such as primary profiles, or secondary profiles. The index can be comprised of any type of data. For example, an index of profiles could be comprised of just an N-DMR data point, it could be a P-DMR data point, or both or it could be an impedence data point. It could be all of the data points associated with the profile curve.

### 12. CELL

The term “cell” as used herein also refers to individual cells, cell lines, or cultures derived from such cells. A “culture” refers to a composition comprising isolated cells of the same or a different type. The term co-culture is used to designate when more than one type of cell are cultured together in the same dish with either full or partial contact with each other.

### 13. CELL CULTURE

“Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

### 14. CELL PANEL

A “cell panel” or like terms is a panel which comprises at least two types of cells. The cells can be of any type or combination disclosed herein.

### 15. CELLULAR RESPONSE

A “cellular response” or like terms is any reaction by the cell to a stimulation.

### 16. CELLULAR PROCESS

A cellular process or like terms is a process that takes place in or by a cell. Examples of cellular process include, but not limited to, proliferation, apoptosis, necrosis, differentiation, cell signal transduction, polarity change, migration, or transformation.

### 17. CELLULAR TARGET

A “cellular target” or like terms is a biopolymer such as a protein or nucleic acid whose activity can be modified by an external stimulus. Cellular targets are most commonly proteins such as enzymes, kinases, ion channels, and receptors.

### 18. CHARACTERIZING

Characterizing or like terms refers to gathering information about any property of a substance, such as a ligand, molecule, marker, or cell, such as obtaining a profile for the ligand, molecule, marker, or cell.

### 19. COMPOUND INTERCHANGEABILITY

For the purposes of the present disclosure the terms “compound,” “analog,” and “composition of matter” stand equally well for the chemical entities described herein, including all enantiomeric forms, diastereomeric forms, salts, and the like, and the terms “compound,” “analog,” and “composition of matter” are used interchangeably throughout the present specification.

### 20. COMPRISE

Throughout the description and claims of this specification, the word “comprise” and variations of the word, such as “comprising” and “comprises,” means “including but not limited to,” and is not intended to exclude, for example, other additives, components, integers or steps.

### 21. CONSISTING ESSENTIALLY OF

“Consisting essentially of” in embodiments refers, for example, to a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, aberrant affinity of a stimulus for a cell surface receptor or for an intracellular receptor, anomalous or contrary cell activity in response to a ligand candidate or like stimulus, and like characteristics.

### 22. COMPONENTS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### 23. CONTACTING

Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e., such that they are in proximity to mix or touch. For example, having a solution of composition A and cultured cell B and pouring solution of composition A over cultured cell B would be bringing solution of composition A in contact with cell culture B. Contacting a cell with a ligand would be bringing a ligand to the cell to ensure the cell have access to the ligand.

It is understood that anything disclosed herein can be brought into contact with anything else. For example, a cell can be brought into contact with a marker or a molecule, a biosensor, and so forth.

### 24. COMPOUNDS AND COMPOSITIONS

Compounds and compositions have their standard meaning in the art. It is understood that wherever, a particular designation, such as a molecule, substance, marker, cell, or reagent compositions comprising, consisting of, and consisting essentially of these designations are disclosed. Thus, where the particular designation marker is used, it is understood that also disclosed would be compositions comprising that marker, consisting of that marker, or consisting essentially of that marker. Where appropriate wherever a particular designation is made, it is understood that the compound of that designation is also disclosed. For example, if particular biological material, such as EGF, is disclosed EGF in its compound form is also disclosed.

### 25. CONTROL

The terms “control” or “control levels” or “control cells” are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard.

### 26. CLATHRATE

A compound for use in the invention may form a complex such as a “clathrate”, a drug-host inclusion complex, wherein, in contrast to solvates, the drug and host are present in stoichiometric or non-stoichiometric amounts. A compound used herein can also contain two or more organic and/or inorganic components which can be in stoichiometric or non-stoichiometric amounts. The resulting complexes can be ionised, partially ionised, or non-ionised. For a review of such complexes, see J. Pharm. ScL, 64 (8), 1269-1288, by Haleblian (August 1975).

### 27. DETECT

Detect or like terms refer to an ability of the apparatus and methods of the disclosure to discover or sense a molecule- or a marker-induced cellular response and to distinguish the sensed responses for distinct molecules.

### 28. DIRECT ACTION (OF A DRUG CANDIDATE MOLECULE)

A “direct action” or like terms is a result (of a drug candidate molecule”) acting independently on a cell.

### 29. DMR INDEX

A “DMR index” or like terms is a biosensor index made up of a collection of DMR data.

### 30. DMR SIGNAL

A “DMR signal” or like terms refers to the signal of cells measured with an optical biosensor that is produced by the response of a cell upon stimulation.

### 31. DMR RESPONSE

A “DMR response” or like terms is a biosensor response using an optical biosensor. The DMR refers to dynamic mass redistribution or dynamic cellular matter redistribution. A P-DMR is a positive DMR response, a N-DMR is a negative DMR response, and a RP-DMR is a recovery P-DMR response.

### 32. DISEASE MARKER

A disease marker is any reagent, molecule, substance, etc, that can be used for identifying, diagnosing, or prognosing is for the hERG channel related disease, or the GPR35 related disease, or the GPR35-hERG complex related disease.

### 33. DRUG CANDIDATE MOLECULE

A drug candidate molecule or like terms is a test molecule which is being tested for its ability to function as a drug or a pharmacophore. This molecule may be considered as a lead molecule.

### 34. EFFICACY

Efficacy or like terms is the capacity to produce a desired size of an effect under ideal or optimal conditions. It is these conditions that distinguish efficacy from the related concept of effectiveness, which relates to change under real-life conditions. Efficacy is the relationship between receptor occupancy and the ability to initiate a response at the molecular, cellular, tissue or system level.

### 35. HERG MODULATOR

A hERG modulator is a molecule that can modulate the activity of hERG ion channel directly or indirectly. A hERG-specific modulator that modulates the activity of hERG channel directly is a molecule that binds to hERG channels, thus causing the alteration in hERG activity, such as hERG current, ion flux via hERG, and/or cell signaling via hERG. A hERG pathway modulator that modulates the activity of hERG channel indirectly is a molecule that binds to a hERG-associated signaling complex in cells, thus causing the alteration in hERG activity, such as hERG current, ion flux via hERG, and/or cell signaling via hERG channel or hERG-associated signaling complex. The alteration in hERG activity is referenced to the basal activity of hERG channel or hERG-associated signaling complex in cells in the absence of a modulator.

### 36. hERG ACTIVATOR

A hERG activator is a molecule that increases the current via hERG channel at appropriate applied voltages, and/or increases the ion flux via hERG channel in the presence of appropriate KCl concentrations, and/or triggers cell signaling via hERG channel or hERG-associated signaling complex in cells. Examples are mallotoxin, flufenamic acid, and niflumic acid.

### 37. hERG PATHWAY ACTIVATOR

A hERG pathway activator is a molecule that triggers cell signaling via hERG channel or hERG-associated signaling complex in cells. A hERG pathway activator may or may not cause any alteration in hERG current, and/or ion flux via hERG channel Alteration can either increase or decrease. Examples are diflunisal, AG126, and tyrphostin 51.

### 38. hERG ION CHANNEL ACTIVATOR

A hERG ion channel activator is a molecule that directly binds to and activates hERG channel, thus leading to increase in hERG current, and/or increase in hERG ion flux, and/or cell signaling via hERG channel Examples are mallotoxin, flufenamic acid, and niflumic acid. A hERG ion channel activator may or may not trigger cell signaling.

### 39. hERG INHIBITOR

A hERG inhibitor is a molecule that binds to hERG channel, or hERG protein in a hERG-associated signaling complex, thus inhibiting hERG current and/or hERG ion flux.

### 40. hERG PATHWAY INHIBITOR

A hERG inhibitor is a molecule that binds to a receptor, rather than hERG protein, in a hERG-associated signaling complex, thus inhibiting hERG current, and/or hERG ion flux. Example includes tyrphostin 51.

### 41. hERG ION CHANNEL INHIBITOR

A hERG ion channel inhibitor is a molecule that binds to hERG channel directly and thus inhibits hERG current, and/or hERG ion flux. Example includes dofetilide.

### 42. hERG CHANNEL RELATED DISEASE

A hERG channel related disease is a disease in which the cause of the disease or the treatment of the disease can be altered by modulation of the hERG channel. Exemplary diseases are cancers, such as leukemia, colon cancer, gastric cancer, breast cancer, or lung cancer. Exemplary diseases are genetic mutation caused inherited long QT syndrome (LQTS), drug molecule-caused acquired LQTS, and class III arrhymics.

### 43. HIGHER AND INHIBIT AND LIKE WORDS

The terms higher, increases, elevates, or elevation or like terms or variants of these terms, refer to increases above basal levels, e.g., as compared a control. The terms low, lower, reduces, decreases or reduction or like terms or variation of these terms, refer to decreases below basal levels, e.g., as compared to a control. For example, basal levels are normal in vivo levels prior to, or in the absence of, or addition of a molecule such as an agonist or antagonist to a cell. Inhibit or forms of inhibit or like terms refers to to reducing or suppressing.

### 44. HIGHER, INCREASE, ELEVATE, ELEVATION

The terms “higher”, “increases”, “elevates”, or “elevation”, or like terms or variants of these terms, refer to increases above basal levels, e.g., as compared a control. The terms “low”, “lower”, “reduces”, “decreases” or “reduction”, or variation of these terms, refer to decreases below basal levels, e.g., as compared to a control. For example, basal levels are normal in vivo levels prior to, or in the absence of, or addition of an agent such as an agonist or antagonist to activity. For example, decreases or increases can be used to describe the binding of a molecule to a receptor. In this context, decreases would describe a situation of where the binding could be defined as having a Kd of 10⁻⁹ M, if this interaction decreased, meaning the binding lessened, the Kd could decrease to 10⁻⁶ M. It is understood that wherever one of these words is used it is also disclosed that it could be 1%, 5%, 10%, 20%, 50%, 100%, 500%, or 1000% increased or decreased from a control.

### 45. IN THE PRESENCE OF THE MOLECULE

“in the presence of the molecule” or like terms refers to the contact or exposure of the cultured cell with the molecule. The contact or exposure can be taken place before, or at the time, the stimulus is brought to contact with the cell.

### 46. INDEX

An index or like terms is a collection of data. For example, an index can be a list, table, file, or catalog that contains one or more modulation profiles. It is understood that an index can be produced from any combination of data. For example, a DMR profile can have a P-DMR, a N-DMR, and a RP-DMR. An index can be produced using the completed date of the profile, the P-DMR data, the N-DMR data, the RP-DMR data, or any point within these, or in combination of these or other data. The index is the collection of any such information. Typically, when comparing indexes, the indexes are of like data, i.e. P-DMR to P-DMR data.

### 47. INHIBIT

The term “inhibit” or like words means to hinder or restrain a particular characteristic. It is understood that this is typically in relation to some standard or expected value, in other words it is relative, but that it is not always necessary for the standard or relative value to be referred to. For example, “inhibits phosphorylation” means hindering or restraining the amount of phosphorylation that takes place relative to a standard or a control.

### 48. KNOWN MOLECULE

A known molecule or like terms is a molecule with known pharmacological/biological/physiological/pathophysiological activity whose precise mode of action(s) may be known or unknown.

### 49. KNOWN MODULATOR

A known modulator or like terms is a modulator where at least one of the targets is known with a known affinity. For example, a known modulator could be a GPR35 agonist, a GPR35 antagonist, etc.

### 50. KNOWN MODULATOR BIOSENSOR INDEX

A “known modulator biosensor index” or like terms is a modulator biosensor index produced by data collected for a known modulator. For example, a known modulator biosensor index can be made up of a profile of the known modulator acting on the panel of cells, and the modulation profile of the known modulator against the panels of markers, each panel of markers for a cell in the panel of cells.

### 51. KNOWN MODULATOR DMR INDEX

A “known modulator DMR index” or like terms is a modulator DMR index produced by data collected for a known modulator. For example, a known modulator DMR index can be made up of a profile of the known modulator acting on the panel of cells, and the modulation profile of the known modulator against the panels of markers, each panel of markers for a cell in the panel of cells.

### 52. hERG-SPECIFIC MODULATOR

A hERG-specific modulator is a molecule that can modulate the activity of hERG ion channel via direct binding to the hERG channel. The hERG-specific modulator can cause the alteration in hERG activity, such as hERG current, ion flux via hERG, and/or cell signaling via hERG.

### 53. hERG EXPRESSING CELL

A hERG expressing cell is a cell either endogenously or recombinantly expressing hERG channel. The hERG expressing cell can be a leukemia cell line, a gastric cancer cell line, a neuroblastoma cell line, a mammary carcinoma cell line, and a human colon carcinoma cell line, a cardiovascular cell line, and a neuronal cell line. A cell expressing hERG can be HL60, cell line SGC7901, cell line MGC803, cell line SH-SY5Y, cell line MCF-7, cell line HT-29 and HCT8, and cell line HCT116

### 54. hERG NON-EXPRESSING CELL

A hERG non-expressing cell is a cell that does not express any functional hERG proteins. Examples are human embryonic kidney cell line and Chinese Ovary hamster cell line, such as HEK-293 and cell line CHO-K1.

### 55. LABEL-FREE BIOSENSOR CELLULAR ASSAY

A label free biosensor cellular assay or like terms is any assay that uses a label free biosensor to detect or monitor a cellular response.

### 56. ELECTROPHYSIOLOGY METHOD

An electrophysiology method is any method which study the electrical properties of biological cells and tissues. It involves measurements of voltage change or electric current on a wide variety of scales from single ion channel proteins to whole organs like the heart. In neuroscience, it includes measurements of the electrical activity of neurons, and particularly action potential activity. Recordings of large-scale electric signals from the nervous system such as electroencephalography, may also be referred to as electrophysiological recordings.

### 57. KNOWN hERG MODULATOR

A known hERG activator is any hERG activator that at the time it is used in an assay was known to be a hERG activator, as shown in any way. Known hERG blockers include, but not limited to, arsenic trioxide, astemizole, bepridil, chloroquine, cisapride, clarithromycin, disopyramide, dofetilide, domperidone, droperidol, erythromycin, fluoxetine, fluvoxamine, halofantrine, haloperidol, ibutilide, levomethadyl, mesoridazine, methadone, norfluoxetine, pentamidine, pimozide, probucol, procainamide, quinidine, sotalol, sparfloxacin, terfenadine, fexofenadine, thioridazine, verapamil. Known hERG activators include RPR260243, PD-118057, PD-307243, mallotoxin, niflumic acid, flufenamic acid, NS1643, NS3623, A-935142 and ICA-105574.

### 58. GPR35-SPECIFIC MODULATOR

A GPR35-specific moldulator or GPR35 modulator or the like term is any modulator which direct binds to GPR35 and thus modulates the activity of GPR35. A typical GPR35-specific modulator can modulate GPR35 activity in one of three cellular assays: (1) Ca²⁺ mobilization assays in an engineered cell such as HEK-GPR35 with and without co-expressing G_{qo5}. G_{qo5} is a G protein whose activation results in Ca²⁺ mobilization, and the G_{qo5} protein can be activated by the agonist-induced activation of a non-G_{q}-coupled receptor when expressed in the cell. Since GPR35 is believed to be a non-G_{q}-coupled receptor, the co-expression of G_{qo5} is necessary to detect the GPR35 agonist induced Ca²⁺ mobilization signal. (2) Receptor internalization assays. Receptor internalization is quick universal to almost all GPCRs. (3) Label-free dynamic mass redistribution (DMR) assays, as promised by optical biosensors such as resonant waveguide grating biosensor. The GPR35 modulator can be an agonist, an antagonist, an inverse agonist, and a biased agonism.

### 59. GPR35 EXPRESSING CELL

A GPR35 expressing cell is any cell which produces a functional GPR35 in the cell membrane of the cell.

### 60. GPR35 AGONIST

A GPR35 agonist is any molecule which binds to and thus activates the GPR35 receptor in the cells. Examples, as disclosed in the present invention and/or in published liatetures, include, but not limited to, diflunisal, flufenamic acid, flunxin, furosemdie, niflumic acid, NPPB, tolfenamic acid, zaprinast, YE210, or DNQX.

### 61. KNOWN GPR35 AGONIST

A known GPR35 agonist is any GPR35 agonist that at the time it is used in an assay was known to be a GPR35 agonist, as shown in any way.

### 62. GPR35 ANTAGONIST

A GPR35 antagonist is any molecule that binds but thus inhibits the activity of GPR35 receptor.

### 63. KNOWN GPR35 ANTAGONIST

A known GPR35 antagonist is any GPR35 antagonist that at the time it is used in an assay was known to be a GPR35 antagonist, as shown in any way. To date, there is no GPR35 antagonist reported in literature.

### 64. PATHOPHYSIOLOGICALLY RELATED TO GPR35

Something is “pathophysiologically related to GPR35” if GPR35 is involved in the functional changes in body associated with or resulting from disease or injury.

### 65. INFLAMMATION

Inflammation is any specific or non-specific immune response. Inflammation is part of the complex biological response of vascular tissues to harmful stimuli, such as pathogens, damaged cells, or irritants. Inflammation is a protective attempt by the organism to remove the injurious stimuli and to initiate the healing process. Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

### 66. METABOLIC DISORDER

A metabolic disorder is a disorder of metabolism, such as diabetes, Type I diabetes, Type II diabetes, inadequate glucose tolerance, insulin resistance, hyperglycemia, hyperinsulinemia, hyperlipidemia, hypertriglyceridemia, hypercholesterolemia, dyslipidemia, obesity, aging, Syndrome X, atherosclerosis, heart disease, stroke, hypertension and peripheral vascular disease.

### 67. CONGESTIVE HEART FAILURE

Congestive heart failure (CHF) is a condition in which the heart's function as a pump to deliver oxygen rich blood to the body is inadequate to meet the body's needs. Congestive heart failure can be caused by diseases that weaken the heart muscle, or diseases that cause stiffening of the heart muscles, or diseases that increase oxygen demand by the body tissue beyond the capability of the heart to deliver. Many diseases can impair the pumping action of the ventricles. For example, the muscles of the ventricles can be weakened by heart attacks or infections (myocarditis). The diminished pumping ability of the ventricles due to muscle weakening is called systolic dysfunction. After each ventricular contraction (systole) the ventricle muscles need to relax to allow blood from the atria to fill the ventricles. This relaxation of the ventricles is called diastole. Diseases such as hemochromatosis or amyloidosis can cause stiffening of the heart muscle and impair the ventricles' capacity to relax and fill; this is referred to as diastolic dysfunction. The most common cause of this is longstanding high blood pressure resulting in a thickened (hypertrophied) heart. Additionally, in some patients, although the pumping action and filling capacity of the heart may be normal, abnormally high oxygen demand by the body's tissues (for example, with hyperthyroidism) may make it difficult for the heart to supply an adequate blood flow (called high output heart failure). In some patients one or more of these factors can be present to cause congestive heart failure. Congestive heart failure can affect many organs of the body. For example, the weakened heart muscles may not be able to supply enough blood to the kidneys, which then begin to lose their normal ability to excrete salt (sodium) and water. This diminished kidney function can cause to body to retain more fluid. The lungs may become congested with fluid (pulmonary edema) and the person's ability to exercise is decreased. Fluid may likewise accumulate in the liver, thereby impairing its ability to rid the body of toxins and produce essential proteins. The intestines may become less efficient in absorbing nutrients and medicines. Over time, untreated, worsening congestive heart failure will affect virtually every organ in the body.

### 68. CANCER

38. Cancer is a disease of inadequately controlled differentiation or division of cells, such as prostate cancer, leukemia, hormone dependent cancers, breast cancer, colon cancer, lung cancer, epidermal cancer, liver cancer, esophageal cancer, stomach cancer, cancer of the brain, and cancer of the kidney. Cancer is a collection of diseases that arise from the progressive accumulation of genetic alterations in somatic cells. Cancer is also viewed as a pathway dysregulated disease—a small number of core pathways are dominate in aberrant cell growth leading to cancer. The ability of tumor cells to outgrow their neighboring cells is often driven by constitutive activation of downstream proteins. Genetic studies over several decades have discovered a wide range of tumor-associated genes and their mutations, many of which preferentially occur in signaling proteins involved in a small number of pathways. Genetic mutations are often enriched in positive regulatory loops (gain of function), and methylated genes in negative regulatory loops (loss of function), leading to the disruption of the normal cooperative behavior of cells and thus promoting tumor phenotypes. A hallmark in the onset of cancer is how mutated proteins alter and govern signaling of cancer cells in the context of intracellular or intercellular signaling networks.

### 69. GPR35-hERG SIGNALING COMPLEX MODULATOR

A hERG-GPR35 complex modulators (or GPR35-hERG signalling complex modulator) is a molecule that binds to either GPR35, or hERG, or both, thus modulating the activity of the GPR35-hERG signaling complex. A hERG-GPR35 complex modulators (or GPR35-hERG signalling complex modulator) is capable of producing a cross-desensitization in DMR assay in a hERG-GPR35 co-expressing cell.

### 70. CROSS-DESENSITIZATION DMR ASSAY

A cross-desensitization DMR assay is a label-free optical biosensor cellular assay that measures the ability of a molecule to desensitize the cellular response mediated by either a hERG activator such as mallotoxin or a GPR35 agonist such as zaprinast or YE210, in a hERG and GPR35 co-expressing cell, wherein the molecule itself also exhibits agonism activity in said cell.

### 71. AGONISM ACTION

Agonism action refers to the binding of a molecule to a receptor that leads to the activation of the receptor, thus triggering a cellular response similar to the cellular response for a known agonist for the receptor.

### 72. ANTAGONISM ACTION

Antagonism action refers to the binding of a molecule to a receptor that leads to the inhibition of the receptor.

### 73. hERG TRANSACTIVING GPR35 AGONIST

A hERG transactivating GPR35 agonist is a GPR35 agonist that can transactivate the hERG channel once it binds to GPR35 when the hERG channel is complexed with GPR35 in a hERG and GPR35 co-expressing cell.

### 74. hERG NON-TRANSACTIVATING GPR35 AGONIST A hERG non-transactivating GPR35 agonsit is a GPR35 agonist that does not transactivate the hERG channe once it binds to GPR35 when the hERG channel is complexed with GPR35 in a hERG and GPR35 co-expressing cell.

### 75. GPR35 TRANSACTIVATING hERG ACTIVATOR

A GPR35 transactivating hERG activator is a hERG activator that can transactivate the GPR35 receptor once it binds to the hERG channel when the GPR35 receptor is complexed with hERG in a hERG and GPR35 co-expressing cell.

### 76. GPR35 NON-TRANSACTIVATING hERG ACTIVATOR

A GPR35 non-transactivating hERG activator is a hERG activator that does not transactivate the GPR35 receptor once it binds to the hERG channel when the GPR35 receptor is complexed with hERG in a hERG and GPR35 co-expressing cell.

### 77. NORMAL INDIVIDUAL THERAPEUTIC DOSE

In certain compositions, more than one active therapeutic agent is present. This is called a combination composition. Thus, within a combination composition, a normal individual therapeutic dose is the dosage that one of the active therapeutic agents is administered at as a single active therapeutic agent.

### 78. PATHOPHYSIOLOGICALLY RELATED TO GPR35-hERG COMPLEX

Something is “pathophysiologically related to GPR35-hERG complex” if the GPR35-hERG complex is involved in the functional changes in body associated with or resulting from disease or injury.

### 79. THERAPEUTIC AGENT

A therapeutic agent is any agent which has been determined to have a therapeutic effect.

### 80. ANTI-INFLAMMATION AGENT

An antiinflammatory agent is any agent that has an anti-inflammatory activity. Examples of anti-inflammation agent are Cox inhibitors such as ibuprofen, aspirin, tylenol, or GPR35 agonists, or GPR35-hERG complex activators.

### 81. ANTI-METABOLIC-DISORDER AGENT

An anti-metabolic disorder agent is any agent that has an effect in suppressing, reducing, or preventing diseases associated with metabolic disorders. Metabolism is the process human body uses to get or make energy from the food. Food is made up of proteins, carbohydrates and fats. Chemicals in digestive system break the food parts down into sugars and acids, thus providing fuels. The body can use this fuel right away, or it can store the energy in tissues, such as liver, muscles and body fat. A metabolic disorder occurs when abnormal chemical reactions in human body disrupt this process. When this happens, one might have too much of some substances or too little of other ones that one need to stay healthy.

### 82. ANTI-CONGESTIVE-HEART-FAILURE AGENT

An anti-congestive heart failure agent is any agent that has an effect in suppressing, reducing, or preventing diseases associated with congestive heart failure, such as a diuretic.

### 83. ANTI-CANCER AGENT

An anti-cancer agent is any agent that has an anti-cancer effect, such as vinblastin or taxol.

### 84. ENGINEERED CELL

An engineered cell is any cell in which one or more genes have been added or removed (via genetic blockage, such as homolgous recombination or siRNAa plasmid) or altered in either a transient or permenant fashion. The term “engineered cell” refers to a cell which has been manipulated to comprise exogenous material, such as nucleic acid. For example, disclosed herein are engineered cells which have been manipulated to comprise exogenous GPR35, exogenous hERG or both.

### 85. CELL

The term “cell” as used herein also refers to individual cells, cell lines, or cultures derived from such cells. A “culture” refers to a composition comprising isolated cells of the same or a different type. The term co-culture is used to designate when more than one type of cell are cultured together in the same dish with either full or partial contact with each other. A cell can be a recombinantly engineered cell wherein the cell comprises exogenous nucleic acid.

Cell refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

### 86. FUSION PROTEIN

A “fusion protein” is a protein or a peptide located either on the C- or N-terminal of the target protein, which facilitates one or several of the following characteristics: (1) improved solubility—Fusion of the N-terminus of the target protein to the C-terminus of a soluble fusion partner often improves the solubility of the target protein; (2) improved detection—Fusion of the target protein to either terminus of a short peptide (epitope tag) or protein which is recognized by an antibody (Western blot analysis) or by biophysical methods (e.g. GFP by fluorescence) facilitates the detection of the resulting protein during expression or purification; (3) improved purification—Simple purification schemes have been developed for proteins used at either terminus which bind specifically to affinity resins; (4) Localization—Tag, usually located on N-terminus of the target protein, which acts as address for sending protein to a specific cellular compartment; (5) improved Expression (E)—Fusion of the N-terminus of the target protein to the C-terminus of a highly expressed fusion partner results in high level expression of the target protein.

A “GPR35 fusion protein” refers to a protein or peptide located either on the C- or N-terminal of the target protein GPR35. Examples are GFP-GPR35 fusion protein that the green fluorescent protein (GFP) is located on the N-terminal of GPR35, while GPR35-GFP fusion protein that GFP is located on the C-terminal of GPR35.

### 87. INTERACT

“Interacts”, “interaction”, or the like, means that two (or more) molecules touch one another in a way beyond the touching that takes place because of random contacts between molecules. “Interacts” can be thought of as “binding” between two or more molecules, and therefore can have dissociation and association constants as well as equilibrium constants.

### 88. GPR35-hERG SIGNALING COMPLEX

A GPR35-hERG signaling complex or GPR35-hERG complex or GPR35-hERG oligomer or the like term refer to the complex formed via physical interaction between hERG channel protein and GPR35 protein at the cell surface in a hERG and GPR35 co-expressing cell s.

### 89. REGULATABLE PROMOTER

The term “promoter” or like terms is used to designate a region in the genome sequence upstream of a gene transcription start site (TSS), although sequences downstream of TSS may also affect transcription initiation. Promoter elements select the transcription initiation point, transcription specificity and rate. Depending on the distance from the TSS, the terms of ‘proximal promoter’ (several hundreds nucleotides around the TSS) and ‘distal promoter’ (thousands and more nucleotides upstream of the TSS) are also used. Both proximal and distal promoters include sets of various elements participating in the complex process of cell-, issue-, organ-, developmental stage- and environmental factors-specific regulation of transcription. Most promoter elements regulating TSS selection are localized in the proximal promoter (PlantProm: a database of plant promoter sequences, Shahmuradov et al. (2003) Nucleic Acids Res. 31(1): 114-117).

A regulatable promoter is a promoter which can be regulated by another molecule. For instance, the presence or absence of a molecule can either initiate promoter activity or prevent promoter activity.

### 90. SELECTABLE MARKER

A selectable marker is a molecule used to select the exogenous gene-positive cells during clone selection of a transfection process. The selectable marker can be, but is not limited to, tetracycline, ampicillin, neomycin, G418 or gentamicin.

### 91. LABEL

The terms label and tag as used herein refer to its presence as a moiety covalently or non-covalently bound to another residue such as the GPR35-hERG complex, wherein the label enables the location and or activity of the other residue to be monitored. In one example, the label can be fluorescent.

### 92. GPR35-hERG COMPLEX BINDING MOLECULES

The term “GPR35-hERG complex binding molecule” refers to any molecule that can bind to the GPR35-hERG complex.

### 93. TEST MOLECULE

A test molecule or test agent or the like is any molecule for which one or more activities or characteristics is being assayed.

### 94. 155. GPR35-hERG INTERACTION

The term “GPR35-hERG interaction” refers to the interaction of one or more GPR35 molecules with one or more hERG molecules. The interaction is a form of touching in a way beyond the touching that takes place because of random contacts between molecules.

### 95. GPR35-hERG COMPLEX INTERFERING MOLECULE

The term “GPR35-hERG complex interfering molecule” refers to any molecule that interferes with the GPR35-hERG interaction. For example, the molecule can prevent the interaction or disrupt an already occurring interaction between GPR35 and hERG. An example of a GPR35-hERG interfering molecule would be a peptide which mimics either the hERG interaction site on GPR35 or the GPR35 interaction site on hERG.

### 96. GPR35 AND hERG CO-EXPRESSING CELL

A “GPR35 and hERG co-expressing cell is a cell which expresses both GPR35 and hERG. The cell can be a primary cell or a cell line. The GPR35 and hERG expressing cell can also be a recombinantly engineered cell which expresses exogenous GPR35 and hERG, or a combination of exogenous GPR35 and endogenous hERG (or vice versa). A GPR35-hERG co-expressing engineered cell line is any cell line in which has been engineered to operably express both GPR35 and hERG.

### 97. GPR35-hERG COMPLEX-ASSOCIATED DISORDER

A GPR35-hERG complex-associated disorder is a disorder in which the GPR35-hERG complex plays important roles in the initiation and progression of the disorder. Possible GPR35-hERG complex-associated disorder includes metabolic disorders, congestive heart failure, inflammation (e.g., viral infection-induced inflammation, joint inflammation or others), cancers (e.g., colon cancers and gastric cancers), and neurological disorders.

### 98. GPR35-hERG EXPRESSING ENGINEERED CELL LINE

A GPR35-hERG expressing engineered cell line is any cell line in which has been engineered to operably express both GPR35 and hERG. The GPR35 and hERG expressing engineered cell expresses both exogenous GPR35 and hERG, or a combination of exogenous GPR35 and endogenous hERG (or vice versa).

### 99. LABEL-FREE BIOSENSOR hERG ACTIVATOR

A label-free biosensor hERG activator or like terms is a molecule that is a hERG activator and is capable of triggering a detectable biosensor signal in a hERG expressing cell using a label-free biosensor cellular assay. The biosensor hERG activator can be a hERG activator, a hERG pathway activator, or a hERG ion channel activator. Examples are mallotoxin, RPR260243, NS1643, NS3623, PD-118057, PD-307243, A-935142, flufenamic acid, niflumic acid, or diflunisal.

### 100. LIGAND

A ligand or like terms is a substance or a composition or a molecule that is able to bind to and form a complex with a biomolecule to serve a biological purpose. Actual irreversible covalent binding between a ligand and its target molecule is rare in biological systems. Ligand binding to receptors alters the chemical conformation, i.e., the three dimensional shape of the receptor protein. The conformational state of a receptor protein determines the functional state of the receptor. The tendency or strength of binding is called affinity. Ligands include substrates, blockers, inhibitors, activators, and neurotransmitters. Radioligands are radioisotope labeled ligands, while fluorescent ligands are fluorescently tagged ligands; both can be considered as ligands are often used as tracers for receptor biology and biochemistry studies. Ligand and modulator are used interchangeably.

### 101. LIBRARY

A library or like terms is a collection. The library can be a collection of anything disclosed herein. For example, it can be a collection, of indexes, an index library; it can be a collection of profiles, a profile library; or it can be a collection of DMR indexes, a DMR index library; Also, it can be a collection of molecule, a molecule library; it can be a collection of cells, a cell library; it can be a collection of markers, a marker library; A library can be for example, random or non-random, determined or undetermined. For example, disclosed are libraries of DMR indexes or biosensor indexes of known modulators.

### 102. MAINTAINING

The word “maintaining” or like words refers to continuing a state. In the context of a treatment, maintaining can be refer to less than 20%, 15%, 10%, 9%, 8%, 7%, 6%, 5%, 4%, 3%, 2%, 1%, or 0.1% change from a control, such a basal level, often a level in the absence of a treatment or in the presence of treatment with a placebo or standard.

### 103. MATERIAL

Material is the tangible part of something (chemical, biochemical, biological, or mixed) that goes into the makeup of a physical object.

### 104. MARKER

A marker or like terms is a ligand which produces a signal in a biosensor cellular assay. The signal is, must also be, characteristic of at least one specific cell signaling pathway(s) and/or at least one specific cellular process(es) mediated through at least one specific target(s). The signal can be positive, or negative, or any combinations (e.g., oscillation).

### 105. MARKER PANEL

A “marker panel” or like terms is a panel which comprises at least two markers. The markers can be for different pathways, the same pathway, different targets, or even the same targets.

### 106. MARKER BIOSENSOR INDEX

A “marker biosensor index” or like terms is a biosensor index produced by data collected for a marker. For example, a marker biosensor index can be made up of a profile of the marker acting on the panel of cells, and the modulation profile of the marker against the panels of markers, each panel of markers for a cell in the panel of cells.

### 107. MARKER DMR INDEX

A “marker biosensor index” or like terms is a biosensor DMR index produced by data collected for a marker. For example, a marker DMR index can be made up of a profile of the marker acting on the panel of cells, and the modulation profile of the marker against the panels of markers, each panel of markers for a cell in the panel of cells.

### 108. MIMIC

As used herein, “mimic” or like terms refers to performing one or more of the functions of a reference object. For example, a molecule mimic performs one or more of the functions of a molecule.

### 109. MODULATE

To modulate, or forms thereof, means either increasing, decreasing, or maintaining a cellular activity mediated through a cellular target. It is understood that wherever one of these words is used it is also disclosed that it could be 1%, 5%, 10%, 20%, 50%, 100%, 500%, or 1000% increased from a control, or it could be 1%, 5%, 10%, 20%, 50%, or 100% decreased from a control.

### 110. MODULATE

The term modulate or like terms refers to its standard meaning of increasing or decreasing.

### 111. MODULATOR

A modulator or like terms is a ligand that controls the activity of a cellular target. It is a signal modulating molecule binding to a cellular target, such as a target protein.

### 112. MODULATION COMPARISON

A “modulation comparison” or like terms is a result of normalizing a primary profile and a secondary profile.

### 113. MODULATOR BIOSENSOR INDEX

A “modulator biosensor index” or like terms is a biosensor index produced by data collected for a modulator. For example, a modulator biosensor index can be made up of a profile of the modulator acting on the panel of cells, and the modulation profile of the modulator against the panels of markers, each panel of markers for a cell in the panel of cells.

### 114. MODULATOR DMR INDEX

A “modulator DMR index” or like terms is a DMR index produced by data collected for a modulator. For example, a modulator DMR index can be made up of a profile of the modulator acting on the panel of cells, and the modulation profile of the modulator against the panels of markers, each panel of markers for a cell in the panel of cells.

### 115. MODULATE THE BIOSENSOR SIGNAL OF A MARKER

“Modulate the biosensor signal or like terms is to cause changes of the biosensor signal or profile of a cell in response to stimulation with a marker.

### 116. MODULATE THE DMR SIGNAL

“Modulate the DMR signal or like terms is to cause changes of the DMR signal or profile of a cell in response to stimulation with a marker.

### 117. MOLECULE

As used herein, the term “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

### 118. MOLECULE MIXTURE

A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

### 119. MOLECULE BIOSENSOR INDEX

A “molecule biosensor index” or like terms is a biosensor index produced by data collected for a molecule. For example, a molecule biosensor index can be made up of a profile of the molecule acting on the panel of cells, and the modulation profile of the molecule against the panels of markers, each panel of markers for a cell in the panel of cells.

### 120. MOLECULE DMR INDEX

A “molecule DMR index” or like terms is a DMR index produced by data collected for a molecule. For example, a molecule biosensor index can be made up of a profile of the molecule acting on the panel of cells, and the modulation profile of the molecule against the panels of markers, each panel of markers for a cell in the panel of cells.

### 121. MOLECULE INDEX

A “molecule index” or like terms is an index related to the molecule.

### 122. MOLECULE-TREATED CELL

A molecule-treated cell or like terms is a cell that has been exposed to a molecule.

### 123. MOLECULE MODULATION INDEX

A “molecule modulation index” or like terms is an index to display the ability of the molecule to modulate the biosensor output signals of the panels of markers acting on the panel of cells. The modulation index is generated by normalizing a specific biosensor output signal parameter of a response of a cell upon stimulation with a marker in the presence of a molecule against that in the absence of any molecule.

### 124. MOLECULE PHARMACOLOGY

Molecule pharmacology or the like terms refers to the systems cell biology or systems cell pharmacology or mode(s) of action of a molecule acting on a cell. The molecule pharmacology is often characterized by, but not limited, toxicity, ability to influence specific cellular process(es) (e.g., proliferation, differentiation, reactive oxygen species signaling), or ability to modulate a specific cellular target.

### 125. NORMALIZING

Normalizing or like terms means, adjusting data, or a profile, or a response, for example, to remove at least one common variable. For example, if two responses are generated, one for a marker acting a cell and one for a marker and molecule acting on the cell, normalizing would refer to the action of comparing the marker-induced response in the absence of the molecule and the response in the presence of the molecule, and removing the response due to the marker only, such that the normalized response would represent the response due to the modulation of the molecule against the marker. A modulation comparison is produced by normalizing a primary profile of the marker and a secondary profile of the marker in the presence of a molecule (modulation profile).

### 126. OPTIONAL

“Optional” or “optionally” means that the subsequently described event or circumstance may or may not occur, and that the description includes instances where said event or circumstance occurs and instances where it does not.

### 127. OR

The word “or” or like terms as used herein means any one member of a particular list and also includes any combination of members of that list.

### 128. PREVENT

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. Similarly, something could be reduced and inhibited, but not prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

### 129. PROFILE

A profile or like terms refers to the data which is collected for a composition, such as a cell. A profile can be collected from a label free biosensor as described herein.

i. Primary Profile

A “primary profile” or like terms refers to a biosensor response or biosensor output signal or profile which is produced when a molecule contacts a cell. Typically, the primary profile is obtained after normalization of initial cellular response to the net-zero biosensor signal (i.e., baseline)

ii. Secondary Profile

A “secondary profile” or like terms is a biosensor response or biosensor output signal of cells in response to a marker in the presence of a molecule. A secondary profile can be used as an indicator of the ability of the molecule to modulate the marker-induced cellular response or biosensor response.

iii. Modulation Profile

A “modulation profile” or like terms is the comparison between a secondary profile of the marker in the presence of a molecule and the primary profile of the marker in the absence of any molecule. The comparison can be by, for example, subtracting the primary profile from secondary profile or subtracting the secondary profile from the primary profile or normalizing the secondary profile against the primary profile.

### 130. PANEL

A panel or like terms is a predetermined set of specimens (e.g., markers, or cells, or pathways). A panel can be produced from picking specimens from a library.

### 131. POSITIVE CONTROL

A “positive control” or like terms is a control that shows that the conditions for data collection can lead to data collection.

### 132. POTENTIATE

Potentiate, potentiated or like terms refers to an increase of a specific parameter of a biosensor response of a marker in a cell caused by a molecule. By comparing the primary profile of a marker with the secondary profile of the same marker in the same cell in the presence of a molecule, one can calculate the modulation of the marker-induced biosensor response of the cells by the molecule. A positive modulation means the molecule to cause increase in the biosensor signal induced by the marker.

### 133. POTENCY

Potency or like terms is a measure of molecule activity expressed in terms of the amount required to produce an effect of given intensity. For example, a highly potent drug evokes a larger response at low concentrations. The potency is proportional to affinity and efficacy. Affinity is the ability of the drug molecule to bind to a receptor.

### 134. PUBLICATIONS

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### 135. RANGES

Ranges can be expressed herein as from “about” one particular value, and/or to “about” another particular value. When such a range is expressed, some forms includes from the one particular value and/or to the other particular value. Similarly, when values are expressed as approximations, by use of the antecedent “about,” it will be understood that the particular value constitutes one of the encompassed values. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that these data represent endpoints and starting points, and ranges for any combination of the data points. For example, if a particular datum point “10” and a particular datum point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

### 136. RECEPTOR

A receptor or like terms is a protein molecule embedded in either the plasma membrane or cytoplasm of a cell, to which a mobile signaling (or “signal”) molecule may attach. A molecule which binds to a receptor is called a “ligand,” and may be a peptide (such as a neurotransmitter), a hormone, a pharmaceutical drug, or a toxin, and when such binding occurs, the receptor goes into a conformational change which ordinarily initiates a cellular response. However, some ligands merely block receptors without inducing any response (e.g. antagonists). Ligand-induced changes in receptors result in physiological changes which constitute the biological activity of the ligands.

### 137. REDUCE

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

### 138. “ROBUST BIOSENSOR SIGNAL”

A “robust biosensor signal” is a biosensor signal whose amplitude(s) is significantly (such as 3×, 10×, 20×, 100×, or 1000×) above either the noise level, or the negative control response. The negative control response is often the biosensor response of cells after addition of the assay buffer solution (i.e., the vehicle). The noise level is the biosensor signal of cells without further addition of any solution. It is worthy of noting that the cells are always covered with a solution before addition of any solution.

### 139. “ROBUST DMR SIGNAL”

A “robust DMR signal” or like terms is a DMR form of a “robust biosensor signal.”

### 140. RESPONSE

A response or like terms is any reaction to any stimulation.

### 141. SAMPLE

By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

### 142. SALT(S) AND PHARMACEUTICALLY ACCEPTABLE SALT(S)

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I or II with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, 1′-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate. Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl(CrC₆)halides (e.g., methyl, ethyl, propyl, and butyl chlorides, bromides, and iodides), dialkyl sulfates (i.e., dimethyl, diethyl, dibuytl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hemisulphate and hemicalcium salts.

The compounds of the invention and their salts may exist in both unsolvated and solvated forms.

### 143. SIGNALING PATHWAY(S)

A “defined pathway” or like terms is a path of a cell from receiving a signal (e.g., an exogenous ligand) to a cellular response (e.g., increased expression of a cellular target). In some cases, receptor activation caused by ligand binding to a receptor is directly coupled to the cell's response to the ligand. However, for many cell surface receptors, ligand-receptor interactions are not directly linked to the cell's response. The activated receptor must first interact with other proteins inside the cell before the ultimate physiological effect of the ligand on the cell's behavior is produced. Often, the behavior of a chain of several interacting cell proteins is altered following receptor activation. The entire set of cell changes induced by receptor activation is called a signal transduction mechanism or pathway. The signaling pathway can be either relatively simple or quite complicated.

### 144. SYNERGY

The term “synergistic effect” or “synergy” as used herein means that the therapeutic effect of a combination comprising two or more agents is more effective than the therapeutic effect of a treatment where only a single agent alone is applied. Further, a synergistic effect of a combination of two or more agents permits the use of lower dosages of one or more of the agents and/or less frequent administration of said agents to a patient. The ability to utilize lower dosages of an agent and/or to administer said agent less frequently reduces the toxicity associated with the administration of said agent to a patient without reducing the efficacy of said agent in the prevention, management or treatment of the diseases or conditions. In addition, a synergistic effect can result in improved efficacy of agents in the prevention, management or treatment of the diseases or conditions. Moreover, a synergistic effect of a combination of two or more agents may avoid or reduce adverse or unwanted side effects associated with the use of either agent alone.

Throughout this application, various definitions of terms are disclosed. Although some terms are defined under one category, the definitions of those terms can be applied to other parts of the whole disclosure.

### 145. SIMILARITY OF INDEXES

“Similarity of indexes” or like terms is a term to express the similarity between two indexes, or among at least three indices, one for a molecule, based on the patterns of indices, and/or a matrix of scores. The matrix of scores are strongly related to their counterparts, such as the signatures of the primary profiles of different molecules in corresponding cells, and the nature and percentages of the modulation profiles of different molecules against each marker. For example, higher scores are given to more-similar characters, and lower or negative scores for dissimilar characters. Because there are only three types of modulation, positive, negative and neutral, found in the molecule modulation index, the similarity matrices are relatively simple. For example, a simple matrix will assign identical modulation (e.g., a positive modulation) a score of +1 and non-identical modulation a score of −1.

### 146. SUBJECT

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. The subject can also be a non-human.

### 147. SOLVATE

The compounds herein, and the pharmaceutically acceptable salts thereof, may exist in a continuum of solid states ranging from fully amorphous to fully crystalline. They may also exist in unsolvated and solvated forms. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, d₆-acetone, d₆-DMSO).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will be the norm.

The compounds herein, and the pharmaceutically acceptable salts thereof, may also exist as multi-component complexes (other than salts and solvates) in which the compound and at least one other component are present in stoichiometric or non-stoichiomethc amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun., 17:1889-1896 (2004). For a general review of multi-component complexes, see J. K. Haleblian, J. Pharm. Sci. 64(8):1269-88 (1975).

### 148. STABLE

When used with respect to pharmaceutical compositions, the term “stable” or like terms is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

### 149. SUBSTANCE

A substance or like terms is any physical object. A material is a substance. Molecules, ligands, markers, cells, proteins, and DNA can be considered substances. A machine or an article would be considered to be made of substances, rather than considered a substance themselves.

### 150. TEST MOLECULE

A test molecule or like terms is a molecule which is used in a method to gain some information about the test molecule. A test molecule can be an unknown or a known molecule.

### 151. TISSUE

Tissue or like terms refers to a collection of cells. Typically a tissue is obtained from a subject.

### 152. TREATING

By “treating” or “treatment” is meant the medical management of a patient with the intent to cure, ameliorate, stabilize, or prevent a disease, pathological condition, or disorder. These terms include active treatment, that is, treatment directed specifically toward the improvement of a disease, pathological condition, or disorder, and also includes causal treatment, that is, treatment directed toward removal of the cause of the associated disease, pathological condition, or disorder. These terms can mean that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. In certain situations a treatment can inadvertently cause harm. In addition, these terms include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. These terms mean both treatment having a curing or alleviating purpose and treatment having a preventive purpose. The treatment can be made either acutely or chronically. It is understood that treatment can mean a reduction or one or more symptoms or characteristics by at least 10%, 20%, 30%, 40%, 50%, 60%, 70%, 80%, 90%, 95%, 99%, 99.9%, 99.99%, 100%, relative to a control. In the context of these terms, preventing refers to the ability of a compound or composition (such as the disclosed compounds and compositions) to prevent a disease identified herein in patients diagnosed as having the disease or who are at risk of developing such disease. In this context, preventing includes the delaying the onset of the disease relative to a control. These terms do not require that the treatment in fact be effective to produce any of the intended results. It is enough that the results are intended.

### 153. THERAPEUTIC AGENT

A therapeutic agent or like term is any molecule or composition in which the molecule or composition is useful in preventing or treating conditions or diseases within the therapeutic field. For example, anti-cancer agents can be any agent that can prevent the formation of cancer cell in a subject, reduce the number of cancer cells in a subject, or eliminate all cancer cells in a subject.

### 154. THERAPEUTICALLY EFFECTIVE

The term “therapeutically effective” means that the amount of the composition used is of sufficient quantity to treat a subject as defined herein.

### 155. TOXICITY

Toxicity is the degree to which a substance, molecule, is able to damage something, such as a cell, a tissue, an organ, or a whole organism, that has been exposed to the substance or molecule. For example, the liver, or cells in the liver, hepatocytes, can be damaged by certain substances.

### 156. TOXICITY MARKER

A toxicity marker is any reagent, molecule, substance etc. that can be used for identifying, diagnosing, prognosing a level of toxicity of a substance, in for example, an organism or cell or tissue or organ.

### 157. TRANSACTIVATE

“Transactivate” refers to the process that the activation of a receptor in a cell can also activate another receptor in the same cell. Such transactivation can be direct (i.e., both receptors form a complex such as dimer or oligomer, such that the activation of the 1^{st} receptor cause a conformational change in the 2^{nd} receptor, thus leading to the activation of the 2^{nd} receptor) or indirect (i.e., the two receptors are not necessarily within a signaling complex; however, the activation of 1^{st} receptor leads to a pathway in which a signaling protein within the pathway activates the 2^{nd} receptor).

### 158. TRIGGER

A trigger or like terms refers to the act of setting off or initiating an event, such as a response.

### 159. VALUES

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Thus, the disclosed methods, compositions, articles, and machines, can be combined in a manner to comprise, consist of, or consist essentially of, the various components, steps, molecules, and composition, and the like, discussed herein. They can be used, for example, in methods for characterizing a molecule including a ligand as defined herein; a method of producing an index as defined herein; or a method of drug discovery as defined herein.

### 160. UNKNOWN MOLECULE

An unknown molecule or like terms is a molecule with unknown biological/pharmacological/physiological/pathophysiological activity.

### 161. WEIGHT %

References in the specification and concluding claims to parts by weight, of a particular element or component in a composition or article, denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### A. Example 1

### 1. Preparation of Compounds of Formula (I), (II), (III), (IV), (V) and (VI)

The following are examples of preparation of compounds of formula (I), (II), (III), (IV), (V) or (VI). This example is intended to be purely exemplary and is not intended to limit the disclosure.

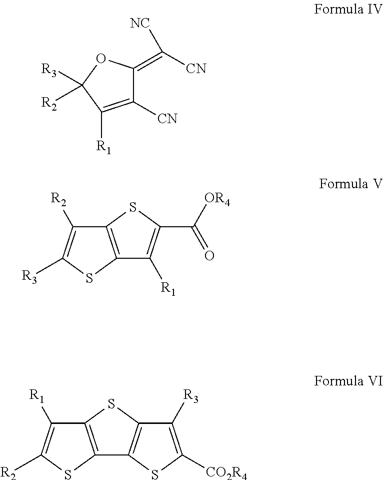

### i. 2-dicyanomethylene-3-cyano-4,5-dimethyl-5-[4′-n-butylphenyl]-2,5-dihydrofuran

3-hydroxy-3-[4′-n-butylphenyl]-2-butanone 10.0 g (0.0455 mole), malononitrile 6.0 g (0.091 mole), lithium ethoxide 2.3 ml (2.3 mmole) and THF 50 ml were mixed and boiled at reflux overnight. Pure product (2-dicyanomethylene-3-cyano-4,5-dimethyl-5-[4′-n-butylphenyl]-2,5-dihydrofuran) was obtained from recrystallization in ethanol to give 9.95 g: yield 69.1%. mp: 129.3-130.5° C. ¹H NMR: δ 7.259 (d, 2H), 7.112 (d, 2H), 2.638 (t, 2H, CH₂), 2.225 (s, 3H, Me), 1.997 (s, 3H, Me), 1.60-1.28 (m, 4H, CH₂), 0.931 (t, 3H, CH₃). ¹³C NMR: 182.476, 175.753, 145.841, 131.069, 129.692 (2C), 125.075 (2C), 110.914, 110.406, 109.088, 104.728, 101.724, 58.830, 35.236, 33.277, 22.335 (2C), 14.558, 13.873. HPLC: 100%.

### ii. 2-dicyanomethylene-3-cyano-4,5-dimethyl-5-[2′,4′-difluorophenyl]-2,5-dihydrofuran

3-hydroxy-3-[2′,4′-difluorophenyl]-2-butanone 8 g (0.04 mole), malononitrile 5.3 g (0.08 mole) and lithium ethoxide 2 ml (2 mmole) were refluxed in 50 ml of THF overnight. After workup, pure product (2-dicyanomethylene-3-cyano-4,5-dimethyl-5-[2′,4′-difluorophenyl]-2,5-dihydrofuran) was obtained by crystallization from ethanol. It afforded 4.5 grams: yield 37.9%. Mp: 219.0-221.4. ¹H NMR: δ 7.441 (d, 1H), 7.066 (dd, 1H), 6.945 (d, 1H), 2.274 (s, 3H, Me), 2.041 (s, 3H, Me). ¹⁹F NMR: −104.81 (d, 1F), 107.294 (d, 1F). HPLC: 100%.

### iii. 2-dicyanomethylene-3-cyano-4-methyl-5-spiro-cyclohexyl-2,5-dihydrofuran

1-hydroxy-1-cyclohexyl-ethanone 14.2 g (0.1 mole), malononitrile 13.2 g (0.2 mole), Sodium ethoxide 100 ml (0.1 mole, 1M solution in ethanol) and ethanol 100 ml were mixed and reacted overnight at room temperature. The pure product (2-dicyanomethylene-3-cyano-4-methyl-5-spiro-cyclohexyl-2,5-dihydrofuran) was obtained from crystallization in ethanol to give 16.1 grams: yield 67.5%. mp: 236.5-237.5° C. ¹H NMR: δ 2.337 (s, 3H, Me), 1.86-1.70 (m, 11H, ring). ¹³C NMR: 182.387, 175.230, 110.980, 110.458, 108.999, 104.883, 101.437, 58.572, 33.123 (2C), 23.918, 21.289 (2C), 14.499. HPLC: 100%.

### iv. 2-dicyanomethylene-3-cyano-4-methyl-5-phenyl-5-perfluoromethyl-2,5-dihydrofuran

3-hydroxy-3-phenyl-4,4,4-trifluoro-2-butanone 10.0 g (0.046 mole), malononitrile 6.1 g (0.092 mole), lithium ethoxide 2.5 ml (2.5 mmole) and THF 20 ml were mixed and refluxed overnight. The pure product (2-dicyanomethylene-3-cyano-4-methyl-5-phenyl-5-perfluoromethyl-2,5-dihydrofuran) was obtained through a column chromatography (100% dichloromethane on silica gel, 60-200 mesh) to give 3.75 grams: yield 25.9%. mp: 133-135° C. ¹H NMR: 7.577-7.546 (m, 3H, Ar), 7.444-7.410 (m, 2H, Ar), 2.479 (s, 3H, Me). ¹⁹F NMR: −72.852. ¹³C NMR: 174.224, 172.148, 131.908, 130.191, 127.467, 125.742, 121.682, 109.793, 109.511, 109.098, 108.099, 98.521, 62.938, 15.439. GC/MS: 317 (M+2), 247 (M-CF3). HPLC: 100%.

### v. 2-dicyanomethylene-3-cyano-4,5-dimethyl-5-[3′,4′-dichlorophenyl]-2,5-dihydrofuran

3-hydroxy-3-[3′,4′-dichlorophenyl]-2-butanone 15 g (0.064 mole), malononitrile 8.5 g (0.129 mole) and lithium ethoxide 3.2 ml (3.2 mmole, 1M solution in ethanol) were stirred in 80 ml of THF solution and allowed to boil under reflux conditions overnight. The solution was concentrated by removing the majority of the THF on a rotary evaporator under aspirator vacuum. The remaining residue was taken up in methylene chloride, washed with brine (2×) then DI water (2×). The organic layer was dried over anhydrous MgSO4, filtered and the solvent removed. The crude product was recrystallized from denatured alcohol to yield the product (2-dicyanomethylene-3-cyano-4,5-dimethyl-5-[3′,4′-dichlorophenyl]-2,5-dihydrofuran) to give 5.5 grams: yield 25.9%. mp: 226.4-228.6° C. ¹H NMR: δ 7.579 (d, 1H), 7.324 (d, 1H), 7.070 (dd, 1H), 2.254 (s, 3H, Me), 2.001 (s, 3H, Me). ¹³C NMR: 180.253, 175.015, 135.676, 134.630, 134.382, 131.982, 127.552, 124.595, 110.580, 109.966, 108.810, 105.822, 100.107, 60.573, 22.894, 22.894, 14.637. HPLC: 100%.

### vi. 2-dicyanomethylene-3-cyano-4-methyl-5-spiro-fluorenylidine-2,5-dihydrofuran

9-hydroxy-9-acetyl-fluorene 5.0 g (0.0223 mole), malononitrile 2.94 g (0.0446 mole), anhydrous potassium carbonate 3.1 g (0.0223 mole), 18-crown-6 ether (catalytic amount) and dry THF 50 ml were mixed and refluxed over night. The pure product (2-dicyanomethylene-3-cyano-4-methyl-5-spiro-fluorenylidine-2,5-dihydrofuran) was collected by crystallization from ethanol to give 3.22 g: yield 45.0%. mp: 302-303° C. ¹HNMR: δ 7.760 (d, 2H), 7.566 (t, 2H), 7.393 (t, 2H), 7.187 (d, 2H), 1.935 (s, 3H). ¹³C NMR: 177.504, 140.459. HPLC: 100%.

### vii. 2-Dicyanomethylene-3-cyano-4,5,5-trimethyl-2,5-dihydrofuran

According to scheme 1, compound a (14.59 g, 142.9 mmol), malononitrile (29.87 g, 452.2 mmol), and magnesium ethoxide (18.5, 2 mmol) in ethanol (120 mL) were mixed and refluxed overnight. Pure 10 (2-Dicyanomethylene-3-cyano-4,5,5-trimethyl-2,5-dihydrofuran) was obtained after column chromatography to give 8.30 g. Yield: 29%. ¹H NMR: δ 2.36 (s, 3H), (1.63 (s, 3H); GC-MS 199. Pure 8 (2-cyano-2-(1-amino-2,2-dicyanovinyl)methylene-3-cyano-4,5,5-trimethyl-2,5-dihydrofuran) was obtained as a by product, with a GC-MS of 265.

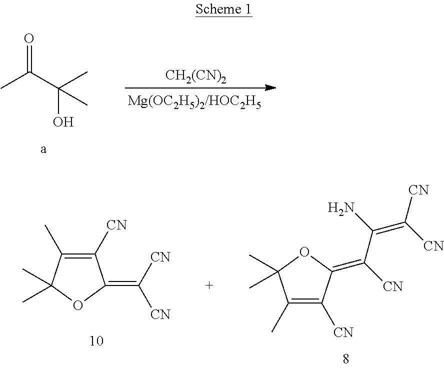

### viii. 2-Dicyanomethylene-3-cyano-4,5-dimethyl-5-[2′3′4′5′6′-pentafluorophenyl]-2,5-dihydrofuran

According to Scheme 2, compound b (10.0 g, 0.04 mol), malononitrile (5.3 g, 0.08 mol), and lithium ethoxide (2 mL, 2 mmol) in THF (30 mL) were mixed and refluxed overnight. Pure 9 (2-Dicyanomethylene-3-cyano-4,5-dimethyl-5-[2′3′4′5′6′-pentafluorophenyl]-2,5-dihydrofuran) was obtained after column chromatography (100% dichloride methane on 60-200-mesh silica gel) to give 2.7 g. Yield: 19.2%. mp 175.5-177.6° C. ¹H NMR: δ 2.340 (s, 3H, Me), 2.124 (t, 3H, Me, coupled with F). ¹⁹F NMR: −138.048 (m, 2F), −148.058 (m, 1F), −158.890 (m, 2F). ¹³C NMR: 177.895, 174.651, 145.996 (2C), 143.004, 138.601 (2C), 110.327, 109.800, 108.618, 107.992, 106.275, 97.567, 61.044, 24.986, 14.363. Purity by HPLC, λ=300 nm, 100%.

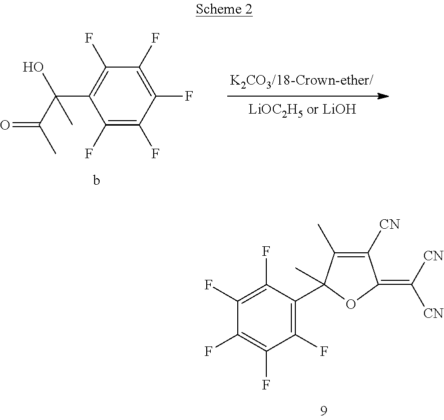

### ix. Thieno[3,2-b]thiophene-2-carboxylic acid

According to Scheme 3, first, compound c (27 g, 0.141 mol) was mixed with K₂CO₃ (82.80 g, 0.60 mol) and DMF (300 mL) in a three neck flask equipped with a condenser and addition funnel. To this mixture ethyl mercaptoacetate (17.5 g, 0.141 mol) was added dropwise at 60-70° C. The mixture was heated at 60-70° C. overnight until no starting materials were detected by GC/MS. The mixture then was poured into water (600 mL) and extracted by diethyl ether (2×100 ml). Organic washed by brine (3×400 ml), and dried over anhydrous MgSO₄. After evaporating the solvent, the brownish crude target was obtained and found to be pure enough for the next reaction (71.0 g, 100%), leading to compound d with a GC/MS of 212. Compound d (29 g, 0.137 mol) was dissolved into a mixture of THF (200 mL), methanol and LiOH (1M, 200 mL). This mixture was refluxed overnight and poured into concentrated hydrochloric acid (50 mL) The acid mixture was then diluted to 500 mL with water. Solid was filtrated and washed with water (3×100 mL). The light yellow solid of Compound 11 was washed with methanol (100 mL) and dried under vacuum overnight (21 g, 83%). GC-MS 140 [M-COOH].

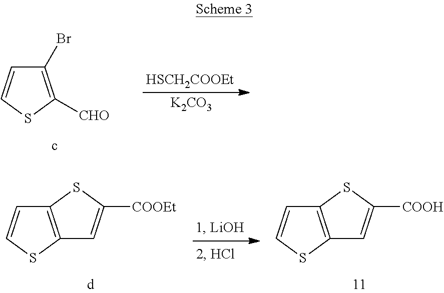

### x. 3-Methyl-thieno[3,2-b]thiophene-2-carboxylic acid

According to Scheme 4, compound e (15.00 g, 0.073 mol) was mixed with K2CO3 (40.30 g, 0.29 mol) and DMF (200 mL) in a three neck flask equipped with a condenser and addition funnel. To this mixture ethyl mercaptoacetate (9.06 g, 0.073 mol) was added dropwise at 60-70° C. The mixture was heated at 60-70° C. overnight until no starting materials was detected by GC/MS. The mixture was then poured into water (400 mL) and extracted by diethyl ether (2×200 ml). Organic washed by brine (3×150 ml), and dried over anhydrous MgSO4. After evaporating the solvent, the brownish crude target compound f (13.10 g) was obtained and found to be pure enough for the next reaction. Compound f (13.10 g, 0.0579 mol) was dissolved into a mixture of THF (100 mL), methanol and LiOH (1M, 100 mL). This mixture was refluxed overnight and poured into concentrated hydrochloric acid (30 mL). The acid mixture was then diluted to 400 mL with water. Solid was filtrated and washed with water (3×100 mL). The light yellow solid of compound 12 was washed with methanol (2×30 mL) and dried under vacuum overnight (9.73 g). GC-MS 154 [M-COOH].

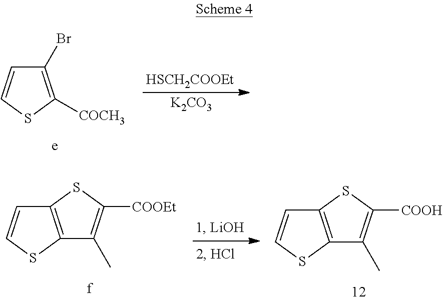

### xi. 3,6-Dibromo-thieno[3,2-b]thiophene 2-undecanone

According to Scheme 5, to a mixture of compound g (12.43 g, 0.0417 mol) and AlCl₃ (12.78 g, 0.0959 mol) in CH₂Cl₂ (70 mL) at 0° C., n-Undecanoyl chloride (10.25 g, 0.0500 mol) was added dropwise under a nitrogen stream. This was stirred at 0° C. for about 12 hours until only small amount of starting materials could be detected by GC/MS. The mixture was then poured into HCl (200 mL, 6M) and the organic was extracted with hexanes/CH₂Cl₂ (1:1) (2×200 mL). The combined organic solution was washed with brine (2×100 mL) and water (100 mL). After drying over anhydrous MgSO₄, the solvent was evaporated. A low melting point solid of compound 15 was collected and was recrystallized from EtOH to yield a purified product (18.32 g, 94%). GC-MS 465 [M+].

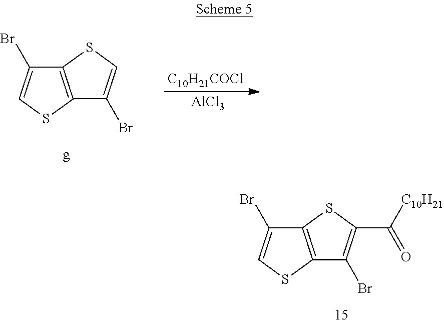

### xii. Diethyl dithieno[3,2-b:2′,3′-d]thiophene-2,6-dicarboxylate

Diethyl dithieno[3,2-b:2′,3′-d]thiophene-2,6-dicarboxylate was synthesized according to literature (Frey, Joseph; Bond, Andrew D.; Holmes, Andrew B. Improved synthesis of dithieno[3,2-b:2′,3′-d]thiophene (DTT) and derivatives for cross coupling. Chemical Communications 2002, 20, 2424-2425).

### xiii. N,N-bis(2-hydroxyethyl)-4-[2-(thiophene-2-yl)vinyl]aniline

N,N-bis(2-hydroxyethyl)-4-[2-(thiophene-2-yl)vinyl]aniline was synthesized according to literature (Jen, K.-Y. A.; Drost, K. J. Preparation of polyimides having nonlinear optical properties. Eur. Pat. Appl. 1995, EP 647874 A1).

### xiv. 4-{[N-Methyl,N-2-hydroexyethyl-amino]-pheylene-thien-5}-formate lithium

According to Scheme 6, under N₂ protection, a solution of n-BuLi in hexane (8 mL, 2.5 M) was added dropwise in to a solution of compound h (4.58 g, 0.020 mol) in THF (50 mL) at −78° C. The solution was turned to green rapidly. After the addition, this reaction solution was warmed up to −10° C. slowly and it was then cooled to at −78° C. again. Few grams of dry ice was added rapidly. The cloudy solution was changed to a yellowish color with yellow solid formed. This mixture was then warmed to room temperature. 10 mL of water was then added to this mixture and a clear solution was formed. After evaporation of most of THF solvent, the solid in the residual cloudy solution was collected and recrystallized from ETOH to yield compound 26 (2.50 g). 2.50 g of dried 26 was refluxed in MeOH/H2SO4 (c) for overnight. After the addition of water, MeOH was removed and ethyl acetate was used to extract the desired product 25. After a recrystallization from EtOH, the desired product 25 was obtained (2.40 g). GC-MS 317.

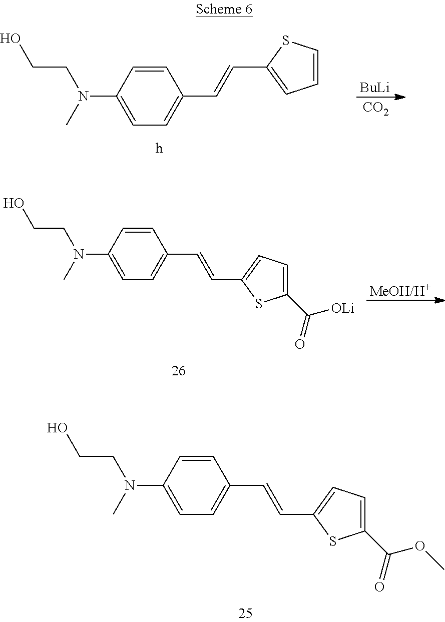

### xv. 2,2′-Bithiophene-5-carboxaldehyde (compound 30) and 2,2′-Bithiophene-5-methanol (compound 27)

According to Scheme 7, under N₂ protection, a mixture of compound i (45 g, 0.184 mol) and Mg (4.42 g, 0.184 mol) in THF (100 mL) were refluxed for hours. A few mL of DMF was added and the resulting solution was stirred overnight. After reaction, ice-water cooled aqueous hydrochloric acid was added until this solution is acidic. Ethyl acetate (2×100 ml) was used to extract the organic from this acidic aqueous solution. This organic layer was washed by aqueous NaHCO₃ solution and brine, and dried over anhydrous MgSO₄. After the evaporation of ethyl acetate, the residual was mixed with 5% ethyl acetate/95% hexane. A hot filtration was carried out to solid (dialdehyde). The filtrate was cooled to room temperature to yield crystals of compound 30 (35.6 g, 64.6%). Under N₂ protection, 4.00 g (0.105 mol) of NaBH₄ was added in small portions into a methanol solution of 30 (41 g, 0.211 mol). This reaction was run overnight. MeOH was removed to yield a residue which was dissolved into ethyl acetate. This ethyl acetate was washed by brine and dried over anhydrous MgSO₄. Solvent ethyl acetate was removed to yield compound 27 (35.6 g, 64.6%). ¹H NMR and GC-MS confirmed that it is the correct compound.

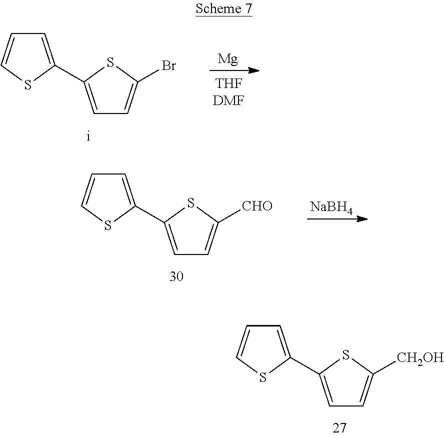

### xvi. 5-Hydroxymethyl-5′-formyl-2,2′-bithiophene

5-Hydroxymethyl-5′-formyl-2,2′-bithiophene was synthesized according to literature (Chang, C. T.; Lee, C.-T.; Lin, F.-L. (Hydroxymethyl)polythiophene derivatives useful for preventing inflammation and edema. U.S. Pat. No. 5,508,440A (1996)).

### xvii. 5′-{2-[4-[bis(2-hydroxyethyl)amino]phenyl]ethenyl]}-(2,2′-Bithiophene)-5-formate sodium

According to Scheme 8, under N₂ protection, a solution of n-BuLi in hexane (3.3 mL, 2.5 M) was added dropwise in to a solution of compound j (1.00 g, 2.7 mmol) in THF (50 mL) at −78° C. After the addition, this reaction solution was warmed up to −10° C. slowly and it was then cooled to at −78° C. again. Few grams of dry ice were added rapidly. The cloudy solution was changed to a yellowish color with yellow solid formed. This mixture was then warmed to room temperature. 10 mL of water was then added to this mixture and a clear solution was formed. To this solution, 30 ml of aqueous NaHCO₃ solution was added. After evaporation of most of THF solvent, the solid in the residual cloudy solution was collected and recrystallized from ETOH to yield 29 (0.20 g).

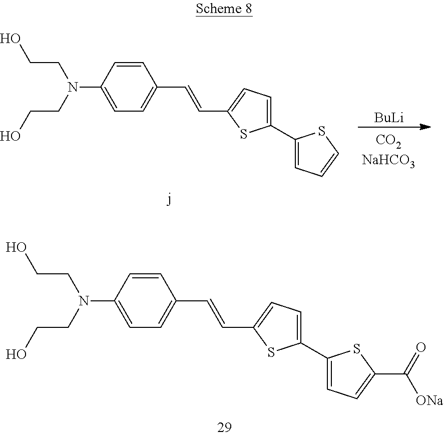

### xviii. Synthesis of Compounds A, B, C, D, E, and F

These compounds were synthesized using similar protocol as shown in Scheme 9 (the generic synthetic strategy for thiophene derivatives). The detail synthesis procedure of compound A (“YE210”) was used as an example. All compounds were purified using High Performance Liquid Chromatograph (HPLC) and characterized using both ¹H NMR and ¹³C NMR.

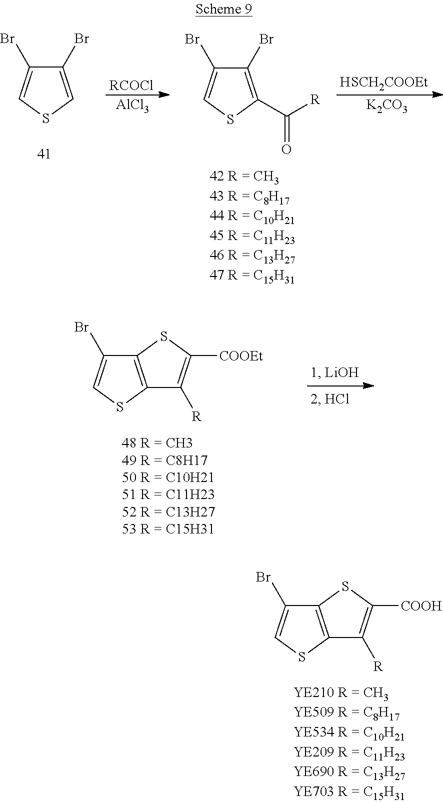

Synthesis of 3,4-Dibromothienyl-2-methyl ketone (42). To a mixture of 3,4-dibromothiophene 41 (72.60 g, 0.30 mol) and AlCl₃ (92.46 g, 0.69 mol) in CH₂Cl₂ (300 mL) at 0° C., acetyl chloride (24.73 g, 0.32 mol) was added dropwise under a nitrogen stream. This was stirred for 2 to 3 hours until no starting materials could be detected by GC/MS. The mixture was then poured into HCl (500 mL, 6M) and the organic was extracted with CH₂Cl₂ (2×300 mL). The combined organic solution was washed with brine (2×150 mL) and water (150 mL). After drying over anhydrous MgSO₄, the solvent was evaporated. A low melting point solid was collected and was pure enough to be used without further purification (80.80 g, 95%). mp 75-78° C. ¹H NMR (300 MHz, CD₂Cl₂) δ 7.67 (s, 1H), 2.69 (s, 3H). ¹³C NMR (300 MHz, CD₂Cl₂) 189.6, 140.6, 130.2, 118.0, 117.3, 29.7, HRMS (ESI) m/z calcd for [C₆H₄Br₂OS] 281.8300, observed; 282.9000.

Synthesis of 6-bromo-3-methyl-ethylthieno[3,2-b]thiophene-2-carboxylate (48). Compound 42 (80.80 g, 0.29 mol) was mixed with K₂CO₃ (196.70 g, 1.43 mol) and DMF (250 mL) in a three neck flask equipped with a condenser and addition funnel. To this mixture ethyl mercaptoacetate (32.80 mL, 0.30 mol) was added dropwise at 60-70° C. A catalytic amount of 18-crown-6 (20 mg) was used as catalyst. The mixture was heated at 60-70° C. overnight until no starting materials were detected by GC/MS. The mixture then was poured into water (1000 mL) and a light YEllow solid was formed. After filtration, the solid washed with water (3×500 mL) and filtrated. The collected solid was washed with methanol (300 mL) and found to be pure enough for the next reaction (78.40 g, 90%). mp 91-92° C. ¹H NMR (300 MHz, CD₂Cl₂) δ 7.48 (s, 1H), 4.36 (q, 2H), 2.63 (s, 3H), 1.38 (t, 3H). ¹³C NMR (300 MHz, CD₂Cl₂) 159.4, 137.9, 137.6, 135.1, 125.7, 124.2, 99.7, 57.8, 11.1, 10.7. HRMS (ESI) m/z calcd for [C₁₀H₉BrO₂S₂] 303.9200. found 303.3000.

Synthesis of 6-bromo-3-methyl-thieno[3,2-b]thiophene-2-carboxylic acid (A). Compound 48 (78.40 g, 0.26 mol) was dissolved into a mixture of THF (400 mL), methanol (50 mL) and LiOH (100 mL, 10% solution). This mixture was refluxed overnight and poured into concentrated hydrochloric acid (300 mL). The acid mixture was then diluted to 1000 mL with water. Solid was filtrated and washed with water (3×500 mL). The light YEllow solid was washed with methanol (300 mL) and dried under vacuum overnight (68.10 g. 96%). mp 280-282° C. ¹H NMR (300 MHz, DMSO) δ 8.08 (s, 1H), 2.60 (s, 3H). ¹³C NMR (300 MHz, DMSO) 163.6, 140.7, 140.1, 137.7, 129.7, 129.2, 102.1, 14.2. HRMS (MALDI) m/z calcd for [C₈H₅BrO₂S₂—H₂O+H] 258.8887. found 258.8883.

2. Materials and Methods

i. Materials

Zaprinast was obtained from BioMol International Inc (Plymouth Meeting, Pa.). Epic® 384 biosensor microplates cell culture compatible were obtained from Corning Inc. (Corning, N.Y.). Both HEK293 and HT-29 were obtained from American Type Cell Culture (Manassas, Va.). The cell culture medium was as follows: (1) Eagle's medium (MEM) supplemented with 10% fetal bovine serum (FBS), 4.5 g/liter glucose, 2 mM glutamine, and antibiotics for HEK293; and (2) McCoy's 5a Medium Modified supplemented with 10% FBS, 4.5 g/liter glucose, 2 mM glutamine, and antibiotics for human colorectal adenocarcinoma HT29.

ii. Calcium Flux Assay

Fluo-4 Direct™ Calcium Assay Kit was purchased from Invitrogen (Starter pack, Cat. no. F10471). HEK293 cells (15000 cells/well) and HT29 cells (30000 cells/well) were seeded in polyD-lysine coated 384well plates (Corning Inc., Cat#3845), culture overnight at 37° C. The next day, Ca²⁺ flux assay was performed following manufacturer's instruction and fluorescence ratio (340 nm/380 nm) was measured on FDSS (Functional Drug Screening System, Hamamatsu Photonics, Japan). Compounds were prepared as 10× stock (final concentration 10 μM) in the Ca²⁺ assay buffer.

Ca²⁺ flux assay with transient transfected cells were carried out in 96-well plates (Corning Inc, Cat#3664). HEK293 cells (25000 cells/well) were seeded on day 1. Cells were transfected with either myc-GPR35 or HA-G_{qo5} or co-transfected with both plasmids (myc-GPR35/HA-G_{qo5} DNA ratio 2:1) using Lipofectamine LTX (Invitrogen) on day 2. Ca²⁺ flux assay was performed 24 hours after transfection.

iii. Cloning of HA-G_{qo5} plasmid

Human G_{q} cDNA plasmid was purchased from Missouri S&T cDNA Resource Center (www.cDNA.org). HA-tag was inserted to the N-terminal of human G_{q} cDNA by PCR (J. Takasaki, T. Saito, M. Taniguchi, T. Kawasaki, Y. Moritani, K. Hayashi and M. Kobori (2004) A Novel Gαq/11-selective Inhibitor. J. Biol. Chem. 279: 47438-47445). The last five amino acids were also replaced to GCGLY by PCR to generate HA-G_{qo5} plasmid (B. R. Conklin, Z. Farfel, K. D. Lustig, D. Julius, and H. R. Bourne (1993) Substitution of three amino acids switches receptor specificity of G_{q} to that of Gᵢ. Nature 1993, 363: 274-276). The G_{qo5} cDNA was then subcloned into expression vector pcDNA3.1(+).

iv. Western Blot Using HT29 and Transfected HEK293 Cells

HT29 cells (10⁷ cells per sample) were harvested and lysed in 1% NP40 lysis buffer (150 mM NaCl, 25 mM Tris, 1% NP 40, pH 7.5) with protease inhibitors cocktail (Roche). Proteins were separated on 15% SDS gel. Membrane was blotted with rabbit anti-GPR35 (1:1000) (Abcam, Ab76217) at 4° C. overnight, then with 2nd HRP conjugated Goat anti-rabbit or Horse anti-goat antibody (1:2000 dilution) for 30 minutes. Western blots were developed using the ECL plus kit (GE Healthcare) on a Fujifilm Luminescent Image Analyzer LAS 3000 (Fujifilm, Valhalla, N.Y.).

v. cAMP Assay

Cells were plated in 384well plates (BD Bioscience, Cat#354660) (10000 cells/well for HEK293, 20000 cells/well for HT29). Cells were cultured in complete growth medium overnight. The next day cAMP-Glo assay was performed according to manufacturer's instruction (Promega, Cat#V1502). Cells were incubated with 20 μM compounds in induction buffer with or without 0.5 μM or 5 μM forskolin for 30 minutes before adding lysis buffer. Luminescence was measured using Tecan SafireII reader.

vi. Immunofluorescence Confocal Imaging

HT29 cells were plated on a 8-well chamber slide (10,000 cells/well) and incubated at 37° C. for 24 hrs. Next day, cells were fixed with 4% formaldehyde in 1×PBS for 15 min, followed by blocking and permeabilization in a buffer that contains 4% goat serum, 0.1% BSA, 0.1% Triton X100 in 1×PBS for 2 hrs. After 5 min wash with PBS, fixed cells were incubated with primary antibody anti-GPR35 (Abcam) (1:300) in 3% BSA/PBS buffer for 24 hrs, followed by incubation with secondary antibody Alexa Fluor® 488 goat anti-rabbit IgG (H+L) (Invitrogen) (1:250) in 3% BSA/PBS for 1 hr at room temperature. Cells were finally washed once with PBS and sealed with 1.5 mm thick glass cover-slip. Dried slides were stored at 4° C. until imaging. Confocal imaging was performed with Zeiss confocal microscope Axiovert 40. For GPR35 internalization study, after cells were incubated at 37° C. for 24 hrs, they were treated with different ligands for GPR35 at 37° C. for 1 hr before fixation (Zaprinast, 10 μM; Niflumic acid, 20 μM; 6-bromo-3-methylthieno[3,2-b]thiophene-2-carboxylic acid, 20 μM; Talniflumate, 20 μM; Diclofenac, 20 μM; Furosemide, 20 μM). Confocal images were analyzed using MacBiophotonics Image J software (http://www.macbiophotonics.ca/downloads.htm).

vii. RNAi Knockdown of GPR35 on Epic® Plate In Situ

GPR35 knockdown in HT29 cells was performed on 384-well Epic® plate with transiently transfected small hairpin (sh) RNA targeted at the human GPR35 mRNA. HT29 cells were seeded at 20,000 cells/well on day 1. On day 2, cells were transiently transfected with plamid DNA pGFP-V—RS-shRNA targeting GPR35 using Effectene (Qiagen) according to manufacturer's instruction. Cells were washed on day 3 and Epic® cell assays were performed on day 4 after 48 hrs of transfection.

viii. Optical Biosensor System and Cell Assays (Label-Free Biosensor Cellular Assays)

Epic® beta version wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15 sec. Also Epic® commercial systems were used, wherein a liquid handler accessory was attached to Epic® reader system.

The RWG biosensor is capable of detecting minute changes in local index of refraction near the sensor surface. Since the local index of refraction within a cell is a function of density and its distribution of biomass (e.g., proteins, molecular complexes), the biosensor exploits its evanescent wave to non-invasively detect ligand-induced dynamic mass redistribution in native cells. The evanescent wave extends into the cells and exponentially decays over distance, leading to a characteristic sensing volume of ˜150 nm, implying that any optical response mediated through the receptor activation only represents an average over the portion of the cell that the evanescent wave is sampling. The aggregation of many cellular events downstream the receptor activation determines the kinetics and amplitudes of a ligand-induced DMR.

For biosensor cellular assays, cells were typically grown using ˜1 to 2×10⁴ cells per well at passage 3 to 15 suspended in 50 μl of the corresponding culture medium in the biosensor microplate, and were cultured at 37° C. under air/5% CO₂ for ˜1 day. The confluency for all cells at the time of assays was ˜95% to 100%. The molecule solutions were made by diluting the stored concentrated solutions with the HBSS (1× Hanks balanced salt solution, plus 20 mM Hepes, pH 7.1), and transferred into a 384well polypropylene molecule storage plate to prepare a molecule source plate. Both molecule and marker source plates were made separately when a two-step assay was performed. In parallel, the cells were washed twice with the HBSS and maintained in 30 μl of the HBSS to prepare a cell assay plate. Both the cell assay plate and the molecule and marker source plate(s) were then incubated in the hotel of the reader system. After ˜1 hr of incubation the baseline wavelengths of all biosensors in the cell assay microplate were recorded and normalized to zero. Afterwards, a 2 to 10 minute continuous recording was carried out to establish a baseline, and to ensure that the cells reached a steady state. Cellular responses were then triggered by pipetting 10 μl of the marker solutions into the cell assay plate using the on-board liquid handler.

To study the influence of molecules on a marker-induced response, a second stimulation with the marker at a fixed dose (typically at EC80 or EC100) was applied. The resonant wavelengths of all biosensors in the microplate were normalized again to establish a second baseline, right before the second stimulation. The two stimulations were usually separated by ˜1 hr.

All studies were carried out at a controlled temperature (28° C.). At least two independent sets of experiments, each with at least three replicates, were performed. The assay coefficient of variation was found to be <10%.

3. Results

i. Expression and Location of GPR35 in HT29 Cells

G protein-coupled receptors (GPCRs) comprise one of the largest families of cell surface proteins and represent a major target for both current therapeutic agents and drugs under development. Many cDNA clones are predicted to code for GPCRs based on high sequence similarity, especially in the transmembrane domains, to established GPCRs. For such orphan GPCRs, the identification of agonists, antagonists, and signal transduction pathways represents a major effort by industry for the discovery of novel drug targets. GPR35, an orphan GPCR first discovered during a human genomic DNA screen, shares limited sequence homology with purinergic P2Y receptors, nicotinic acid receptor HM74, lysophosphatidic acid receptor GPR23, and an orphan receptor, GPR55. The highest levels of GPR35 mRNA were found in immune and gastrointestinal tissues with only limited expression in lung and neuronal tissues. Subsequent to the initial description of GPR35 (now denoted GPR35a), Okumura et al. (2004) isolated a splice variant (GPR35b) from a human gastric cancer cDNA library that coded for an additional 31 amino acids at the N terminus (Okumura, S., Baba, H., Kumada, T., Nanmoku, K., Nakajima, H., Nakane, Y., Hioki, K., and Ikenaka, K. (2004) Cloning of a G-protein-coupled receptor that shows an activity to transform NIH3T3 cells and is expressed in gastric cancer cells. Cancer Sci, 95: 131-135). GPR35a and GPR35b mRNA levels were up-regulated in gastric cancer tissue, suggesting a role for both splice variants in malignant transformation. However, the expression of GPR35 in human colon cancer cells has not been examined.

Real time PCR using GPR35 primers from SA Biosciences showed that HT29 expresses relatively high level of GPR35, at least at mRNA level. Subsequent western blotting showed that HT29 lysates contain GPR35 isoforms, whose molecular weight is close to the expected values for both GPR35a and GPR35b, respectively (FIG. 1a). HT29 is a human colon adenocarcinoma grade II cell line. Confocal imaging further showed that GPR35 is primarily located at the cell surface plasma membrane (FIG. 1b). Interestingly, the staining pattern of GPR35, using anti-GPR35 antibody, suggest that instead of uniformly distributing at the cell surface, GPR35 is primarily located within certain microdomains, possibly lipid rafts and/or complexed with other receptors. Nonetheless, these results suggest that HT29 expresses relatively high level of GPR35a and GPR35b, at both mRNA and protein levels; and GPR35 is primarily located at the cell surface.

ii. The Receptor Internalization and DMR Signals of GPR35 in HT29 Caused by the Known GPR35 Agonist Zaprinast.

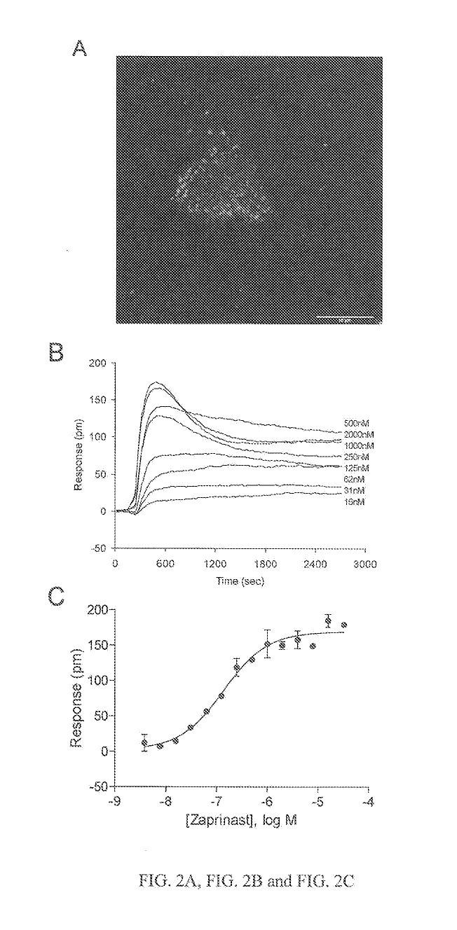

Almost common to all GPCRs is the agonist-induced receptor internalization process. Thus, the ability of the known GPR35 agonist zaprinast to cause receptor internalization was first examined. Zaprinast is also a known cGMP-dependent phosphodiesterase inhibitor. As shown in FIG. 2A, zaprinast at 10 micromolar indeed caused receptor internalization, as evidenced by the confocal imaging of HT29 cells 30 min after stimulation with zaprinast. The green dots indicate that the receptors are internalized and associated with endosome particles, while the blue indicates the nucleus DAPI staining.

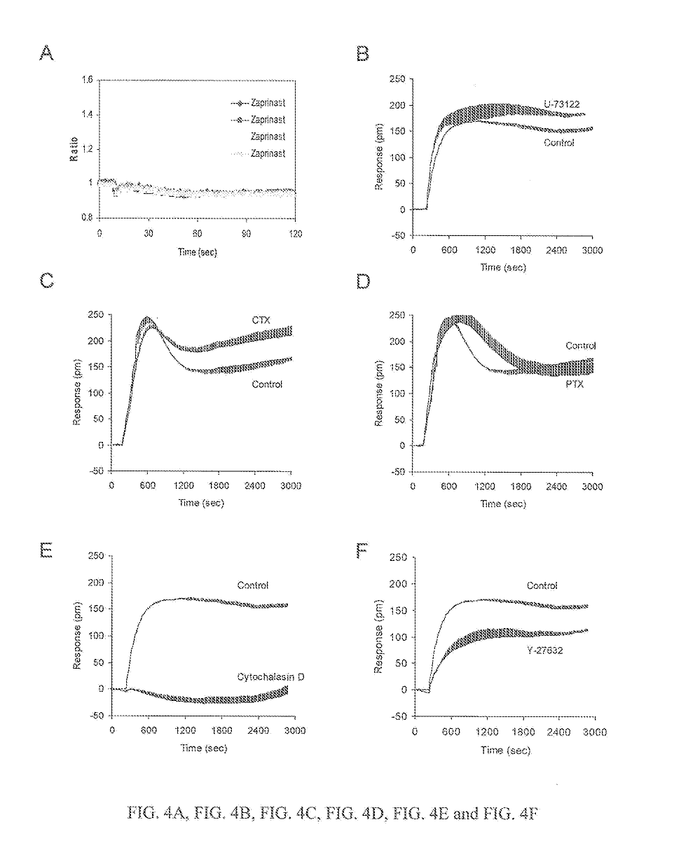

Using Ca²⁺ mobilization assays, several studies suggest that several known GPR35 agonists, including zaprinast, LPA, NPPB and kynurenic acid, were incapable of triggering Ca²⁺ mobilization in the engineered HEK293 cells only expressing GPR35; and the co-expression of GPR35 and G_{qo5} is required for these agonists to trigger robust Ca²⁺ signals. Here Fluo-4 Ca²⁺ assays were used to examine HT29 cells. Results showed that zaprinast failed to cause any significant Ca²⁺ mobilization in HT29 cells (FIG. 4A).

Since optical biosensors primarily employ a surface-bound electromagnetic wave to characterize cellular responses, the resultant optical signal, also termed DMR (dynamic mass redistribution), is an integrated response, reflecting the cell signaling mediated through a receptor in a pathway-sensitive but pathway-unbiased nature. Thus, assaying GPR35 signaling in HT29 cells was conducted. Results showed that zaprinast triggered a dose-dependent and saturable DMR signal in HT29 cells (FIG. 2B and FIG. 2C). Its EC₅₀ to cause the P-DMR event was found to be 137 nM. Follow-up studies using RNAi knockdown suggest that the zaprinast DMR signal is specific to GPR35, since the GPR35 knockdown partially suppressed the zaprinast DMR signal (data not shown). These results suggest that zaprinast acts as an agonist for the endogenous GPR35 in HT29, and is able to cause receptor internalization and a saturable DMR signal. However, it is unclear that zaprinast is specific to GPR35a, or GPR35b, or both.

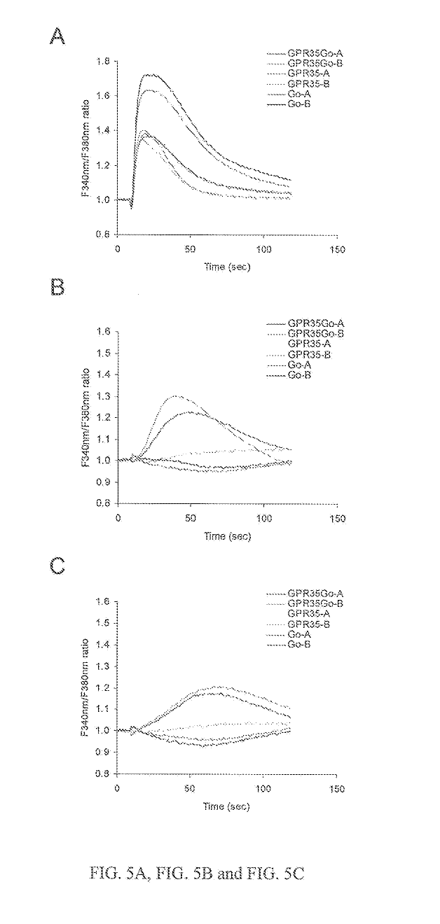

As expected, in engineered HEK293 cells, zaprinast failed to cause any obvious Ca²⁺ mobilization signal in the cells expressing only G_{qo5} or GPR35, but triggered quite obvious Ca²⁺ signal in the cells co-expressing GPR35 and G_{qo5} (FIG. 5B). Taken together, these results suggest that zaprinast indeed is a GPR35 agonist.

iii. Compound Library Screening Using Epic® System

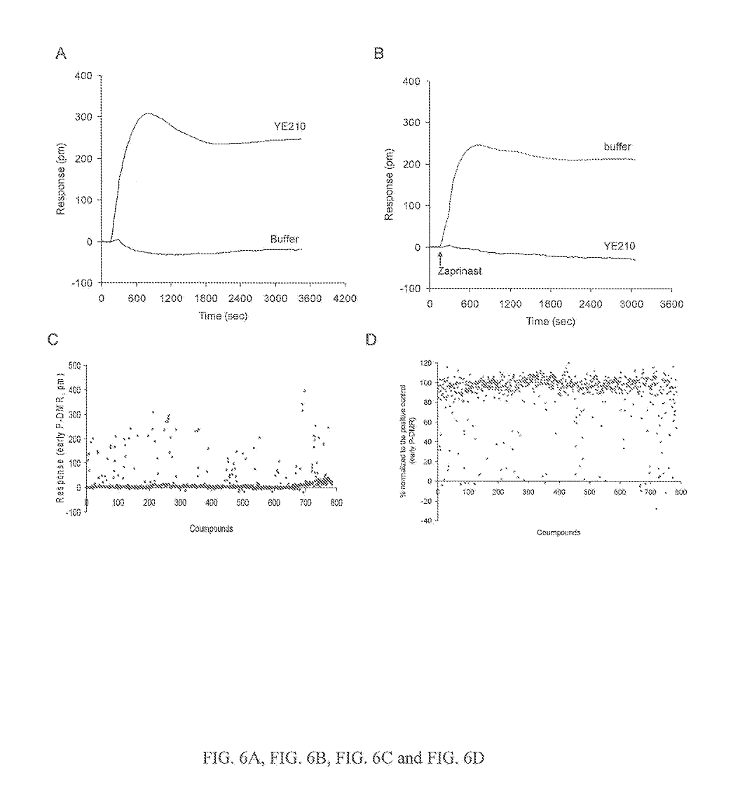

Since Epic® system was found to be able to detect the zaprinast DMR signal in HT29 cells and the zaprinast signal is related to GPR35, it would be interesting in screening compound library using the HT29 Epic® cellular assays. Based on the similarity between a compound-induced DMR signal and the zaprinast DMR signal, a number of potential GPR35 agonists, including all compounds indicated in formula (I) to (VI), were discovered. FIG. 6A showed an example wherein 6-bromo-3-methylthieno[3,2-b]thiophene-2-carboxylic acid at 10 micromolar caused a DMR signal almost identical to the zaprinast DMR signal, while FIG. 6C showed the DMR signal, calculated based on the amplitudes the DMR signals induced by each compound 5 min post stimulation, as a function of compound. Results showed that many compounds triggered a DMR signal that is similar to either the zaprinast or the YE210-induced DMR signal, with an amplitude greater than 50 pm (FIG. 6C). Almost all of these compounds that showed agonism activity were also found to be able to cause the desensitization of HT29 cells in response to sequential stimulation with zaprinast (an example was showed in FIGS. 6B and D), suggesting that these compounds act as GPR35 specific agonists.

iv. a Representative Compound of the Compounds of Formula (I) to (VI) as Present Disclosure is a GPR35 Agonist

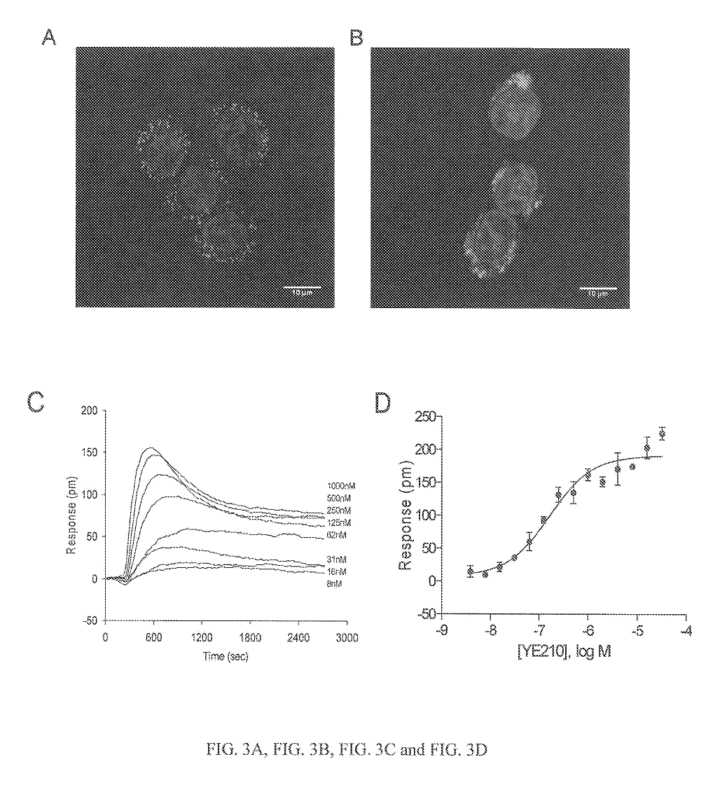

To further characterize these compounds identified from Epic® cellular assay screening, several assays were used. One example was summarized in FIG. 3. Confocal imaging showed that similar to zaprinast, 6-bromo-3-methylthieno[3,2-b]thiophene-2-carboxylic acid caused receptor internalization (FIG. 3B), although there are some GPR35 receptors remained at the cell surface (FIG. 3A). 6-bromo-3-methylthieno[3,2-b]thiophene-2-carboxylic acid also triggered a dose-dependent and saturable DMR signal, with an EC₅₀ of 165 nM (FIG. 3C and FIG. 3D). RNAi knockdown studies also showed that RNAi knockdown of GPR35 partially attenuated the 6-bromo-3-methylthieno[3,2-b]thiophene-2-carboxylic acid DMR signal. In engineered HEK293 cells, 6-bromo-3-methylthieno[3,2-b]thiophene-2-carboxylic acid was found to behave similar to zaprinast-6-bromo-3-methylthieno[3,2-b]thiophene-2-carboxylic acid failed to cause any obvious Ca²⁺ mobilization signal in the cells expressing only G_{qo5} or GPR35, but triggered quite obvious Ca²⁺ signal in the cells co-expressing GPR35 and G_{qo5} (FIG. 5C). Taken together, these results suggest that 6-bromo-3-methylthieno[3,2-b]thiophene-2-carboxylic acid also acts as a potent GPR35 agonist.

v. GPR35 Signaling in HT29 Cells is Linked to the G_{12/13}-Rock Pathway

The signaling pathway of GPR35 is largely unknown today, particularly in HT29 cells. In engineered cells such as CHO-K1 and HEK293 cells, the expressed GPR35 was found to be incapable of triggering Ca²⁺ mobilization when the receptor was expressed alone. The co-transfection of cells with both GPR35 and a promiscuous G protein (G_{qo5}) was found to be required to cause Ca²⁺ mobilization once the receptor is activated. It was also found that in the native HT29 cells, the activation of GPR35 by zaprinast or 6-bromo-3-methylthieno[3,2-b]thiophene-2-carboxylic acid was unable to cause any detectable Ca²⁺ signal (FIG. 4A). The same was true in engineered HEK293 cells only expressing G_{qo5} or GPR35 (FIG. 5).

Interestingly, Epic® cellular assays also indicated that the zaprinast DMR signal was insensitive to the pretreatment of HT29 cells with either phopsholipase C inhibitor U73122, or cholera toxin (CTX), or pertussis toxin (PTX) (FIG. 4B-D, respectively). PLC is a downstream target of G_{q} pathway, but upstream of Ca²⁺ mobilization; the inability to modulate the zaprinast DMR signal by U73122 suggest that the zaprinast signal is largely not due to G_{q} pathway. CTX is known to kill Gₛ signaling pathway; the inability of CTX to attenuate the zaprinast DMR signal suggest that the zaprinast signal is not due to Gₛ pathway. Consistent were the observations that neither zaprinast nor 6-bromo-3-methylthieno[3,2-b]thiophene-2-carboxylic acid was able to cause increase in intracellular cAMP concentration—a classical second messenger readout for Gₛ pathway. Similarly, the Gᵢ pathway killer PTX also had little impact on the zaprinast DMR. Also consistent with this were the observations that neither zaprinast nor 6-bromo-3-methylthieno[3,2-b]thiophene-2-carboxylic acid cause any suppression in intracellular cAMP concentration in the forskolin co-stimulated HT29 cells—a classical second messenger readout for Gᵢ signaling (data not shown). Taken together, these results suggest that GPR35 mediates signaling via non-conventional pathways.

Interestingly, the actin filament disrupting agent cytochalasin D was found to be able to completely block the zaprinast DMR signal, and the ROCK inhibitor Y27632 also partially attenuated the zaprinast signal (FIG. 4E and FIG. 4F). These results suggest that GPR35 may preferentially mediate signaling via G_{12/13}-ROCK pathway.

Similarly, 6-bromo-3-methylthieno[3,2-b]thiophene-2-carboxylic acid, the GPR35 agonist discovered according to the present disclosure, was also found to be unable to cause Ca²⁺ signal in HT29 cells. The 6-bromo-3-methylthieno[3,2-b]thiophene-2-carboxylic acid DMR in HT29 was found to be insensitive to the PLC inhibitor U73122, CTX, or PTX. However, the 6-bromo-3-methylthieno[3,2-b]thiophene-2-carboxylic acid DMR was blocked by cytochalasin D, and partially attenuated by Y27632. Taken together, these results suggest that GPR35 mediates signaling primarily via G_{12/13} pathway.

### B. Example 2

### 1. Material and Method

i. Materials

Zaprinast was obtained from BioMol International Inc (Plymouth Meeting, Pa.). Cell culture compatible Epic® 384 biosensor microplates were obtained from Corning Inc. (Corning, N.Y.).

Both HEK293 and HT-29 cells were obtained from American Type Cell Culture (Manassas, Va.). The cell culture medium was as follows: (1) Eagle's medium (MEM) supplemented with 10% fetal bovine serum (FBS), 4.5 g/liter glucose, 2 mM glutamine, and antibiotics for HEK293; and (2) McCoy's 5a Medium Modified supplemented with 10% FBS, 4.5 g/liter glucose, 2 mM glutamine, and antibiotics for human colorectal adenocarcinoma HT29.

Both the HEK hERG stable cell line (HEK-hERG) and the CHO hERG stable cell line (CHO-hERG) were maintained according to Sun et al. (J. Biol. Chem. 2006, 281:5877). Cells were subcultured 1-2 times per week and cells passaged less than 15 times were used for all experiments.

ii. Calcium Flux Assay

Fluo-4 Direct™ Calcium Assay Kit was purchased from Invitrogen (Starter pack, Cat. no. F10471). HEK293 cells (15,000 cells/well) and HT29 cells (30,000 cells/well) were seeded in polyD-lysine coated 384 well plates (Corning Inc., Cat#3845), the cells were cultured overnight at 37° C. The next day, Ca²⁺ flux assay was performed following manufacturer's instruction and fluorescence ratio (340 nm/380 nm) was measured on FDSS (Functional Drug Screening System, Hamamatsu Photonics, Japan). The compounds were prepared as 10× stock (final concentration 10 μM) in the Ca²⁺ assay buffer.

The Ca²⁺ flux assay with transient transfected cells was carried out in 96-well plates (Corning Inc, Cat#3664). HEK293 cells (25,000 cells/well) were seeded on day 1. The cells were transfected with either myc-GPR35 or HA-G_{qo5} or co-transfected with both plasmids (myc-GPR35/HA-G_{qo5} DNA ratio 2:1) using Lipofectamine LTX (Invitrogen) on day 2. The Ca²⁺ flux assay was performed 24 hours after transfection.

iii. Cloning of HA-G_{qo5} Plasmid

Human G_{q} cDNA plasmid was purchased from Missouri S&T cDNA Resource Center (www.cDNA.org). A HA-tag was inserted to the N-terminal of human G_{q} cDNA by PCR (J. Takasaki, et al., (2004) J. Biol. Chem. 279: 47438-47445). The last five amino acids were also replaced to GCGLY by PCR to generate HA-G_{qo5} plasmid (B. R. Conklin, Z. et al., (1993), Nature 1993, 363: 274-276). The G_{qo5} cDNA was then subcloned into expression vector pcDNA3.1(+).

iv. Western Blot Using HT29 and Transfected HEK293 Cells

HT29 cells (10⁷ cells per sample) were harvested and lysed in 1% NP40 lysis buffer (150 mM NaCl, 25 mM Tris, 1% NP 40, pH 7.5) with protease inhibitors cocktail (Roche). Proteins were separated on 15% SDS gel and then transferred to a membrane. The membrane was blotted with rabbit anti-GPR35 (1:1000) (Abcam, Ab76217) at 4° C. overnight, then with a secondary antibody, HRP conjugated Goat anti-rabbit or Horse anti-goat antibody (1:2000 dilution), for 30 minutes. Western blots were developed using the ECL plus kit (GE Healthcare) on a Fujifilm Luminescent Image Analyzer LAS 3000 (Fujifilm, Valhalla, N.Y.).

v. cAMP Assay

Cells were plated in 384 well plates (BD Bioscience, Cat# 354660) (10000 cells/well for HEK293, 20000 cells/well for HT29). Cells were cultured in complete growth medium overnight. The next day cAMP-Glo assay was performed according to manufacturer's instruction (Promega, Cat#V 1502). Cells were incubated with 20 μM compounds in induction buffer with or without 0.5 μM or 5 μM forskolin for 30 minutes before adding lysis buffer. Luminescence was measured using Tecan SafireII reader.

vi. Immunofluorescence Confocal Imaging

HT29 cells were plated on a 8-well chamber slide (10,000 cells/well) and incubated at 37° C. for 24 hrs. The next day, cells were fixed with 4% formaldehyde in 1×PBS for 15 mM, followed by blocking and permeabilization in a buffer that contains 4% goat serum, 0.1% BSA, 0.1% Triton X100 in 1×PBS for 2 hrs. After a 5 mM wash with PBS, fixed cells were incubated with primary antibody anti-GPR35 (Abcam) (1:300) in 3% BSA/PBS buffer for 24 hrs, followed by incubation with secondary antibody Alexa Fluor® 488 goat anti-rabbit IgG (H+L) (Invitrogen) (1:250) in 3% BSA/PBS for 1 hr at room temperature. Cells were finally washed once with PBS and sealed with a 1.5 mm thick glass cover-slip. Dried slides were stored at 4° C. until imaging. Confocal imaging was performed with a Zeiss confocal microscope Axiovert 40. For GPR35 internalization studies, after cells were incubated at 37° C. for 24 hrs, they were treated with different ligands for GPR35 at 37° C. for 1 hr before fixation (Zaprinast, 10 μM; Niflumic acid, 20 μM; YE210, 20 μM; Talniflumate, 20 μM; Diclofenac, 20 μM; Furosemide, 20 μM). Confocal images were analyzed using MacBiophotonics Image J software (http://www.macbiophotonics.ca/downloads.htm).

vii. RNAi Knockdown of GPR35 on Epic® Plate In Situ

GPR35 knockdown in HT29 cells was performed on 384-well Epic® plate with transiently transfected small hairpin (sh) RNA targeted at the human GPR35 mRNA. HT29 cells were seeded at 20,000 cells/well on day 1. On day 2, cells were transiently transfected with plamid DNA pGFP-V—RS-shRNA targeting GPR35 using Effectene (Qiagen) according to manufacturer's instruction. The cells were washed on day 3 and Epic® cell assays were performed on day 4 after 48 hrs of transfection.

viii. Optical Biosensor System and Cell Assays

The Epic® beta version wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15 sec. Also Epic® commercial systems were used, wherein a liquid handler accessory was attached to the Epic® reader system.

The RWG biosensor is capable of detecting minute changes in local index of refraction near the sensor surface. Since the local index of refraction within a cell is a function of density and its distribution of biomass (e.g., proteins, molecular complexes), the biosensor exploits its evanescent wave to non-invasively detect ligand-induced dynamic mass redistribution in native cells. The evanescent wave extends into the cells and exponentially decays over distance, leading to a characteristic sensing volume of ˜150 nm, implying that any optical response mediated through the receptor activation only represents an average over the portion of the cell that the evanescent wave is sampling. The aggregation of many cellular events downstream of the receptor activation determines the kinetics and amplitudes of a ligand-induced DMR.

For biosensor cellular assays, cells were typically grown using ˜1 to 2×10⁴ cells per well at passage 3 to 15 suspended in 50 μl of the corresponding culture medium in the biosensor microplate, and were cultured at 37° C. under air/5% CO₂ for ˜1 day. The confluency for all cells at the time of assays was ˜95% to 100%. The molecule solutions were made by diluting the stored concentrated solutions with the HBSS (1× Hanks balanced salt solution, plus 20 mM Hepes, pH 7.1), and then transferred into a 384 well polypropylene molecule storage plate to prepare a molecule source plate. Both molecule and marker source plates were made separately when a two-step assay was performed. In parallel, the cells were washed twice with the HBSS and maintained in 30 μl of the HBSS to prepare a cell assay plate. Both the cell assay plate and the molecule and marker source plate(s) were then incubated in the hotel of the reader system. After ˜1 hr of incubation the baseline wavelengths of all biosensors in the cell assay microplate were recorded and normalized to zero. Afterwards, a 2 to 10 minute continuous recording was carried out to establish a baseline, and to ensure that the cells reached a steady state. Cellular responses were then triggered by pipetting 10 μl of the marker solutions into the cell assay plate using the on-board liquid handler.

To study the influence of molecules on a marker-induced response, a second stimulation with the marker at a fixed dose (typically at EC80 or EC100) was applied. The resonant wavelengths of all biosensors in the microplate were normalized again to establish a second baseline, right before the second stimulation. The two stimulations were usually separated by ˜1 hr.

All studies were carried out at a controlled temperature (28° C.). At least two independent sets of experiments, each with at least three replicates, were performed. The assay coefficient of variation was found to be <10%.

ix. Co-Immunoprecipitation and Western Blotting

The HEK-hERG cells were seeded in T175 flasks. The cells were transfected with myc-GPR35 using Lipofectamine LTX (Invitrogen) according to manufacturer's instruction. After 24 hrs transfection, the cells were used for co-immunoprecipitation (Co-IP) studies.

For Co-IP studies, HT29 cells (10⁷ cells per sample) were harvested and lysed in 1% NP40 lysis buffer (150 mM NaCl, 25 mM Tris, 1% NP 40, pH 7.5) with protease inhibitors cocktail (Roche). The cell lysate was immunoprecipitated with either rabbit anti-GPR35 (Abeam, Ab76217) or rabbit anti-HERG (Alomone Labs, APC-062) conjugated with Protein A sepharose. Proteins were separated on a 15% SDS gel and then transferred to a membrane. The membrane was blotted with rabbit anti-GPR35 (1:1000) at 4° C. overnight, then with secondary antibody HRP conjugated Goat anti-rabbit or Horse anti-goat antibody (1:2000 dilution) for 30 minutes. Western blots were developed using the ECL plus kit (GE Healthcare) on a Fujifilm Luminescent Image Analyzer LAS 3000 (Fujifilm, Valhalla, N.Y.).

x. Automated Patch Clamp Recording Using IonWorks

CHO-K1 cells stably expressing HERG channel were cultured in T175 flasks until about 70% confluent. The cells were washed twice with PBS, then 2.5 ml 0.25% Trypsin/EDTA was mixed with 2.5 ml PBS and added to the T175 flask. The cells were incubated about 2 minutes with the diluted Trypsin/EDTA solution at 37° C., then were continuously incubated about 3 minutes at room temperature. 20 ml fresh medium were added to suspend the cells and transfer to a 50 ml tube. Cells were centrifuged down at 750 rpm for 5 minutes. The extra medium was removed and cells were resuspended in 6 ml External Buffer (137 mM NaCl, 4 mM KCl, 1.8 mM CaCl₂, 1 mM MgCl₂, 10 mM HEPES, 10 mM glucose, pH 7.4). Then cells were centrifuged again at 450 rpm for another 5 minutes. Finally, cells were resuspended in 4 ml External Buffer and the cell number was counted using a hemacytometer. The cell suspension was diluted to 2.5×10⁶ cells/ml with External Buffer. 4 ml of the resuspended cells were added to the cell reservoir in IonWorks. The Internal solution used contains: 40 mM KCl, 100 mM K-Gluconate, 3.2 mM MgCl₂, 2 mM CaCl₂, 5 mM HEPES, pH 7.25 (adjusted with KOH). 5 mg Amphotericin B from 200 ul DMSO stock was added to 65 ml Internal solution and mixed well to achieve electrical access to the interior of cells on the patch plate.

Compounds were prepared from 10 mM DMSO stock and diluted in External Buffer to make 3× compound solutions. 60 μl/well of the 3× compound solution was transferred to a 384 well plate in Row A, B, C and D. PD11857 (Sigma-Aldrich), a reported HERG channel activator was used as an activator positive control (final concentration 30 and 50 μM). HERG blocker dofetilide (Fisher Scientific) was used as a blocker positive control (final concentration 100 nM). The final DMSO concentration in the IonWorks Quattro PatchPlate PPC (Cat#9000-0902, Molecular Devices) was 0.5%. Final compound concentration was 50 uM. Each compound was added to four wells of one PPC plate.

HERG currents were recorded on IonWorks Quattro (Molecular Devices). To record the HERG current, the cells were clamped at −80 mV initially, then followed by a 5-s depolarization at +40 mV to activate the channels. Tail currents were measured during an ensuing return to −35 mV. Data analysis were done using IonWorks Quattro® System Software version 2.0.4.4. Data from wells with seal resistance less than 50 M) or HERG tail currents less than 0.1 nA were filtered out. Activator hits were selected if the hERG tail currents ratio (post/pre-compound) was greater than the mean+2SD of the average DMSO control. Inhibitor hits were selected if hERG tail currents ratio (post/pre-compound) were less than mean-2SD of the average DMSO control.

### 2. Results

i. HT29 Cell Expresses Both GPR35 and hERG Channel

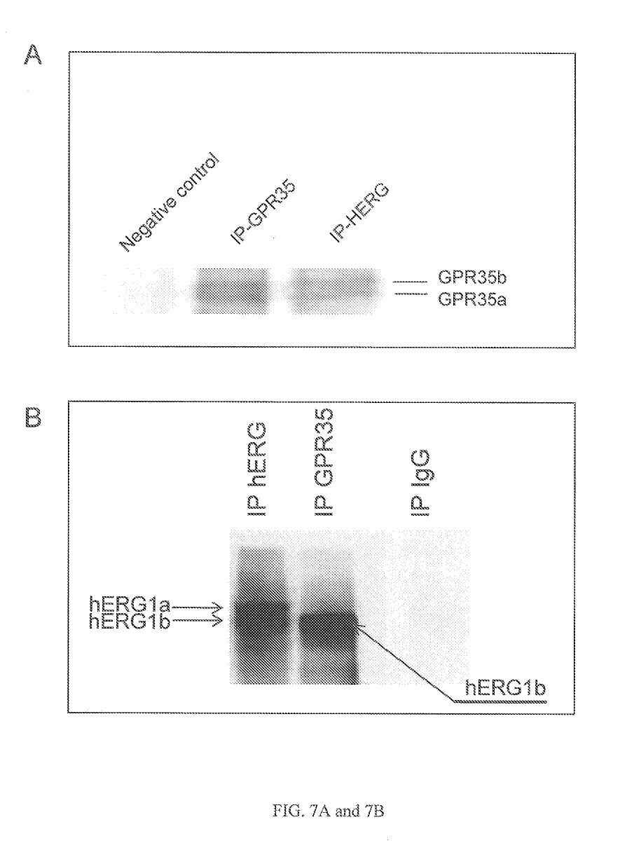

Real time PCR using GPR35 primers from SA Biosciences showed that HT29 expresses relatively high levels of GPR35, at least at the mRNA level. Subsequent western blotting showed that HT29 lysates contain GPR35 isoforms, whose molecular weight is close to the expected values for both GPR35a and GPR35b, respectively (FIG. 1a and FIG. 7A). HT29 is known to express the hERG ion channel. Western blotting studies indicate that HT29 expresses both hERG1a and hERG1b (FIG. 7B).

Confocal imaging further showed that GPR35 is primarily located at the cell surface plasma membrane (FIG. 1b). Interestingly, the staining pattern of GPR35, using an anti-GPR35 antibody, indicate that instead of uniformly distributing at the cell surface, GPR35 is primarily located within certain microdomains, such as lipid rafts and/or complexed with other receptors. Nonetheless, these results indicate that HT29 expresses relatively high levels of GPR35a and GPR35b, at both mRNA and protein levels; and GPR35 is primarily located at the cell surface.

ii. GPR35 Physically Interacts with hERG Ion Channel in HT29 Cells

Co-immunoprecipitation (Co-IP) assays have been viewed as a gold standard to determine the physical interaction between a pair of receptors. As shown in FIG. 7, the anti-GPR35 column can specifically pull down both hERG1b and GPR35a/b. Similarly, the anti-hERG column can specifically pull down both hERG1a/b and GPR35a/b. These results indicate that in HT29, hERG1b is physically associated with GPR35a and/or GPR35b to a signaling complex.

iii. GPR35 Also Physically Interacts with hERG Ion Channel in Engineered HEK293 Cells

To further determine the specificity of the GPR35/hERG signaling complex, HEK-hERG stable cells were transiently transfected with myc-tagged GPR35. The stable line expresses relatively high levels of hERG1. A co-immunoprecipitation (co-IP) assay was further used to characterize potential interactions between GPR35 and hERG1. Results showed that the anti-myc column can specifically pull down both hERG1b and GPR35 (data not shown). Similarly, the anti-hERG column can specifically pull down both hERG1b and GPR35 (data not shown). These results indicate that in engineered HEK cells, hERG1b is physically associated with GPR35.

iv. High Content Image Assays to Characterize GPR35/hERG Signaling Complexes

Almost common to all GPCRs is the agonist-induced receptor internalization process. Thus, the ability of the known GPR35 agonist zaprinast to cause receptor internalization was examined. Zaprinast is a known cGMP-dependent phosphodiesterase inhibitor, and also a GPR35 agonist. As shown in FIG. 2A, zaprinast at 10 micromolar indeed caused receptor internalization, as evidenced by the confocal imaging of HT29 cells 30 min after stimulation with zaprinast. The dots indicate that the receptors are internalized and associated with endosome particles, while the blue indicates the nucleus DAPI staining These results indicate that GPR35, once activated, can undergo internalization, and high content imaging or screening assays can be used to characterize the GPR35/hERG complexes.

v. Label-Free Biosensor DMR Assays to Characterize GPR35/hERG Signaling Complexes

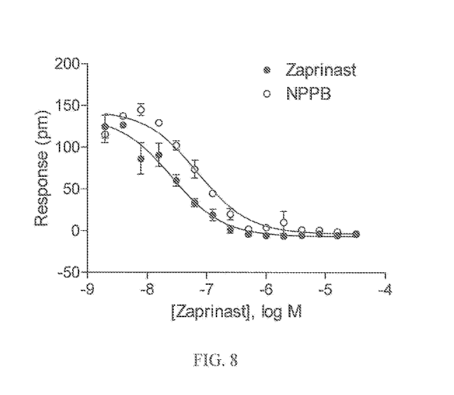

Label-free biosensor cellular assays offer a non-invasive and integrated measure of receptor signaling. Particularly, optical biosensors including resonant waveguide grating biosensors measure dynamics mass redistribution (DMR) of cells in response to stimulation. These biosensor cellular assays provide a pathway unbiased but pathway sensitive measure of receptor signaling. Although the signaling of GPR35, particularly GPR35/hERG complexes, is largely unknown, it is possible to use label free receptor assays to characterize GPR35/hERG signaling complexes. Results showed that zaprinast triggered a dose-dependent and saturable DMR signal in HT29 cells (FIG. 2A and FIG. 2B). Its EC₅₀ to cause the P-DMR event was found to be 137 nM. Zaprinast also dose-dependently caused desensitization of HT29 to the subsequent stimulation with two known GPR35 agonists, zaprinast and NPPB (FIG. 8). Both agonists were examined at EC₁₀₀. These results indicate that zaprinast acts as a GPR35 agonist, and label-free cellular assays can be used to examine the GPR35/hERG signaling complexes.

vi. Patch Clamping Assays to Characterize GPR35/hERG Signaling Complexes

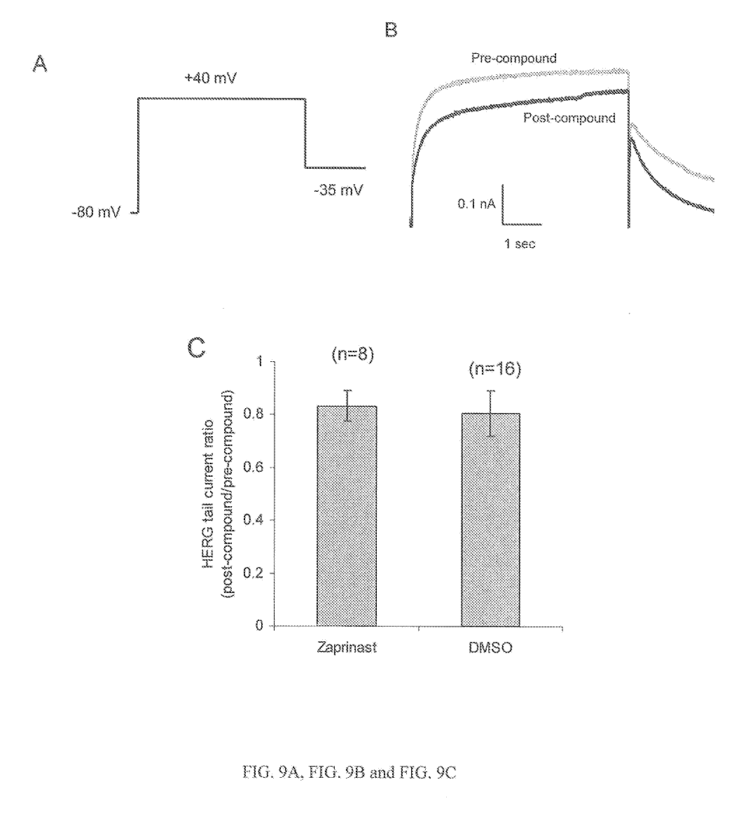

Since hERG is a voltage-gated ion channel, an automated patch clamping assay was performed. Results showed that zaprinast acts as a non-modulator (i.e., neither activator nor inhibitor) of hERG activity in CHO-hERG cells (FIG. 9). These results indicate that zaprinast does not directly modulate the channel activity of hERG.

vii. DMR Assays to Characterize GPR35/hERG Signaling Complexes

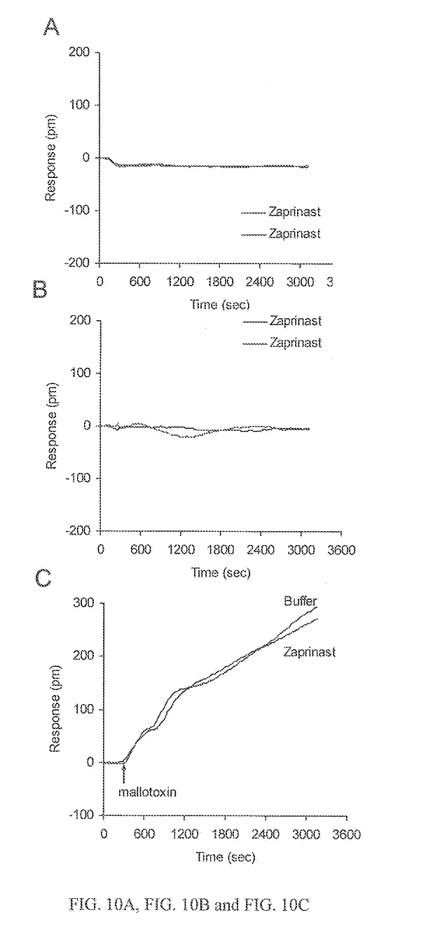

Using Epic® cellular assays, zaprinast was found to trigger a net-zero DMR signal in HEK-293 cells (FIG. 10A), as well as in HEK-hERG cells (FIG. 10B). Zaprinast was also found to have a little or no impact on the mallotoxin DMR signal in HEK-hERG cells (FIG. 10C). Mallotoxin is a known hERG activator. These results further indicate that zaprinast does not directly modulate the channel activity and signaling of hERG when GPR35 is not presented.

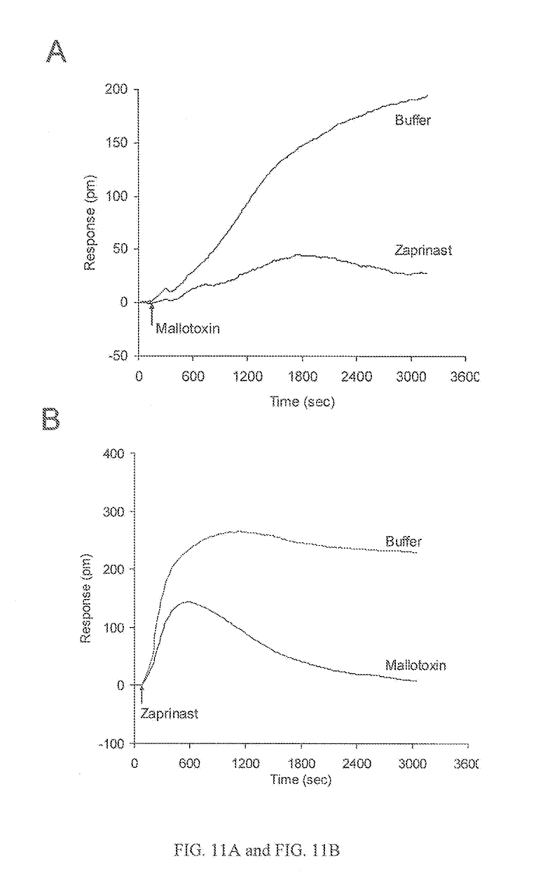

Interestingly, in HT29 cells wherein both GPR35 and hERG are endogenously expressed, zaprinast can partially attenuate the mallotoxin DMR signal (FIG. 11A). Vice versa is also true—mallotoxin can partially attenuate the zaprinast DMR signal (FIG. 11B). Furthermore, the mallotoxin DMR was found to be significantly different from the zaprinast DMR signal. Taken together, these results indicate that zaprinast is a GPR35 agonist, a GPR35/hERG signaling complex agonist, but not a direct hERG modulator.

### C. Example 3

### 1. Materials and Methods

i. Materials

Zaprinast was obtained from BioMol International Inc (Plymouth Meeting, Pa.). Epic® 384 biosensor microplates cell culture compatible were obtained from Corning Inc. (Corning, N.Y.). All tyrphostin compounds were obtained from Sigma Chemical Co. (St. Louis, Mo.).

Both HEK293 and HT-29 were obtained from American Type Cell Culture (Manassas, Va.). The cell culture medium was as follows: (1) Eagle's medium (MEM) supplemented with 10% fetal bovine serum (FBS), 4.5 g/liter glucose, 2 mM glutamine, and antibiotics for HEK293; and (2) McCoy's 5a Medium Modified supplemented with 10% FBS, 4.5 g/liter glucose, 2 mM glutamine, and antibiotics for human colorectal adenocarcinoma HT29.

ii. Calcium Flux Assay

Fluo-4 Direct™ Calcium Assay Kit was purchased from Invitrogen (Starter pack, Cat. no. F10471). HEK293 cells (15000 cells/well) and HT29 cells (30000 cells/well) were seeded in polyD-lysine coated 384 well plates (Corning Inc., Cat#3845) and cultured overnight at 37° C. The next day, the Ca²⁺ flux assay was performed following manufacturer's instruction and fluorescence ratio (340 nm/380 nm) was measured on FDSS (Functional Drug Screening System, Hamamatsu Photonics, Japan). Compounds were prepared as 10× stock (final concentration 10 μM) in the Ca²⁺ assay buffer.

Ca²⁺ flux assays with transient transfected cells were carried out in 96-well plates (Corning Inc, Cat#3664). HEK293 cells (25000 cells/well) were seeded on day 1. Cells were transfected with either myc-GPR35 or HA-G_{qo5} or co-transfected with both plasmids (myc-GPR35/HA-G_{qo5} DNA ratio 2:1) using Lipofectamine LTX (Invitrogen) on day 2. Ca²⁺ flux assays were performed 24 hours after transfection.

iii. Cloning of HA-G_{qo5} Plasmid

Human G_{q} cDNA plasmid was purchased from Missouri S&T cDNA Resource Center (www.cDNA.org). HA-tag was inserted to the N-terminus of human G_{q} cDNA by PCR (J. Takasaki et al. (2004) J. Biol. Chem. 279: 47438-47445). The last five amino acids were also replaced to GCGLY by PCR to generate HA-G_{qo5} plasmid (B. R. Conklin et al. (1993) Nature 363: 274-276). The G_{qo5} cDNA was then subcloned into expression vector pcDNA3.1(+).

iv. Western Blot Using HT29 and Transfected HEK293 Cells

HT29 cells (10⁷ cells per sample) were harvested and lysed in 1% NP40 lysis buffer (150 mM NaCl, 25 mM Tris, 1% NP 40, pH 7.5) with protease inhibitors cocktail (Roche). Proteins were separated on 15% SDS gel and then transferred to a membrane. Membrane was blotted with rabbit anti-GPR35 (1:1000) (Abcam, Ab76217) at 4° C. overnight, then with a secondary antibody, HRP conjugated Goat anti-rabbit or Horse anti-goat antibody (1:2000 dilution), for 30 minutes. Western blots were developed using the ECL plus kit (GE Healthcare) on a Fujifilm Luminescent Image Analyzer LAS 3000 (Fujifilm, Valhalla, N.Y.).

v. cAMP Assay

Cells were plated in 384 well plates (BD Bioscience, Cat# 354660) (10000 cells/well for HEK293, 20000 cells/well for HT29). The cells were cultured in complete growth medium overnight. The next day a cAMP-Glo assay was performed according to manufacturer's instruction (Promega, Cat#V1502). The cells were incubated with 20 μM compounds in induction buffer with or without 0.5 μM or 5 μM forskolin for 30 minutes before adding lysis buffer. Luminescence was measured using Tecan SafireII reader.

vi. Immunofluorescence Confocal Imaging

HT29 cells were plated on a 8-well chamber slide (10,000 cells/well) and incubated at 37° C. for 24 hrs. The next day, cells were fixed with 4% formaldehyde in 1×PBS for 15 min, followed by blocking and permeabilization in a buffer that contains 4% goat serum, 0.1% BSA, 0.1% Triton X100 in 1×PBS for 2 hrs. After a 5 min wash with PBS, fixed cells were incubated with primary antibody, anti-GPR35 (Abcam) (1:300), in 3% BSA/PBS buffer for 24 hrs, followed by incubation with secondary antibody, Alexa Fluor® 488 goat anti-rabbit IgG (H+L) (Invitrogen) (1:250), in 3% BSA/PBS for 1 hr at room temperature. Cells were finally washed once with PBS and sealed with 1.5 mm thick glass cover-slips. Dried slides were stored at 4° C. until imaging. Confocal imaging was performed with a Zeiss confocal microscope Axiovert 40. For GPR35 internalization studies, after cells were incubated at 37° C. for 24 hrs, they were treated with different ligands for GPR35 at 37° C. for 1 hr before fixation (Zaprinast, 10 μM; Niflumic acid, 20 μM; YE210, 20 μM; Talniflumate, 20 μM; Diclofenac, 20 μM; Furosemide, 20 μM). Confocal images were analyzed using MacBiophotonics Image J software (http://www.macbiophotonics.ca/downloads.htm).

vii. RNAi Knockdown of GPR35 on Epic® Plate In Situ

GPR35 knockdown in HT29 cells was performed on 384-well Epic® plates with transiently transfected small hairpin (sh) RNA targeted at the human GPR35 mRNA. HT29 cells were seeded at 20,000 cells/well on day 1. On day 2, cells were transiently transfected with plamid DNA pGFP-V—RS-shRNA targeting GPR35 using Effectene (Qiagen) according to manufacturer's instruction. Cells were washed on day 3 and Epic® cell assays were performed on day 4 after 48 hrs of transfection.

viii. Optical Biosensor System and Cell Assays

Epic® beta version wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15 sec. Also Epic® commercial systems were used, wherein a liquid handler accessory was attached to Epic® reader system.

The RWG biosensor is capable of detecting minute changes in local index of refraction near the sensor surface. Since the local index of refraction within a cell is a function of density and its distribution of biomass (e.g., proteins, molecular complexes), the biosensor exploits its evanescent wave to non-invasively detect ligand-induced dynamic mass redistribution in native cells. The evanescent wave extends into the cells and exponentially decays over distance, leading to a characteristic sensing volume of ˜150 nm, implying that any optical response mediated through the receptor activation only represents an average over the portion of the cell that the evanescent wave is sampling. The aggregation of many cellular events downstream of the receptor activation determines the kinetics and amplitudes of a ligand-induced DMR.

For biosensor cellular assays, cells were typically grown using ˜1 to 2×10⁴ cells per well at passage 3 to 15 suspended in 50 μl of the corresponding culture medium in the biosensor microplate, and were cultured at 37° C. under air/5% CO₂ for ˜1 day. The confluency for all cells at the time of the assays was ˜95% to 100%. The molecule solutions were made by diluting the stored concentrated solutions with the HBSS (1× Hanks balanced salt solution, plus 20 mM Hepes, pH 7.1), and transferred into a 384 well polypropylene molecule storage plate to prepare a molecule source plate. Both molecule and marker source plates were made separately when a two-step assay was performed. In parallel, the cells were washed twice with the HBSS and maintained in 30 μl of the HBSS to prepare a cell assay plate. Both the cell assay plate and the molecule and marker source plate(s) were then incubated in the hotel of the reader system. After ˜1 hr of incubation the baseline wavelengths of all biosensors in the cell assay microplate were recorded and normalized to zero. Afterwards, a 2 to 10 minute continuous recording was carried out to establish a baseline, and to ensure that the cells reached a steady state. Cellular responses were then triggered by pipetting 10 μl of the marker solutions into the cell assay plate using the on-board liquid handler.

To study the influence of molecules on a marker-induced response, a second stimulation with the marker at a fixed dose (typically at EC80 or EC100) was applied. The resonant wavelengths of all biosensors in the microplate were normalized again to establish a second baseline, right before the second stimulation. The two stimulations were usually separated by ˜1 hr.

All studies were carried out at a controlled temperature (28° C.). At least two independent sets of experiments, each with at least three replicates, were performed. The assay coefficient of variation was found to be <10%.

ix. Co-Immunoprecipitation and Western Blotting

HEK-hERG cells were seeded in T175 flasks. Cells were transfected with myc-GPR35 using Lipofectamine LTX (Invitrogen) according to manufacturer's instruction. After 24 hrs transfection, the cells were used for co-immunoprecipitation studies.

A co-IP assay protocol was used. For example, HT29 cells (10⁷ cells per sample) were harvested and lysed in 1% NP40 lysis buffer (150 mM NaCl, 25 mM Tris, 1% NP 40, pH 7.5) with protease inhibitors cocktail (Roche). The cell lysate was immunoprecipitated with either rabbit anti-GPR35 (Abeam, Ab76217) or rabbit anti-HERG (Alomone Labs, APC-062) conjugated with Protein A sepharose. Proteins were separated on 15% SDS gel. Membrane was blotted with rabbit anti-GPR35 (1:1000) at 4° C. overnight, then with the secondary antibody, HRP conjugated Goat anti-rabbit or Horse anti-goat antibody (1:2000 dilution), for 30 minutes. Western blots were developed using the ECL plus kit (GE Healthcare) on a Fujifilm Luminescent Image Analyzer LAS 3000 (Fujifilm, Valhalla, N.Y.).

x. Automated Patch Clamp Recording Using IonWorks

CHO-K1 cells stably expressing HERG channel were cultured in T175 flasks until about 70% confluency. Cells were washed twice with PBS, then 2.5 ml 0.25% Trypsin/EDTA was mixed with 2.5 ml PBS and added to the T175 flasks. Cells were incubated about 2 minutes with the diluted Trypsin/EDTA solution at 37° C., then were continuously incubated about 3 minutes at room temperature. 20 ml fresh medium were added to suspend the cells and transfer to a 50 ml tube. Cells were centrifuged down at 750 rpm for 5 minutes. The extra medium was removed and cells were resuspended in 6 ml External Buffer (137 mM NaCl, 4 mM KCl, 1.8 mM CaCl₂, 1 mM MgCl₂, 10 mM HEPES, 10 mM glucose, pH 7.4). Then cells were centrifuged again at 450 rpm for another 5 minutes. Finally cells were resuspended in 4 ml External Buffer, and the cell number was counted using a hemacytometer. The cell suspension was diluted to 2.5×10⁶ cells/ml with External Buffer. 4 ml of the resuspended cells were added to the cell reservoir in IonWorks. The Internal solution used contains: 40 mM KCl, 100 mM K-Gluconate, 3.2 mM MgCl₂, 2 mM CaCl₂, 5 mM HEPES, pH 7.25 (adjusted with KOH). 5 mg Amphotericin B from 200 ul DMSO stock was added to 65 ml Internal solution and mixed well to achieve electrical access to the interior of cells on the patch plate.

Compounds were prepared from 10 mM DMSO stock and diluted in External Buffer to make 3× compound solution. 60 ul/well of the 3× compound solution was transferred to a 384 well plate in Row A, B, C and D. PD11857 (Sigma-Aldrich), a reported HERG channel activator was used as an activator positive control (final concentration 30 and 50 uM). HERG blocker dofetilide (Fisher Scientific) was used as a blocker positive control (final concentration 100 nM). The final DMSO concentration in the IonWorks Quattro PatchPlate PPC (Cat#9000-0902, Molecular Devices) was 0.5%. Final compound concentration was 50 uM. Each compound was added to four wells of one PPC plate.

HERG currents were recorded on IonWorks Quattro (Molecular Devices). To record the HERG current, the cells were clamped at −80 mV initially, then followed by a 5-s depolarization at +40 mV to activate the channels. Tail currents were measured during an ensuing return to −35 mV. Data analysis were done using IonWorks Quattro® System Software version 2.0.4.4. Data from wells with seal resistance less than 50 MΩ or HERG tail currents less than 0.1 nA were filtered out. Activator hits were selected if hERG tail currents ratios (post/pre-compound) were greater than mean+2SD of average DMSO control. Inhibitor hits were selected if hERG tail currents ratios (post/pre-compound) were less than mean-2SD of average DMSO control.

### 2. Results

i. Chemical Synthesis and Characterization

The following chemicals were synthesized:
1 (2-dicyanomethylene-3-cyano-4,5-dimethyl-5-[4′-n-butylphenyl]-2,5-dihydrofuran)
2 (2-dicyanomethylene-3-cyano-4,5-dimethyl-5-[2′,4′-difluorophenyl]-2,5-dihydrofuran)
3 (2-dicyanomethylene-3-cyano-4-methyl-5-spiro-cyclohexyl-2,5-dihydrofuran)
4 (2-dicyanomethylene-3-cyano-4-methyl-5-phenyl-5-perfluoromethyl-2,5-dihydrofuran)
5 (2-dicyanomethylene-3-cyano-4-methyl-5-spiro-fluorenylidine-2,5-dihydrofuran)
6 (2-dicyanomethylene-3-cyano-4,5-dimethyl-5-[2′,3′,4′-trichlorophenyl]-2,5-dihydrofuran)
7 (2-dicyanomethylene-3-cyano-4,5-dimethyl-5-[3′,4′-dichlorophenyl]-2,5-dihydrofuran)
8 (2-cyano-2-(1-amino-2,2-dicyanovinyl)methylene-3-cyano-4,5,5-trimethyl-2,5-dihydrofuran)
9 (2-dicyanomethylene-3-cyano-4,5-dimethyl-5-[2′3′4′5′6′-pentafluorophenyl]-2,5-dihydrofuran)
10 (2-dicyanomethylene-3-cyano-4,5,5-trimethyl-2,5-dihydrofuran)
11 (thieno[3,2-b]thiophene-2-carboxylic acid)
12 (3-methyl-thieno[3,2-b]thiophene-2-carboxylic acid)
13 (3-methyl-6-bromo-thieno[3,2-b]thiophene-2-carboxylic acid)
14 (6-hexyl-thieno[3,2-b]thiophene-2-carboxylic acid)
15 (3,6-dibromo-thieno[3,2-b]thiophene-2-undecanone)
16 (3-n-undecyl-6-bromo-thieno[3,2-b]thiophene-2-carboxylic acid)
17 (3-n-tridecyl-6-bromo-thieno[3,2-b]thiophene-2-carboxylic acid)
18 (3-n-decyl-6-bromo-thieno[3,2-b]thiophene-2-carboxylic acid)
19 (3-n-pentadecyl-6-bromo-thieno[3,2-b]thiophene-2-carboxylic acid)
20 (3-n-tetraecyl-6-bromo-thieno[3,2-b]thiophene-2-carboxylic acid)
21 (3-n-octyl-6-bromo-thieno[3,2-b]thiophene-2-carboxylic acid)
22 (5-methyl-dithieno[3,2-b:2′,3′-d]thiophene-2-carboxylic acid)
23 (diethyl dithieno[3,2-b:2′,3′-d]thiophene-2,6-dicarboxylate)
24 (N,N-bis(2-hydroxyethyl)-4-[2-(thiophene-2-yl)vinyl]aniline)
25 (methyl-5-{2-[4-(N-methyl,N-2-hydroxyethyl-amino)phenyl]ethenyl}-thiophene-2-formate)
26 (5-{2-[4-(N-methyl, N-2-hydroxyethyl-amino)phenyl]ethenyl}-thiophene-2-formate lithium)
27 (2,2′-bithiophene-5-methanol)
28 (5-hydroxymethyl-5′-formyl-2,2′-bithiophene)
29 (5′-{2-[4-[bis(2-hydroxyethyl)amino]phenyl]ethenyl]}-(2,2’-bithiophene)-5-formate sodium)
30 (2,2′-bithiophene-5-carboxaldehyde)

The synthesis and characterization of compounds 1, 2, 3, 4, 5, and 7 were detailed in the previous patent application (U.S. provisional patent application No. 61/291,742. Fang, Y., He, M. Q., Sun, H., Ferrie, A. M, Tran, E., Wei, Y., Deng, H., Niu, W., Molecules related hERG ion channels and the use thereof', filed on Dec. 31, 2009; and is incorporated by reference in its entirety).

The synthesis and characterization of chemicals 13, 14, 16, 17, 18, 19, 20, 21, and 22 were detailed in the previous patent application (U.S. provisional patent application No. 61/291,747. Fang, Y., He, M., Sun, H., Ferrie, A. M., Tran, E., Deng, H., Niu, W., and Wei, Y. “EGFR/VEGFR dual modulators and methods of using”, filed on Dec. 31, 2009; and is incorporated by reference in its entirety).

Compound 23 (diethyl dithieno[3,2-b:2′,3′-d]thiophene-2,6-dicarboxylate) was synthesized according to literature (Frey et al. Chemical Communications 2002, 20:2424-2425).

Compound 24 (N,N-bis(2-hydroxyethyl)-4-[2-(thiophene-2-yl)vinyl]aniline) was synthesized according to literature (Jen et al. Eur. Pat. Appl. 1995, EP 647874 A1).

Compound 28 (5-hydroxymethyl-5′-formyl-2,2′-bithiophene) was synthesized according to literature (Chang et al. U.S. Pat. No. 5,508,440 A (1996)).

ii. HT29 Cell Expresses Both GPR35 and hERG Channel

G protein-coupled receptors (GPCRs) comprise one of the largest families of cell surface proteins and represent a major target for both current therapeutic agents and drugs under development. Many cDNA clones are predicted to code for GPCRs based on high sequence similarity, especially in the transmembrane domains, to established GPCRs. For such orphan GPCRs, the identification of agonists, antagonists, and signal transduction pathways represents a major effort by industry for the discovery of novel drug targets. GPR35, an orphan GPCR first discovered during a human genomic DNA screen, shares limited sequence homology with purinergic P2Y receptors, nicotinic acid receptor HM74, lysophosphatidic acid receptor GPR23, and an orphan receptor, GPR55. The highest levels of GPR35 mRNA were found in immune and gastrointestinal tissues with only limited expression in lung and neuronal tissues. Subsequent to the initial description of GPR35 (now denoted GPR35a), Okumura et al. (2004) isolated a splice variant (GPR35b) from a human gastric cancer cDNA library that coded for an additional 31 amino acids at the N terminus (Okumura et al. (2004) Cancer Sci, 95: 131-135). GPR35a and GPR35b mRNA levels were up-regulated in gastric cancer tissue, suggesting a role for both splice variants in malignant transformation. However, the expression of GPR35 in human colon cancer cells has not been examined.

Real time PCR using GPR35 primers from SA Biosciences showed that HT29 cells express relatively high levels of GPR35, at least at the mRNA level (data not shown). Subsequent western blotting showed that HT29 cell lysates contain GPR35 isoforms, whose molecular weight is close to the expected values for both GPR35a and GPR35b, respectively (FIG. 1a and FIG. 7A).

HT29 is known to express hERG ion channel Western blotting studies indicate that HT29 expresses both hERG1a and hERG1b (FIG. 7B). HT29 is a human colon adenocarcinoma grade II cell line.

Confocal imaging further showed that GPR35 is primarily located at the cell surface plasma membrane (FIG. 1b). Interestingly, the staining pattern of GPR35, using anti-GPR35 antibody, indicates that instead of uniformly distributing at the cell surface, GPR35 is primarily located within certain microdomains, possibly within lipid rafts and/or complexed with other receptors. Nonetheless, these results indicate that HT29 expresses relatively high levels of GPR35a and GPR35b, at both mRNA and protein levels; and GPR35 is primarily located at the cell surface.

iii. GPR35 Physically Interacts with hERG Ion Channel in HT29 Cells

Co-immunoprecipitation (Co-IP) assays have been viewed as a gold standard to determine the physical interaction between a pair of receptors. As shown in FIG. 7, the anti-GPR35 column can specifically pull down both hERG1b and GPR35a/b. Similarly, the anti-hERG column can specifically pull down both hERG1a/b and GPR35a/b. These results indicate that in HT29 cells, hERG1b is physically associated with GPR35a and/or GPR35b to a signaling complex.

iv. GPR35 Also Physically Interacts with hERG Ion Channel in Engineered HEK293 Cells

To further determine the specificity of the GPR35/hERG signaling complex, HEK-hERG stable cells were transiently transfected with myc-tagged GPR35. The stable cell line expresses relatively high levels of hERG1. A co-immunoprecipitation (co-IP) assay was further used to characterize potential interactions between GPR35 and hERG1. Results showed that the anti-myc column can specifically pull down both hERG1b and GPR35 (data not shown). Similarly, the anti-hERG column can specifically pull down both hERG1b and GPR35 (data not shown). These results indicate that in engineered HEK cells, hERG1b is physically associated with GPR35.

v. Identification of GPR35 Agonists

Three different cellular assays were used to determine the GPR35 agonism activity of a molecule since there is no direct binding assay available. The assays are: (1) Ca²⁺ mobilization assays in an engineered cell such as HEK-GPR35 with and without co-expressing G_{qo5}. G_{qo5} is a G protein whose activation results in Ca²⁺ mobilization, and the G_{qo5} protein can be activated by the agonist-induced activation of a non-G_{q}-coupled receptor when expressed in the cell. Since GPR35 is believed to be a non-G_{q}-coupled receptor, the co-expression of G_{qo5} is necessary to detect the GPR35 agonist induced Ca²⁺ mobilization signal. (2) Receptor internalization assays. Receptor internalization is quick and universal to almost all GPCRs. (3) Label-free dynamic mass redistribution (DMR) assays, as promised by optical biosensors such as resonant waveguide grating biosensor. The DMR assay is an integrative cellular assay, since DMR signal represents an integrated cellular response upon stimulation. The DMR assay is pathway-sensitive but pathway unbiased (meaning no prerequisite of pre-determined pathway for measurement).

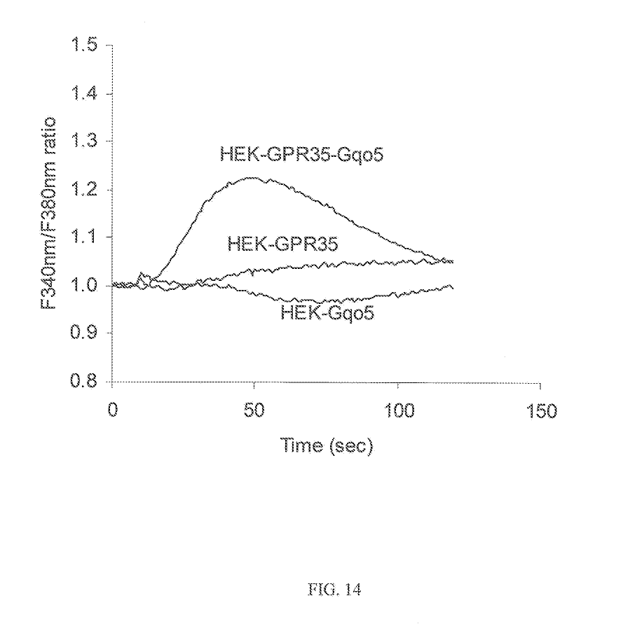

Ca²⁺ mobilization assays showed that the known GPR35 agonist zaprinast (10 micromolar) only triggered a detectable Ca²⁺ mobilization signal in the engineered HEK293 cells expressing both GPR35 and G_{qo5}, but not in either parental cell, or the engineered cells expressing either GPR35 or G_{qo5} (FIG. 14). Furthermore, in the native HT29 cells that endogenously expresses GPR35, zaprinast did not result in any detectable Ca²⁺ mobilization signal (FIG. 4A). These data indicate that zaprinast is a GPR35 specific agonism, and GPR35 indeed is not a G_{q}-coupled receptor. The Ca²⁺ mobilization assays with HEK-GPR35-G_{qo5} cells also showed that the following compounds are active: diflunisal, flufenamic acid, flunxin, furosemdie, niflumic acid, NPPB, tolfenamic acid, zaprinast, DNQX,

Receptor internalization assays showed that zaprinast (400 nanomolar) results in significant receptor internalization in HT29 cells, as indicated by the disappearance of cell plasma membrane-associated fluorescence, and the appearance of intracellular fluorescence vesicles, in comparison with the unstimulated cells (FIG. 2a). Here confocal imaging was obtained after the cells were stimulated with or without zaprinast for 30 min, and fixed and permeabilized and stained with anti-GPR35 antibody and fluorescent secondary antibody.

DMR assays showed that zaprinast triggered a dose-dependent and saturable DMR signal, and its EC50 to cause the P-DMR event was found to be 137 nM. (FIG. 2). Follow-up studies using RNAi knockdown indicate that the zaprinast DMR signal is specific to GPR35, since the GPR35 knockdown partially suppressed the zaprinast DMR signal (data not shown). These results indicate that zaprinast acts as an agonist for the endogenous GPR35 in HT29.

vi. Identification of hERG Modulators

Two different cellular assays were used to identify and confirm hERG modulators. First, automated patch clamping was used to detect hERG currents in the engineered CHO cells (CHO-hERG). hERG is a voltage-gated ion channel Results showed that zaprinast acts as a non-modulator (i.e., neither activator nor inhibitor) of hERG activity in CHO-hERG cells, based on the tail current obtained in the absence and presence of zaprinast (FIG. 9). The tail current value was the ratio of post compound current divided by pre compound current. This value is more predictive for the hERG modulation by the compounds tested, since the absolute current value varies from well to well which is related to the patch seal resistance. These results indicate that zaprinast does not directly modulate the channel activity of hERG.

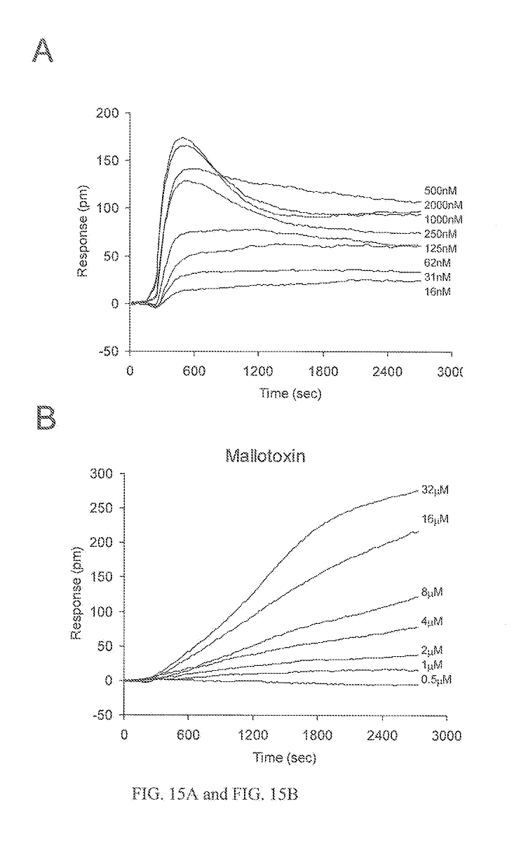

Second, DMR assays were used to detect hERG activators. The known hERG activator mallotoxin resulted in a dose-dependent and saturable DMR signal in HT29 cells (FIG. 15). The mallotoxin DMR signal is distinct from the GPR35 agonist zaprinast DMR signal. To confirm the specificity, DMR assays using HEK-hERG cells were carried out and showed that mallotoxin also resulted in a similar DMR signal in the engineered HEK-293 cells that stably express hERG1, but there was no detectable DMR signal (i.e., net-zero DMR) in the parental HEK-293 cells. These results indicate that mallotoxin is a hERG activator, and the activation of the hERG channel can trigger signaling in the cells (e.g., HT29 cells, or engineered HEK-hERG cells).

vii. Identification of hERG-GPR35 Modulators

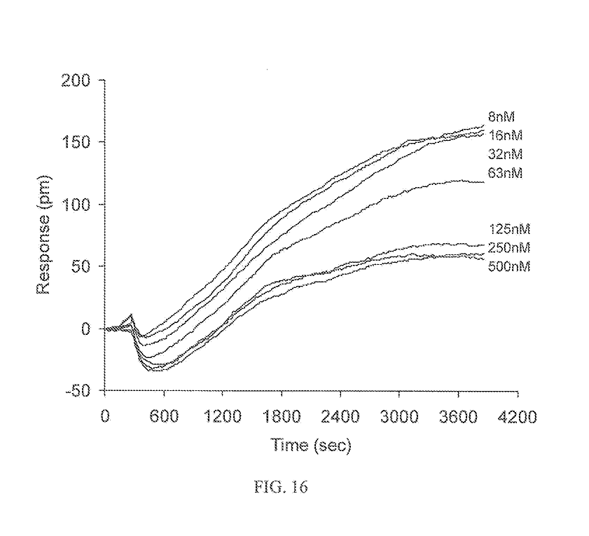

The above-mentioned methods can identify either GPR35 ligands or hERG modulators. To identify hERG-GPR35 complex modulators, cross-desensitization DMR assays were used. Here, a molecule was first examined for its ability to desensitize or attenuate the hERG activator mallotoxin-induced DMR signal in HT29 cells. When a molecule is not a hERG activator but a GPR35 agonist and can cause desensitization of the cells to subsequent stimulation with the known mallotoxin-induced DMR, the molecule is referred to a hERG-transactivating GPR35 agonist. An example is zaprinast, the known GPR35 agonist. Pretreatment of HT29 cells with zaprinast dose-dependently desensitized the cells responding to subsequent stimulation with the hERG activator mallotoxin (FIG. 16).

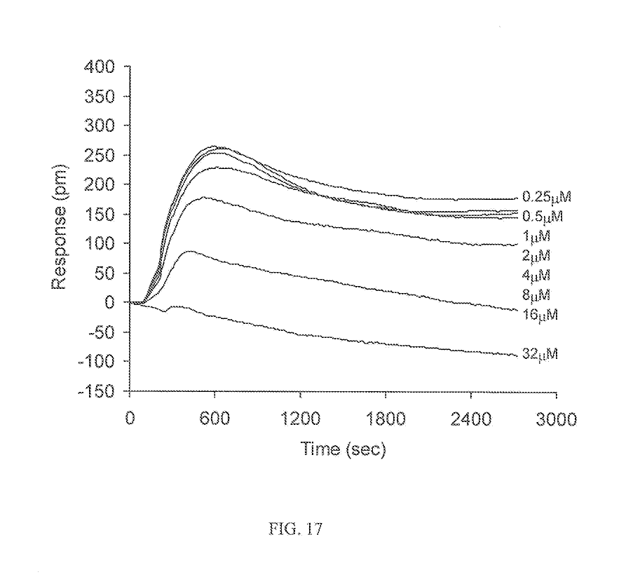

Second, the molecule was examined for its ability to desensitize or attenuate the GPR35 agonist induced DMR signal in HT29 cells. When a molecule is not a GPR35 agonist, but a hERG activator that is able to cause desensitization of the cells to subsequent stimulation with the GPR35 agonist based on the GPR35 DMR, the molecule is referred to as a GPR35-transactivating hERG activator. An example is mallotoxin that is the known hERG activator agonist but not a direct GPR35 agonist. Pretreatment of HT29 cells with mallotoxin dose-dependently desensitized the cells responding to subsequent stimulation with the GPR35 agonist zaprinast (FIG. 17).

viii. Classification of hERG-GPR35 complex Modulators

Using the above-mentioned battery of assays, a series of molecules as shown in Schemes 1 to 6 were identified as either hERG-specific modulators, or GPR35-specific modulators, or hERG-GPR35 complex modulators. Table 1 shows the experimental data and the classifications of these molecules. The structures of the compounds (with chemical names or numerical numbers) referred to in Table 1 are shown below.

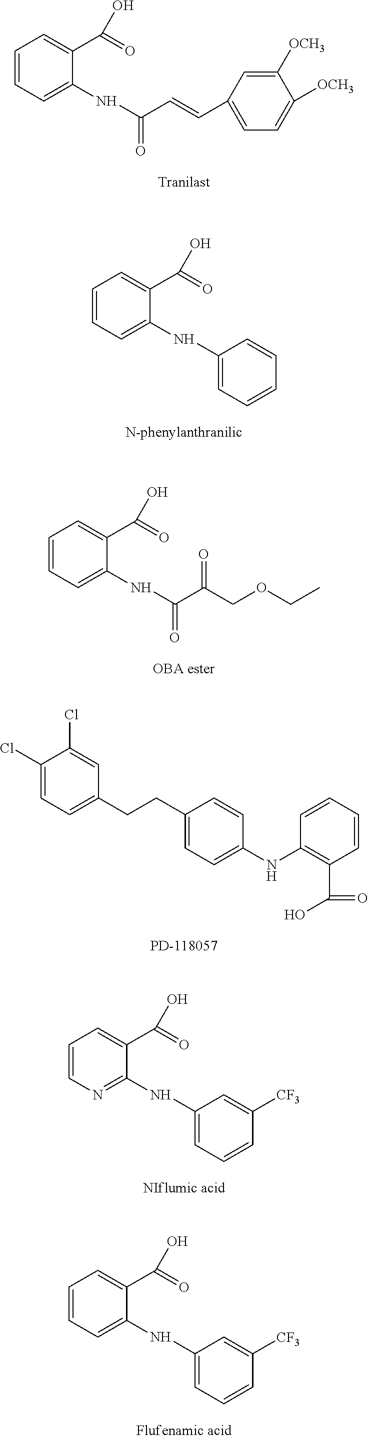

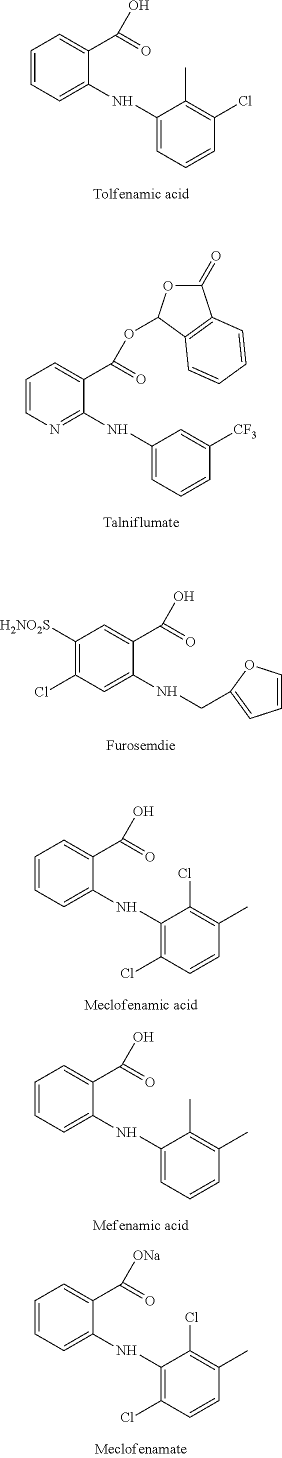

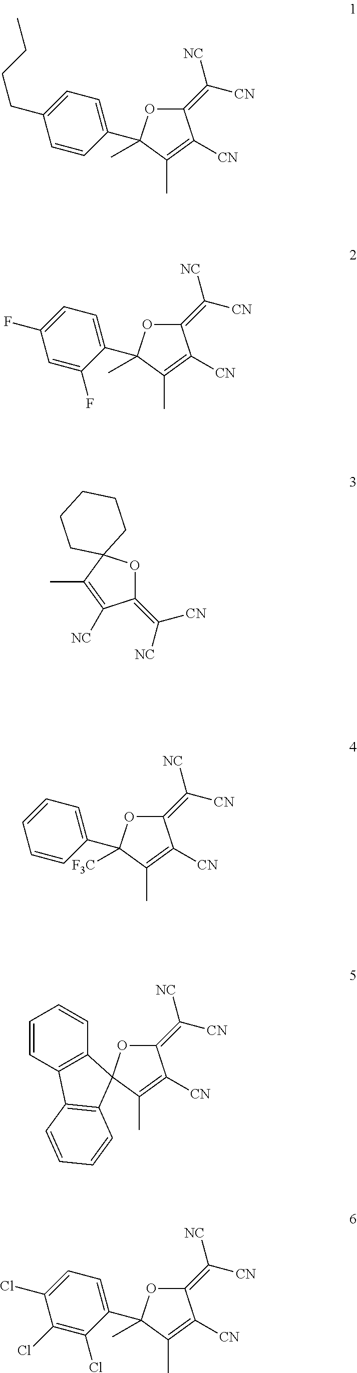

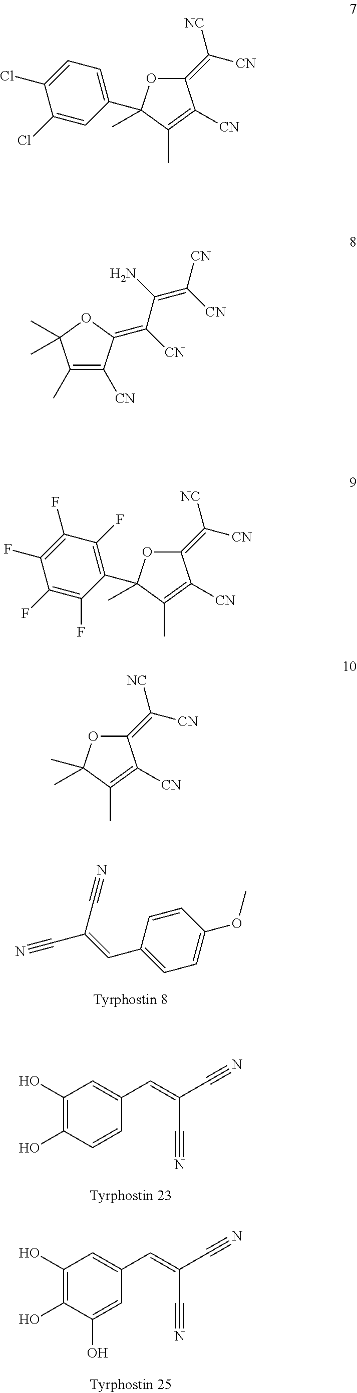

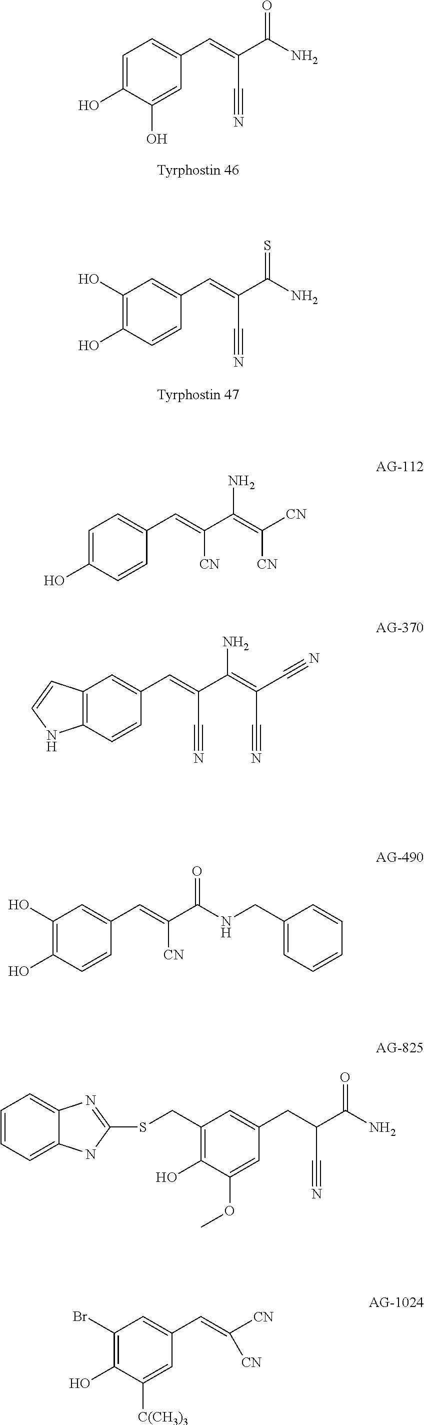

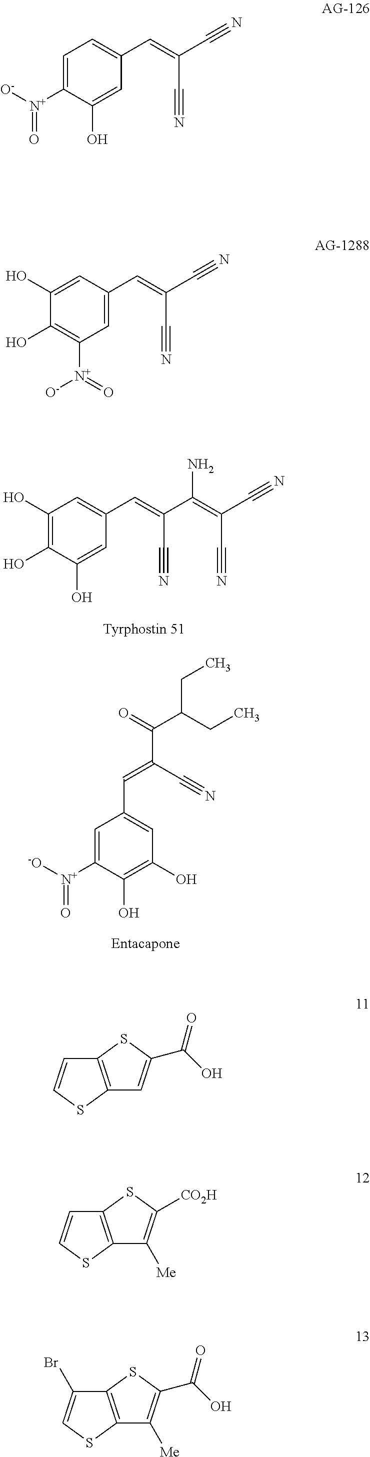

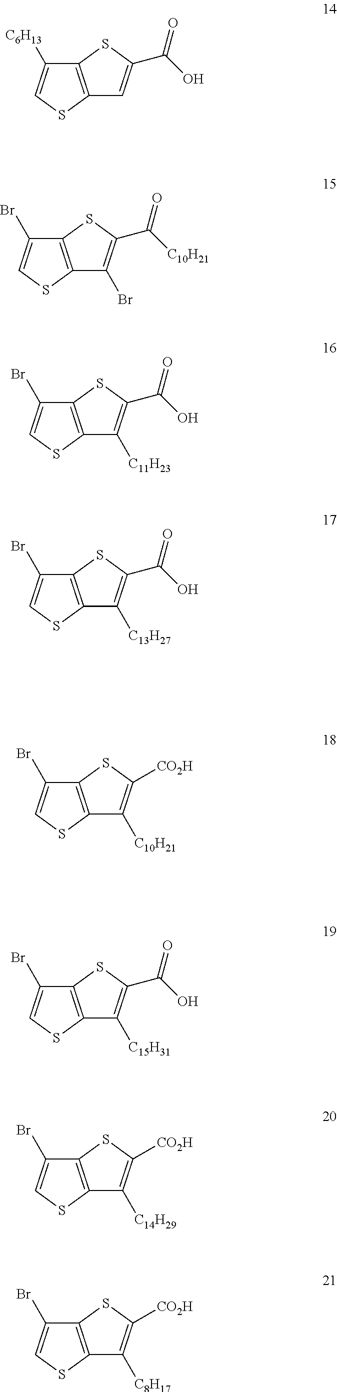

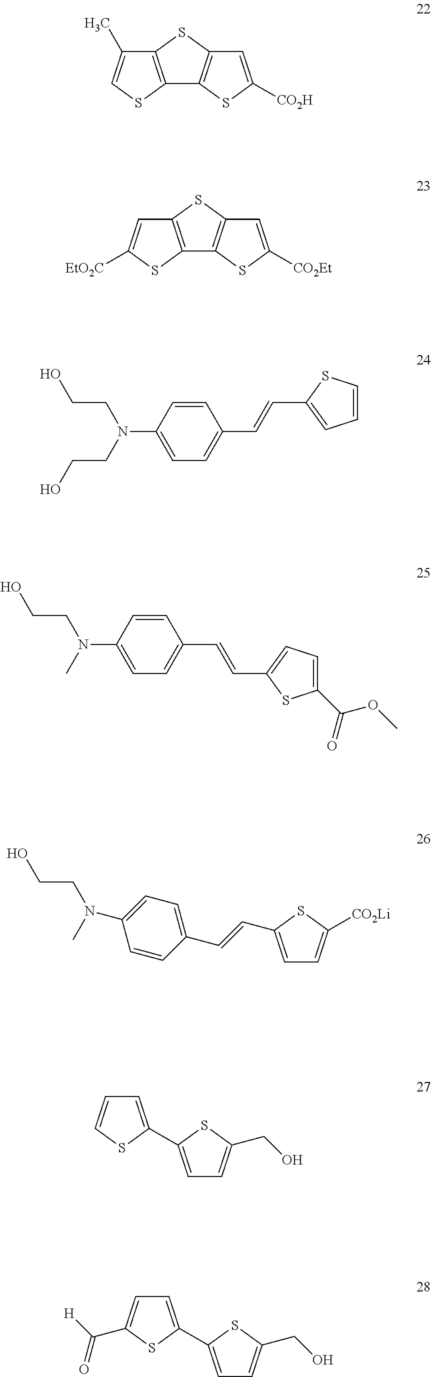

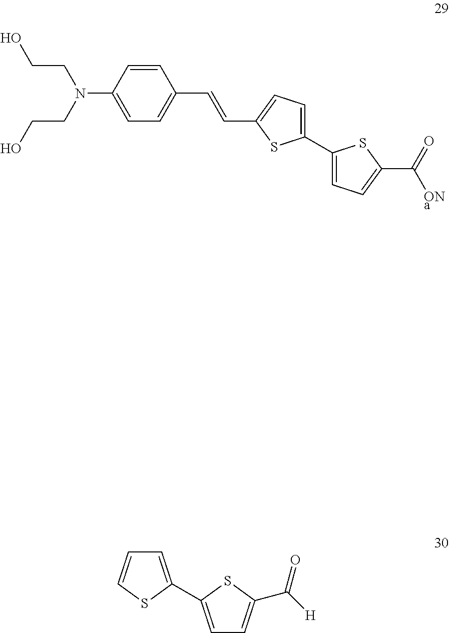

The classifications were obtained based on a battery of assays including Ca²⁺ mobilization assays using HEK-GPR35-G_{qo5} cells, DMR assays using HT29 cells (endogenously expressed hERG1 and GPR35), automated patch clamping electrophysiology recording using CHO-hERG1 cells, and cross-desensitization assays using HT29 cells. The classes found include: Class A: GPR35 agonist and hERG activator, GPR35-hERG complex agonist; Class B: hERG transactivating GPR35 agonist; Class C: hERG non-transactivating GPR35 agonist; Class D: GPR35 transactivating hERG activator Class E: GPR35 non-transactivating hERG activator, and others. It is worth noting that GPR35 agonists can be classified into two subtypes based on its ability to trigger Ca²⁺ mobilization in HEK-GPR35-G_{qo5} cells: active and inactive. This is not surprising since GPR35 is non-G_{q} or Gᵢ-coupled receptor, and many GPCR ligands are known to display functional selectivity.

TABLE 1
The classifications of hERG-GRO35 complex modulators

| | Ca2+ moblization | | Electrophysiology | Desenstization to | Desenstization to | |
|---|---|---|---|---|---|---|
| Compound | (HEK-GPR35-Gqo5) | DMR (HT29) | (HEK-hERG) | GPR35 agonist | hERG activator | Class |
| Niflumic acid | active | ZAP-like | activator | desenstized | desenstized | A |
| Flufenamic acid | active | ZAP-like | activator | desenstized | desenstized | A |
| Flunixin | active | ZAP-like | activator | desenstized | inactive | A |
| NPPB | active | ZAP-like | activator | desenstized | desenstized | A |
| Compound 04 | active | ZAP-like | activator | desenstized | desenstized | A |
| Compound 21 | active | ZAP-like | activator | desenstized | partially desenstized | A |
| Compound 29 | active | ZAP-like | activator | desenstized | partially desenstized | A |
| Diflunisal | active | ZAP-like | activator | desenstized | desenstized | A |
| Diclofenac | inactive | ZAP-like | activator | desenstized | inactive | A |
| Compound 13 | inactive | ZAP-like | activator | desenstized | partially desenstized | A |
| PD-118057 | N.D. | ZAP-like | activator | desenstized | desenstized | A |
| Compound 18 | N.D. | ZAP-like | activator | desenstized | partially desenstized | A |
| Compound 19 | N.D. | ZAP-like | activator | desenstized | partially desenstized | A |
| Tyrphostin 47 | N.D. | ZAP-like | activator | desenstized | partially desenstized | A |
| Compound 17 | N.D. | ZAP-like | activator | desenstized | partially desenstized | A |
| Tolfenamic acid | active | ZAP-like | blocker | desenstized | partially desenstized | B |
| Compound 14 | active | ZAP-like | blocker | desenstized | partially desenstized | B |
| Furosemdie | active | ZAP-like | inactive | desenstized | partially desenstized | B |
| zaprinast | active | ZAP | inactive | desenstized | partially desenstized | B |
| DNQX | active | ZAP-like | inactive | desenstized | partially desenstized | B |
| Compound 02 | active | ZAP-like | inactive | desenstized | partially desenstized | B |
| Compound 07 | active | ZAP-like | inactive | desenstized | partially desenstized | B |
| Compound 08 | active | ZAP-like | inactive | desenstized | partially desenstized | B |
| Compound 09 | active | ZAP-like | inactive | desenstized | partially desenstized | B |
| Compound 10 | active | ZAP-like | inactive | desenstized | partially desenstized | B |
| Compound 12 | active | ZAP-like | inactive | desenstized | partially desenstized | B |
| Compound 25 | active | ZAP-like | inactive | desenstized | partially desenstized | B |
| Compound 26 | active | ZAP-like | inactive | desenstized | partially desenstized | B |
| Meclofenamate | inactive | MTX-like | blocker | desenstized | partially desenstized | other |
| Sulindac | inactive | MTX-like | blocker | desenstized | desenstized | other |
| Compound 27 | inactive | MTX-like | blocker | inactive | partially desenstized | other |
| compound 30 | inactive | MTX-like | blocker | desenstized | partially desenstized | other |
| Compound 22 | inactive | ZAP-like | inactive | desenstized | desenstized | B |
| Mefenamic Acid | inactive | MTX-like | inactive | desenstized | partially desenstized | B |
| Kynurenic acid | inactive | ZAP-like | inactive | desenstized | partially desenstized | B |
| Sulfasalazine | inactive | ZAP-like | inactive | desenstized | partially desenstized | B |
| Compound 01 | inactive | ZAP-like | inactive | desenstized | partially desenstized | B |
| Compound 03 | inactive | ZAP-like | inactive | desenstized | partially desenstized | B |
| Compound 11 | inactive | ZAP-like | inactive | desenstized | partially desenstized | B |
| Compound 23 | inactive | ZAP-like | inactive | desenstized | partially desenstized | B |
| Compound 28 | inactive | ZAP-like | inactive | desenstized | partially desenstized | B |
| Meclofenamic acid | N.D. | ZAP-like | inactive | desenstized | partially desenstized | B |
| Compound 15 | N.D. | ZAP-like | inactive | desenstized | partially desenstized | B |
| Compound 16 | N.D. | ZAP-like | inactive | desenstized | partially desenstized | B |
| AG126 | N.D. | ZAP-like | inactive | desenstized | partially desenstized | B |
| AG370 | N.D. | ZAP-like | inactive | desenstized | partially desenstized | B |
| Entacapone | N.D. | ZAP-like | inactive | desenstized | partially desenstized | B |
| Tyrphostin 25 | N.D. | ZAP-like | inactive | desenstized | partially desenstized | B |
| Tyrphostin 51 | N.D. | ZAP-like | inactive | desenstized | partially desenstized | B |
| Tyrphostin 8 | N.D. | ZAP-like | inactive | desenstized | partially desenstized | B |
| Tranilast | N.D. | ZAP-like | N.D. | desenstized | partially desenstized | B |
| N-phenylanthranilic | N.D. | ZAP-like | N.D. | desenstized | partially desenstized | B |
| OBA ester | N.D. | ZAP-like | N.D. | desenstized | partially desenstized | B |
| AG1024 | N.D. | ZAP-like | N.D. | desenstized | partially desenstized | B |
| AG112 | N.D. | ZAP-like | N.D. | desenstized | partially desenstized | B |
| AG1288 | N.D. | ZAP-like | N.D. | desenstized | partially desenstized | B |
| AG490 | N.D. | ZAP-like | N.D. | desenstized | partially desenstized | B |
| AG825 | N.D. | ZAP-like | N.D. | desenstized | partially desenstized | B |
| Tyrphostin 23 | N.D. | ZAP-like | N.D. | desenstized | partially desenstized | B |
| Talniflumate | inactive | MTX-like | inactive | desenstized | inactive | C |
| Talniflumate | inactive | MTX-like | inactive | desenstized | inactive | C |
| Compound 05 | inactive | ZAP-like | inactive | desenstized | inactive | C |
| Compound 06 | inactive | ZAP-like | inactive | desenstized | inactive | C |
| Tyrphostin 46 | N.D. | ZAP-like | inactive | desenstized | inactive | C |
| Mallotoxin | N.D. | MTX | activator | desenstized | desenstized | D |
| NSC1619 | N.D. | MTX | activator | inactive | desenstized | E |
| Compound 24 | inactive | N-DMR | inactive | inactive | inactive | inactive |

### Sequences

i. Protein Sequence of GPR35a

The protein sequence of GPR35a (SEQ ID NO:9) (UniProtKB/Swiss-Prot:Q9HC97) is

MNGTYNTCGSSDLTWPPAIKLGFYAYLGVLLVLGLLLNSLALWVFCCRM
QQWTETRIYMTNLAVADLCLLCTLPFVLHSLRDTSDTPLCQLSQGIYLT
NRYMSISLVTAIAVDRYVAVRHPLRARGLRSPRQAAAVCAVLWVLVIGS
LVARWLLGIQEGGFCFRSTRHNFNSMAFPLLGFYLPLAVVVFCSLKVVT
ALAQRPPTDVGQAEATRKAARMVWANLLVFVVCFLPLHVGLTVRLAVGW
NACALLETIRRALYITSKLSDANCCLDAICYYYMAKEFQEASALAVAPS
AKAHKSQDSLCVTLA

ii. mRNA Sequence of GOR35a

The Homo sapiens G protein-coupled receptor 35a (GPR35a) (SEQ ID NO:1), mRNA (NCBI Reference Sequence: NM_{—}005301.2) is

| | |
|---|---|
| 1 | caggccagag tcccagctgt cctggactct gctgtgggga agggctgatg caggtgtgga |
| 61 | gtcaaatgtg ggtgcctcct gcagccgggt gccaggaggg gtggaggggc caccctgggc |
| 121 | tttgtccggg agcctggtct tcccgtcctt gggctgacag gtgctgctgc ctctgagccc |
| 181 | tccctgctaa gagctgtgtg ctgggtaagg ctggtggccc tttgggctcc ctgtccagga |
| 241 | tttgtgctct ggagggtagg gcttgctggg ctggggactg gaggggaacg tggagctcct |
| 301 | tctgcctcct ttcctgcccc atgacagcag gcagatccca ggagagaaga gctcaggaga |
| 361 | tgggaagagg atctgtccag gggttagacc tcaagggtga cttggagttc tttacggcac |
| 421 | ccatgctttc tttgaggagt tttgtgtttg tgggtgtggg gtcggggctc acctcctccc |
| 481 | acatccctgc ccagaggtgg gcagagtggg ggcagtgcct tgctccccct gctcgctctc |
| 541 | tgctgacctc cggctccctg tgctgcccca ggaccatgaa tggcacctac aacacctgtg |
| 601 | gctccagcga cctcacctgg cccccagcga tcaagctggg cttctacgcc tacttgggcg |
| 661 | tcctgctggt gctaggcctg ctgctcaaca gcctggcgct ctgggtgttc tgctgccgca |
| 721 | tgcagcagtg gacggagacc cgcatctaca tgaccaacct ggcggtggcc gacctctgcc |
| 781 | tgctgtgcac cttgcccttc gtgctgcact ccctgcgaga cacctcagac acgccgctgt |
| 841 | gccagctctc ccagggcatc tacctgacca acaggtacat gagcatcagc ctggtcacgg |
| 901 | ccatcgccgt ggaccgctat gtggccgtgc ggcacccgct gcgtgcccgc gggctgcggt |
| 961 | cccccaggca ggctgcggcc gtgtgcgcgg tcctctgggt gctggtcatc ggctccctgg |
| 1021 | tggctcgctg gctcctgggg attcaggagg gcggcttctg cttcaggagc acccggcaca |
| 1081 | atttcaactc catggcgttc ccgctgctgg gattctacct gcccctggcc gtggtggtct |
| 1141 | tctgctccct gaaggtggtg actgccctgg cccagaggcc acccaccgac gtggggcagg |
| 1201 | cagaggccac ccgcaaggct gcccgcatgg tctgggccaa cctcctggtg ttcgtggtct |
| 1261 | gcttcctgcc cctgcacgtg gggctgacag tgcgcctcgc agtgggctgg aacgcctgtg |
| 1321 | ccctcctgga gacgatccgt cgcgccctgt acataaccag caagctctca gatgccaact |
| 1381 | gctgcctgga cgccatctgc tactactaca tggccaagga gttccaggag gcgtctgcac |
| 1441 | tggccgtggc tcccagtgct aaggcccaca aaagccagga ctctctgtgc gtgaccctcg |
| 1501 | cctaagaggc gtgctgtggg cgctgtgggc caggtctcgg gggctccggg aggtgctgcc |
| 1561 | tgccagggga agctggaacc agtagcaagg agcccgggat cagccctgaa ctcactgtgt |
| 1621 | attctcttgg agccttgggt gggcagggac ggcccaggta cctgctctct tgggaagaga |
| 1681 | gagggacagg gacaagggca agaggactga ggccagagca aggccaatgt cagagacccc |
| 1741 | cgggatgggg cctcacactt gccaccccca gaaccagctc acctggccag agtgggttcc |
| 1801 | tgctggccag ggtgcagcct tgatgacacc tgccgctgcc cctcggggct ggaataaaac |
| 1861 | tccccaccca gagtc |

iii. cDNA for GPR35a

The cDNA sequence for GPR35a (SEQ ID NO:2):

| | ...................................ATGAATGGCACCTACAACACCTGTG |
|---|---|
| 26 | GCTCCAGCGACCTCACCTGGCCCCCAGCGATCAAGCTGGGCTTCTACGCCTACTTGGGCG |
| 86 | TCCTGCTGGTGCTAGGCCTGCTGCTCAACAGCCTGGCGCTCTGGGTGTTCTGCTGCCGCA |
| 146 | TGCAGCAGTGGACGGAGACCCGCATCTACATGACCAACCTGGCGGTGGCCGACCTCTGCC |
| 206 | TGCTGTGCACCTTGCCCTTCGTGCTGCACTCCCTGCGAGACACCTCAGACACGCCGCTGT |
| 266 | GCCAGCTCTCCCAGGGCATCTACCTGACCAACAGGTACATGAGCATCAGCCTGGTCACGG |
| 326 | CCATCGCCGTGGACCGCTATGTGGCCGTGCGGCACCCGCTGCGTGCCCGCGGGCTGCGGT |
| 386 | CCCCCAGGCAGGCTGCGGCCGTGTGCGCGGTCCTCTGGGTGCTGGTCATCGGCTCCCTGG |
| 446 | TGGCTCGCTGGCTCCTGGGGATTCAGGAGGGCGGCTTCTGCTTCAGGAGCACCCGGCACA |
| 506 | ATTTCAACTCCATGGCGTTCCCGCTGCTGGGATTCTACCTGCCCCTGGCCGTGGTGGTCT |
| 566 | TCTGCTCCCTGAAGGTGGTGACTGCCCTGGCCCAGAGGCCACCCACCGACGTGGGGCAGG |
| 626 | CAGAGGCCACCCGCAAGGCTGCCCGCATGGTCTGGGCCAACCTCCTGGTGTTCGTGGTCT |
| 686 | GCTTCCTGCCCCTGCACGTGGGGCTGACAGTGCGCCTCGCAGTGGGCTGGAACGCCTGTG |
| 746 | CCCTCCTGGAGACGATCCGTCGCGCCCTGTACATAACCAGCAAGCTCTCAGATGCCAACT |
| 806 | GCTGCCTGGACGCCATCTGCTACTACTACATGGCCAAGGAGTTCCAGGAGGCGTCTGCAC |
| 866 | TGGCCGTGGCTCCCAGTGCTAAGGCCCACAAAAGCCAGGACTCTCTGTGCGTGACCCTCG |
| 926 | CCTAA....................................................... |

iv. Protein Sequence of GPR35b

The protein sequence of GPR35b (SEQ ID NO:3) (S. Okumura, H. Baba, T. Kumada, K. Nanmoku, H. Nakajima, Y. Nakane, K. Hioki, K. Ikenaka (2004) Cloning of a G-protein-coupled receptor that shows an activity to transform NIH3T3 cells and is expressed in gastric cancer cells, Cancer Sci. 95: 131-135) is

MLSGSRAVPTPHRGSEELLKYMLHSPCVSLTMNGTYNTCGSSDLTWPPAIKLGFYAYLGVLL
VLGLLLNSLALWVFCCRMQQWTETRIYMTNLAVADLCLLCTLPFVLHSLRDTSDTPLCQLSQ
GIYLTNRYMSISLVTAIAVDRYVAVRHPLRARGLRSPRQAAAVCAVLWVLVIGSLVARWLLG
IQEGGFCFRSTRHNFNSMAFPLLGFYLPLAVVVFCSLKVVTALAQRPPTDVGQAEATRKAAR
MVWANLLVFVVCFLPLHVGLTVRLAVGWNACALLETIRRALYITSKLSDANCCLDAICYYY
MAKEFQEASALAVAPRAKAHKSQDSLCVTLA

v. cDNA for GPR35b

The cDNA sequence of GPR35b (SEQ ID NO:4) is:

| | |
|---|---|
| 1 | ATGCTGAGTGGTTCCCGGGCTGTCCCCACTCCACACCGTGGCAGTGAAGAGCTGCTGAAG |
| 61 | TACATGCTTCATAGTCCTTGCGTCTCTCTGACCATGAATGGCACCTACAACACCTGTGGC |
| 121 | TCCAGCGACCTCACCTGGCCCCCAGCGATCAAGCTGGGCTTCTACGCCTACTTGGGCGTC |
| 181 | CTGCTGGTGCTAGGCCTGCTGCTCAACAGCCTGGCGCTCTGGGTGTTCTGCTGCCGCATG |
| 241 | CAGCAGTGGACGGAGACCCGCATCTACATGACCAACCTGGCGGTGGCCGACCTCTGCCTG |
| 301 | CTGTGCACCTTGCCCTTCGTGCTGCACTCCCTGCGAGACACCTCAGACACGCCGCTGTGC |
| 361 | CAGCTCTCCCAGGGCATCTACCTGACCAACAGGTACATGAGCATCAGCCTGGTCACGGCC |
| 421 | ATCGCCGTGGACCGCTATGTGGCCGTGCGGCACCCGCTGCGTGCCCGCGGGCTGCGGTCC |
| 481 | CCCAGGCAGGCTGCGGCCGTGTGCGCGGTCCTCTGGGTGCTGGTCATCGGCTCCCTGGTG |
| 541 | GCTCGCTGGCTCCTGGGGATTCAGGAGGGCGGCTTCTGCTTCAGGAGCACCCGGCACAAT |
| 601 | TTCAACTCCATGGCGTTCCCGCTGCTGGGATTCTACCTGCCCCTGGCCGTGGTGGTCTTC |
| 661 | TGCTCCCTGAAGGTGGTGACTGCCCTGGCCCAGAGGCCACCCACCGACGTGGGGCAGGCA |
| 721 | GAGGCCACCCGCAAGGCTGCCCGCATGGTCTGGGCCAACCTCCTGGTGTTCGTGGTCTGC |
| 781 | TTCCTGCCCCTGCACGTGGGGCTGACAGTGCGCCTCGCAGTGGGCTGGAACGCCTGTGCC |
| 841 | CTCCTGGAGACGATCCGTCGCGCCCTGTACATAACCAGCAAGCTCTCAGATGCCAACTGC |
| 901 | TGCCTGGACGCCATCTGCTACTACTACATGGCCAAGGAGTTCCAGGAGGCGTCTGCACTG |
| 961 | GCCGTGGCTCCCAGTGCTAAGGCCCACAAAAGCCAGGACTCTCTGTGCGTGACCCTCGCC |
| 1021 | TAA |

vi. Protein Sequence of hERG1a

The protein sequence of hERG1a (SEQ ID NO:5) (NCBI Reference Sequence: NP_{—}000229.1) is:

MPVRRGHVAPQNTFLDTIIRKFEGQSRKFIIANARVENCAVIYCNDGFCELCGYSRAE
VMQRPCTCDFLHGPRTQRRAAAQIAQALLGAEERKVEIAFYRKDGSCFLCLVDVVPV
KNEDGAVIMFILNFEVVMEKDMVGSPAHDTNHRGPPTSWLAPGRAKTFRLKLPALL
ALTARESSVRSGGAGGAGAPGAVVVDVDLTPAAPSSESLALDEVTAMDNHVAGLGP
AEERRALVGPGSPPRSAPGQLPSPRAHSLNPDASGSSCSLARTRSRESCASVRRASSA
DDIEAMRAGVLPPPPRHASTGAMHPLRSGLLNSTSDSDLVRYRTISKIPQITLNFVDLK
GDPFLASPTSDREIIAPKIKERTHNVTEKVTQVLSLGADVLPEYKLQAPRIHRWTILHY
SPFKAVWDWLILLLVIYTAVFTPYSAAFLLKETEEGPPATECGYACQPLAVVDLIVDI
MFIVDILINFRTTYVNANEEVVSHPGRIAVHYFKGWFLIDMVAAIPFDLLIFGSGSEELI
GLLKTARLLRLVRVARKLDRYSEYGAAVLFLLMCTFALIAHWLACIWYAIGNMEQP
HMDSRIGWLHNLGDQIGKPYNSSGLGGPSIKDKYVTALYFTFSSLTSVGFGNVSPNTN
SEKIFSICVMLIGSLMYASIFGNVSAIIQRLYSGTARYHTQMLRVREFIRFHQIPNPLRQ
RLEEYFQHAWSYTNGIDMNAVLKGFPECLQADICLHLNRSLLQHCKPFRGATKGCLR
ALAMKFKTTHAPPGDTLVHAGDLLTALYFISRGSIEILRGDVVVAILGKNDIFGEPLNL
YARPGKSNGDVRALTYCDLHKIHRDDLLEVLDMYPEFSDHFWSSLEITFNLRDTNMI
PGSPGSTELEGGFSRQRKRKLSFRRRTDKDTEQPGEVSALGPGRAGAGPSSRGRPGGP
WGESPSSGPSSPESSEDEGPGRSSSPLRLVPFSSPRPPGEPPGGEPLMEDCEKSSDTCNP
LSGAFSGVSNIFSFWGDSRGRQYQELPRCPAPTPSLLNIPLSSPGRRPRGDVESRLDAL
QRQLNRLETRLSADMATVLQLLQRQMTLVPPAYSAVTTPGPGPTSTSPLLPVSPLPTL
TLDSLSQVSQFMACEELPPGAPELPQEGPTRRLSLPGQLGALTSQPLHRHGSDPGS.

vii. cDNA Sequence of hERG1a

The cDNA sequence of hERG1a (SEQ ID NO:6) is:

| | ..........................................ATGCCGGTGCGGAGGGGC |
|---|---|
| 19 | CACGTCGCGCCGCAGAACACCTTCCTGGACACCATCATCCGCAAGTTTGAGGGCCAGAGC |
| 79 | CGTAAGTTCATCATCGCCAACGCTCGGGTGGAGAACTGCGCCGTCATCTACTGCAACGAC |
| 139 | GGCTTCTGCGAGCTGTGCGGCTACTCGCGGGCCGAGGTGATGCAGCGACCCTGCACCTGC |
| 199 | GACTTCCTGCACGGGCCGCGCACGCAGCGCCGCGCTGCCGCGCAGATCGCGCAGGCACTG |
| 259 | CTGGGCGCCGAGGAGCGCAAAGTGGAAATCGCCTTCTACCGGAAAGATGGGAGCTGCTTC |
| 319 | CTATGTCTGGTGGATGTGGTGCCCGTGAAGAACGAGGATGGGGCTGTCATCATGTTCATC |
| 379 | CTCAATTTCGAGGTGGTGATGGAGAAGGACATGGTGGGGTCCCCGGCTCATGACACCAAC |
| 439 | CACCGGGGCCCCCCCACCAGCTGGCTGGCCCCAGGCCGCGCCAAGACCTTCCGCCTGAAG |
| 499 | CTGCCCGCGCTGCTGGCGCTGACGGCCCGGGAGTCGTCGGTGCGGTCGGGCGGCGCGGGC |
| 559 | GGCGCGGGCGCCCCGGGGGCCGTGGTGGTGGACGTGGACCTGACGCCCGCGGCACCCAGC |
| 619 | AGCGAGTCGCTGGCCCTGGACGAAGTGACAGCCATGGACAACCACGTGGCAGGGCTCGGG |
| 679 | CCCGCGGAGGAGCGGCGTGCGCTGGTGGGTCCCGGCTCTCCGCCCCGCAGCGCGCCCGGC |
| 739 | CAGCTCCCATCGCCCCGGGCGCACAGCCTCAACCCCGACGCCTCGGGCTCCAGCTGCAGC |
| 799 | CTGGCCCGGACGCGCTCCCGAGAAAGCTGCGCCAGCGTGCGCCGCGCCTCGTCGGCCGAC |
| 859 | GACATCGAGGCCATGCGCGCCGGGGTGCTGCCCCCGCCACCGCGCCACGCCAGCACCGGG |
| 919 | GCCATGCACCCACTGCGCAGCGGCTTGCTCAACTCCACCTCGGACTCCGACCTCGTGCGC |
| 979 | TACCGCACCATTAGCAAGATTCCCCAAATCACCCTCAACTTTGTGGACCTCAAGGGCGAC |
| 1039 | CCCTTCTTGGCTTCGCCCACCAGTGACCGTGAGATCATAGCACCTAAGATAAAGGAGCGA |
| 1099 | ACCCACAATGTCACTGAGAAGGTCACCCAGGTCCTGTCCCTGGGCGCCGACGTGCTGCCT |
| 1159 | GAGTACAAGCTGCAGGCACCGCGCATCCACCGCTGGACCATCCTGCATTACAGCCCCTTC |
| 1219 | AAGGCCGTGTGGGACTGGCTCATCCTGCTGCTGGTCATCTACACGGCTGTCTTCACACCC |
| 1279 | TACTCGGCTGCCTTCCTGCTGAAGGAGACGGAAGAAGGCCCGCCTGCTACCGAGTGTGGC |
| 1339 | TACGCCTGCCAGCCGCTGGCTGTGGTGGACCTCATCGTGGACATCATGTTCATTGTGGAC |
| 1399 | ATCCTCATCAACTTCCGCACCACCTACGTCAATGCCAACGAGGAGGTGGTCAGCCACCCC |
| 1459 | GGCCGCATCGCCGTCCACTACTTCAAGGGCTGGTTCCTCATCGACATGGTGGCCGCCATC |
| 1519 | CCCTTCGACCTGCTCATCTTCGGCTCTGGCTCTGAGGAGCTGATCGGGCTGCTGAAGACT |
| 1579 | GCGCGGCTGCTGCGGCTGGTGCGCGTGGCGCGGAAGCTGGATCGCTACTCAGAGTACGGC |
| 1639 | GCGGCCGTGCTGTTCTTGCTCATGTGCACCTTTGCGCTCATCGCGCACTGGCTAGCCTGC |
| 1699 | ATCTGGTACGCCATCGGCAACATGGAGCAGCCACACATGGACTCACGCATCGGCTGGCTG |
| 1759 | CACAACCTGGGCGACCAGATAGGCAAACCCTACAACAGCAGCGGCCTGGGCGGCCCCTCC |
| 1819 | ATCAAGGACAAGTATGTGACGGCGCTCTACTTCACCTTCAGCAGCCTCACCAGTGTGGGC |
| 1879 | TTCGGCAACGTCTCTCCCAACACCAACTCAGAGAAGATCTTCTCCATCTGCGTCATGCTC |
| 1939 | ATTGGCTCCCTCATGTATGCTAGCATCTTCGGCAACGTGTCGGCCATCATCCAGCGGCTG |
| 1999 | TACTCGGGCACAGCCCGCTACCACACACAGATGCTGCGGGTGCGGGAGTTCATCCGCTTC |
| 2059 | CACCAGATCCCCAATCCCCTGCGCCAGCGCCTCGAGGAGTACTTCCAGCACGCCTGGTCC |
| 2119 | TACACCAACGGCATCGACATGAACGCGGTGCTGAAGGGCTTCCCTGAGTGCCTGCAGGCT |
| 2179 | GACATCTGCCTGCACCTGAACCGCTCACTGCTGCAGCACTGCAAACCCTTCCGAGGGGCC |
| 2239 | ACCAAGGGCTGCCTTCGGGCCCTGGCCATGAAGTTCAAGACCACACATGCACCGCCAGGG |
| 2299 | GACACACTGGTGCATGCTGGGGACCTGCTCACCGCCCTGTACTTCATCTCCCGGGGCTCC |
| 2359 | ATCGAGATCCTGCGGGGCGACGTCGTCGTGGCCATCCTGGGGAAGAATGACATCTTTGGG |
| 2419 | GAGCCTCTGAACCTGTATGCAAGGCCTGGCAAGTCGAACGGGGATGTGCGGGCCCTCACC |
| 2479 | TACTGTGACCTACACAAGATCCATCGGGACGACCTGCTGGAGGTGCTGGACATGTACCCT |
| 2539 | GAGTTCTCCGACCACTTCTGGTCCAGCCTGGAGATCACCTTCAACCTGCGAGATACCAAC |
| 2599 | ATGATCCCGGGCTCCCCCGGCAGTACGGAGTTAGAGGGTGGCTTCAGTCGGCAACGCAAG |
| 2659 | CGCAAGTTGTCCTTCCGCAGGCGCACGGACAAGGACACGGAGCAGCCAGGGGAGGTGTCG |
| 2719 | GCCTTGGGGCCGGGCCGGGCGGGGGCAGGGCCGAGTAGCCGGGGCCGGCCGGGGGGGCCG |
| 2779 | TGGGGGGAGAGCCCGTCCAGTGGCCCCTCCAGCCCTGAGAGCAGTGAGGATGAGGGCCCA |
| 2839 | GGCCGCAGCTCCAGCCCCCTCCGCCTGGTGCCCTTCTCCAGCCCCAGGCCCCCCGGAGAG |
| 2899 | CCGCCGGGTGGGGAGCCCCTGATGGAGGACTGCGAGAAGAGCAGCGACACTTGCAACCCC |
| 2959 | CTGTCAGGCGCCTTCTCAGGAGTGTCCAACATTTTCAGCTTCTGGGGGGACAGTCGGGGC |
| 3019 | CGCCAGTACCAGGAGCTCCCTCGATGCCCCGCCCCCACCCCCAGCCTCCTCAACATCCCC |
| 3079 | CTCTCCAGCCCGGGTCGGCGGCCCCGGGGCGACGTGGAGAGCAGGCTGGATGCCCTCCAG |
| 3139 | CGCCAGCTCAACAGGCTGGAGACCCGGCTGAGTGCAGACATGGCCACTGTCCTGCAGCTG |
| 3199 | CTACAGAGGCAGATGACGCTGGTCCCGCCCGCCTACAGTGCTGTGACCACCCCGGGGCCT |
| 3259 | GGCCCCACTTCCACATCCCCGCTGTTGCCCGTCAGCCCCCTCCCCACCCTCACCTTGGAC |
| 3319 | TCGCTTTCTCAGGTTTCCCAGTTCATGGCGTGTGAGGAGCTGCCCCCGGGGGCCCCAGAG |
| 3379 | CTTCCCCAAGAAGGCCCCACACGACGCCTCTCCCTACCGGGCCAGCTGGGGGCCCTCACC |
| 3439 | TCCCAGCCCCTGCACAGACACGGCTCGGACCCGGGCAGTTAG.................. |

viii. Protein Sequence of hERG1b

The protein sequence of hERG1b (SEQ ID NO:7) (NCBI Reference Sequence: NP_{—}742053.1) is:

MPVRRGHVAPQNTFLDTIIRKFEGQSRKFIIANARVENCAVIYCNDGFCELCGYSRAE
VMQRPCTCDFLHGPRTQRRAAAQIAQALLGAEERKVEIAFYRKDGSCFLCLVDVVPV
KNEDGAVIMFILNFEVVMEKDMVGSPAHDTNHRGPPTSWLAPGRAKTFRLKLPALL
ALTARESSVRSGGAGGAGAPGAVVVDVDLTPAAPSSESLALDEVTAMDNHVAGLGP
AEERRALVGPGSPPRSAPGQLPSPRAHSLNPDASGSSCSLARTRSRESCASVRRASSA
DDIEAMRAGVLPPPPRHASTGAMHPLRSGLLNSTSDSDLVRYRTISKIPQITLNFVDLK
GDPFLASPTSDREIIAPKIKERTHNVTEKVTQVLSLGADVLPEYKLQAPRIHRWTILHY
SPFKAVWDWLILLLVIYTAVFTPYSAAFLLKETEEGPPATECGYACQPLAVVDLIVDI
MFIVDILINFRTTYVNANEEVVSHPGRIAVHYFKGWFLIDMVAAIPFDLLIFGSGSEELI
GLLKTARLLRLVRVARKLDRYSEYGAAVLFLLMCTFALIAHWLACIWYAIGNMEQP
HMDSRIGWLHNLGDQIGKPYNSSGLGGPSIKDKYVTALYFTFSSLTSVGFGNVSPNTN
SEKIFSICVMLIGSLMYASIFGNVSAIIQRLYSGTARYHTQMLRVREFIRFHQIPNPLRQ
RLEEYFQHAWSYTNGIDMNAVLKGFPECLQADICLHLNRSLLQHCKPFRGATKGCLR
ALAMKFKTTHAPPGDTLVHAGDLLTALYFISRGSIEILRGDVVVAILGMGWGAGTGL
EMPSAASRGASLLNMQSLGLWTWDCLQGHWAPLIHLNSGPPSGAMERSPTWGEAA
ELWGSHILLPFRIRHKQTLFASLK.

ix. cDNA Sequence of hERG1b

The cDNA sequence of hERG1b (SEQ ID NO:8) is:

| | ...........................................ATGCCGGTGCGGAGGGG |
|---|---|
| 18 | CCACGTCGCGCCGCAGAACACCTTCCTGGACACCATCATCCGCAAGTTTGAGGGCCAGAG |
| 78 | CCGTAAGTTCATCATCGCCAACGCTCGGGTGGAGAACTGCGCCGTCATCTACTGCAACGA |
| 138 | CGGCTTCTGCGAGCTGTGCGGCTACTCGCGGGCCGAGGTGATGCAGCGACCCTGCACCTG |
| 198 | CGACTTCCTGCACGGGCCGCGCACGCAGCGCCGCGCTGCCGCGCAGATCGCGCAGGCACT |
| 258 | GCTGGGCGCCGAGGAGCGCAAAGTGGAAATCGCCTTCTACCGGAAAGATGGGAGCTGCTT |
| 318 | CCTATGTCTGGTGGATGTGGTGCCCGTGAAGAACGAGGATGGGGCTGTCATCATGTTCAT |
| 378 | CCTCAATTTCGAGGTGGTGATGGAGAAGGACATGGTGGGGTCCCCGGCTCATGACACCAA |
| 438 | CCACCGGGGCCCCCCCACCAGCTGGCTGGCCCCAGGCCGCGCCAAGACCTTCCGCCTGAA |
| 498 | GCTGCCCGCGCTGCTGGCGCTGACGGCCCGGGAGTCGTCGGTGCGGTCGGGCGGCGCGGG |
| 558 | CGGCGCGGGCGCCCCGGGGGCCGTGGTGGTGGACGTGGACCTGACGCCCGCGGCACCCAG |
| 618 | CAGCGAGTCGCTGGCCCTGGACGAAGTGACAGCCATGGACAACCACGTGGCAGGGCTCGG |
| 678 | GCCCGCGGAGGAGCGGCGTGCGCTGGTGGGTCCCGGCTCTCCGCCCCGCAGCGCGCCCGG |
| 738 | CCAGCTCCCATCGCCCCGGGCGCACAGCCTCAACCCCGACGCCTCGGGCTCCAGCTGCAG |
| 798 | CCTGGCCCGGACGCGCTCCCGAGAAAGCTGCGCCAGCGTGCGCCGCGCCTCGTCGGCCGA |
| 858 | CGACATCGAGGCCATGCGCGCCGGGGTGCTGCCCCCGCCACCGCGCCACGCCAGCACCGG |
| 918 | GGCCATGCACCCACTGCGCAGCGGCTTGCTCAACTCCACCTCGGACTCCGACCTCGTGCG |
| 978 | CTACCGCACCATTAGCAAGATTCCCCAAATCACCCTCAACTTTGTGGACCTCAAGGGCGA |
| 1038 | CCCCTTCTTGGCTTCGCCCACCAGTGACCGTGAGATCATAGCACCTAAGATAAAGGAGCG |
| 1098 | AACCCACAATGTCACTGAGAAGGTCACCCAGGTCCTGTCCCTGGGCGCCGACGTGCTGCC |
| 1158 | TGAGTACAAGCTGCAGGCACCGCGCATCCACCGCTGGACCATCCTGCATTACAGCCCCTT |
| 1218 | CAAGGCCGTGTGGGACTGGCTCATCCTGCTGCTGGTCATCTACACGGCTGTCTTCACACC |
| 1278 | CTACTCGGCTGCCTTCCTGCTGAAGGAGACGGAAGAAGGCCCGCCTGCTACCGAGTGTGG |
| 1338 | CTACGCCTGCCAGCCGCTGGCTGTGGTGGACCTCATCGTGGACATCATGTTCATTGTGGA |
| 1398 | CATCCTCATCAACTTCCGCACCACCTACGTCAATGCCAACGAGGAGGTGGTCAGCCACCC |
| 1458 | CGGCCGCATCGCCGTCCACTACTTCAAGGGCTGGTTCCTCATCGACATGGTGGCCGCCAT |
| 1518 | CCCCTTCGACCTGCTCATCTTCGGCTCTGGCTCTGAGGAGCTGATCGGGCTGCTGAAGAC |
| 1578 | TGCGCGGCTGCTGCGGCTGGTGCGCGTGGCGCGGAAGCTGGATCGCTACTCAGAGTACGG |
| 1638 | CGCGGCCGTGCTGTTCTTGCTCATGTGCACCTTTGCGCTCATCGCGCACTGGCTAGCCTG |
| 1698 | CATCTGGTACGCCATCGGCAACATGGAGCAGCCACACATGGACTCACGCATCGGCTGGCT |
| 1758 | GCACAACCTGGGCGACCAGATAGGCAAACCCTACAACAGCAGCGGCCTGGGCGGCCCCTC |
| 1818 | CATCAAGGACAAGTATGTGACGGCGCTCTACTTCACCTTCAGCAGCCTCACCAGTGTGGG |
| 1878 | CTTCGGCAACGTCTCTCCCAACACCAACTCAGAGAAGATCTTCTCCATCTGCGTCATGCT |
| 1938 | CATTGGCTCCCTCATGTATGCTAGCATCTTCGGCAACGTGTCGGCCATCATCCAGCGGCT |
| 1998 | GTACTCGGGCACAGCCCGCTACCACACACAGATGCTGCGGGTGCGGGAGTTCATCCGCTT |
| 2058 | CCACCAGATCCCCAATCCCCTGCGCCAGCGCCTCGAGGAGTACTTCCAGCACGCCTGGTC |
| 2118 | CTACACCAACGGCATCGACATGAACGCGGTGCTGAAGGGCTTCCCTGAGTGCCTGCAGGC |
| 2178 | TGACATCTGCCTGCACCTGAACCGCTCACTGCTGCAGCACTGCAAACCCTTCCGAGGGGC |
| 2238 | CACCAAGGGCTGCCTTCGGGCCCTGGCCATGAAGTTCAAGACCACACATGCACCGCCAGG |
| 2298 | GGACACACTGGTGCATGCTGGGGACCTGCTCACCGCCCTGTACTTCATCTCCCGGGGCTC |
| 2358 | CATCGAGATCCTGCGGGGCGACGTCGTCGTGGCCATCCTGGGTATGGGGTGGGGGGCGGG |
| 2418 | CACTGGACTGGAAATGCCCTCTGCAGCCTCAAGAGGTGCGAGCCTTCTGAATATGCAGTC |
| 2478 | ACTGGGGCTGTGGACCTGGGACTGCCTGCAGGGTCACTGGGCTCCTTTAATTCACCTAAA |
| 2538 | CTCAGGCCCTCCAAGCGGGGCCATGGAGAGGAGCCCCACGTGGGGTGAGGCTGCTGAACT |
| 2598 | CTGGGGTTCCCACATTCTCCTTCCCTTCAGGATCCGCCACAAACAGACACTTTTTGCTTC |
| 2658 | CTTAAAGTAG.................................................. |

### REFERENCES

1. US20070265418. He, M., Fused thiophenes, methods for making fused thiophenes, and uses thereof.
2. US20070161776. He, M., Fused thiophenes, methods for making fused thiophenes, and uses thereof
3. U.S. Pat. No. 6,393,190B1 He, M., Leslie, T. M. “Chromophores for polymeric thin films and optical waveguides and devices comprising the same”
4. B. F. O'Dowd, T. Nguyen, A. Marchese, R. Cheng, K. R. Lynch, H. H. Heng, L. F.
Kolakowski Jr, S. R. George (1998) Discovery of three novel G-protein-coupled receptor genes, Genomics 47: 310-313.
5. S. Okumura, H. Baba, T. Kumada, K. Nanmoku, H. Nakajima, Y. Nakane, K. Hioki, and K. Ikenaka. (2004) Cloning of a G-protein-coupled receptor that shows an activity to transform NIH3T3 cells and is expressed in gastric cancer cells. Cancer Sci, 95: 131-135.
6. J. Guo, D. J. Williams, H. L. Puhl III, S. R. Ikeda (2008) Inhibition of N-type calcium channels by activation of GPR35, an orphan receptor, heterologously expressed in rat sympathetic neurons, J. Pharmacol. Exp. Ther. 324: 342-351.
7. H. Ohshiro, H. Tonai-Kachi, K. Ichikawa (2008) GPR35 is a functional receptor in rat dorsal root ganglion neurons, Biochem. Biophys. Res. Commun. 365: 344-348.
8. A. E. Shrimpton, B. R. Braddock, L. L. Thomson, C. K. Stein, J. J. Hoo (2004) Molecular delineation of deletions on 2q37.3 in three cases with an Albright hereditary osteodystrophy-like phenotype, Clin. Genet. 66: 537-544.
9. K. D. Min, M. Asakura, Y. Liao, K. Nakamaru, H. Okazaki, T. Takahashi, K. Fujimoto, S. Ito, A. Takahashi, H. Asanuma, S. Yamazaki, T. Minamino, S. Sanada, O, Seguchi, A. Nakano, Y. Ando, T. Otsuka, H. Furukawa, T. Isomura, S. Takashima, N. Mochizuki, M. Kitakaze (2010) Identification of genes related to heart failure using global gene expression profiling of human failing myocardium, Biochem. Biophys. Res. Commun. 393: 55-60.
10. J. Wang, N. Simonavicius, X. Wu, G. Swaminath, J. Reagan, H. Tian, L. Ling, (2006) Kynurenic acid as a ligand for orphan G protein-coupled receptor GPR35, J. Biol. Chem. 281: 22021-22028.
11. S. Oka, R. Ota, M. Shima, A. Yamashita, T. Sugiura (2010) GPR35 is a novel lysophosphatidic acid receptor. Biochem. Biophys. Res. Comm. 395: 232-237. Z. Su, et al. Electrophysiologic characterization of a novel hERG channel activator. Biochem Pharm 77:1383, 2009.
12. M. Bridgland-Taylor, et al., J. Pharmacol. Toxicol. Methods 2006, 54(2), 189-99. A. Dubin, et al., J. Biomol. Screen. 2005, 10, (2), 168-81.
13. J. W. Warmke, J. W., et al. Proc. Natl. Acad. Sci. 1994. 91(8), 3438-3442.
14. H. Sun et al. J. Biol. Chem. 2006, 281:5877.
15. J. Takasaki, T. Saito, M. Taniguchi, T. Kawasaki, Y. Moritani, K. Hayashi and M. Kobori (2004) A Novel Gαq/11-selective Inhibitor. J. Biol. Chem. 279: 47438-47445.
B. R. Conklin, Z. Farfel, K. D. Lustig, D. Julius, and H. R. Bourne (1993) Substitution of three amino acids switches receptor specificity of Gq to that of Gi. Nature 1993, 363: 274-276.
16. J. Frey, A. D. Bond, A. B. Holmes. Improved synthesis of dithieno[3,2-b:2′,3′-d]thiophene (DTT) and derivatives for cross coupling. Chemical Communications 2002, 20, 2424-2425
17. Eur. Pat. Appl. 1995, EP 647874 A1. K.-Y. A. Jen, K. J. Drost. Preparation of polyimides having nonlinear optical properties.
18. U.S. Pat. No. 5,508,440A. C. T. Chang, C.-T. Lee, F. L. Lin (Hydroxymethyl)polythiophene derivatives useful for preventing inflammation and edema.

What is claimed is:

1. A compound or a pharmaceutically acceptable salt thereof, having a formula (I), (II) or (III): 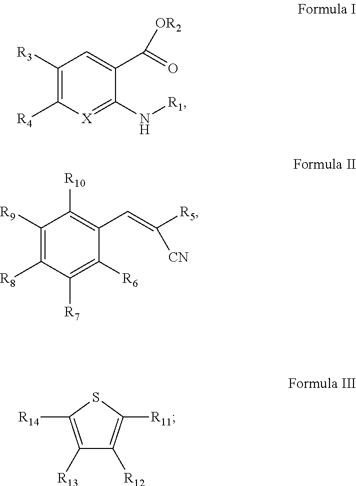
wherein:
X is C or N
R₁, R₂, R₃ and R₄ are each independently selected from a group consisting of hydrogen, halogen, cyano, —NO₂, —OR¹⁰¹, alkyl, alkenyl, alkynyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰²; or R₃ and R₄, together with the adjacent carbon atoms of the ring, form an fused or non-fused mono, bicyclic or tricyclic heterocyclic or carbocyclic ring which is optionally independently substituted with one or more substituents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², NR¹⁰¹S(O)₂R¹⁰², —NR¹⁰¹C(O)R¹⁰², S(O)₂R¹⁰², —SR¹⁰¹, —S(O)₂NR¹⁰¹R¹⁰², R¹⁰¹ and R¹⁰².
R₅ is CN, —C(O)NR¹⁰¹R¹⁰², —C(O)R¹⁰¹, —C(O)OR¹⁰¹, NR¹⁰¹R¹⁰², —NR¹⁰¹S(O)₂R¹⁰², NR¹⁰¹C(O)R¹⁰², —S(O)₂R¹⁰², —SR¹⁰¹, —S(O)NR¹⁰¹R¹⁰², R¹⁰¹, R¹⁰² or 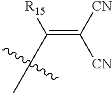
wherein R₁₅ is amino, alkylamino, dialkylamino, alkyl, hydroxy, cyano, or nitro;
R¹⁰¹ and R¹⁰² are each independently selected from the group consisting of hydrogen, alkyl, alkenyl, alkynyl, cycloalkyl, aryl, heterocycloalkyl and heteroaryl; wherein each R¹⁰¹ and R¹⁰² alkyl, alkenyl, alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;
R₆, R₇, R₈, R₉ and R₁₀ are each independently selected from a group consisting of hydrogen, halogen, cyano, —NO₂, —OR¹⁰¹, alkyl, alkenyl, alkynyl, cycloalkyl, cycloalkenyl, aryl, heterocycloalkyl, heteroaryl, —C(O)R¹⁰¹, —C(O)OR¹⁰¹, —C(O)R¹⁰¹, R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰²; or any two of R₆, R₇, R₈, R₉ and R₁₀, together with the adjacent carbon atoms of the phenyl ring, form an fused or non-fused mono, bicyclic or tricyclic heterocyclic or carbocyclic ring which is optionally independently substituted with one or more substituents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰².
R₁₁, R₁₂, R₁₃ and R₁₄ are each independently selected from a group consisting of hydrogen, halogen, cyano, —NO₂, —OR¹⁰¹, alkyl, alkenyl, alkynyl, cycloalkyl, cycloalkenyl, aryl, alkylaryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —S(O)₂NR¹⁰¹R¹⁰², R¹⁰¹ and R¹⁰²;
wherein each of R₁, R₂, R₃, R₄, R₅, R₆, R₇, R₈, R₉, R₁₀, R₁₁, R₁₂, R₁₃, R₁₄, R₁₅, R¹⁰¹ and R¹⁰² is optionally independently substituted with one or more substituents independently selected from the group consisting of hydrogen, halogen, cyano, —OR¹⁰¹, alkyl, alkenyl, cycloalkyl, cycloalkenyl, aryl, heterocycloalkyl, heteroaryl, —C(O)R¹⁰¹, —C(O)OR¹⁰¹, —C(O)NR¹⁰¹R¹⁰², —NR¹⁰¹R¹⁰², —NR¹⁰¹S(O)₂R¹⁰², —NR¹⁰¹C(O)R¹⁰², —S(O)₂R¹⁰², —SR¹⁰¹, —S(O)₂NR¹⁰¹R¹⁰², R¹⁰¹ and R¹⁰².

2. The compound of formula (I) according to claim 1, or a pharmaceutically acceptable salt thereof, wherein the compound of formula (I) is a compound selected from the group consisting of:

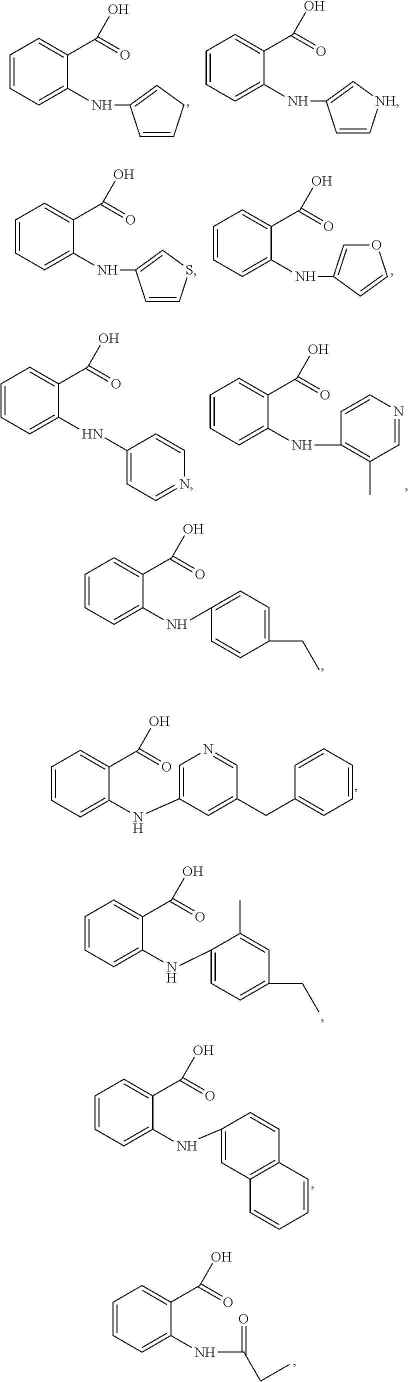

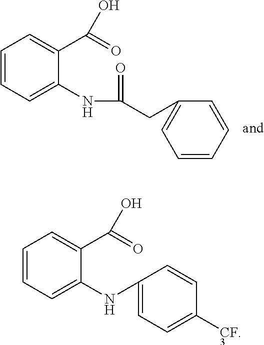

3. The compound of formula (II) according to claim 1, or a pharmaceutically acceptable salt thereof, wherein the compound of formula (II) is a compound selected from the group consisting of:

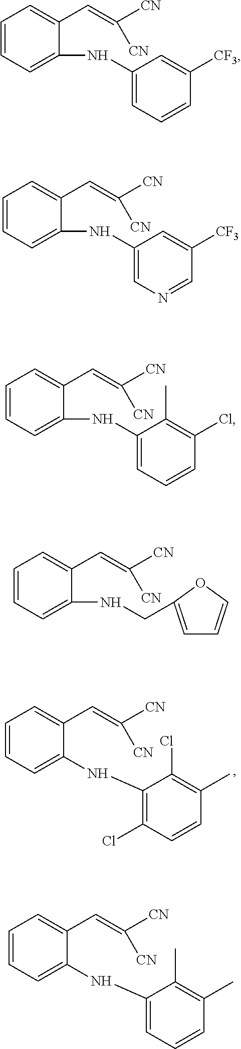

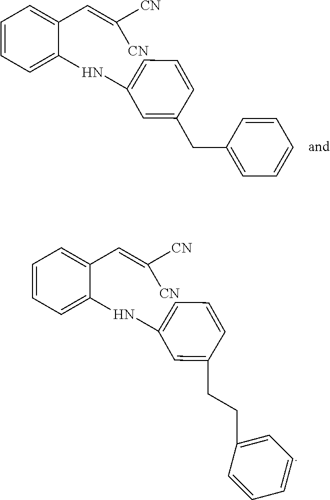

4. The compound of formula (III) according to claim 1, or a pharmaceutically acceptable salt thereof, wherein the compound of formula (III) is a compound selected from the group consisting of:

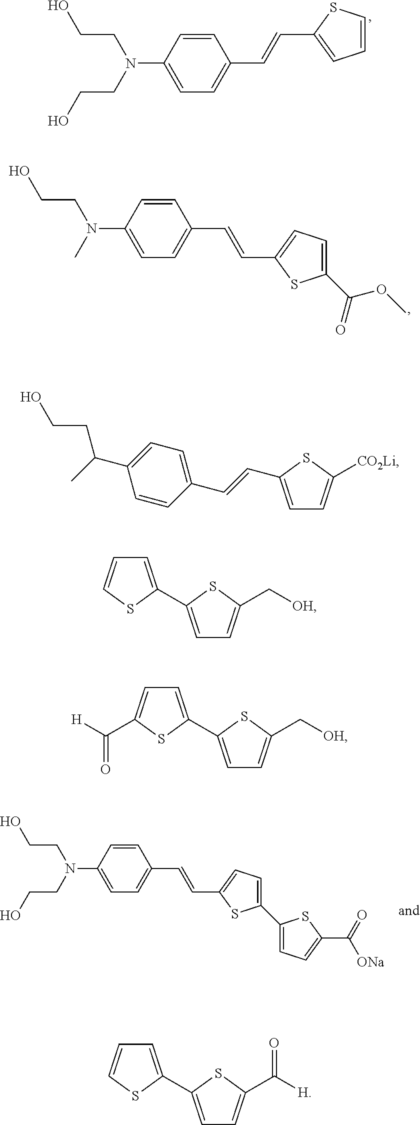

5. A method of screening for a hERG-specific modulator, comprising the steps of:
(a) incubating a compound individually with two different types of cells consisting of a cell expressing hERG and a cell without expressing hERG;
(b) monitoring the compound induced cellular response on each cell type with a label-free biosensor cellular assay;
(c) incubating a label-free biosensor hERG activator with the hERG expressing cell in the presence of the compound;
(d) monitoring the label-free biosensor hERG activator induced cellular response on the hERG expressing cell in the presence of the compound; and
(e) generating a biosensor index of the compound which indicates whether the compound is a hERG modulator or not.

6. A method of identifying GPR35-hERG complex interfering molecules comprising
a) contacting a composition comprising a GPR35-hERG complex with a test agent; and
b) assaying for GPR35-hERG interaction

7. A method of screening a GPR35-specific modulator, comprising the steps of:
(a) providing a cell that express GPR35;
(b) contacting said cell with a compound; and
(c) profiling said compound using a label-free biosensor cellular assay.

8. The method of claim 7, wherein said compound is a GPR35 modulator having a chemical structure selected from the group consisting of:

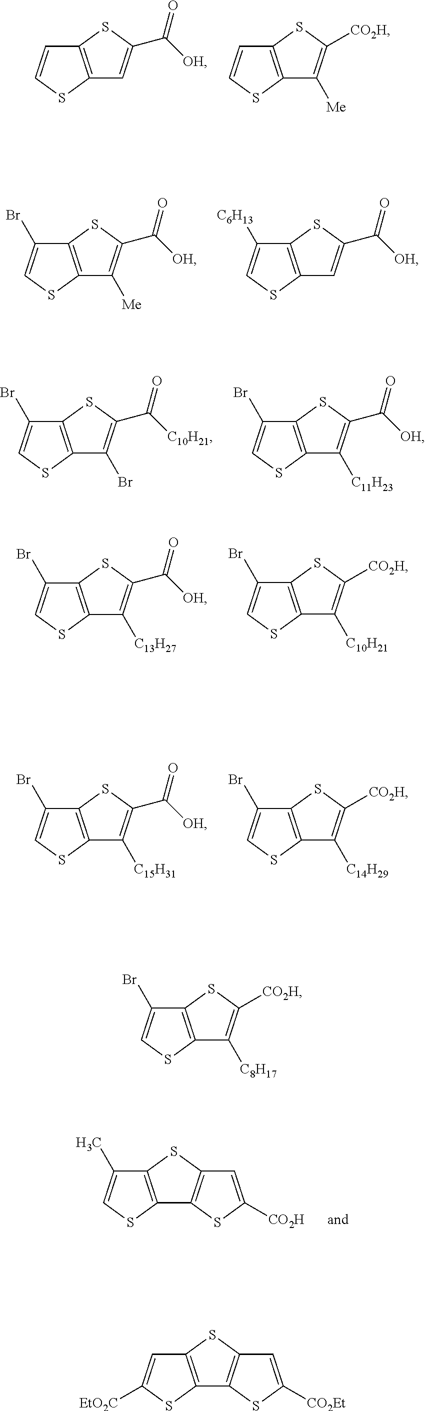

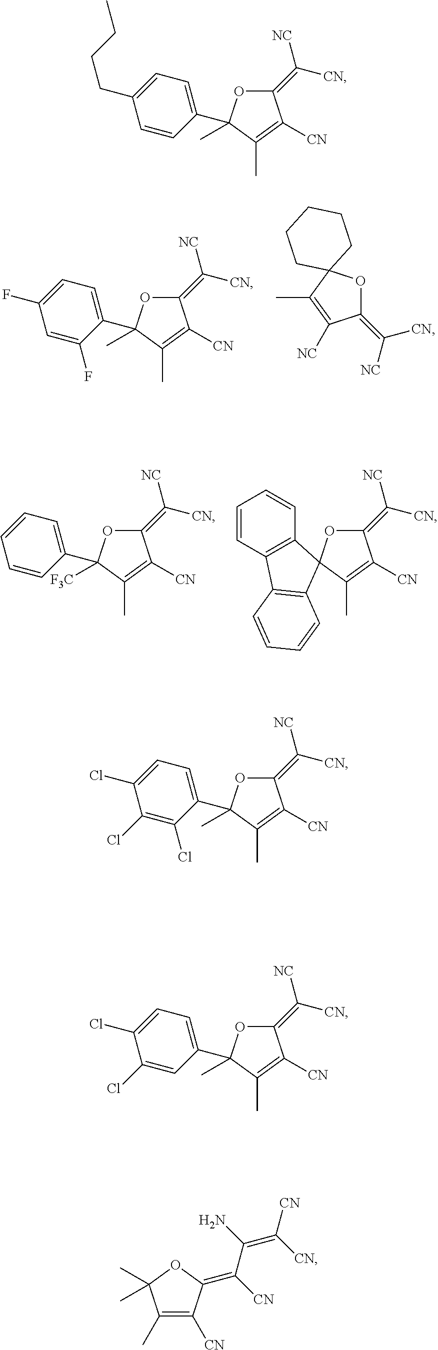

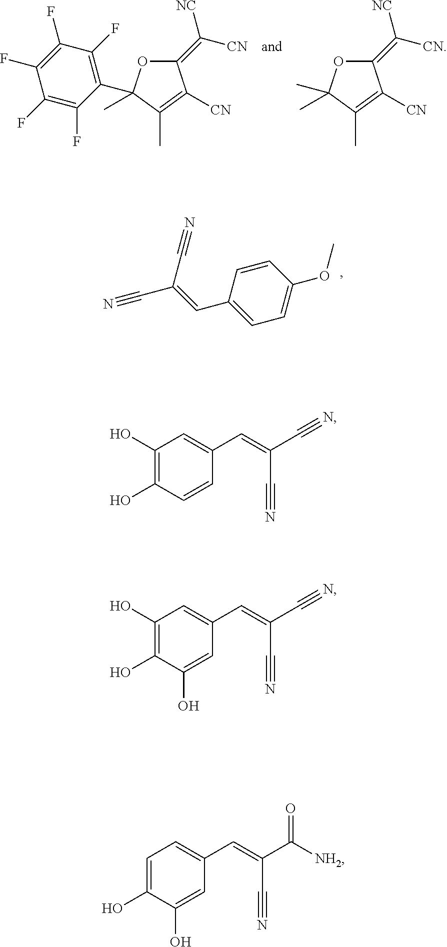

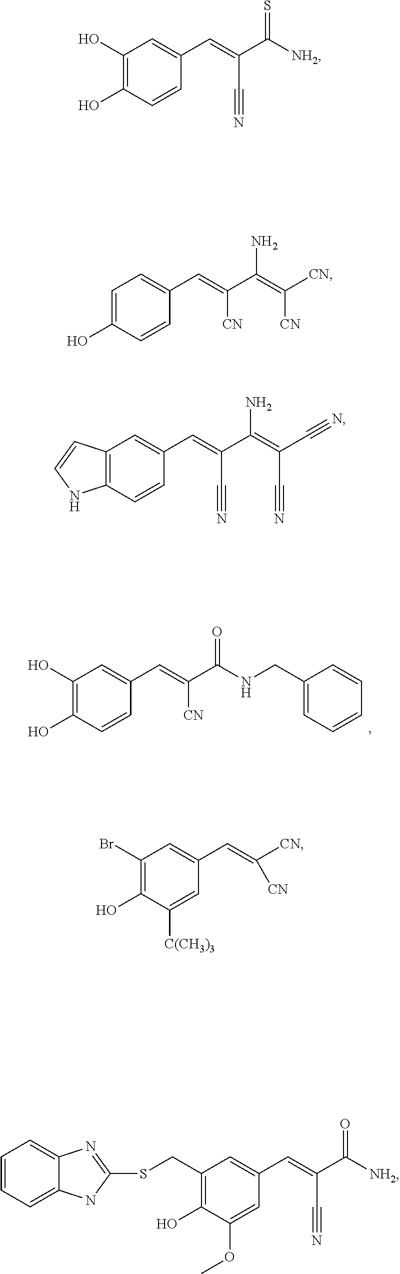

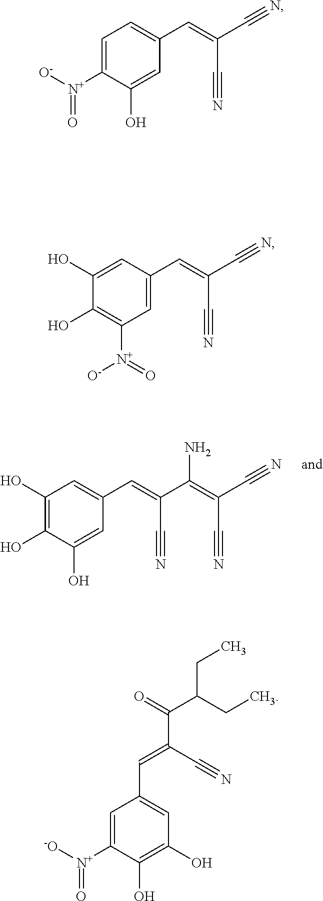

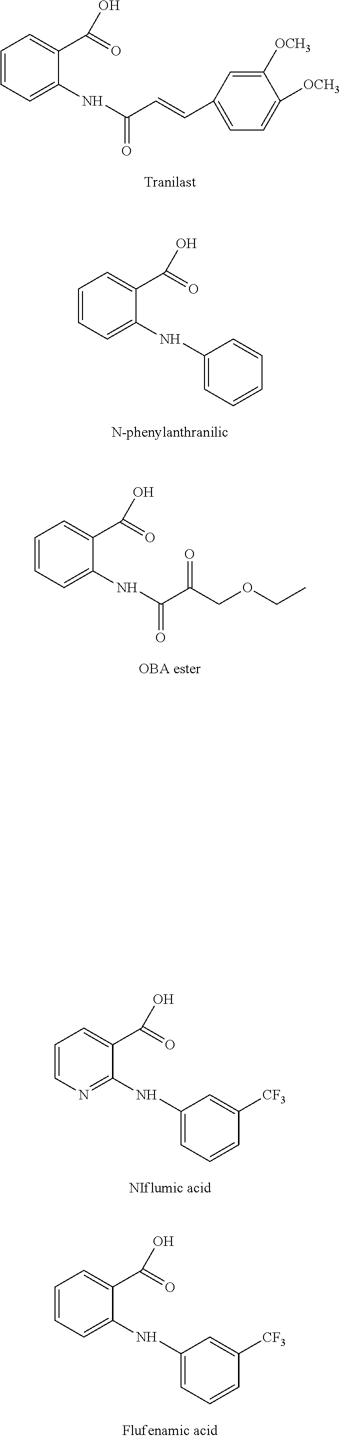

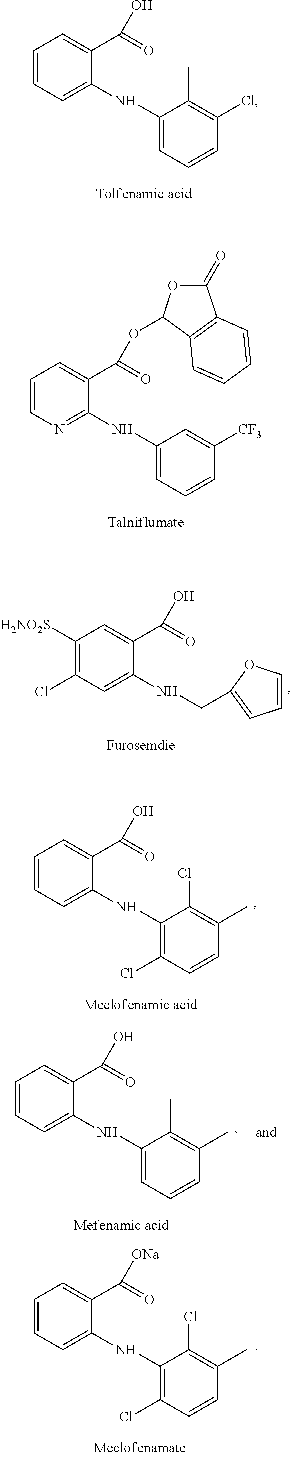

9. A method of preventing and/or treating a subject, comprising administering to said subject a therapeutically effective amount of a compound of formula (I), (II) or (III) according to claim 1, or a pharmaceutically acceptable salt thereof, wherein the subject has a disease which is pathophysiologically related to GPR35.

10. A method of screening for a GPR35-hERG signaling complex modulator, comprising the steps of:
(a) determining if a compound is a GPR35-specific modulator or a hERG-specific modulator or neither; and
(b) determining if said compound is a GPR35-hERG signaling complex modulator.

11. The method of claim 10, wherein step (b) comprises:
(i) providing a cell comprising GPR35-hERG complex;
(ii) contacting said cell with said compound; and
(iii) profiling said compound by using one or more suitable assays.

12. The method of claim 10, wherein said compound is a GPR35-hEGR signaling complex modulator having a chemical structure selected from the group consisting of:

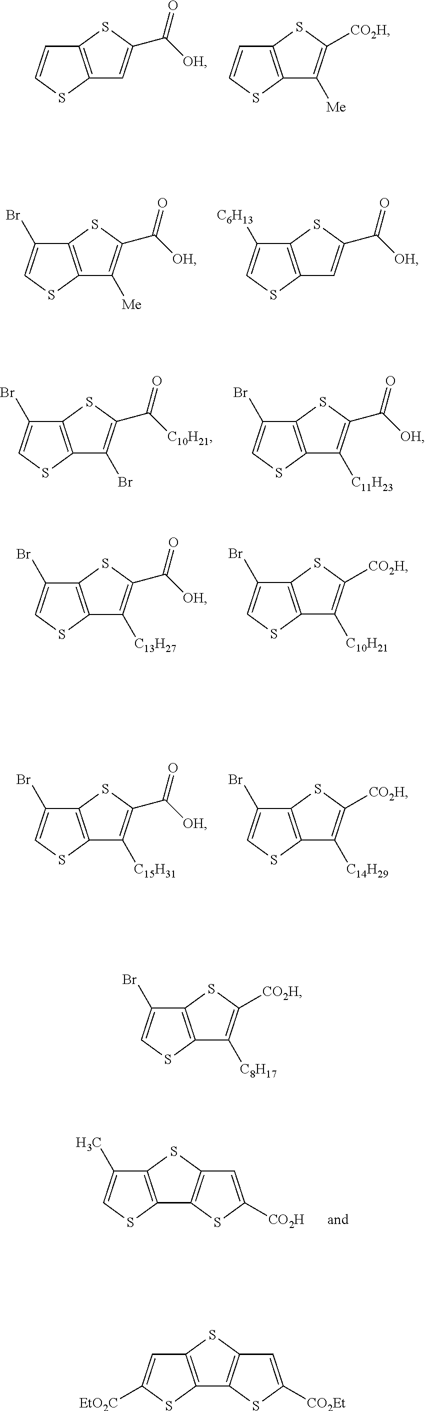

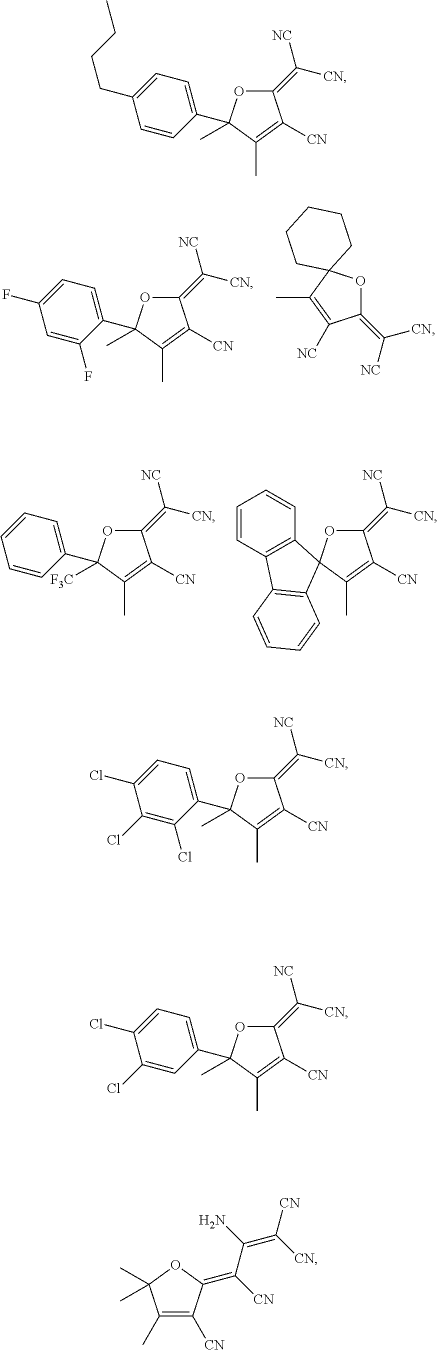

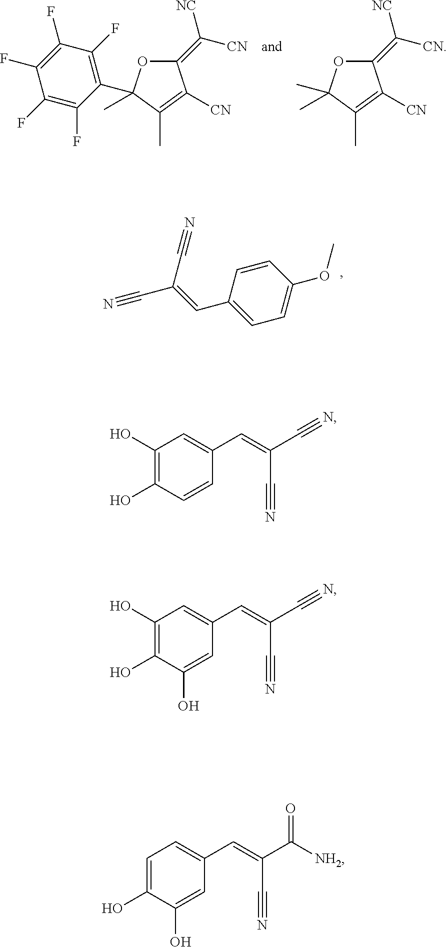

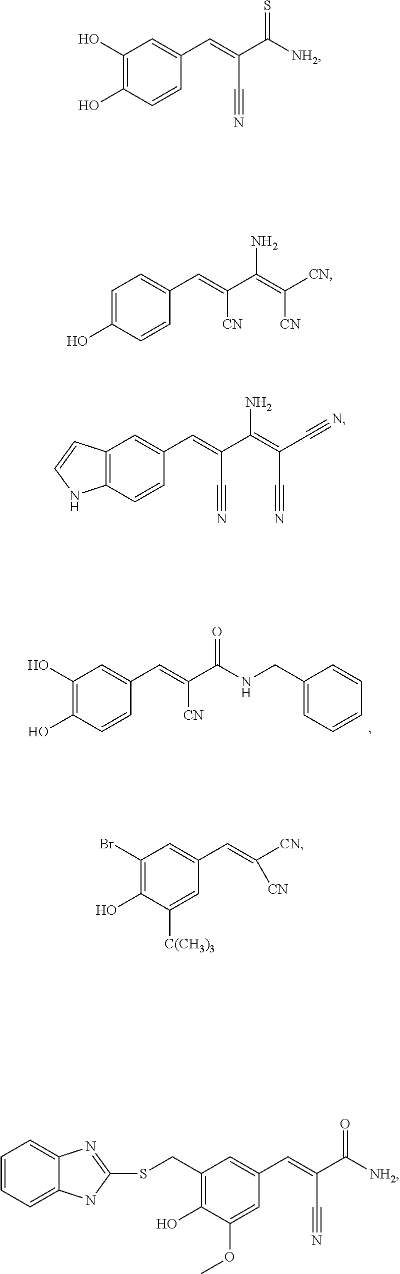

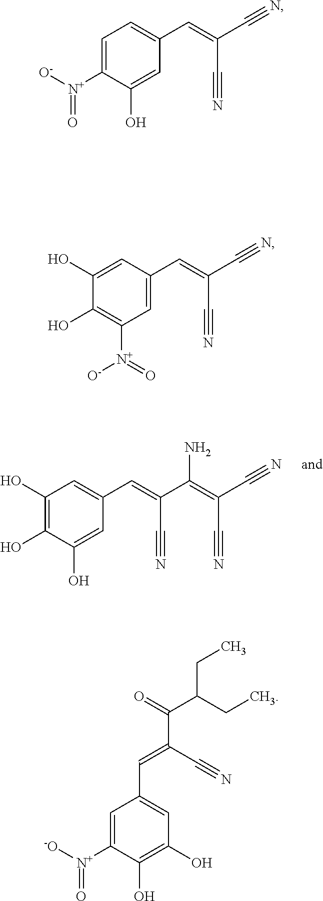

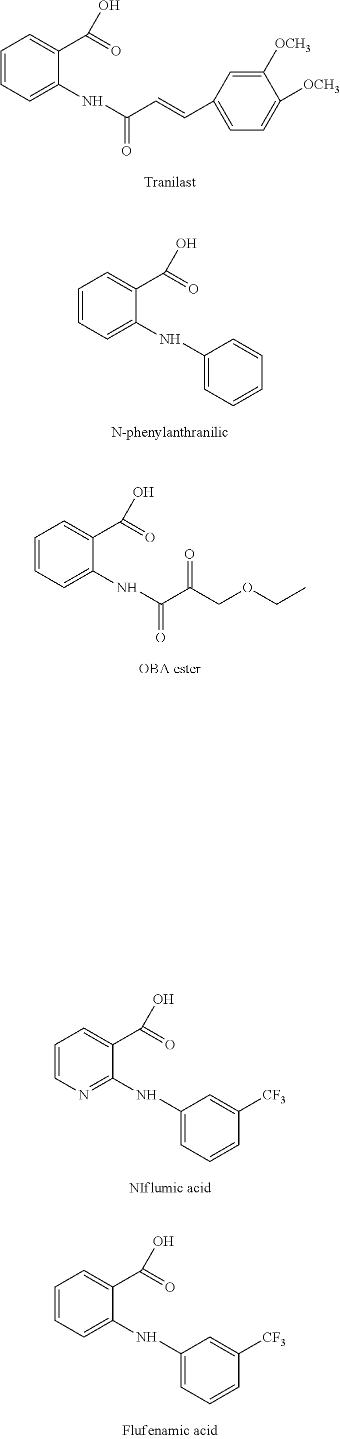

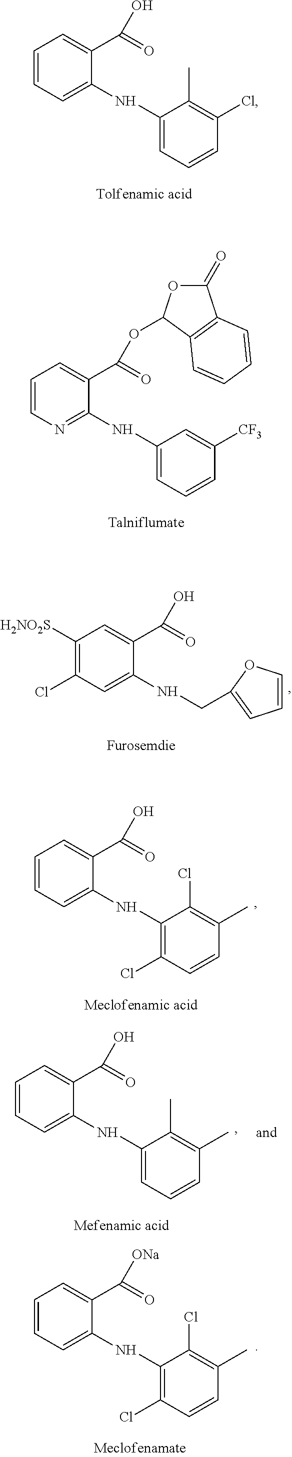

13. A method of preventing and/or treating a subject, comprising administering to said subject a therapeutically effective amount of a compound of formula (I), (II) or (III) according to any one of claim 1, or a pharmaceutically acceptable salt thereof, wherein the subject has a disease which is pathophysiologically related to GPR35-hEGR signaling complex.

14. A pharmaceutical composition for preventing and/or treating a subject comprising a therapeutically effective amount of a compound of formula (I), (II) or (III) according to any one of claim 1, or a pharmaceutically acceptable salt thereof.

15. An engineered cell comprising an exogenous GPR35 gene and an exogenous hERG gene.

16. An engineered cell comprising an exogenous GPR35 gene and endogenous hERG gene.

17. An isolated G-protein coupled receptor (GPCR)-hERG complex comprising one or more GPCRs and hERG.

18. A method of identifying GPR35-hERG complex binding molecules comprising
a) contacting the composition of claim 17 with a test molecule; and
b) determining if the test molecule binds to the GPR35-hERG complex.

19. A kit comprising a GPR35-hERG expressing engineered cell line and instructions for handling the cell line.

20. A method of treating a subject comprising administering to said subject a therapeutically effective amount of a molecule identified in claim 17, wherein the subject has a disease which is pathophysiologically related to the GPR35-hERG complex.


SEQUENCE LISTING (9 sequences)

SEQ ID NO: 1 - DNA, 1875 bp, Homo sapiens
  mRNA (1)..(1875)
caggccagagtcccagctgtcctggactctgctgtggggaagggctgatgcaggtgtggagtcaaatgtgggtgcctcctgcagccgggtgccaggaggggtggaggggccaccctgggctttgtccgggagcctggtcttcccgtccttgggctgacaggtgctgctgcctctgagccctccctgctaagagctgtgtgctgggtaaggctggtggccctttgggctccctgtccaggatttgtgctctggagggtagggcttgctgggctggggactggaggggaacgtggagctccttctgcctcctttcctgccccatgacagcaggcagatcccaggagagaagagctcaggagatgggaagaggatctgtccaggggttagacctcaagggtgacttggagttctttacggcacccatgctttctttgaggagttttgtgtttgtgggtgtggggtcggggctcacctcctcccacatccctgcccagaggtgggcagagtgggggcagtgccttgctccccctgctcgctctctgctgacctccggctccctgtgctgccccaggaccatgaatggcacctacaacacctgtggctccagcgacctcacctggcccccagcgatcaagctgggcttctacgcctacttgggcgtcctgctggtgctaggcctgctgctcaacagcctggcgctctgggtgttctgctgccgcatgcagcagtggacggagacccgcatctacatgaccaacctggcggtggccgacctctgcctgctgtgcaccttgcccttcgtgctgcactccctgcgagacacctcagacacgccgctgtgccagctctcccagggcatctacctgaccaacaggtacatgagcatcagcctggtcacggccatcgccgtggaccgctatgtggccgtgcggcacccgctgcgtgcccgcgggctgcggtcccccaggcaggctgcggccgtgtgcgcggtcctctgggtgctggtcatcggctccctggtggctcgctggctcctggggattcaggagggcggcttctgcttcaggagcacccggcacaatttcaactccatggcgttcccgctgctgggattctacctgcccctggccgtggtggtcttctgctccctgaaggtggtgactgccctggcccagaggccacccaccgacgtggggcaggcagaggccacccgcaaggctgcccgcatggtctgggccaacctcctggtgttcgtggtctgcttcctgcccctgcacgtggggctgacagtgcgcctcgcagtgggctggaacgcctgtgccctcctggagacgatccgtcgcgccctgtacataaccagcaagctctcagatgccaactgctgcctggacgccatctgctactactacatggccaaggagttccaggaggcgtctgcactggccgtggctcccagtgctaaggcccacaaaagccaggactctctgtgcgtgaccctcgcctaagaggcgtgctgtgggcgctgtgggccaggtctcgggggctccgggaggtgctgcctgccaggggaagctggaaccagtagcaaggagcccgggatcagccctgaactcactgtgtattctcttggagccttgggtgggcagggacggcccaggtacctgctctcttgggaagagagagggacagggacaagggcaagaggactgaggccagagcaaggccaatgtcagagacccccgggatggggcctcacacttgccacccccagaaccagctcacctggccagagtgggttcctgctggccagggtgcagccttgatgacacctgccgctgcccctcggggctggaataaaactccccacccagagtc

SEQ ID NO: 2 - DNA, 930 bp, Homo sapiens
  mRNA (1)..(930)
atgaatggcacctacaacacctgtggctccagcgacctcacctggcccccagcgatcaagctgggcttctacgcctacttgggcgtcctgctggtgctaggcctgctgctcaacagcctggcgctctgggtgttctgctgccgcatgcagcagtggacggagacccgcatctacatgaccaacctggcggtggccgacctctgcctgctgtgcaccttgcccttcgtgctgcactccctgcgagacacctcagacacgccgctgtgccagctctcccagggcatctacctgaccaacaggtacatgagcatcagcctggtcacggccatcgccgtggaccgctatgtggccgtgcggcacccgctgcgtgcccgcgggctgcggtcccccaggcaggctgcggccgtgtgcgcggtcctctgggtgctggtcatcggctccctggtggctcgctggctcctggggattcaggagggcggcttctgcttcaggagcacccggcacaatttcaactccatggcgttcccgctgctgggattctacctgcccctggccgtggtggtcttctgctccctgaaggtggtgactgccctggcccagaggccacccaccgacgtggggcaggcagaggccacccgcaaggctgcccgcatggtctgggccaacctcctggtgttcgtggtctgcttcctgcccctgcacgtggggctgacagtgcgcctcgcagtgggctggaacgcctgtgccctcctggagacgatccgtcgcgccctgtacataaccagcaagctctcagatgccaactgctgcctggacgccatctgctactactacatggccaaggagttccaggaggcgtctgcactggccgtggctcccagtgctaaggcccacaaaagccaggactctctgtgcgtgaccctcgcctaa

SEQ ID NO: 3 - protein, 340 aa, Homo sapiens [sequence omitted]

SEQ ID NO: 4 - DNA, 1023 bp, Homo sapiens
  mRNA (1)..(1023)
atgctgagtggttcccgggctgtccccactccacaccgtggcagtgaagagctgctgaagtacatgcttcatagtccttgcgtctctctgaccatgaatggcacctacaacacctgtggctccagcgacctcacctggcccccagcgatcaagctgggcttctacgcctacttgggcgtcctgctggtgctaggcctgctgctcaacagcctggcgctctgggtgttctgctgccgcatgcagcagtggacggagacccgcatctacatgaccaacctggcggtggccgacctctgcctgctgtgcaccttgcccttcgtgctgcactccctgcgagacacctcagacacgccgctgtgccagctctcccagggcatctacctgaccaacaggtacatgagcatcagcctggtcacggccatcgccgtggaccgctatgtggccgtgcggcacccgctgcgtgcccgcgggctgcggtcccccaggcaggctgcggccgtgtgcgcggtcctctgggtgctggtcatcggctccctggtggctcgctggctcctggggattcaggagggcggcttctgcttcaggagcacccggcacaatttcaactccatggcgttcccgctgctgggattctacctgcccctggccgtggtggtcttctgctccctgaaggtggtgactgccctggcccagaggccacccaccgacgtggggcaggcagaggccacccgcaaggctgcccgcatggtctgggccaacctcctggtgttcgtggtctgcttcctgcccctgcacgtggggctgacagtgcgcctcgcagtgggctggaacgcctgtgccctcctggagacgatccgtcgcgccctgtacataaccagcaagctctcagatgccaactgctgcctggacgccatctgctactactacatggccaaggagttccaggaggcgtctgcactggccgtggctcccagtgctaaggcccacaaaagccaggactctctgtgcgtgaccctcgcctaa

SEQ ID NO: 5 - protein, 1159 aa, Homo sapiens
  PEPTIDE (1)..(1159)
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 6 - DNA, 3480 bp, Homo sapiens
  mRNA (1)..(3480)
atgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttag

SEQ ID NO: 7 - protein, 888 aa, Homo sapiens
  PEPTIDE (1)..(888)
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGMGWGAGTGLEMPSAASRGASLLNMQSLGLWTWDCLQGHWAPLIHLNSGPPSGAMERSPTWGEAAELWGSHILLPFRIRHKQTLFASLK

SEQ ID NO: 8 - DNA, 2667 bp, Homo sapiens
  mRNA (1)..(2667)
atgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctgggtatggggtggggggcgggcactggactggaaatgccctctgcagcctcaagaggtgcgagccttctgaatatgcagtcactggggctgtggacctgggactgcctgcagggtcactgggctcctttaattcacctaaactcaggccctccaagcggggccatggagaggagccccacgtggggtgaggctgctgaactctggggttcccacattctccttcccttcaggatccgccacaaacagacactttttgcttccttaaagtag

SEQ ID NO: 9 - protein, 309 aa, Homo sapiens
  PEPTIDE (1)..(309)
MNGTYNTCGSSDLTWPPAIKLGFYAYLGVLLVLGLLLNSLALWVFCCRMQQWTETRIYMTNLAVADLCLLCTLPFVLHSLRDTSDTPLCQLSQGIYLTNRYMSISLVTAIAVDRYVAVRHPLRARGLRSPRQAAAVCAVLWVLVIGSLVARWLLGIQEGGFCFRSTRHNFNSMAFPLLGFYLPLAVVVFCSLKVVTALAQRPPTDVGQAEATRKAARMVWANLLVFVVCFLPLHVGLTVRLAVGWNACALLETIRRALYITSKLSDANCCLDAICYYYMAKEFQEASALAVAPSAKAHKSQDSLCVTLA